(19) United States
(12) Patent Application Publication
(10) Pub. No.: US 2026/0315617 A1
(43) Pub. Date: Oct. 15, 2026

(54) NOVEL HYDROLYZABLE LINKER COMPOUNDS FOR ANTIBODY-DRUG CONJUGATES AND USES THEREOF

(71) Applicant: Bristol-Myers Squibb Company, Princeton, NJ (US)

(72) Inventors: Yam Bahadur POUDEL, Fremont, CA (US); Eugene Lvovich Piatnitski CHEKLER, Los Gatos, CA (US); Sayumi YAMAZOE, Belmont, CA (US); Ming Yao ZHANG, San Mateo, CA (US)

(73) Assignee: Bristol-Myers Squibb Company, Princeton, NJ (US)

(21) Appl. No.: 19/541,826

(22) Filed: Feb. 17, 2026

Related U.S. Application Data
(60) Provisional application No. 63/759,871, filed on Feb. 18, 2025.

(51) Int. Cl. A61K 47/68; A61K 31/519; C07D 207/452; C07D 403/06; C07D 471/10; C07D 487/04; C07D 487/08; C07D 487/10; C07K 16/18

(52) U.S. Cl. CPC A61K 47/6889; A61K 31/519; A61K 47/68031; A61K 47/68037; A61K 47/6843; C07D 207/452; C07D 403/06; C07D 471/10; C07D 487/04; C07D 487/08; C07D 487/10; C07K 16/18

(57) ABSTRACT

Antibody-drug conjugates (ADCs) are gradually becoming more relevant and effective in clinical cancer therapy. The ADC linker molecule in the ADC helps define the efficacy and the adverse effects of the ADC. A preferred linker should be stable in the vial and in the subject, while cleaving, and thus releasing, the cytotoxic payload in the desired tumor. However, many existing linkers may release payloads prematurely or off target and lead to toxicity in undesired cells. The pursuit of ADCs and the discovery and development of novel linkers, such as the novel hydrolyzable linker-payload compounds in the present invention, helps stabilize the ADC and provide improved targeted delivery of the payload.

### CROSS-REFERENCE TO RELATED APPLICATIONS

This application claims the benefit under 35 U.S.C. § 119(e) of U.S. Provisional Application Ser. No. 63/759,871, filed Feb. 18, 2025; the disclosure of which is incorporated herein by reference.

### SEQUENCE LISTING

The instant application contains a Sequence Listing which has been submitted electronically in XML format and is hereby incorporated by reference in its entirety. Said XML copy, created Jan. 15, 2026; is named 20250218_SEQL_14940USPSP_JR.xml and is 15,866 bytes in size.

### FIELD

The present application relates to novel hydrolyzable linker-payload compounds for use in antibody-drug conjugates (ADCs), and methods of making and using the hydrolyzable linker-payload compounds and ADCs. The present application relates to antibody-drug conjugates comprising a hydrolyzable linker-payload compound which can be used with various drugs and antibodies. For exemplary purposes, the present application also relates to hydrolyzable linker-drug compounds comprising TLR7/8 agonists useful in antibody-drug conjugates.

### BACKGROUND

There exists a need for safe and effective agents and methods for treating, preventing and managing cancer, including for cancers that are refractory to standard treatments, while reducing or avoiding the toxicities and/or side effects associated with some existing therapies.

Antibody-drug conjugates (ADCs) has been conceived and developed Wang, Z., Li, H., Gou, L., Wei, L., Wang, Y. Acta Pharmaceutica Sinica B 2023; 13(10):4025-4059. Typically, an ADC is formed by an antibody targeting a tumor-specific antigen or a related antigen and several payloads through appropriate linkers. An ADC combines the high targeting capabilities of a monoclonal antibody (mAb) and the high potency of the payloads in tumor tissues. ADCs have become one of the fastest-developing drug classes in oncology in recent years, because of their lower side effects, broader treatment applications, and higher therapeutic index.

Linkers affect the stability, toxicity, pharmacokinetics, and pharmacodynamics of ADCs, so careful selection of appropriate linkers is crucial in ADC design. The development of new linkers is steadily progressing, further enriching ADC design strategies to enhance their clinical value in the future.

The present disclosure provides novel hydrolyzable linkers for antibody-drug conjugates (ADCs), which stabilizes the ADC and provides improved targeted delivery of the payload.

The linker connects the antibody and the cytotoxic payload and is a key component in the function of ADCs. Su, Z., Xiao, D., Xie, F., Liu, L., Wang, Y., Fan, S., Zhou, X., Li, S. Acta Pharmaceutica Sinica B 2021; 11(12):3889-3907. The linker imparts the following characteristics to ADCs: (1) high stability in the circulation, and (2) specific release of payload in the target tissue. (Id. at 3890) These seemingly contradictory requirements of stability and release lead to the major challenge in the development of linkers. (Id.)

Over 80% of FDA-approved ADCs use maleimide-thiol reactions during their construction. The thiosuccinimide linkage is susceptible to ring-opening hydrolysis and retro-Michael deconjugation. The ring-opened product is resistant to thiol exchange reactions.

However, depending on the structure of the ADC construct, thiosuccinimide ADCs display slow hydrolysis, typically requiring heating the ADCs in a basic buffer, which results in an increased risk of deconjugation and denaturation of the final product.

Identifying a linker-drug that hydrolyzes upon reaction with the antibody without requiring heat or basic conditions would facilitate the generation of ADCs that are resistant to thiol-exchange reactions occurring in serum.

### SUMMARY

The present disclosure relates to a hydrolyzable linker-payload compounds and antibody-drug conjugates (ADCs) for use with a wide range of antibodies and payloads. The present disclosure relates to hydrolyzable linker-payload compounds comprising TLR7/8 agonists useful in antibody-drug conjugates. For example, embodiments of the disclosure include the following:

A compound of any of the structural formulas depicted herein (e.g., Structural Formula I, or II), or a pharmaceutically acceptable salt thereof, wherein values for the variables are as described herein.

A hydrolyzable linker-payload compound refers to a compound of Formula (I):

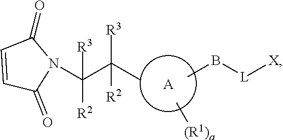

or a pharmaceutically acceptable salt thereof,
wherein:
A is selected from an unsubstituted or substituted 4 to 12 membered heterocyclic or heterospirocyclic ring;
B is independently selected from a bond, —CH₂—, —C═O—, —O—, —(—(CR¹¹R¹²)ₙ—)—O)ₘ—, —C═O—(CR¹¹R¹²)ₙ—O—)ₘ—, —C═O—(NR¹¹)—, —S—, —SO—, —SO₂—, or —SO₂—(NR¹¹)—;
R¹ is independently selected from hydrogen, halogen, —OR¹¹, —N(R¹¹R¹²)—NHR¹¹, —(—(CH₂)₂—)ₙ-O—)ₘ, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —N(R¹¹R¹²)—NHR¹¹ or —OR¹¹;
R² is independently selected from hydrogen, halogen, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —NHR¹¹ or —OR¹¹;
R³ is independently selected from hydrogen, halogen, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —NHR¹¹ or —OR¹¹;
wherein two of any R¹, R² or R³ substituents together with the carbon atoms they are attached to, may join to form a 5 or 6 membered ring that may be unsaturated, saturated, partially saturated;
and may further optionally be substituted with 1 or 2 R¹¹ substituents;
R¹¹ is independently selected from hydrogen, halogen, —C₁-C₆ alkyl, —C₂-C₆ alkenyl, —C₁-C₆ haloalkyl, —(—(CH₂)ₙ—)—O—)ₘ, a 3 to 12 membered cycloalkyl, a 4 to 12 membered heterocyclic, a 5 to 12 membered aryl or a 5 to 12 membered heteroaryl ring;
R¹² is independently selected from hydrogen, halogen, —C₁-C₆ alkyl, —C₂-C₆ alkenyl, —C₁-C₆ haloalkyl, —(—(CH₂)ₙ—)—O—)ₘ, a 3 to 12 membered cycloalkyl, a 4 to 12 membered heterocyclic, a 5 to 12 membered aryl or a 5 to 12 membered heteroaryl ring;
wherein the alkyl, alkenyl, haloalkyl, cycloalkyl, heterocyclic, aryl or heteroaryl rings in R¹¹ and R¹² are each independently unsubstituted or substituted with 1, 2, or 3 R¹³ substituents;
R¹³ is independently hydrogen, halo, —C₁-C₆ alkyl, —C₁-C₆ haloalkyl, —C₁-C₆ alkoxyalkyl, oxo, —CN, —NR¹⁴R¹⁴, —CH₃—NH—CH₂—C(═O)—OH, hydroxyl or —C₁-C₆ alkoxy;
R¹⁴ is independently hydrogen, —C₁-C₆ alkyl, —C₁-C₆ haloalkyl, or —C₁-C₆ alkoxyalkyl;
L is a linker;
X is a payload;
a is 0, 1, 2, 3, or 4;
m is an integer selected from 0 to 36;
n is 1, 2, 3, 4, 5, 6, 7, 8, 9, 10, 11 or 12; and further
wherein the heterocyclic and heteroaryl cyclic ring in each A, R¹, R², R³, R¹¹, and R¹² may include 1, 2 or 3 heteroatoms independently selected from O, N or S.

An antibody-drug conjugate comprising an antibody, or antigen-binding portion thereof, described herein and a cytotoxic agent.

An antibody-drug conjugate of any of the structural formulas depicted herein (e.g., Structural Formula I and XVII), or a pharmaceutically acceptable salt thereof.

A pharmaceutical composition comprising (i) a compound or antibody-drug conjugate and (ii) a pharmaceutically acceptable carrier.

A method of treating cancer in a subject in need thereof, comprising administering to the subject a therapeutically effective amount of an antibody-drug conjugate described herein, or a pharmaceutical composition of either of the foregoing.

Use of an antibody-drug conjugate described herein, or a pharmaceutical composition thereof, for the preparation of a medicament for treating cancer in a subject in need thereof.

An antibody-drug conjugate described herein, or a pharmaceutical composition thereof, for use in treating cancer in a subject in need thereof.

It is to be understood that both the foregoing general description and the following detailed description are exemplary and explanatory only and are not restrictive of the claims. All references cited herein are incorporated in their entirety by reference.

### BRIEF DESCRIPTION OF THE DRAWING

FIG. 1 depicts the enhanced stability of the hydrolyzable linker-payload compound due to ring opening hydrolysis.

### DETAILED DESCRIPTION OF THE INVENTION

### I. Definitions

In order that the present disclosure may be more readily understood, certain terms are first defined. Unless otherwise defined, scientific and technical terms used in connection with the present invention shall have the meanings that are commonly understood by those of ordinary skill in the art. Further, unless otherwise required by context, singular terms shall include pluralities, and plural terms shall include the singular.

The following abbreviations may be used herein:

TABLE 1
Abbreviations

| | |
|---|---|
| AcOH | acetic acid |
| aq or aq. | Aqueous |
| BOC or Boc | tert-butyloxy carbonyl |
| cpme | cyclopentyl methyl ether |
| DCE | 1,2-dichloroethane |
| DABCO | 1,4-diazabicyclo[2.2.2]octane |
| DCM | Dichloromethane |
| DMA | N,N-Dimethylacetamide |
| DMAP | 4-dimethylaminopyridine |
| DME | 1,2-dimethoxyethane |
| DMF | N,N-dimethylformamide |
| DMSO | dimethyl sulfoxide |
| Dppf, DPPF or dppf | 1,1′-bis(diphenylphosphino)ferrocene |
| eq or eq. or equiv. | Equivalent |
| ESI or ES | electrospray ionization |
| Et | Ethyl |
| Et₂O | diethyl ether |
| EtOAc | ethyl acetate |
| g | Grams |
| h | Hour |
| HPLC | high pressure liquid chromatography |
| iPr | Isopropyl |
| iPr₂NEt or DIPEA | N-ethyl diisopropylamine (Hünig's base) |
| KHMDS | potassium hexamethyldisilazide |
| KOAc | potassium acetate |
| Lawesson's reagent | 2,4-bis(4-methoxyphenyl)-2,4-dithioxo-1,3,2,4- |
| | dithiadiphosphetane, 2,4-Bis-(4-methoxyphenyl)-1,3- |
| | dithia-2,4-diphosphetane 2,4-disulfide |
| LC MS, LCMS, LC-MS or | liquid chromatography mass spectroscopy |
| LC/MS | |
| LG | Leaving group (e.g., halogen, mesylate, triflate) |
| LHMDS or LiHMDS | lithium hexamethyldisilazide |
| m/z | mass divided by charge |
| Me | Methyl |
| MeCN | Acetonitrile |
| MeOH | Methanol |
| Met | Metal species for cross-coupling (e.g., MgX, ZnX, |
| | SnR₃, SiR₃, B(OR)₂) |
| mg | Milligrams |
| min | Minutes |
| mL | Milliliters |
| MS | mass spectra |
| NaHMDS | sodium hexamethyldisilazide |
| NBS | N-bromosuccinimide |
| n-BuLi | n-butyllithium |
| NCS | N-chlorosuccinimide |
| NMR | nuclear magnetic resonance |
| PEG | —CH₂CH₂O— |
| Pd₂(dba)₃ | tris(dibenzylideneacetone)dipalladium(0) |
| Pd(dppf)Cl₂•DCM | [1,1′- |
| | Bis(diphenylphosphino)ferrocene]dichloropalladium(II), |
| | complex with dichloromethane |
| Pd(PPh₃)₄ | Tetrakis(triphenylphosphine)palladium(0) |
| Ph | Phenyl |
| PR or PG or Prot. group | protecting group |
| rbf | round-bottom flask |
| RP-HPLC | reverse phase high pressure liquid chromatography |
| RT or rt | room temperature |
| sat. or satd. | saturated |
| SEC | Size Exclusion Chromatography |
| SFC | supercritical fluid chromatography |
| SPhos Pd G3 or SPhos G3 | (2-Dicyclohexylphosphino-2′,6′-dimethoxybiphenyl) [2- |
| | (2′-amino-1,1′-biphenyl)]palladium(II) |
| | methanesulfonate |
| TBAF | tetra-n-butylammonium fluoride |
| TBTU | N,N,N′,N′-Tetramethyl-(9-(benzotriazol-1-yl)uronium |
| | tetrafluoroborate |
| t-BuOH | tert-butanol |
| TEA or Et₃N | Triethylamine |
| TFA | trifluoroacetic acid |
| THF | Tetrahydrofuran |
| UV | Ultraviolet |

In this application, the use of “or” means “and/or” unless stated otherwise. In the context of a multiple dependent claim, the use of “or” refers to more than one preceding independent or dependent claim in the alternative only. Also, terms such as “element” or “component” encompass both elements and components comprising one unit and elements and components that comprise more than one subunit unless specifically stated otherwise.

Exemplary techniques used in connection with recombinant DNA, oligonucleotide synthesis, tissue culture and transformation (e.g., electroporation, lipofection), enzymatic reactions, and purification techniques are described, e.g., in Sambrook et al. Molecular Cloning: A Laboratory Manual (2nd ed., Cold Spring Harbor Laboratory Press, Cold Spring Harbor, N.Y. (1989)), among other places.

As used herein, the term “about” refers to a numeric value, including, for example, whole numbers, fractions, and percentages, whether or not explicitly indicated. The term “about” generally refers to a range of numerical values (e.g., +/5-0 of the recited range) that one of ordinary skill in the art would consider equivalent to the recited value (e.g., having the same function or result). When terms such as at least and about precede a list of numerical values or ranges, the terms modify all the values or ranges provided in the list. In some instances, the term about may include numerical values that are rounded to the nearest significant FIGURE.

The term “polypeptide” refers to a polymer of amino acid residues and is not limited to a minimum length. A “protein” may comprise one or more polypeptides. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” or “protein” refers to a polypeptide or protein, respectively, which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts that produce the proteins or errors due to PCR amplification. A protein may comprise two or more polypeptides.

The term “antibody” herein refers to a molecule comprising at least complementarity-determining region (CDR) CDR1, CDR2, and CDR3 of a heavy chain and at least CDR1, CDR2, and CDR3 of a light chain, wherein the molecule is capable of binding to antigen. The term is used in the broadest sense and encompasses various antibody structures, including but not limited to monoclonal antibodies, polyclonal antibodies, multispecific antibodies (e.g., bispecific antibodies, diabodies, etc.), full length antibodies, single-chain antibodies, antibody conjugates, and antibody fragments, so long as they exhibit the desired antigen-specific binding activity.

An “isolated” antibody is one that has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

An “antigen” refers to the target of an antibody, i.e., the molecule to which the antibody specifically binds. The term “epitope” denotes the site on an antigen, either proteinaceous or non-proteinaceous, to which an antibody binds. Epitopes on a protein can be formed both from contiguous amino acid stretches (linear epitope) or comprise non-contiguous amino acids (conformational epitope), e.g., coming in spatial proximity due to the folding of the antigen, i.e., by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents.

Unless indicated otherwise or clear from the context, an antibody defined by its target specificity (e.g. an “anti-LIV1 antibody”) refers to antibodies that can bind to its human target (human LIV1). Such antibodies may or may not bind to LIV1 from other species.

LIV1, also known as LIV-1 and ZIP6, is encoded at human solute carrier family 39 member 6 (SLC39A6). Further information is found at Gene ID: 25800. The protein sequence for human LIV1 is found as isoform 1 (NP_036451.4; SEQ ID NO: 11) and isoform 2 (NP_001092876.1; SEQ ID NO: 12).

An “anti-LIV1 antibody” or a “LIV1-antibody” or an “antibody that specifically binds to LIV1” or an “antibody that binds to LIV1” and similar phrases refer to an antibody that specifically binds to LIV1 as defined herein.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The term “complementarity determining regions” (“CDRs”) as used herein refers to each of the regions of an antibody variable region which are hypervariable in sequences, and which determine antigen binding specificity. Generally, antibodies comprise six CDRs: three in the VH (CDR-H1 or heavy chain CDR1, CDR-H2, CDR-H3), and three in the VL (CDR-L1, CDR-L2, CDR-L3). Unless otherwise indicated, the CDRs of exemplary anti-LIV1 antibodies of the present invention are determined according to the sequence table herein (Table 1).

“Framework” or “FR” refers to the residues of the variable region residues that are not part of the complementary determining regions (CDRs). The FR of a variable region generally consists of four FRs: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). See, e.g., Kindt et al. Kuby Immunology, 6^{th} ed., W.H. Freeman and Co., page 91 (2007). A variable domain may comprise heavy chain (HC) CDR1-FR2-CDR2-FR3-CDR3 with or without all or a portion of FR1 and/or FR4; and light chain (LC) CDR1-FR2-CDR2-FR3-CDR3 with or without all or a portion of FR1 and/or FR4. That is, a variable domain may lack a portion of FR1 and/or FR4 so long as it retains antigen-binding activity. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The light chain and heavy chain “constant regions” of an antibody refer to additional sequence portions outside of the FRs and CDRs and variable regions. Certain antibody fragments may lack all or some of the constant regions. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain at Gly446 and Lys447 (EU numbering). Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, a “full-length heavy chain constant region” or a “full length antibody” for example, which is a human IgG1 antibody, includes an IgG1 with both a C-terminal glycine and lysine, without the C-terminal lysine, or without both the C-terminal glycine and lysine. Unless otherwise specified herein, numbering of amino acid residues in the Fc region or constant region is according to the EU numbering system, also called the EU index, as described in Kabat et al., Sequences of Proteins of Immunological Interest, 5^{th} Ed. Public Health Service, National Institutes of Health, Bethesda, MD, 1991. Reside numbering for sequences in the Sequence Listing is necessarily sequential and thus may deviate from the EU numbering system.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

An “antibody fragment” or “antigen-binding fragment” or “antigen-binding portion” refers to a fragment or portion of an antibody other than an intact antibody that binds the antigen (i.e., LIV1) to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The terms “full length antibody”, “intact antibody”, and “whole antibody” are used herein interchangeably to refer to an antibody having a structure substantially similar to a native antibody structure or, in the case of an IgG antibody, having heavy chains that contain an Fc region as defined herein above.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” as used herein refers to antibodies produced from human immunoglobulin sequences, such as antibodies produced in non-human animals that comprise human immunoglobulin genes (such as XenoMouse® and VelocImmune® mice), and antibodies selected using in vitro methods, such as phage display, wherein the antibody repertoire is based on a human immunoglobulin sequences.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method.

A “multispecific” antibody is one that binds specifically to more than one target antigen, while a “bispecific” antibody is one that binds specifically to two antigens. An “antibody conjugate” is an antibody conjugated to one or more heterologous molecule(s), including but not limited to a therapeutic agent or a label.

Antibodies may be modified as part of the production process in certain host cells or through metabolism in vivo. An antibody or antibody region amino acid sequence herein is intended to encompass not only the specific amino acid sequence, but also that sequence as post-translationally modified, for instance, including side chain modifications and cleavages. Such a post-translational modification can occur, for instance, as a result of production of the antibody in a host cell and/or as a result of post-translational modification in vivo in an animal (e.g., a human).

In some embodiments, an antibody disclosed herein comprises a post-translational modification (e.g., one or more post-translational modifications). Post-translational modifications can include, e.g., ubiquitination, phosphorylation, acetylation, hydroxylation, methylation, glycosylation, AMPylation, prenylation, deamidation, elimylation, citrullination, and carbamoylation. In some embodiments, the antibody is not post-translationally modified.

As noted above, antibodies can undergo post-translational cleavage of one or more, particularly one or two, amino acids from the C-terminus of the heavy chain, often a Gly-Lys. This cleavage can occur, for instance, as a result of the process of production of the antibody in a host cell. An antibody produced by expression of a specific nucleic acid molecule encoding a full-length heavy chain can include the full-length heavy chain, or it can include a cleaved variant of the full-length heavy chain, such as a heavy chain lacking a C-terminal Lys or a C-terminal Gly-Lys.

Other types of post-translational modifications can occur during production of antibodies, or otherwise in vivo, such as the modification of an amino acid side chain. For instance, an N-terminal Glu or Gln residue on an antibody chain can be post-translationally modified to an N-terminal pyroglutamate (also known as pyrrolidine carboxylate; abbreviated pE).

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “signal sequence” or “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached. Nonlimiting exemplary leader sequences also include leader sequences from heterologous proteins. In some embodiments, an antibody lacks a leader sequence. In some embodiments, an antibody comprises at least one leader sequence, which may be selected from native antibody leader sequences and heterologous leader sequences.

The term “nucleic acid molecule” or “polynucleotide” includes any compound and/or substance that comprises a polymer of nucleotides. Each nucleotide is composed of a base, specifically a purine- or pyrimidine base (i.e. cytosine I, guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA, circular RNA) vectors, can be unmodified or modified.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-antigen antibody” refers to one or more nucleic acid molecules encoding anti-antigen antibody heavy and light chains (or fragments thereof), including such nucleic acid molecule(s) in a single vector or separate vectors, and such nucleic acid molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells.

In this disclosure, “binds” or “binding” or “specific binding” and similar terms, when referring to a protein and its ligand or an antibody and its antigen target for example, or some other binding pair, means that the binding affinity between the members of the binding pair is sufficiently strong that the interaction cannot be due to random molecular associations (i.e. “nonspecific binding”). Such binding typically requires a dissociation constant (K_{D}) of 1 μM or less and may often involve a K_{D} of 100 nM or less.

“Affinity” refers to the strength of the sum of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). Affinity can generally be represented by the dissociation constant (K_{D}). Affinity of an antibody for an antigen can be measured by common methods known in the art, such as surface plasmon resonance (SPR), for instance.

As used herein, the term “payload” refers to a drug that may be selected from a small molecule, oligonucleotide, or a peptide.

As used herein, and commonly used with ADCs, the term “payload” can refer to a drug.

The “payload” can be an anti-cancer agent.

“Anti-cancer agents” refers to anti-metabolites (e.g., 5-fluoro-uracil, methotrexate, fludarabine), antimicrotubule agents (e.g., vinca alkaloids such as vincristine, vinblastine; taxanes such as paclitaxel, docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, JM-216 or satraplatin, CI-973), anthracyclines (e.g., doxorubicin, daunorubicin), antitumor antibiotics (e.g., mitomycin, idarubicin, adriamycin, daunomycin), topoisomerase inhibitors (e.g., etoposide, camptothecins), anti-angiogenesis agents (e.g., Sutent® and Bevacizumab) or any other cytotoxic agents, (estramustine phosphate, prednimustine), hormones or hormone agonists, antagonists, partial agonists or partial antagonists, kinase inhibitors, and radiation treatment.

Suitable drugs or payloads that can be used in the present invention include, but are not limited to, methotrexate, vinblastine, doxorubicin, tubulin inhibitors, such as maytansinoids, i.e. maytansine, DM1, DM4, auristatin compounds, such as MMAE, MMAF, eribulin mesylate (HALAVEN®), tubulysins, cryptophycins, EG5 inhibitors, such as SB-715992 (Ispinesib®) and Filanesib® (ARRY-520), DNA damaging agents, such as enediyne, calicheamicin, uncialamycin, topoisomerase I (TOPO-1) inhibitors, such as topotecan, camptothecin, irinotecan, exatecan and exatecan derivative (Dxd), and pyrrolo benzodiazepines (PBD), duocarmycins, RNA inhibitors, including RNA slicing inhibitors (Thailanstatin and its analogs, A, B. and C) and RNA polymerase II inhibitors (amatoxins), BET or bromo-domain inhibitors, Bcl-xL inhibitors, NAMPT inhibitors, and carmaphycins, GSPT1 degraders, Toll Like Receptor (TLR 7, 8 and 9) agonists, such as the TLR 7/8 agonist represented by the structure:

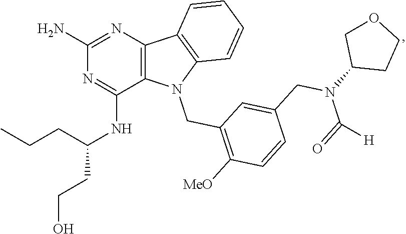 [SMILES: CCC[C@@H](CCO)Nc1nc(N)nc2c3ccccc3n(Cc3cc(CN(C=O)[C@H]4CCOC4)ccc3OC)c12]

STING agonists, glucocorticoid receptor modulators (GRMs), peptide drug conjugates (PDCs), siRNA, oligonucleotides and dual drug payloads. In addition, immune ADCs using immunomodulators as payloads have attracted significant attention because of their key roles in tumor immunotherapy.

Instead of using single simple molecules as their drugs or payloads, several novel strategies for designing ADCs using more complex drugs or payloads have emerged. For example, PROTACs or photosensitizers have been used as ADC payloads, and methods to incorporate several drugs, each with a different target, into a single antibody are being developed and can be utilized with the present invention.

As used herein, and commonly used with ADCs, the term “L” refers to linkers as described herein.

For ADCs, linkers need to fulfill two tasks: first, they must ensure good stability of the ADC rug-conjugates, in the bloodstream; second, they must ensure that the ADC can precisely release the payload at the target site.

Therefore, linkers are required to have the following three characteristics: Good stability to maintain the drug concentration of the ADC in the bloodstream and prevent premature release of the cytotoxic payload before reaching the target, thereby minimizing off-target effects and improving the safety of ADC drug-conjugates.

ADCs have two types of linkers: non-cleavable linkers and cleavable linkers. Non-cleavable linkers remain intact during intracellular metabolism. ADCs with these linkers require lysosomal degradation of the antibody to release the payload. Cleavable linkers, on the other hand, can be split during intracellular metabolism, producing metabolites containing the cytotoxic payload, which may include part of the linker.

Non-cleavable linkers are divided into two types: thioether or maleimidocaproyl (MC), composed of stable bonds that prevent proteolytic cleavage. ADCs with this type of linker rely on lysosomal enzyme degradation to release the internalized payload, resulting in the simultaneous separation of the linker.

Early exploration of this type of linker was successfully conducted by Genentech/ImmunoGen, such as in the ADC drug-conjugate trastuzumab emtansine (T-DM1 or Kadcyla®) for treating HER2-positive metastatic breast cancer. This ADC contains a non-cleavable SMCC (N-succinimidyl-4-(maleimidomethyl)cyclohexane-1-carboxylate) linker that connects the DM1 cytotoxin to trastuzumab.

Chemically cleavable linkers include acid-cleavable linkers (hydrazone bonds) and reducible linkers (disulfide bonds). The former are highly sensitive to acidic environments in the body, utilizing the acidic environment of endosomes and lysosomes to trigger linker cleavage and release the payload. However, hydrazone linkers can also undergo slow hydrolysis under physiological conditions (pH 7.4, 37° C.), leading to the slow release of the toxic drug payload. A representative ADC drug-conjugate is MYLOTARG®, the first approved ADC. However, due to the instability of the linker and the heterogeneity of the drug complex, the payload was prematurely released before reaching the target site, leading to its voluntary withdrawal by the FDA in 2010.

Another type is reducible linkers—disulfide bonds, which are dependent on reduced glutathione. Compared to plasma (~5 mol/L), the cytoplasm has higher levels of glutathione (1-10 mmol/L), making reducible disulfide linkers relatively stable in the bloodstream and cleavable by intracellular glutathione to release the payload.

Unlike chemically unstable linkers, enzyme-cleavable linkers utilize the unique high concentration of hydrolases in cells to cleave and release the payload, achieving clinical success in controlled payload release.

The first type is peptide linkers, mainly including dipeptide and tetrapeptide linkers. The cleavage mechanism of peptide linkers involves selective cleavage by cathepsin B after ADC internalization and transport to lysosomes, releasing the payload.

The most commonly used dipeptide linkers in approved ADC drugs include Val-Cit and Val-Ala dipeptides. Both linkers have comparable stability and cellular activity. However, due to precipitation and aggregation, Val-Cit is challenging to achieve high DAR (drug-to-antibody ratio), whereas Val-Ala linkers can achieve DAR as high as 7.4 with limited aggregation (<10%). Compared to Val-Cit, Val-Ala has higher hydrophilicity, making it advantageous in the context of lipophilic payloads, such as PBD dimers. Approved ADCs using these dipeptide linkers include Adcetris®, Polivy®, Padcev®, and Disitamab Vedotin® (RC48).

In addition to dipeptide linkers, the tetrapeptide Gly-Gly-Phe-Gly has also been successfully applied in ADC drugs. Compared to dipeptides, tetrapeptide linkers are more stable in the bloodstream. The approved ADC drug Enhertu uses this type of linker, and Hengrui's soon-to-be-launched HER2-targeting ADC drug SHR-A1811 also employs this tetrapeptide linker.

The second type of enzyme-cleavable linkers is glucuronide linkers, mainly including β-glucuronidase linkers and β-galactosidase linkers. β-glucuronidase-sensitive ADCs covalently bind cytotoxic payloads and antibodies through β-glucuronidase linkers and self-immolative PABC spacers. Similar to β-glucuronidase, β-galactosidase is overexpressed in certain tumors, hydrolyzing β-galactosidic bonds to release the payload. However, β-galactosidase is only present in lysosomes, whereas β-glucuronidase is expressed in lysosomes and the microenvironment of solid tumors.

The present invention is a novel hydrolyzable linker-payload compound, which upon conjugation with an appropriate antibody, hydrolyzes the thiosuccinimide ring, which then creates a stable linker, while reducing deconjugation and aggregation of the ADC. The present invention is suitable to synthesize with commercially available linkers including, but not limited to, the linkers exemplified and described herein, and linkers that are pegylated and have variable substituent groups, regardless of point of attachment.

The terms “reduce” or “inhibit” more generally refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

“Treatment” or “treating” as used herein, covers any administration or application of a therapeutic for disease in a human, and includes inhibiting the disease or progression of the disease or one or more disease symptoms, inhibiting or slowing the disease or its progression or one or more of its symptoms, arresting its development, partially or fully relieving the disease or one or more of its symptoms, or preventing a recurrence of one or more symptoms of the disease.

The terms “subject” and “patient” are used interchangeably herein to refer to a human unless expressly indicated otherwise (i.e., a murine subject or the like).

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells.

Examples of cancers applicable to methods of treatment herein include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck).

As used herein, the term “tumor,” refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

As used herein, “hematologic malignancy” refers to cancer of the body's blood-forming and immune system—the bone marrow and lymphatic tissue. Such cancers include leukemias, lymphomas (Non-Hodgkin's Lymphoma), Hodgkin's disease (also called Hodgkin's Lymphoma) and myeloma.

The term “leukemia” refers to malignant neoplasms of the blood-forming tissues. The leukemia includes, but is not limited to, chronic lymphocytic leukemia, chronic myelocytic leukemia, acute lymphoblastic leukemia, acute myeloid leukemia, and acute myeloblastic leukemia. The leukemia can be relapsed, refractory or resistant to conventional therapy.

As used herein, “promyelocytic leukemia” or “acute promyelocytic leukemia” refers to a malignancy of the bone marrow in which there is a deficiency of mature blood cells in the myeloid line of cells and an excess of immature cells called promyelocytes. It is usually marked by an exchange of regions of chromosomes 15 and 17.

As used herein, “acute lymphocytic leukemia (ALL)”, also known as “acute lymphoblastic leukemia” refers to a malignant disease caused by the abnormal growth and development of early nongranular white blood cells, or lymphocytes.

As used herein, “T-cell leukemia” refers to a disease in which certain cells of the lymphoid system called T lymphocytes or T cells are malignant. T cells are white blood cells that normally can attack virus-infected cells, foreign cells, and cancer cells and produce substances that regulate the immune response.

The term “relapsed” refers to a situation where patients who have had a remission of leukemia after therapy have a return of leukemia cells in the marrow and a decrease in normal blood cells.

The term “refractory or resistant” refers to a circumstance where patients, even after intensive treatment, have residual leukemia cells in their marrow.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

A “biological sample” as used herein refers to a sample taken from a subject or from an animal. Examples of biological samples include tissue samples and liquid biological samples, such as whole blood, serum, plasma, blood supernatant, or synovial fluid. A biological sample may be taken directly from a subject or may be first chemically or physically modified in some fashion prior to use, for example, in order to assist in analysis of the sample.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

“Aliphatic” refers to a straight or branched and/or cyclic hydrocarbon chain group consisting solely of carbon and hydrogen atoms, having from one to 25, one to 20, one to 15, one to ten, one to eight, one to six, one to five, or one to four carbon atoms, and which is attached to the rest of the molecule by a single bond, e.g., alkyl, alkenyl, cycloalkyl, and the like. In some embodiments, aliphatic contains no unsaturation. In some embodiments, aliphatic is a straight or branched chain. In some embodiments, aliphatic comprises a cycle. In some embodiments, aliphatic comprises a straight or branched chain and a cycle.

“Alkyl” refers to a straight or branched hydrocarbon chain group consisting solely of carbon and hydrogen atoms, containing no unsaturation, having from one to ten, one to eight, one to six, one to four, or one to three carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like.

“Alkenyl” refers to a straight or branched chain unsaturated group consisting solely of carbon and hydrogen atoms, having from two to eight, two to six or two to four carbon atoms, wherein the unsaturation is present only as double bonds and wherein the double bond can exist between any two carbon atoms in the chain, e.g., ethenyl, prop-1-enyl, but-2-enyl, and the like. The straight or branched chain unsaturated group is attached to the rest of the molecule by a single bond.

“Alkoxy” refers to the group having the formula —OR wherein R is alkyl. An “optionally substituted alkoxy” refers to the group having the formula —OR wherein R is an optionally substituted alkyl as defined herein.

“Alkoxyalkyl” refers to an alkoxy group as defined herein which is attached to the rest of the molecule by an alkyl group as defined herein.

“Amino” refers to a radical having the formula —NR′R″ wherein R′ and R″ are each independently hydrogen, alkyl or haloalkyl. An “optionally substituted amino” refers to a radical having the formula —NR′R″ wherein one or both of R′ and R″ are optionally substituted alkyl as defined herein.

“Aryl” refers to a 5- to 18-membered, 6- to 18-membered, 6- to 10-membered, or 6-membered carbocylic ring system, including monocyclic, bicyclic, tricyclic, tetracyclic ring systems, wherein at least one of the rings is aromatic. The aryl may be fully aromatic, examples of which are phenyl, naphthyl, anthracenyl, acenaphthylenyl, azulenyl, fluorenyl, indenyl and pyrenyl. The aryl may also contain an aromatic ring in combination with a non-aromatic ring, examples of which are acenaphene, indene, and fluorene.

“Cycloalkyl” refers to a stable monovalent monocyclic or bicyclic hydrocarbon group consisting solely of carbon and hydrogen atoms, having from three to 12, three to ten, or three to seven carbon atoms, which is saturated, e.g., cyclopropyl, cyclobutyl, cyclopentyl, cyclohexyl, decalinyl, norbornane, norbornene, adamantyl, bicyclo[2.2.2]octane and the like.

“Cycloalkoxy” refers to a cycloalkyl group as defined herein which is attached to the rest of the molecule by an oxygen atom.

“Ene” or “enyl,” when used as a suffix herein, means that the group being modified with the suffix is attached to the rest of the molecule through two or more points of attachment (typically, two), e.g., alkylene, alkenylene, and the like. The group may be attached to the rest of the molecule through any two suitable atoms in the group.

“Halo, “halogen” or “halide” refers to F, Cl, Br or I. In some embodiments, halo is fluoro or chloro. In a particular embodiment, halo is fluoro.

“Haloalkyl” refers to an alkyl group, in certain embodiments, C₁₋₆ alkyl group, in which one or more of the hydrogen atoms are replaced by halogen. Such groups include, but are not limited to, chloromethyl, trifluoromethyl 1-chloro-2-fluoroethyl, 2,2-difluoroethyl, 2-fluoropropyl, 2-fluoropropan-2-yl, 2,2,2-trifluoroethyl, 1,1-difluoroethyl, 1,3-difluoro-2-methylpropyl, 2,2-difluorocyclopropyl, (trifluoromethyl)cyclopropyl, 4,4-difluorocyclohexyl and 2,2,2-trifluoro-1,1-dimethylethyl.

“Haloalkoxy” refers to the group having the formula OR wherein R is haloalkyl, as defined herein.

“Heterocycle” or “Heterocyclyl” refers to a stable 3- to 15-membered, 3- to 12-membered, 4- to 12-membered, 4- to 7-membered, 5- to 12-membered, or 5- to 6-membered non-aromatic ring radical which consists of carbon atoms and from one to five heteroatoms selected from a group consisting of nitrogen, oxygen and sulfur. In one embodiment, the heterocyclic ring system radical may be a monocyclic, bicyclic or tricyclic ring or tetracyclic ring system, which may include fused or bridged ring systems; and the nitrogen or sulfur atoms in the heterocyclic ring system radical may be optionally oxidized; the nitrogen atom may be optionally quaternized; and the heterocyclyl radical may be partially or fully saturated. The heterocyclic ring system may be attached to the main structure at any heteroatom or carbon atom which results in the creation of a stable compound. Exemplary heterocyclic radicals include, morpholinyl, piperidinyl, piperazinyl, pyranyl, pyrrolidinyl, oxetanyl, azetidinyl, quinuclidinyl, octahydroquinolizinyl, decahydroquinolizinyl, azabicyclo[3.2.1]octanyl, azabicyclo[2.2.2]octanyl, isoindolinyl, indolinyl and others.

“Heterocyclyloxy” refers to a heterocyclyl group as defined herein which is attached to the rest of the molecule by an oxygen atom.

“Heteroaryl” refers to a heterocyclyl group as defined above which is aromatic. The heteroaryl groups include, but are not limited to monocyclyl, bicyclyl and tricyclyl groups, and may be attached to the main structure at any heteroatom or carbon atom which results in the creation of a stable compound. Examples of such heteroaryl groups include, but are not limited to: furanyl, imidazolyl, oxazolyl, isoxazolyl, pyrimidinyl, pyridinyl, pyridazinyl, thiazolyl, thienyl, benzimidazolyl, imidazo[4,5-b]pyridinyl, imidazo[1,2-a]pyridinyl, imidazo[1,2-b]pyridazinyl, imidazo[1,2-a]pyrazinyl and others.

“Hydrolyzable” and “hydrolyzed” refer to the opening of the maleimide ring between the N and C═O to generate an acyclic compound.

“Non-hydrolyzed” refers to the closed maleimide ring.

“Oxo” refers to ═O.

“Sugar acid” refers to a monosaccharide with a carboxyl (—COO/—COOH) group at one or both ends of its chain, e.g., glyceric acid, xylonic acid, gluconic acid, ascorbic acid, neuraminic acid, ketodeoxyoctulosonic acid, glucuronic acid, galacturonic acid, iduronic acid, tartaric acid, mucic acid, and saccharic acid.

“EC₅₀” refers to an amount, concentration or dosage of a particular test compound that achieves a 50% potency or effect of a maximal response, such as cell growth or proliferation measured via any of the in vitro or cell-based assay described herein.

“IC₅₀” refers to an amount, concentration or dosage of a particular test compound that achieves a 50% inhibition of a maximal response, such as cell growth or proliferation, measured via any of the in vitro or cell-based assay described herein.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, fumarates and organic sulfonates.

As used herein and unless otherwise indicated, the term “hydrate” means a compound provided herein or a salt thereof, that further includes a stoichiometric or non-stoichiometric amount of water bound by non-covalent intermolecular forces.

As used herein and unless otherwise indicated, the term “solvate” means a solvate formed from the association of one or more solvent molecules to a compound provided herein. The term “solvate” includes hydrates (e.g., monohydrate, dihydrate, trihydrate, tetrahydrate and the like).

Unless stated otherwise specifically described in the specification, it is understood that the substitution can occur on any atom of the alkyl, alkenyl, alkynyl, cycloalkyl, heterocyclyl, aryl or heteroaryl group.

Where the number of any given substituent is not specified (e.g., haloalkyl), there may be one or more substituents present. For example, “haloalkyl” may include one or more of the same or different halogens.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aryloxyamine, aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aryloxyamine, aralkoxyamine; N-oxide; hydrazine; hydrazide; hydrazone; azide; isocyanate; isothiocyanate; cyanate; thiocyanate; B(OH)₂, or O(alkyl)aminocarbonyl.

Unless specifically stated otherwise, where a compound may assume alternative tautomeric, regioisomeric and/or stereoisomeric forms, all alternative isomers are intended to be encompassed within the scope of the claimed subject matter. For example, where a compound is described as having one of two tautomeric forms, it is intended that both tautomers be encompassed herein.

Thus, the compounds provided herein may be enantiomerically pure or be stereoisomeric or diastereomeric mixtures.

It is to be understood that the compounds provided herein may contain chiral centers. Such chiral centers may be of either the (R) or (S) configuration or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

Optically active (+) and (−), (R)- and (S)-, or (D)- and (L)-isomers may be prepared using chiral synthons or chiral reagents, or resolved using conventional techniques, such as chromatography on a chiral stationary phase.

Also provided herein are isotopically enriched analogs of the compounds provided herein. Isotopic enrichment (for example, deuteration) of pharmaceuticals to improve pharmacokinetics (“PK”), pharmacodynamics (“PD”), and toxicity profiles, has been demonstrated previously with some classes of drugs. See, for example, Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D, Chem. Biol. Interact. 117: 191 (1999).

Without being limited by any particular theory, isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). (e.g., Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. Without being limited by a particular theory, high DKIE values may be due in part to a phenomenon known as tunnelling, which is a consequence of the uncertainty principle. Tunnelling is ascribed to the small mass of a hydrogen atom and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has two neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and typically gives numerically larger isotope effects.

Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may provide kinetic isotope effects.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, which react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. As a result, these drugs often require the administration of multiple or high daily doses.

Isotopic enrichment at certain positions of a compound provided herein may produce a detectable KIE that affects the pharmacokinetic, pharmacologic, and/or toxicological profiles of a compound provided herein in comparison with a similar compound having a natural isotopic composition. In one embodiment, the deuterium enrichment is performed on the site of C—H bond cleavage during metabolism.

In the description herein, if there is any discrepancy between a chemical name and chemical structure, the structure controls.

### II. Exemplary Linker-Payload Compounds and Antibody-Drug Conjugates

An antibody-drug conjugate typically comprises three components: antibody, payload, and linker. In some embodiments, the antibody targets the ADC to a particular cell type and may also elicit a therapeutic response, the payload elicits a desired therapeutic response, and the linker attaches the payload to the antibody. Often, the payload is coupled via the linker to an antibody that specifically targets a certain tumor antigen (e.g., a protein that is expressed at higher levels on tumor cells compared to normal cells). In some embodiments, an antibody binds its antigen on the surface of a cell, such as a cancer cell, and the ADC is internalized by the cell. In some such embodiments, after the ADC is internalized, the payload elicits the desired therapeutic response, for example, in the case of a payload which is an anticancer agent, the payload inhibits the expansion of, or kills, the cancer cell.

In the present invention, the partial structure consisting of a linker and a payload in the antibody-drug conjugate is referred to as a “hydrolyzable linker-payload.” The linker-payload is connected to a thiol group (in other words, the sulfur atom of a cysteine residue) formed at an interchain disulfide bond site (two sites between heavy chains, and two sites between a heavy chain and a light chain) in the antibody.

In some embodiments, the payload is an anticancer drug, such as any of the anticancer drugs described herein in connection with combination therapy. In certain embodiments of the present invention and for exemplary purposes, the payload is a TLR7/8 agonist compound.

Nonlimiting examples of hydrolyzable linker-payload compounds are described herein. The hydrolyzable linker-payload compound of the present invention includes a TLR 7/8 agonist. The TLR7/8 agonist is the compound represented by formula:

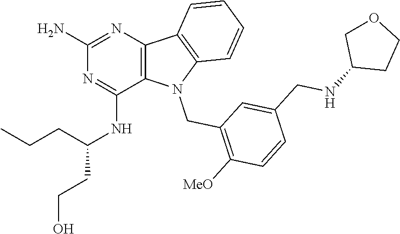 [SMILES: CCC[C@@H](CCO)Nc1nc(N)nc2c3ccccc3n(Cc3cc(CN[C@H]4CCOC4)ccc3OC)c12]

and possesses an antitumor effect. Additional examples of potential payload compounds that may be used in the present invention include, but is not limited to: exatecan, DXd, MMAE, MMAF, and eribulin.

The scope of the present invention comprises the hydrolyzable linker-payload compound claimed herein to be attached to any appropriate payload and conjugated to any appropriate antibody. The anti-LIV1 antibody described herein may be included in an antibody-drug conjugate and is used for exemplification of the superior stability of the novel hydrolyzable linker-payload compound.

Nonlimiting examples of antibody-drug conjugates are described herein. Thus, in some embodiments, an antibody-drug conjugate comprising an anti-LIV1 antibody provided herein, a TLR7/8 agonist compound described herein (e.g., any of Examples 1-15), and a hydrolyzable linker that attaches the TLR 7/8-degrading compound to the anti-LIV1 antibody, is provided. In some embodiments, provided herein is an antibody-drug conjugate comprising an anti-LIV1 antibody provided herein, and a TLR7/8-agonist-hydrolyzable linker compound described herein (e.g., any of Examples ADC #1-15).

Nonlimiting examples of hydrolyzable linker-payload compounds and antibody-drug conjugates are described herein. For example, provided in an aspect is a hydrolyzable linker-payload compound of the following formula:

### Embodiments

1. In a first embodiment, the present invention comprises a compound of Formula (I):

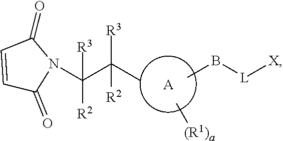

or a pharmaceutically acceptable salt thereof,
wherein:
A is selected from an unsubstituted or substituted 4 to 12 membered heterocyclic or heterospirocyclic ring;
B is independently selected from a bond, —CH₂—, —C═O—, —O—, —(—(CR¹¹R¹²)ₙ—)—O)ₘ—, —C═O—(CR¹¹R¹²)ₙ—O—)ₘ—, —C═O—(NR¹¹)—, —S—, —SO—, —SO₂—, or —SO₂—(NR¹¹)—;
R¹ is independently selected from hydrogen, halogen, —OR¹¹, —N(R¹¹R¹²)—NHR¹¹, —(—(CH₂)₂—)ₙ-O—)ₘ, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —N(R¹¹R¹²)—NHR¹¹ or —OR¹¹;
R² is independently selected from hydrogen, halogen, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —NHR¹¹ or —OR¹¹;
R³ is independently selected from hydrogen, halogen, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —NHR¹¹ or —OR¹¹;
wherein two of any R¹, R² or R³ substituents together with the carbon atoms they are attached to, may join to form a 5 or 6 membered ring that may be unsaturated, saturated, partially saturated;
and may further optionally be substituted with 1 or 2 R¹¹ substituents;
R¹¹ is independently selected from hydrogen, halogen, —C₁-C₆ alkyl, —C₂-C₆ alkenyl, —C₁-C₆ haloalkyl, —(—(CH₂)ₙ—)—O—)ₘ, a 3 to 12 membered cycloalkyl, a 4 to 12 membered heterocyclic, a 5 to 12 membered aryl or a 5 to 12 membered heteroaryl ring;
R¹² is independently selected from hydrogen, halogen, —C₁-C₆ alkyl, —C₂-C₆ alkenyl, —C₁-C₆ haloalkyl, —(—(CH₂)ₙ—)—O—)ₘ, a 3 to 12 membered cycloalkyl, a 4 to 12 membered heterocyclic, a 5 to 12 membered aryl or a 5 to 12 membered heteroaryl ring;
wherein the alkyl, alkenyl, haloalkyl, cycloalkyl, heterocyclic, aryl or heteroaryl rings in R¹¹ and R¹² are each independently unsubstituted or substituted with 1, 2, or 3 R¹³ substituents;
R¹³ is independently hydrogen, halo, —C₁-C₆ alkyl, —C₁-C₆ haloalkyl, —C₁-C₆ alkoxyalkyl, oxo, —CN, —NR¹⁴R¹⁴, —CH₃—NH—CH₂—C(═O)—OH, hydroxyl or —C₁-C₆ alkoxy;
R¹⁴ is independently hydrogen, —C₁-C₆ alkyl, —C₁-C₆ haloalkyl, or —C₁-C₆ alkoxyalkyl;
L is a linker;
X is a payload;
a is 0, 1, 2, 3, or 4;
m is an integer selected from 0 to 36;
n is 1, 2, 3, 4, 5, 6, 7, 8, 9, 10, 11 or 12; and further
wherein the heterocyclic and heteroaryl cyclic ring in each A, R¹, R², R³, R¹¹, and R¹² may include 1, 2 or 3 heteroatoms independently selected from O, N or S.

2. In another embodiment, the compound of embodiment 1, wherein A is a heterocyclic ring substituted with at least one N.

3. In another embodiment, the compound of embodiment 1, wherein A is a heterospirocyclic ring substituted with at least one N.

4. In another embodiment, the compound of embodiment 1, wherein A is a 6 to 9 membered heterocyclic ring substituted with at least one N.

5. In another embodiment, the compound of embodiment 1, wherein A is a 6 to 9 membered heterospirocyclic ring substituted with at least one N.

6. In another embodiment, the compound of any one of embodiments 1-5, wherein

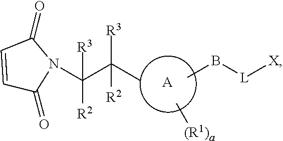

wherein
A is selected from an unsubstituted or substituted 6 to 9 membered heterocyclic or heterospirocyclic ring;
B is independently selected from a bond, —CH₂—, —C═O—, —(—(CR¹¹R¹²)ₙ—)—O)ₘ—, —C═O—(CR¹¹R¹²)ₙ—O—)ₘ—;
R¹ is independently selected from hydrogen, halogen, —OR¹¹, —(—(CH₂)₂—)ₙ-O—)ₘ, —C₁-C₆ alkyl, a 3 to 6-membered cycloalkyl, a 4 to 12-membered heterocyclic, a 5 to 12-membered aryl or a 5 to 12-membered heteroaryl ring, wherein the alkyl, cycloalkyl, heterocyclic and heteroaryl may be optionally substituted with —R¹¹, —N(R¹¹R¹²)—NHR¹¹ or —OR¹¹;
R² is independently selected from hydrogen, halogen, —C₁-C₆ alkyl, a 3 to 6 membered cycloalkyl ring, wherein the alkyl, cycloalkyl ring may be optionally substituted with —R¹¹, —NHR¹¹ or —OR¹¹; and
R³ is independently selected from hydrogen, halogen, —C₁-C₆ alkyl, a 3 to 6 membered cycloalkyl ring wherein the alkyl, cycloalkyl ring may be optionally substituted with —R¹¹, —NHR¹¹ or —OR¹¹.

7. In another embodiment, the compound of embodiment 6, wherein A is a 6 to 9 membered heterocyclic ring.

8. In another embodiment, the compound of any one of embodiments 1 and 6, wherein A is a 6 to 9 membered heterospirocyclic ring.

9. In another embodiment, the compound of any one of embodiments 1-8, wherein B is —CH₂—.

10. In another embodiment, the compound of any one of embodiments 1-8, wherein B is —C═O—.

11. In another embodiment, the compound of any one of embodiments 1-8, wherein B is —(—(CR¹¹R¹²)ₙ—O)ₘ.

12. In another embodiment, the compound of any one of embodiments 1-8, wherein B is —C═O—(CR¹¹R¹²)ₙ—O—)ₘ—.

13. In another embodiment, the compound of any one of embodiments 1-11, wherein R¹ is hydrogen.

14. In another embodiment, the compound of any one of embodiments 1-11, wherein R¹ is, halogen.

15. In another embodiment, the compound of any one of embodiments 1-11, wherein R¹ is —OR¹¹.

16. In another embodiment, the compound of any one of embodiments 1-11, wherein R¹ is —(—(CH₂)₂—)ₙ-O—)ₘ.

17. In another embodiment, the compound of any one of embodiments 1-15, wherein R² is hydrogen.

18. In another embodiment, the compound of any one of embodiments 1-15, wherein R² is halogen.

19. In another embodiment, the compound of any one of embodiments 1-15, wherein R² is —C₁-C₆ alkyl.

20. In another embodiment, the compound of any one of embodiments 1-18, wherein R³ is hydrogen.

21. In another embodiment, the compound of any one of embodiments 1-18, wherein R³ is halogen.

22. In another embodiment, the compound of any one of embodiments 1-18, wherein R³ is —C₁-C₆ alkyl.

23. In another embodiment, the compound of any one of embodiments 1-18, wherein R³ is a 3 to 6 membered cycloalkyl ring.

24. In another embodiment, the compound of any one of embodiments 1-23, wherein a is 0.

25. In another embodiment, the compound of any one of embodiments 1-23, wherein a is 1.

26. In another embodiment, the compound of any one of embodiments 1-25, wherein m is 1.

27. In another embodiment, the compound of any one of embodiments 1-25, wherein m is 2.

28. In another embodiment, the compound of any one of embodiments 1-25, wherein m is 4.

29. In another embodiment, the compound of any one of embodiments 1-25, wherein m is 6.

30. In another embodiment, the compound of any one of embodiments 1-25, wherein m is 8.

31. In another embodiment, the compound of any one of embodiments 1-30, wherein n is 1.

32. In another embodiment, the compound of any one of embodiments 1-30, wherein n is 2.

33. In another embodiment, the compound of embodiment 1, of Formula (II):

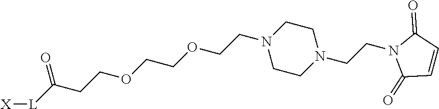

or a pharmaceutically acceptable salt thereof, wherein:
L is a linker, and
X is a payload.

34. In another embodiment, the compound of embodiment 1, having Formula (III):

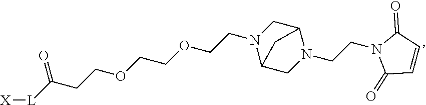

or a pharmaceutically acceptable salt thereof, wherein:
L is a linker, and
X is a payload.

35. In another embodiment, the compound of embodiment 1, having Formula (IV):

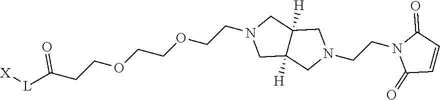

or a pharmaceutically acceptable salt thereof, wherein:
L is a linker, and
X is a payload.

36. In another embodiment, the compound of embodiment 1, having Formula (V):

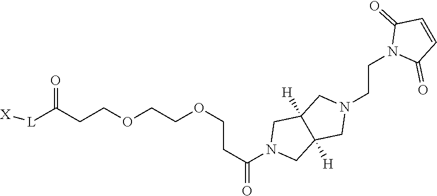

or a pharmaceutically acceptable salt thereof, wherein:
L is a linker, and
X is a payload.

37. In another embodiment, the compound of embodiment 1, having Formula (VI):

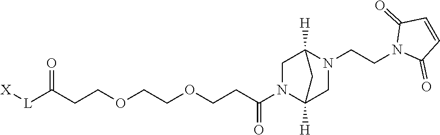

or a pharmaceutically acceptable salt thereof, wherein:
L is a linker, and
X is a payload.

38. In another embodiment, the compound of embodiment 1, having Formula (VII):

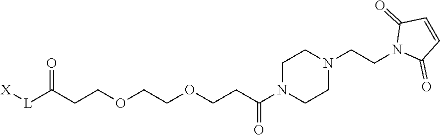

or a pharmaceutically acceptable salt thereof, wherein:
L is a linker, and
X is a payload.

39. In another embodiment, the compound of embodiment 1, having Formula (VIII):

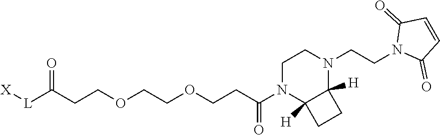

or a pharmaceutically acceptable salt thereof, wherein:
L is a linker, and
X is a payload.

40. In another embodiment, the compound of embodiment 1, having Formula (IX):

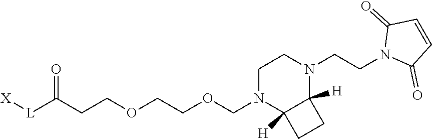

or a pharmaceutically acceptable salt thereof, wherein:
L is a linker, and
X is a payload.

41. In another embodiment, the compound of embodiment 1, having Formula (X):

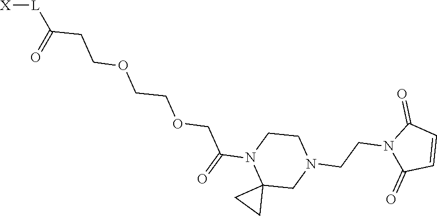

or a pharmaceutically acceptable salt thereof, wherein:
L is a linker, and
X is a payload.

42. In another embodiment, the compound of embodiment 1, having Formula (XI):

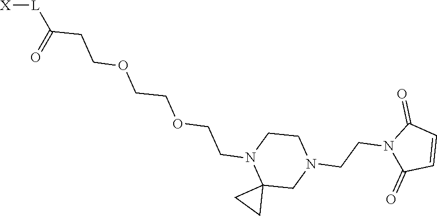

or a pharmaceutically acceptable salt thereof, wherein:
L is a linker, and
X is a payload.

43. In another embodiment, the compound of embodiment 1, having Formula (XII):

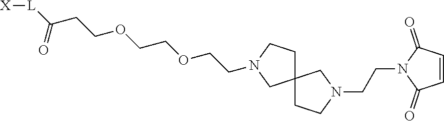

or a pharmaceutically acceptable salt thereof, wherein:
L is a linker, and
X is a payload.

44. In another embodiment, the compound of embodiment 1, having Formula (XIII)

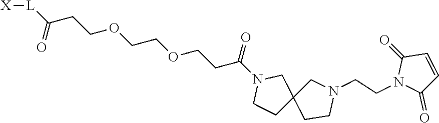

or a pharmaceutically acceptable salt thereof, wherein:
L is a linker, and
X is a payload.

45. In another embodiment, the compound of embodiment 1, having Formula (IVX):

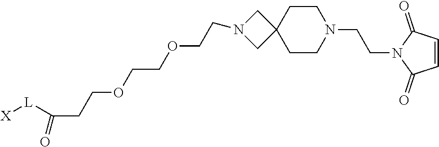

or a pharmaceutically acceptable salt thereof, wherein:
L is a linker, and
X is a payload.

46. In another embodiment, the compound of embodiment 1, having Formula (XV):

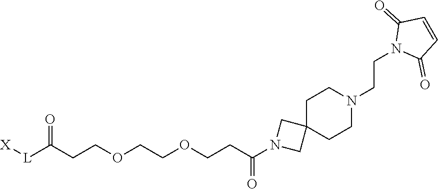

or a pharmaceutically acceptable salt thereof, wherein:
L is a linker, and
X is a payload.

47. In another embodiment, the compound of embodiment 1, having Formula (XVI):

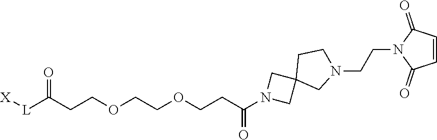

or a pharmaceutically acceptable salt thereof, wherein:
L is a linker, and
X is a payload.

48. In another embodiment, the compound of any one of embodiments 1-47, wherein L is Val-Cit.

49. In another embodiment, the compound of any one of embodiments 1-47, wherein L is Val-Ala.

50. In another embodiment, the compound of any one of embodiments 1-47, wherein L is Ala-Ala.

51. In another embodiment, the compound of any one of embodiments 1-47, wherein L is GGFG.

52. In another embodiment, the compound of any one of embodiments 1-47, wherein L is Val-Cit-PABC.

53. In another embodiment, the compound of any one of embodiments 1-47, wherein L is Glucoronide.

54. In another embodiment, the compound of embodiment 53, wherein L is beta Glucoronide.

55. In another embodiment, the compound of any one of embodiments 1-47, wherein L is selected from:

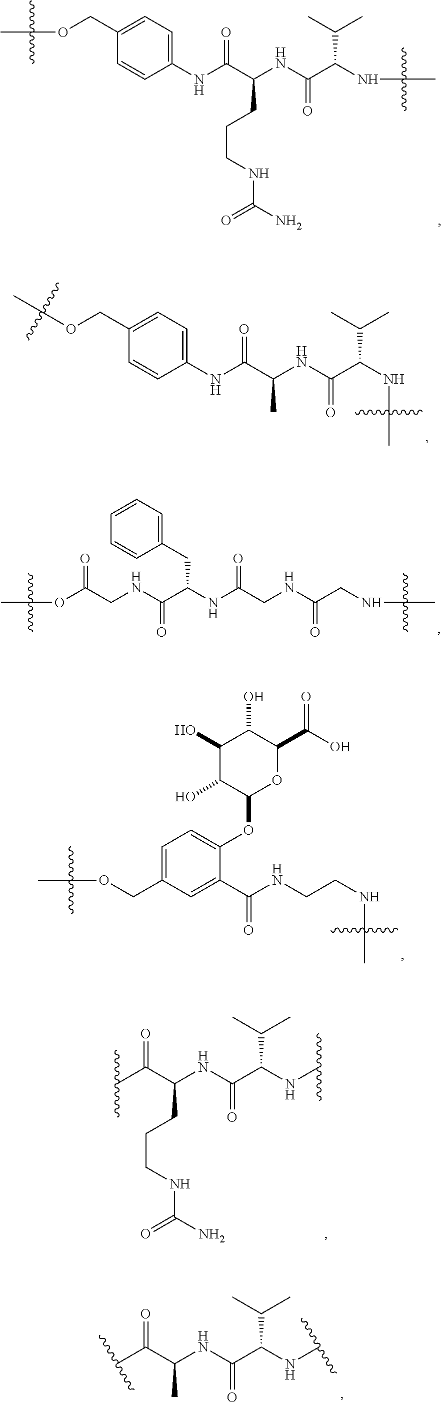

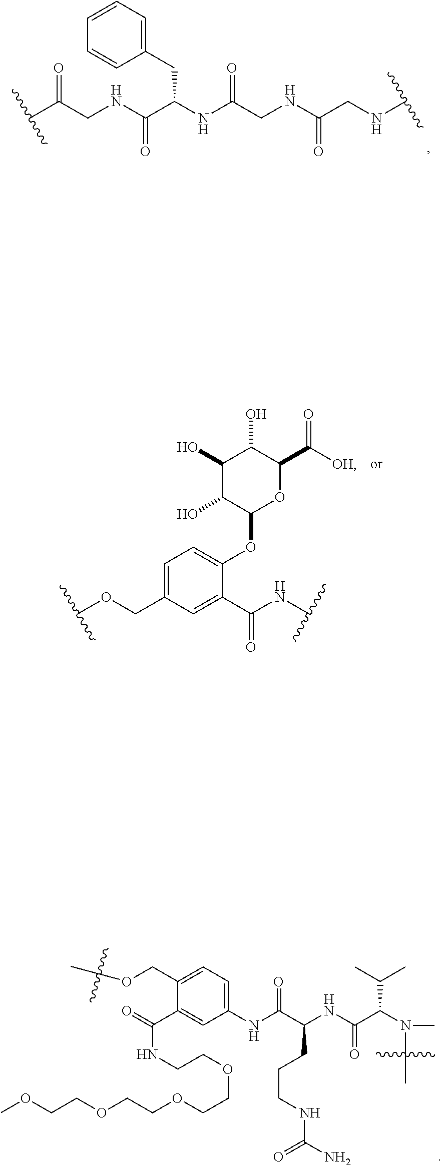

56. In a further embodiment, the compound of any of embodiments 1-55, wherein X is a payload, wherein the payload is an anti-cancer agent.

57. In another embodiment, the compound of any of embodiments 1-56, wherein X is a payload, wherein the payload is a small molecule, oligonucleotide, or a peptide.

58. In another embodiment, the compound of embodiment 57, wherein the payload is a small molecule.

59. In another embodiment, the compound of embodiment 58, wherein the small molecule is selected from exatecan, DXd, MMAE, MMAF,

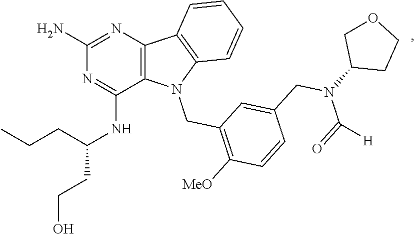 [SMILES: CCC[C@@H](CCO)Nc1nc(N)nc2c3ccccc3n(Cc3cc(CN(C=O)[C@H]4CCOC4)ccc3OC)c12]

and eribulin.

60. In another embodiment, the compound of embodiment 57, wherein the small molecule is exatecan.

61. In another embodiment, the compound of embodiment 57, wherein the small molecule is Dxd.

62. In another embodiment, the compound of embodiment 57, wherein the small molecule is MMAE.

63. In another embodiment, the compound of embodiment 57, wherein the small molecule is MMAF.

64. In another embodiment, the compound of embodiment 57, wherein the small molecule is

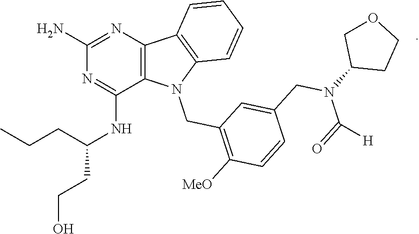 [SMILES: CCC[C@@H](CCO)Nc1nc(N)nc2c3ccccc3n(Cc3cc(CN(C=O)[C@H]4CCOC4)ccc3OC)c12]

65. In another embodiment, the compound of embodiment 57, wherein the small molecule is exatecan.

66. In a further embodiment, the present invention comprises a compound selected from:

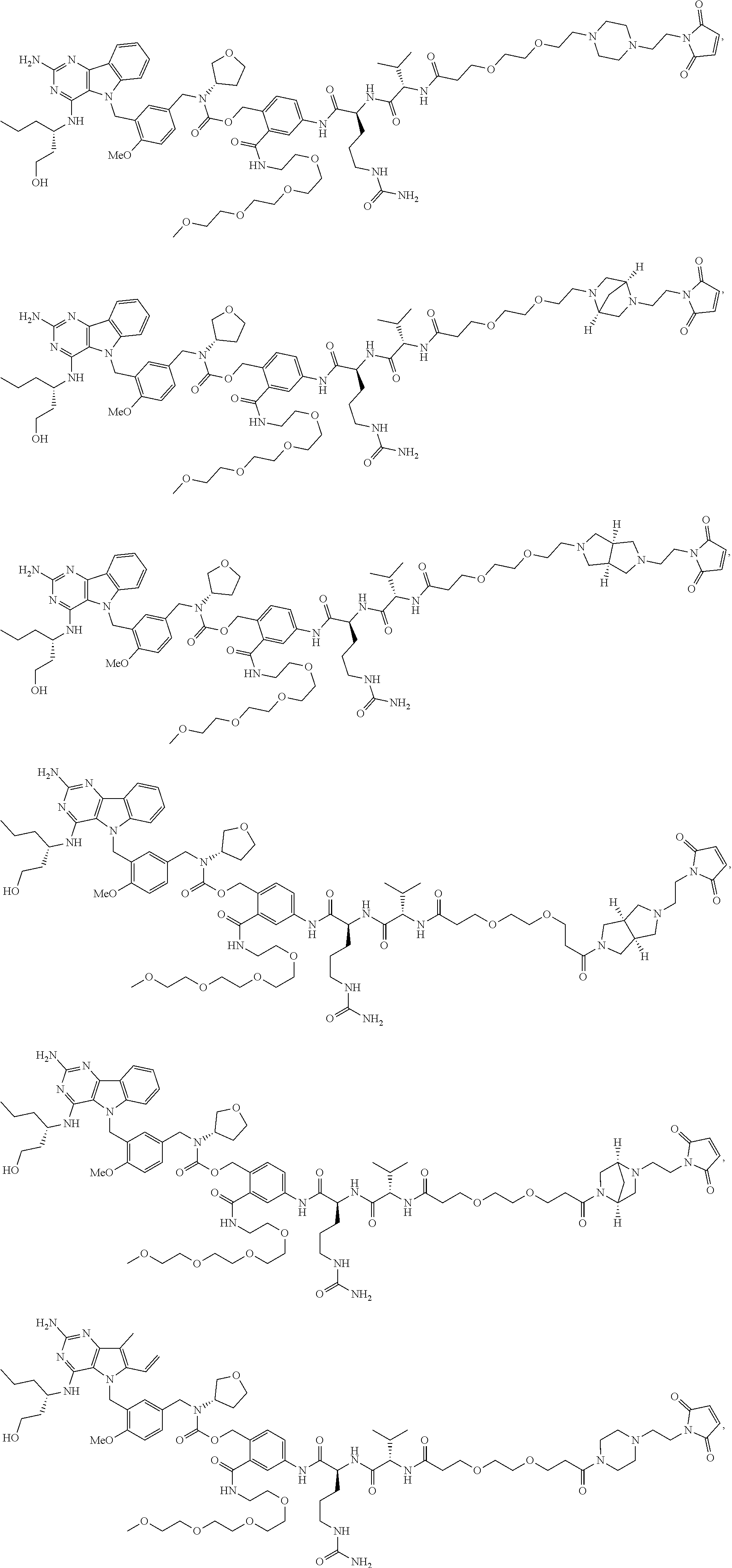

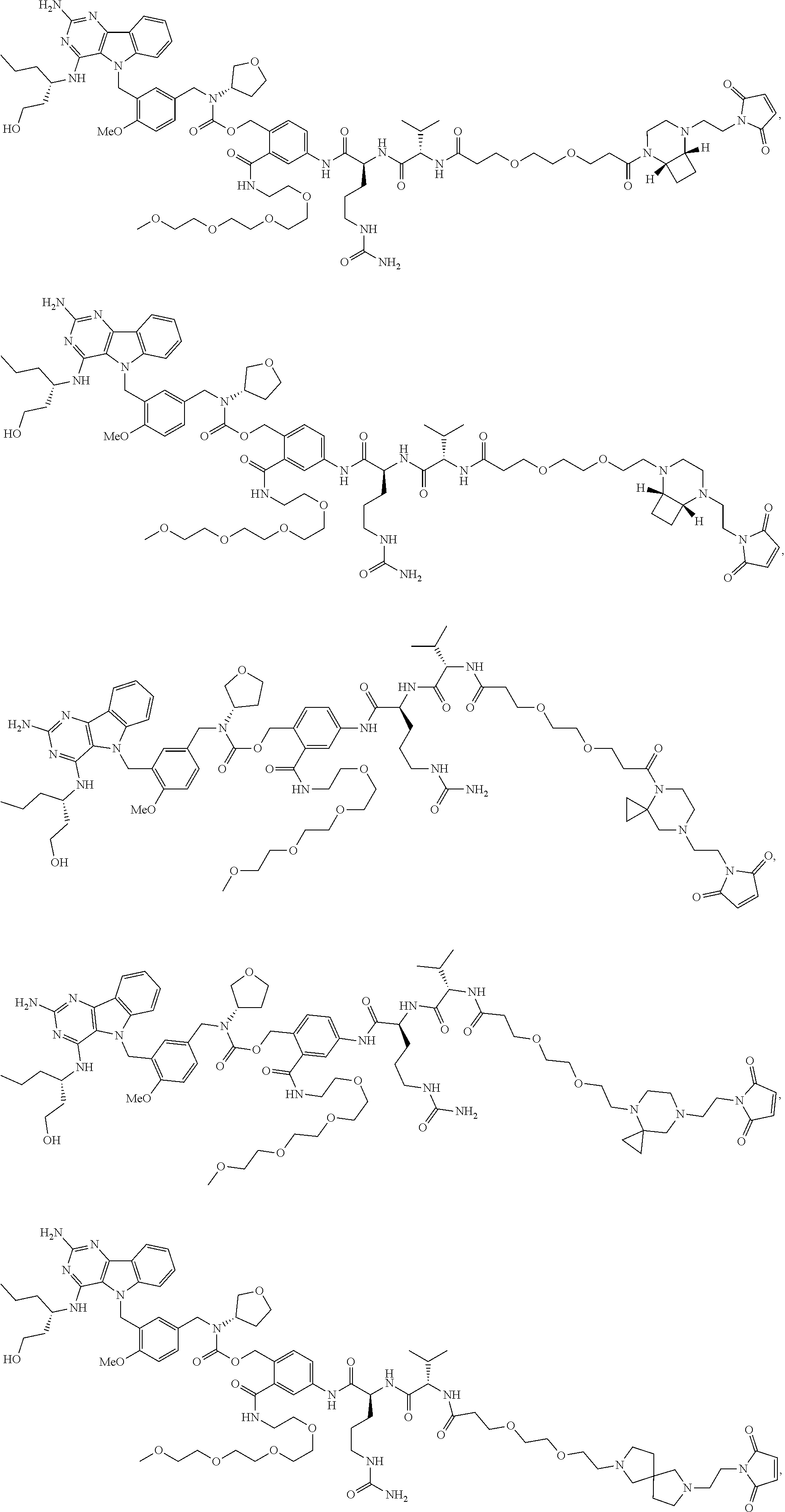

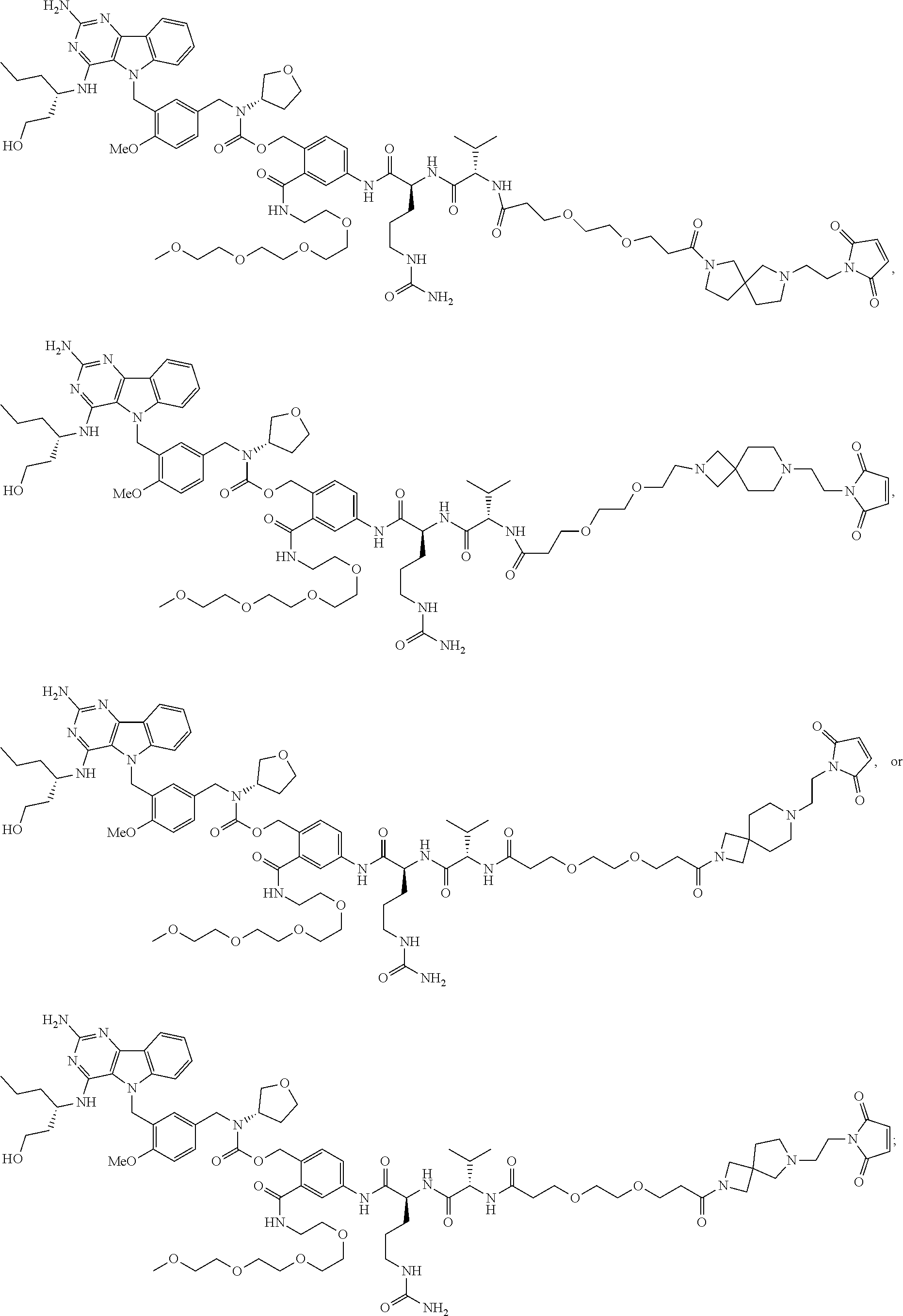

or a pharmaceutically acceptable salt thereof.

67. In a further embodiment, the present invention comprises an antibody-drug conjugate, of Formula XVII:

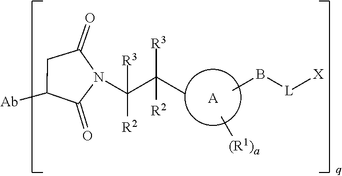

or a pharmaceutically acceptable salt thereof, wherein:
the maleimide ring may be a mixture of a hydrolyzed and a non-hydrolyzed ring;
   A is selected from an unsubstituted or substituted 4 to 12 membered heterocyclic or heterospirocyclic ring;
   B is independently selected from a bond, —CH₂—, —C═O—, —O—, —(—(CR¹¹R¹²)ₙ—)—O)ₘ—, —C═O—(CR¹¹R¹²)ₙ—O—)ₘ—, —C═O—(NR¹¹)—, —S—, —SO—, —SO₂—, or —SO₂—(NR¹¹)—;
   R¹ is independently selected from hydrogen, halogen, —OR¹¹, —N(R¹¹R¹²)—NHR¹¹, —(—(CH₂)₂—)ₙ-O—)ₘ, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —N(R¹¹R¹²)—NHR¹¹ or —OR¹¹;
   R² is independently selected from hydrogen, halogen, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —NHR¹¹ or —OR¹¹;
   R³ is independently selected from hydrogen, halogen, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —NHR¹¹ or —OR¹¹;
   wherein two of any R¹, R² or R³ substituents together with the carbon atoms they are attached to, may join to form a 5 or 6 membered ring that may be unsaturated, saturated, partially saturated;
   and may further optionally be substituted with 1 or 2 R¹¹ substituents;
   R¹¹ is independently selected from hydrogen, halogen, —C₁-C₆ alkyl, —C₂-C₆ alkenyl, —C₁-C₆ haloalkyl, —(—(CH₂)ₙ—)—O—)ₘ, a 3 to 12 membered cycloalkyl, a 4 to 12 membered heterocyclic, a 5 to 12 membered aryl or a 5 to 12 membered heteroaryl ring;
   R¹² is independently selected from hydrogen, halogen, —C₁-C₆ alkyl, —C₂-C₆ alkenyl, —C₁-C₆ haloalkyl, —(—(CH₂)ₙ—)—O—)ₘ, a 3 to 12 membered cycloalkyl, a 4 to 12 membered heterocyclic, a 5 to 12 membered aryl or a 5 to 12 membered heteroaryl ring;
   wherein the alkyl, alkenyl, haloalkyl, cycloalkyl, heterocyclic, aryl or heteroaryl rings in R¹¹ and R¹² are each independently unsubstituted or substituted with 1, 2, or 3 R¹³ substituents;
   R¹³ is independently hydrogen, halo, —C₁-C₆ alkyl, —C₁-C₆ haloalkyl, —C₁-C₆ alkoxyalkyl, oxo, —CN, —NR¹⁴R¹⁴, —CH₃—NH—CH₂—C(═O)—OH, hydroxyl or —C₁-C₆ alkoxy;
   R¹⁴ is independently hydrogen, —C₁-C₆ alkyl, —C₁-C₆ haloalkyl, or —C₁-C₆ alkoxyalkyl;
   L is a linker;
   X is a payload;
   a is 0, 1, 2, 3, or 4;
   m is an integer selected from 0 to 36;
   n is 1, 2, 3, 4, 5, 6, 7, 8, 9, 10, 11 or 12;
   q is 1, 2, 3, 4, 5, 6, 7 or 8; and further
   wherein the heterocyclic and heteroaryl cyclic ring in each A, R¹, R², R³, R¹¹, and R¹² may include 1, 2 or 3 heteroatoms independently selected from O, N or S; and Ab is an antibody or antigen binding fragment thereof.

68. In another embodiment, the antibody-drug conjugate of embodiment 67, wherein the antibody is an anti-LIV1 antibody, or antigen binding fragment thereof, comprising heavy and light chain variable regions of mAb 5A11 comprising:
a. CDRH1 consisting of SEQ ID NO: 1;
b. CDRH2 consisting of SEQ ID NO: 2;
c. CDRH3 consisting of SEQ ID NO: 3;
d. CDRL1 consisting of SEQ ID NO: 6;
e. CDRL2 consisting of SEQ ID NO: 7; and
f. CDRL3 consisting of SEQ ID NO: 8.

69. In another embodiment, the anti-LIV1 antibody, or antigen binding fragment, of Embodiment 67 comprising a heavy chain variable region (VH) sequence of SEQ ID NO: 4 and a light chain variable region (VL) sequence of SEQ ID NO: 9.

70. In another embodiment, the anti-LIV1 antibody of Embodiment 70 comprising: a heavy chain (HC) sequence of SEQ ID NO: 5 and a light chain (LC) sequence of SEQ ID NO: 10.

71. In another embodiment, the antibody-drug conjugate of any one of embodiments 67-70, wherein L is Val-Cit.

72. In another embodiment, the antibody-drug conjugate of any one of embodiments 67-70, wherein L is Val-Ala.

73. In another embodiment, the antibody-drug conjugate of any one of embodiments 67-70, wherein L is Ala-Ala.

74. In another embodiment, the antibody-drug conjugate of any one of embodiments 67-70, wherein L is GGFG.

75. In another embodiment, the antibody-drug conjugate of any one of embodiments 67-70, wherein L is Val-Cit-PABC.

76. In another embodiment, the antibody-drug conjugate of any one of embodiments 67-70, wherein L is Glucoronide.

77. In another embodiment, the antibody-drug conjugate of embodiment 76, wherein L is beta-Glucoronide.

78. In another embodiment, the antibody-drug conjugate of any of embodiments 67-70, wherein L is selected from:

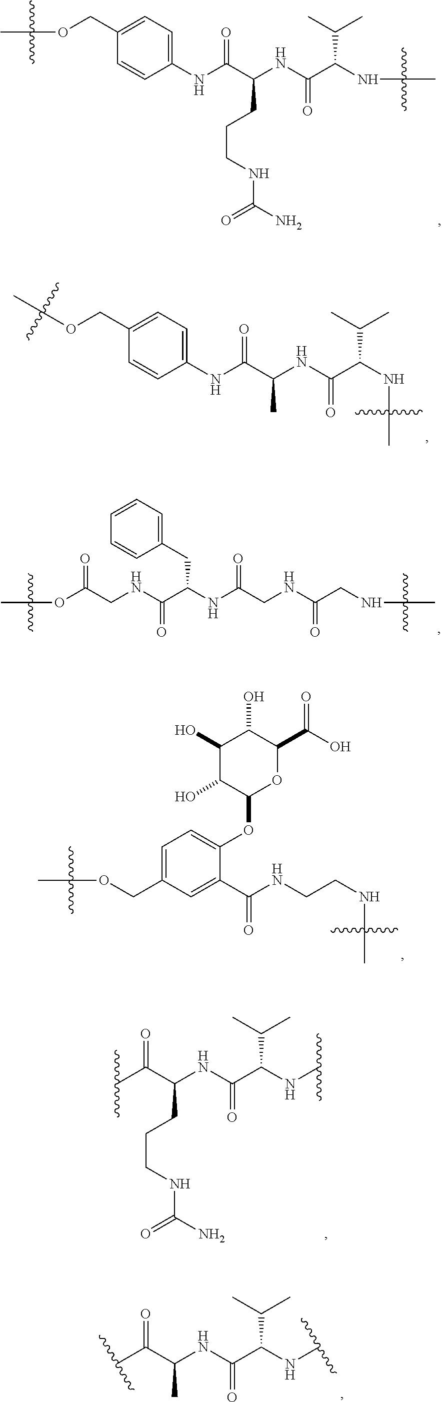

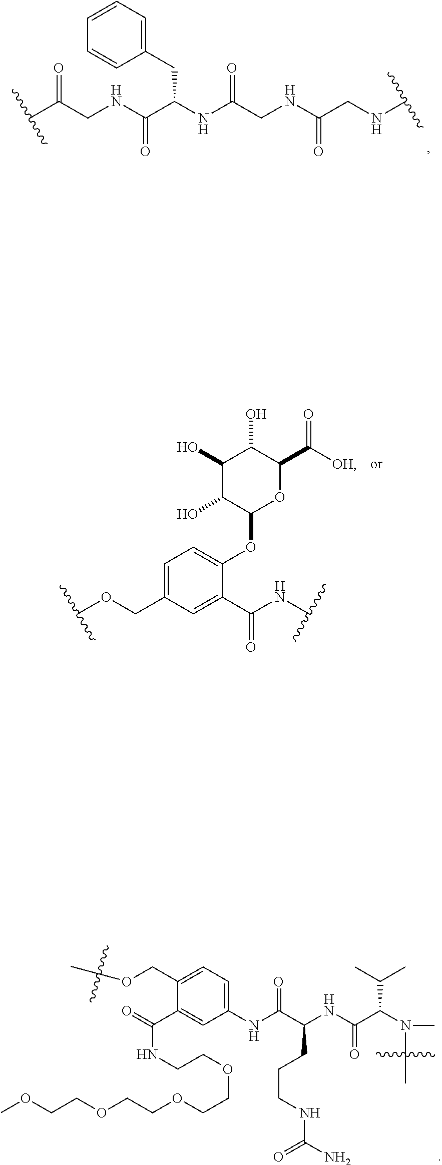

79. In another embodiment, the antibody-drug conjugate of any one of embodiments 67-70, having Formula XVIII, selected from:

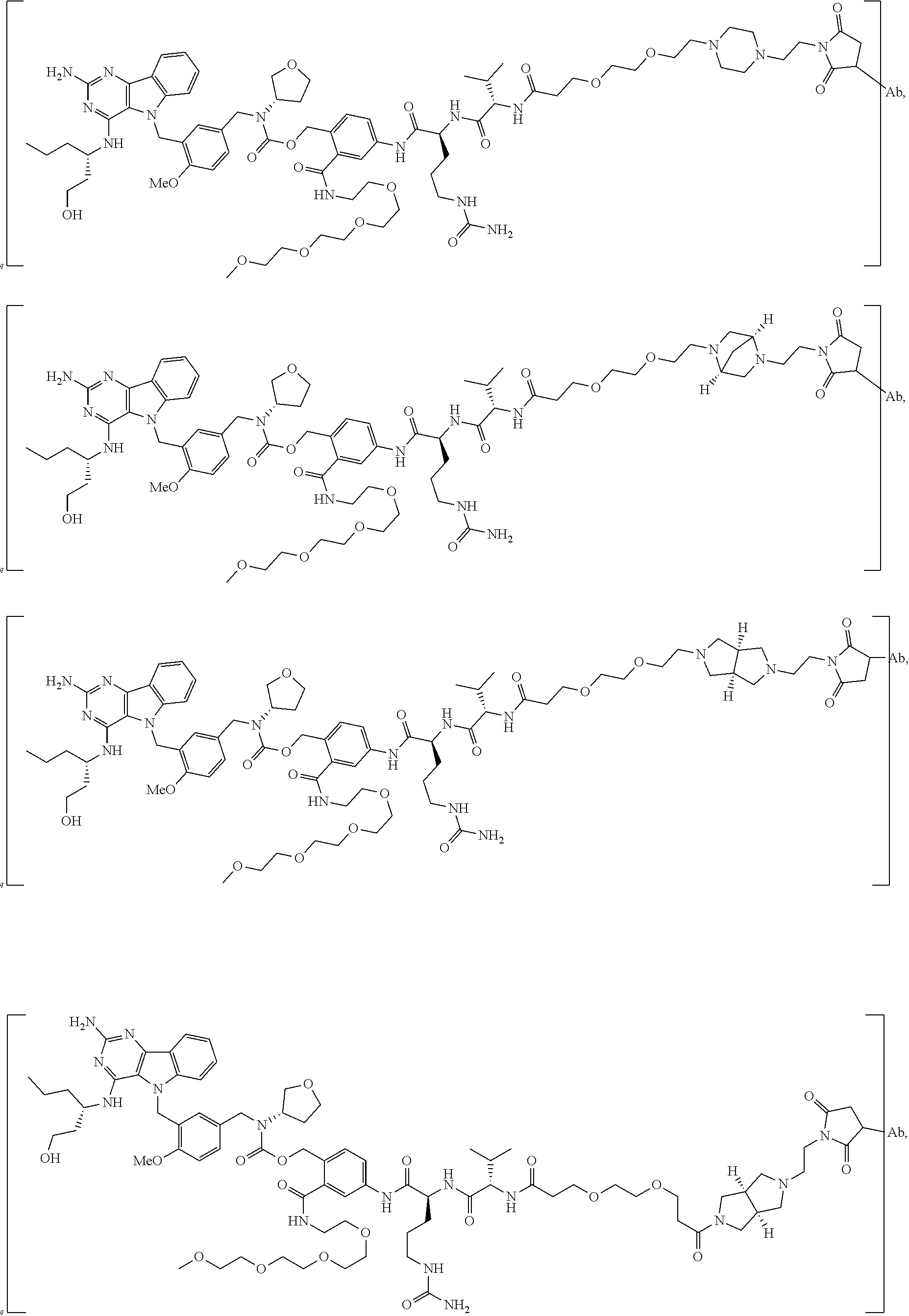

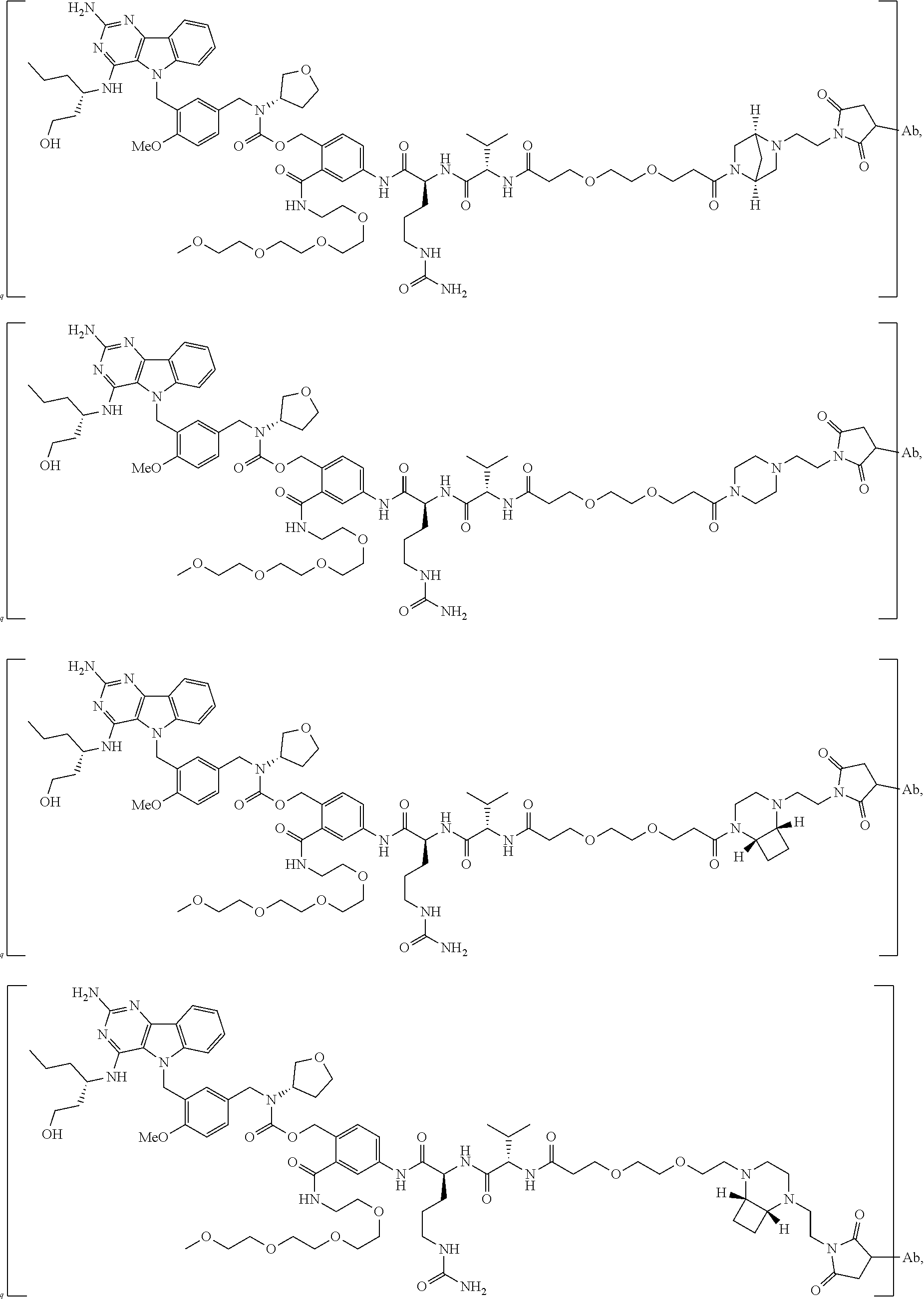

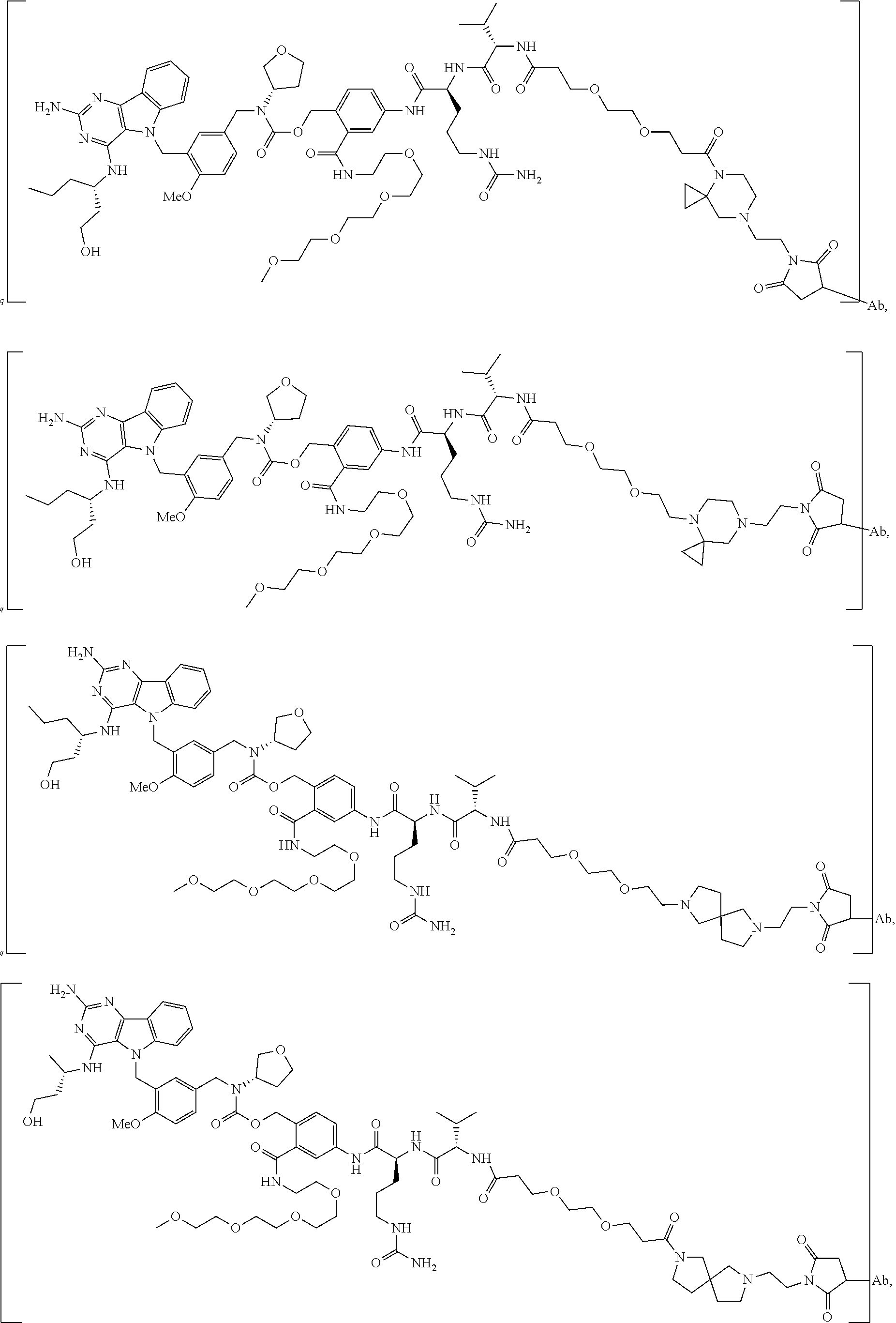

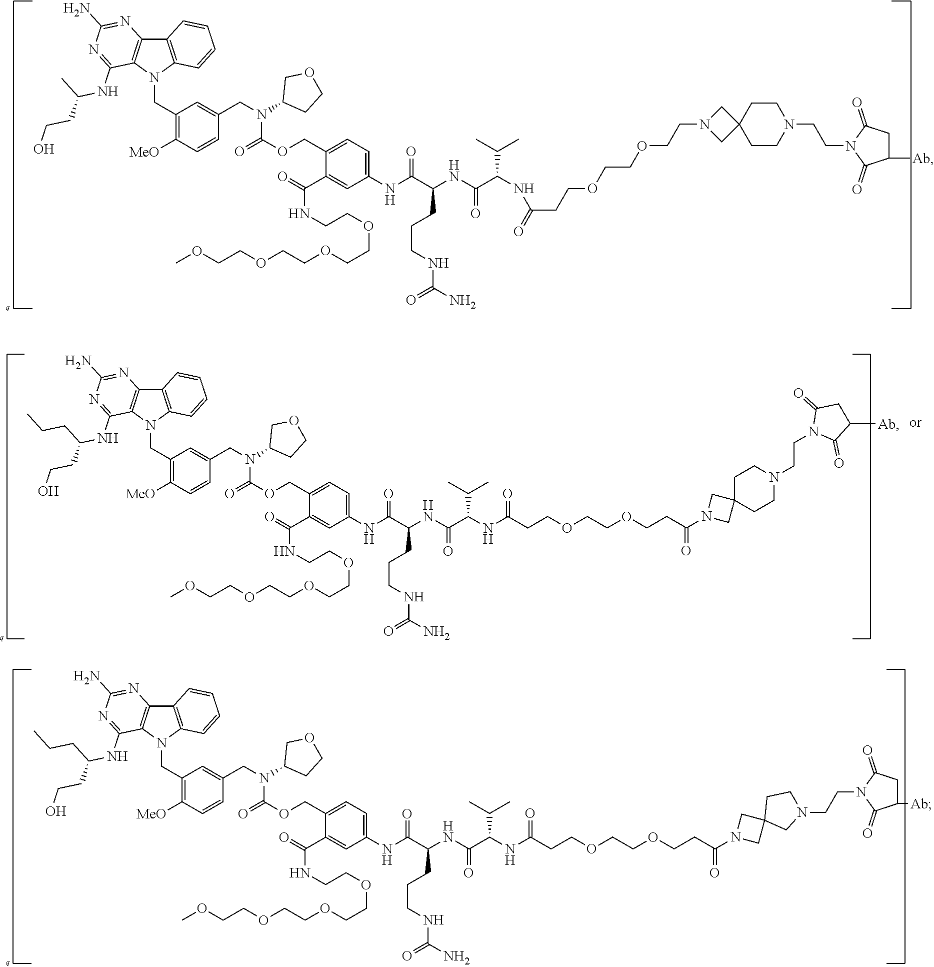

or a pharmaceutically acceptable salt thereof.

80. In another embodiment, the antibody-drug conjugate of any one of embodiments 67-79, wherein q is 1-8, or 1-10, or 2-8, or 2-10, or 4-8, or 4-10, or 6-8, or 6-10.

81. In another embodiment, the antibody-drug conjugate of any one of embodiments 67-79, wherein the antibody or antigen-binding portion thereof binds LIV1.

82. In another embodiment, a pharmaceutical composition comprising the antibody-drug conjugate of any one of embodiments 67-79 and a pharmaceutically acceptable carrier.

83. In another embodiment, a method of treating cancer in a subject in need thereof, comprising administering to the subject a therapeutically effective amount of the antibody-drug conjugate of any one of embodiments 67-80, or the pharmaceutical composition of embodiment 82.

84. In another embodiment, the method of embodiment 83, wherein the cancer is leukemia.

85. In another embodiment, the method of embodiment 84, wherein the leukemia is chronic lymphocytic leukemia, chronic myelocytic leukemia, acute lymphoblastic leukemia, or acute myeloid leukemia.

86. In another embodiment, the method of embodiment 83, wherein the cancer is solid tumor.

87. In another embodiment, the method of embodiment 86, wherein the solid tumor cancer is bladder, renal, breast, ovarian, lung, colorectal, or glioblastoma.

88. In another embodiment, the method of any one of embodiments 83-87, wherein the subject is a human.

89. In another embodiment, the use of the antibody-drug conjugate of any one of embodiments 67-81, or the pharmaceutical composition of embodiment 82, for the preparation of a medicament for treating cancer in a subject in need thereof.

90. In another embodiment, the antibody-drug conjugate of any one of embodiments 67-81, or the pharmaceutical composition of embodiment 82, for use in treating cancer in a subject in need thereof.

### III. Therapeutic Compositions and Methods

### A. Methods of Treating Diseases

In one embodiment, provided herein is a method of treating and/or preventing cancer, which comprises administering to a patient a compound or antibody-drug conjugate provided herein.

In another embodiment, provided herein is method of managing cancer, which comprises administering to a patient a compound or antibody-drug conjugate provided herein.

Also provided herein are methods of treating patients who have been previously treated for cancer but are non-responsive to standard therapies, as well as those who have not previously been treated. Also encompassed are methods of treating patients regardless of patient's age, although some diseases or disorders are more common in certain age groups. Further encompassed are methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

As used herein, the term “cancer” includes, but is not limited to, solid tumors and blood borne tumors. The term “cancer” refers to disease of skin tissues, organs, blood, and vessels, including, but not limited to, cancers of the bladder, bone, blood, brain, breast, cervix, chest, colon, endometrium, esophagus, eye, head, kidney, liver, lymph nodes, lung, mouth, neck, ovaries, pancreas, prostate, rectum, stomach, testis, throat, and uterus. Specific cancers include, but are not limited to, advanced malignancy, amyloidosis, neuroblastoma, meningioma, hemangiopericytoma, multiple brain metastases, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, urachal cancer, papillary thyroid carcinoma, follicular thyroid carcinoma, medullary thyroid carcinoma, and leiomyoma.

In certain embodiments, the cancer is a solid tumor. In certain embodiments, the solid tumor is metastatic. In certain embodiments, the solid tumor is drug-resistant. In certain embodiments, the solid tumor is hepatocellular carcinoma, prostate cancer, ovarian cancer, or glioblastoma.

In certain embodiments, the cancer is a blood borne tumor. In certain embodiments, the blood borne tumor is metastatic. In certain embodiments, the blood borne tumor is drug resistant. In certain embodiments, the cancer is leukemia.

In one embodiment, methods provided herein encompass treating, preventing or managing various types of leukemias such as chronic lymphocytic leukemia (CLL), chronic myelocytic leukemia (CML), acute lymphoblastic leukemia (ALL), acute myeloid leukemia (AML), and acute myeloblastic leukemia (AML) by administering a therapeutically effective amount of a compound or antibody-drug conjugate provided herein.

In some embodiments, the methods provided herein encompass treating, preventing or managing acute leukemia in a subject. In some embodiments, the acute leukemia is acute myeloid leukemia (AML), which includes, but is not limited to, undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), and megakaryoblastic leukemia (M7). In one embodiment, the acute myeloid leukemia is undifferentiated AML (M0). In one embodiment, the acute myeloid leukemia is myeloblastic leukemia (M1). In one embodiment, the acute myeloid leukemia is myeloblastic leukemia (M2). In one embodiment, the acute myeloid leukemia is promyelocytic leukemia (M3 or M3 variant [M3V]). In one embodiment, the acute myeloid leukemia is myelomonocytic leukemia (M4 or M4 variant with eosinophilia [M4E]). In one embodiment, the acute myeloid leukemia is monocytic leukemia (M5). In one embodiment, the acute myeloid leukemia is erythroleukemia (M6). In one embodiment, the acute myeloid leukemia is megakaryoblastic leukemia (M7). Thus, the methods of treating, preventing or managing acute myeloid leukemia in a subject comprise the step of administering to the subject an amount of a compound or antibody-drug conjugate provided herein effective to treat, prevent or manage acute myeloid leukemia alone or in combination. In some embodiments, the methods comprise the step of administering to the subject a compound or antibody-drug conjugate provided herein in combination with a second active agent in amounts effective to treat, prevent or manage acute myeloid leukemia.

In some embodiments, the methods provided herein encompass treating, preventing or managing acute lymphocytic leukemia (ALL) in a subject. In some embodiments, acute lymphocytic leukemia includes leukemia that originates in the blast cells of the bone marrow (B-cells), thymus (T-cells), and lymph nodes. The acute lymphocytic leukemia can be categorized according to the French-American-British (FAB) Morphological Classification Scheme as L1—Mature-appearing lymphoblasts (T-cells or pre-B-cells), L2—Immature and pleomorphic (variously shaped) lymphoblasts (T-cells or pre-B-cells), and L3—Lymphoblasts (B-cells; Burkitt's cells). In one embodiment, the acute lymphocytic leukemia originates in the blast cells of the bone marrow (B-cells). In one embodiment, the acute lymphocytic leukemia originates in the thymus (T-cells). In one embodiment, the acute lymphocytic leukemia originates in the lymph nodes. In one embodiment, the acute lymphocytic leukemia is L1 type characterized by mature-appearing lymphoblasts (T-cells or pre-B-cells). In one embodiment, the acute lymphocytic leukemia is L2 type characterized by immature and pleomorphic (variously shaped) lymphoblasts (T-cells or pre-B-cells). In one embodiment, the acute lymphocytic leukemia is L3 type characterized by lymphoblasts (B-cells; Burkitt's cells). In certain embodiments, the acute lymphocytic leukemia is T-cell leukemia. In one embodiment, the T-cell leukemia is peripheral T-cell leukemia. In another embodiment, the T-cell leukemia is T-cell lymphoblastic leukemia. In another embodiment, the T-cell leukemia is cutaneous T-cell leukemia. In another embodiment, the T-cell leukemia is adult T-cell leukemia. Thus, the methods of treating, preventing or managing acute lymphocytic leukemia in a subject comprise the step of administering to the subject an amount of a compound or antibody-drug conjugate provided herein effective to treat, prevent or manage acute lymphocytic leukemia alone or in combination with a second active agent. In some embodiments, the methods comprise the step of administering to the subject a compound or antibody-drug conjugate provided herein in combination with a second active agent in amounts effective to treat, prevent or manage acute lymphocytic leukemia.

In some embodiments, the methods provided herein encompass treating, preventing or managing chronic myelogenous leukemia (CML) in a subject. The methods comprise the step of administering to the subject an amount of a compound or antibody-drug conjugate provided herein effective to treat, prevent or manage chronic myelogenous leukemia. In some embodiments, the methods comprise the step of administering to the subject a compound or antibody-drug conjugate provided herein in combination with a second active agent in amounts effective to treat, prevent or manage chronic myelogenous leukemia.

In some embodiments, the methods provided herein encompass treating, preventing or managing chronic lymphocytic leukemia (CLL) in a subject. The methods comprise the step of administering to the subject an amount of a compound or antibody-drug conjugate provided herein effective to treat, prevent or manage chronic lymphocytic leukemia. In some embodiments, the methods comprise the step of administering to the subject a compound or antibody-drug conjugate provided herein in combination with a second active agent in amounts effective to treat, prevent or manage chronic lymphocytic leukemia.

In some embodiments, the methods provided herein encompass treating, preventing or managing myelodysplastic syndrome (MDS) in a subject. MDS is characterized by immature blood cells in the bone marrow that do not mature and may progress to AML. In some embodiments, the subject has very low, low, intermediate, high, or very high risk MDS according to the International Prognostic Scoring System (IPSS—R). The methods comprise the step of administering to the subject an amount of a compound or antibody-drug conjugate provided herein effective to treat, prevent or manage MDS. In some embodiments, the methods comprise the step of administering to the subject a compound or antibody-drug conjugate provided herein in combination with a second active agent in amounts effective to treat, prevent or manage MDS.

In certain embodiments, provided herein are methods of treating, preventing, and/or managing disease in patients with impaired renal function. In certain embodiments, provided herein are method of treating, preventing, and/or managing cancer in patients with impaired renal function. In certain embodiments, provided herein are methods of providing appropriate dose adjustments for patients with impaired renal function due to, but not limited to, disease, aging, or other patient factors.

In certain embodiments, provided herein are methods of treating, preventing, and/or managing lymphoma, including non-Hodgkin's lymphoma. In some embodiments, provided herein are methods for the treatment or management of non-Hodgkin's lymphoma (NHL), including but not limited to diffuse large B-cell lymphoma (DLBCL), using prognostic factors.

In certain embodiments, provided herein are methods of treating, preventing, and/or managing multiple myeloma, including relapsed/refractory multiple myeloma in patients with impaired renal function or a symptom thereof, comprising administering a therapeutically effective amount of a compound or antibody-drug conjugate provided herein to a patient having relapsed/refractory multiple myeloma with impaired renal function.

In certain embodiments, the patient to be treated with one of the methods provided herein has not been treated with anticancer therapy prior to the administration of a compound or antibody-drug conjugate provided herein. In certain embodiments, the patient to be treated with one of the methods provided herein has been treated with anticancer therapy prior to the administration of a compound or antibody-drug conjugate provided herein. In certain embodiments, the patient to be treated with one of the methods provided herein has developed drug resistance to the anticancer therapy.

The methods provided herein encompass treating a patient regardless of patient's age, although some diseases or disorders are more common in certain age groups.

### B. Routes of Administration and Carriers

Provided herein are compositions (e.g., pharmaceutical compositions) comprising a hydrolyzable linker-payload compound or antibody-drug conjugate provided herein and one or more pharmaceutically acceptable carriers.

In various embodiments, a compound or antibody-drug conjugate provided herein may be administered in vivo by various routes, including, but not limited to, oral, intra-arterial, parenteral (including intravenous and subcutaneous), intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations, such as liquid formulations or formulations suitable for injections, inhalations, and the like. Alternatively, in some embodiments, the composition may be provided as a lyophilized powder that may be reconstituted upon addition of an appropriate liquid or carrier, for example, sterile water. The appropriate formulation and route of administration may be selected according to the intended application.

In various embodiments, compositions comprising a compound or antibody-drug conjugate provided herein are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available.

### IV. Synthetic Examples of TLR 7/8 Agonists and Hydrolyzable Linker-Payload Compounds

### Example A1. N-(3-((2-amino-4-(((S)-1-hydroxyhexan-3-yl)amino)-5H-pyrimido[5,4-b]indol-5-yl)methyl)-4-methoxybenzyl)-1-(3-(2,5-dioxo-2,5-dihydro-1H-pyrrol-1-yl)propanamido)-N—((S)-tetrahydrofuran-3-yl)-3,6,9,12-tetraoxapentadecan-15-amide

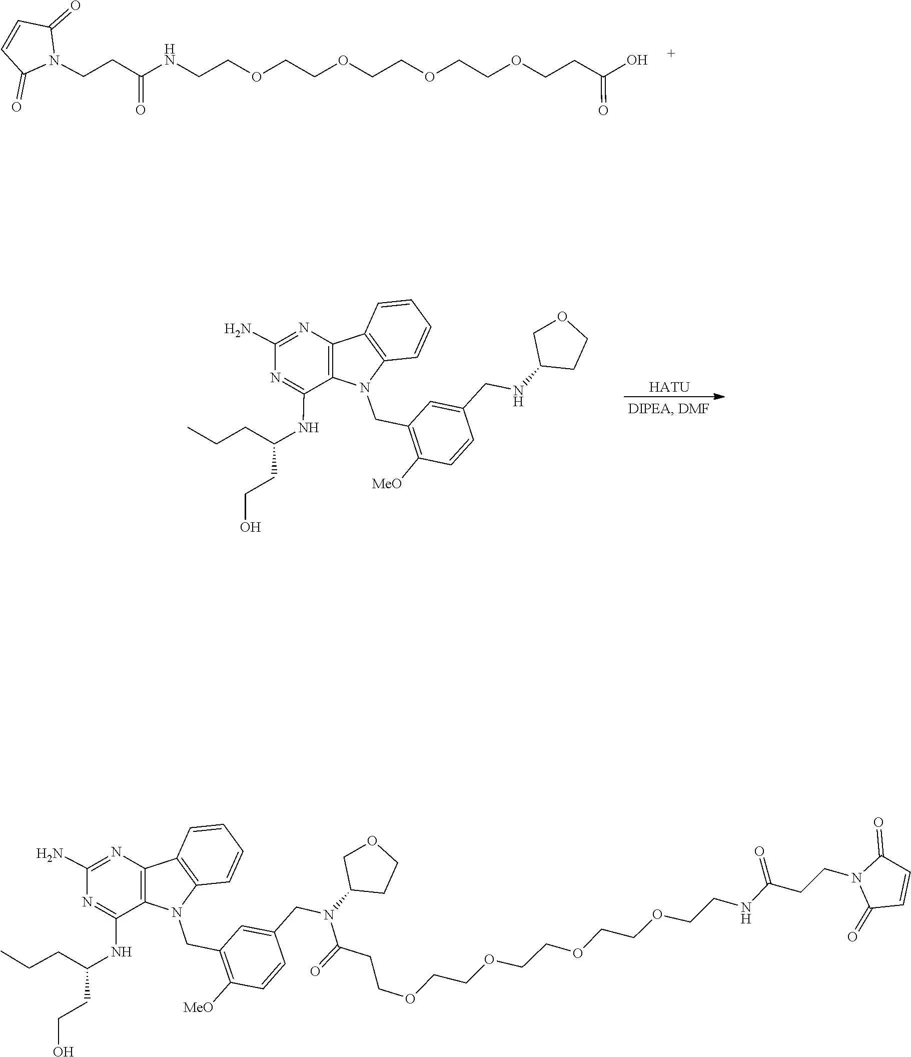 [SMILES: O=C(O)CCOCCOCCOCCOCCNC(=O)CCN1C(=O)C=CC1=O ; CCC[C@@H](CCO)Nc1nc(N)nc2c3ccccc3n(Cc3cc(CN[C@H]4CCOC4)ccc3OC)c12 ; "HATU" ; CCC[C@@H](CCO)Nc1nc(N)nc2c3ccccc3n(Cc3cc(CN(C(=O)CCOCCOCCOCCOCCNC(=O)CCN4C(=O)C=CC4=O)[C@H]4CCOC4)ccc3OC)c12]

A scintillation vial was charged with (S)-3-((2-amino-5-(2-methoxy-5-((((S)-tetrahydrofuran-3-yl)amino)methyl)benzyl)-5H-pyrimido[5,4-b]indol-4-yl)amino)hexan-1-ol (15 mg, 0.029 mmol), 1-Maleimido-3-oxo-7,10,13,16-tetraoxa-4-azanonadecan-19-oic acid (13.25 mg, 0.032 mmol), HATU (12.10 mg, 0.032 mmol) and DMF (1 mL). DIPEA (0.015 mL, 0.087 mmol) was added, and the reaction stirred at room temperature for 1 hour. The reaction mixture was quenched using 2% TFA in water (1 mL), filtered and purified using preparative HPLC (30×150 mm C18 column, 10 to 50% MeCN in water containing 0.05% TFA). Product fractions were combined and lyophilized to give N-(3-((2-amino-4-(((S)-1-hydroxyhexan-3-yl)amino)-5H-pyrimido[5,4-b]indol-5-yl)methyl)-4-methoxybenzyl)-1-(3-(2,5-dioxo-2,5-dihydro-1H-pyrrol-1-yl)propanamido)-N—((S)-tetrahydrofuran-3-yl)-3,6,9,12-tetraoxapentadecan-15-amide, trifluoroacetate salt (6 mg, 5.8 μmol, 20.1% yield) as a solid. LC-MS (ES, m/z): [M+H]⁺=917.5, r.t. 1.20 mins.

### Example A2. 4-((2S,5S)-20-amino-5-isopropyl-4,7-dioxo-2-(3-ureidopropyl)-9,12,15,18-tetraoxa-3,6-diazaicosanamido)-2-((26-oxo-2,5,8,11,14,17,20,23-octaoxa-27-azanonacosan-29-yl)carbamoyl)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Formic Acid Salt

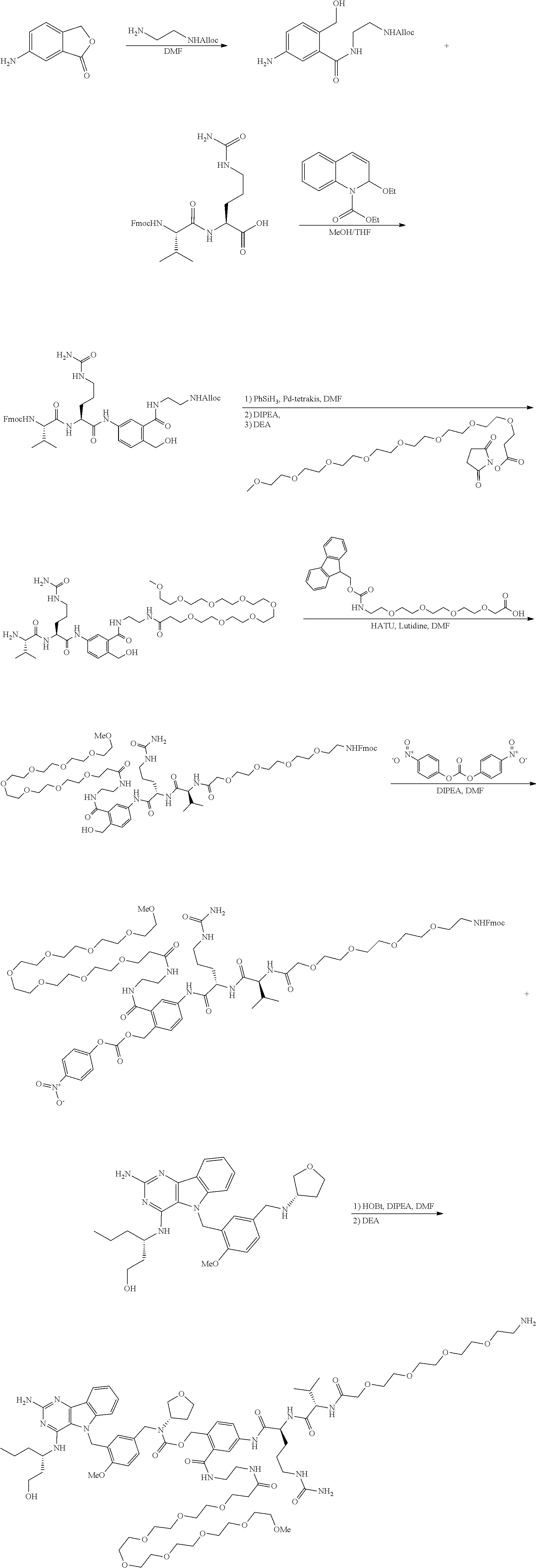

Step A2-1. Allyl (2-(5-amino-2-(hydroxymethyl)benzamido)ethyl)carbamate A scintillation vial was charged with 6-aminoisobenzofuran-1(3H)-one (7 g, 46.9 mmol), allyl (2-aminoethyl)carbamate (10.15 g, 70.4 mmol) and DMF (7 mL). The reaction was stirred for 72 hours at 60° C. After cooling, the reaction mixture was evaporated to dryness. The residue was purified using flash chromatography (330 g SiO₂ column, 0 to 100% EtOAc in hexanes), giving allyl (2-(5-amino-2-(hydroxymethyl)benzamido)ethyl)carbamate (12.4 g, 42.1 mmol, 90% yield) as an oil. LC-MS (ES, m/z): [M+H]⁺=276.2 (product—H₂O).

### Step A2-2. Allyl (2-(5-((S)-2-((S)-2-((((9H-fluoren-9-yl)methoxy)carbonyl)amino)-3-methylbutanamido)-5-ureidopentanamido)-2-(hydroxymethyl)benzamido)ethyl)carbamate

A solution of (S)-2-((S)-2-((((9H-fluoren-9-yl)methoxy)carbonyl)amino)-3-methylbutanamido)-5-ureidopentanoic acid (5.00 g, 10.07 mmol), allyl (2-(5-amino-2-(hydroxymethyl)benzamido)ethyl)carbamate (5.91 g, 20.14 mmol) and ethyl 2-ethoxyquinoline-1(2H)-carboxylate (4.98 g, 20.14 mmol) in THF (50 mL) and MeOH (25 mL) was stirred for 72 hours at room temperature. The reaction mixture was evaporated to dryness and redissolved in DMF (20 mL), diluted with DCM (20 mL) and purified using flash chromatography (330 g SiO₂ column, 50 to 100% EtOAc in hexanes, then 0 to 25% MeOH in DCM), giving allyl (2-(5-((S)-2-((S)-2-((((9H-fluoren-9-yl)methoxy)carbonyl)amino)-3-methylbutanamido)-5-ureidopentanamido)-2-(hydroxymethyl)benzamido)ethyl)carbamate (4.95 g, 6.41 mmol, 63.7% yield) as a solid. LC-MS (ES, m/z): [M+H]⁺=772.4. ¹H NMR (400 MHz, DMSO-d₆) δ 10.12 (s, 1H), 8.43 (br t, J=5.6 Hz, 1H), 8.11 (br d, J=7.4 Hz, 1H), 7.90 (d, J=7.5 Hz, 2H), 7.75 (t, J=7.0 Hz, 2H), 7.69 (d, J=1.8 Hz, 1H), 7.64 (dd, J=8.3, 2.1 Hz, 1H), 7.48-7.38 (m, 4H), 7.36-7.31 (m, 2H), 7.25 (br t, J=5.7 Hz, 1H), 5.99-5.85 (m, 2H), 5.40 (s, 2H), 5.28 (dd, J=17.3, 1.7 Hz, 1H), 5.24-5.14 (m, 2H), 4.54-4.40 (m, 5H), 4.36-4.21 (m, 3H), 3.94 (dd, J=9.0, 7.1 Hz, 1H), 3.31-3.25 (m, 2H), 3.16 (q, J=6.1 Hz, 2H), 3.07-2.90 (m, 2H), 2.00 (dq, J=13.6, 6.7 Hz, 1H), 1.75-1.65 (m, 1H), 1.65-1.55 (m, 1H), 1.51-1.42 (m, 1H), 1.42-1.33 (m, 1H), 0.89 (br d, J=6.8 Hz, 3H), 0.86 (br d, J=6.8 Hz, 3H).

### Step A2-3. 5—((S)-2-((S)-2-amino-3-methylbutanamido)-5-ureidopentanamido)-2-(hydroxymethyl)-N-(26-oxo-2,5,8,11,14,17,20,23-octaoxa-27-azanonacosan-29-yl)benzamide, Trifluoroacetate Salt

A scintillation vial was charged with allyl (2-(5-((S)-2-((S)-2-((((9H-fluoren-9-yl)methoxy)carbonyl)amino)-3-methylbutanamido)-5-ureidopentanamido)-2-(hydroxymethyl)benzamido)ethyl)carbamate (350 mg, 0.453 mmol), tetrakis(triphenylphosphine)palladium(0) (52.4 mg, 0.045 mmol) and DMF (2 mL). After cooling in an ice bath, phenyl silane (0.200 μl, 1.587 mmol) was added, and the reaction stirred at room temperature for 3 hours. A solution of m-PEG8-NHS ester (277 mg, 0.544 mmol) in DMF (1 mL) was added, and the reaction allowed to warm slowly to room temperature and stirred for 16 hours. Diethylamine (0.474 mL, 4.53 mmol) was added, and the reaction stirred at room temperature for 30 minutes, then evaporated to dryness. The crude material was purified using reverse-phase flash chromatography (50 g C18 column, 0 to 30% MeCN in water containing 0.05% TFA). Product fractions were combined and lyophilized to give 5-((S)-2-((S)-2-amino-3-methylbutanamido)-5-ureidopentanamido)-2-(hydroxymethyl)-N-(26-oxo-2,5,8,11,14,17,20,23-octaoxa-27-azanonacosan-29-yl)benzamide, trifluoroacetate salt (199 mg, 0.204 mmol, 45.1% yield) as an oil. LC-MS (ES, m/z): [M+H]⁺=860.5.

### Step A2-4. (9H-fluoren-9-yl)methyl ((6S,9S)-1-amino-6-((4-(hydroxymethyl)-3-((26-oxo-2,5,8,11,14,17,20,23-octaoxa-27-azanonacosan-29-yl)carbamoyl)phenyl)carbamoyl)-9-isopropyl-1,8,11-trioxo-13,16,19,22-tetraoxa-2,7,10-triazatetracosan-24-yl)carbamate

To a stirred solution of 5-((S)-2-((S)-2-amino-3-methylbutanamido)-5-ureidopentanamido)-2-(hydroxymethyl)-N-(26-oxo-2,5,8,11,14,17,20,23-octaoxa-27-azanonacosan-29-yl)benzamide, trifluoroacetate salt (199 mg, 0.204 mmol), 1-(9H-fluoren-9-yl)-3-oxo-2,7,10,13,16-pentaoxa-4-azaoctadecan-18-oic acid (116 mg, 0.245 mmol) and HATU (93 mg, 0.245 mmol) in DMF (2 mL) was added 2,6-lutidine (0.071 mL, 0.613 mmol). The reaction was stirred at room temperature for 1 hour. The reaction mixture was diluted with water (1 mL) and MeCN (0.5 mL), filtered and purified using reverse-phase flash chromatography (50 g C₁₈ column, 0 to 60% MeCN in water containing 10 mM NH₄OAc). Product fractions were combined and lyophilized to give (9H-fluoren-9-yl)methyl ((6S,9R)-1-amino-6-((4-(hydroxymethyl)-3-((26-oxo-2,5,8,11,14,17,20,23-octaoxa-27-azanonacosan-29-yl)carbamoyl)phenyl)carbamoyl)-9-isopropyl-1,8,11-trioxo-13,16,19,22-tetraoxa-2,7,10-triazatetracosan-24-yl)carbamate (134 mg, 0.102 mmol, 49.9% yield). LC-MS (ES, m/z): [M+H]⁺=1315.6.

### Step A2-5. (9H-fluoren-9-yl)methyl ((6S,9S)-1-amino-9-isopropyl-6-((4-((((4-nitrophenoxy)carbonyl)oxy)methyl)-3-((26-oxo-2,5,8,11,14,17,20,23-octaoxa-27-azanonacosan-29-yl)carbamoyl)phenyl)carbamoyl)-1,8,11-trioxo-13,16,19,22-tetraoxa-2,7,10-triazatetracosan-24-yl)carbamate

A scintillation vial was charged with (9H-fluoren-9-yl)methyl ((6S,9S)-1-amino-6-((4-(hydroxymethyl)-3-((26-oxo-2,5,8,11,14,17,20,23-octaoxa-27-azanonacosan-29-yl)carbamoyl)phenyl)carbamoyl)-9-isopropyl-1,8,11-trioxo-13,16,19,22-tetraoxa-2,7,10-triazatetracosan-24-yl)carbamate (160 mg, 0.122 mmol), bis(4-nitrophenyl) carbonate (148 mg, 0.486 mmol) and DMF (2 mL). The reaction mixture was stirred at room temperature for 1 hour. The reaction mixture was diluted with DCM (2 mL) and purified using flash chromatography (50 g SiO₂ column, 0 to 100% (20% MeOH in DCM)), giving (9H-fluoren-9-yl)methyl ((6S,9S)-1-amino-9-isopropyl-6-((4-((((4-nitrophenoxy)carbonyl)oxy)methyl)-3-((26-oxo-2,5,8,11,14,17,20,23-octaoxa-27-azanonacosan-29-yl)carbamoyl)phenyl)carbamoyl)-1,8,11-trioxo-13,16,19,22-tetraoxa-2,7,10-triazatetracosan-24-yl)carbamate (101 mg, 0.068 mmol, 56.1% yield) as a gum. LC-MS (ES, m/z): [[M+2H]/2]⁺=741.3.

### Step A2-6. 4-((2S,5S)-20-amino-5-isopropyl-4,7-dioxo-2-(3-ureidopropyl)-9,12,15,18-tetraoxa-3,6-diazaicosanamido)-2-((26-oxo-2,5,8,11,14,17,20,23-octaoxa-27-azanonacosan-29-yl)carbamoyl)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Formic Acid Salt

A scintillation vial was charged with (S)-3-((2-amino-5-(2-methoxy-5-((((S)-tetrahydrofuran-3-yl)amino)methyl)benzyl)-5H-pyrimido[5,4-b]indol-4-yl)amino)hexan-1-ol (50 mg, 0.096 mmol), (9H-fluoren-9-yl)methyl ((6S,9S)-1-amino-9-isopropyl-6-((4-((((4-nitrophenoxy)carbonyl)oxy)methyl)-3-((26-oxo-2,5,8,11,14,17,20,23-octaoxa-27-azanonacosan-29-yl)carbamoyl)phenyl)carbamoyl)-1,8,11-trioxo-13,16,19,22-tetraoxa-2,7,10-triazatetracosan-24-yl)carbamate (171 mg, 0.116 mmol), HOBt (7.38 mg, 0.048 mmol) and DMF (2 mL). DIPEA (0.051 mL, 0.289 mmol) was added, and the reaction stirred for 72 hours at room temperature. Diethylamine (0.101 mL, 0.964 mmol) was added, and the reaction stirred at room temperature for 1 hour. The reaction mixture was quenched with 5% formic acid in water (2 mL), solubilized with MeCN (1 mL), filtered and purified using reverse-phase flash chromatography (50 g C18 column, 0 to 50% MeCN in water containing 0.05% TFA). Product fractions were combined and lyophilized. The material was further purified using preparative HPLC (30×150 mm C18 column, 10 to 47% MeCN in water containing 0.05% formic acid). Product fractions were combined and lyophilized to give 4-((2S,5S)-20-amino-5-isopropyl-4,7-dioxo-2-(3-ureidopropyl)-9,12,15,18-tetraoxa-3,6-diazaicosanamido)-2-((26-oxo-2,5,8,11,14,17,20,23-octaoxa-27-azanonacosan-29-yl)carbamoyl)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, formic acid salt (11 mg, 6.53 μmol, 6.78% yield). LC-MS (ES, m/z): [M+H]⁺=1638.7, r.t. 1.11 mins.

### Example A3. 4-((2S,5S)-24-(2,5-dioxo-2,5-dihydro-1H-pyrrol-1-yl)-5-isopropyl-4,7,22-trioxo-2-(3-ureidopropyl)-9,12,15,18-tetraoxa-3,6,21-triazatetracosanamido)-2-((26-oxo-2,5,8,11,14,17,20,23-octaoxa-27-azanonacosan-29-yl)carbamoyl)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate

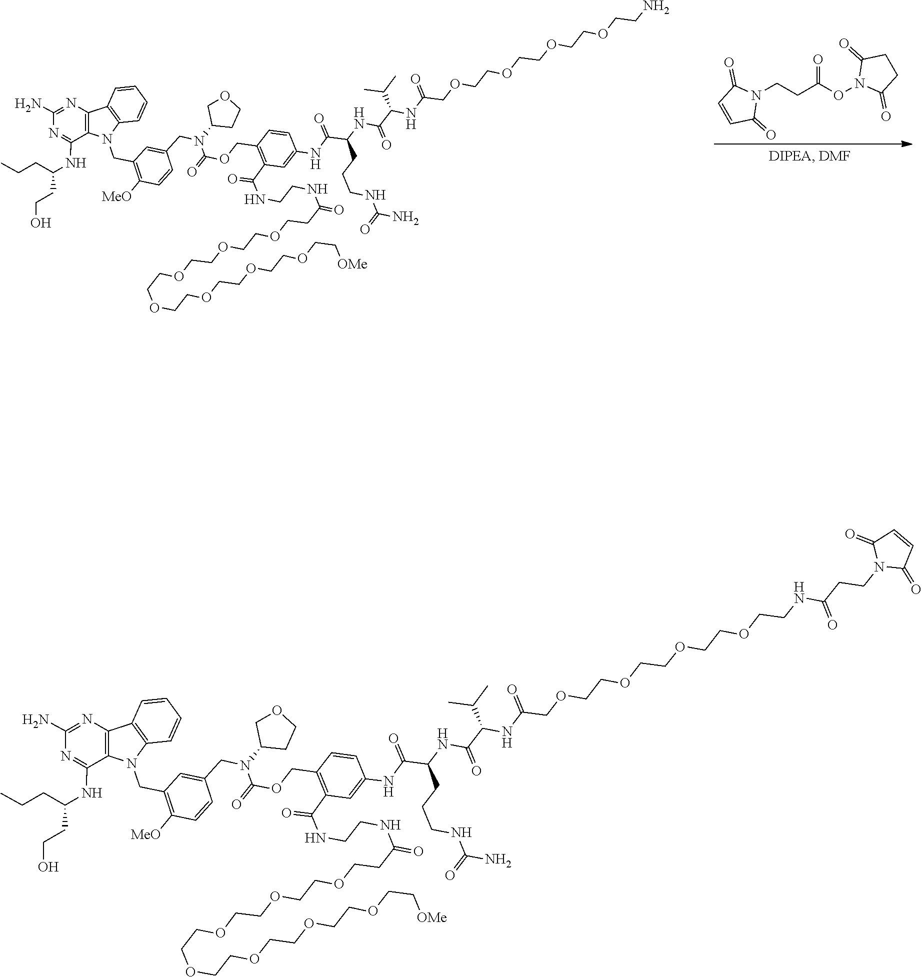 [SMILES: CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](NC(=O)COCCOCCOCCOCCN)C(C)C)cc4C(=O)NCCNC(=O)CCOCCOCCOCCOCCOCCOCCOCCOC)[C@H]4CCOC4)ccc3OC)c12 ; O=C(CCN1C(=O)C=CC1=O)ON1C(=O)CCC1=O ; CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](NC(=O)COCCOCCOCCOCCNC(=O)CCN5C(=O)C=CC5=O)C(C)C)cc4C(=O)NCCNC(=O)CCOCCOCCOCCOCCOCCOCCOCCOC)[C@H]4CCOC4)ccc3OC)c12]

To a stirred solution of 4-((2S,5S)-20-amino-5-isopropyl-4,7-dioxo-2-(3-ureidopropyl)-9,12,15,18-tetraoxa-3,6-diazaicosanamido)-2-((26-oxo-2,5,8,11,14,17,20,23-octaoxa-27-azanonacosan-29-yl)carbamoyl)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, formic acid salt (17 mg, 10.10 μmol) and 2,5-dioxopyrrolidin-1-yl 3-(2,5-dioxo-2,5-dihydro-1H-pyrrol-1-yl)propanoate (2.69 mg, 10.10 μmol) in DMF (0.5 mL) was added DIPEA (3.53 μl, 0.020 mmol). The reaction was stirred at room temperature for 30 minutes, then quenched with 5% formic acid in water (3 mL). MeCN (0.5 mL) was added, and the reaction was filtered and purified using preparative HPLC (30×150 mm C₁₈ column, 10 to 40% MeCN in water containing 0.05% formic acid). Product fractions were combined and lyophilized to give 4-((2S,5S)-24-(2,5-dioxo-2,5-dihydro-1H-pyrrol-1-yl)-5-isopropyl-4,7,22-trioxo-2-(3-ureidopropyl)-9,12,15,18-tetraoxa-3,6,21-triazatetracosanamido)-2-((26-oxo-2,5,8,11,14,17,20,23-octaoxa-27-azanonacosan-29-yl)carbamoyl)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, formic acid salt (5.4 mg, 2.94 μmol, 29.1% yield). LC-MS (ES, m/z): [[M+2H]/2]⁺=895.4.

### Example A4. 2-((2,5,8,11-tetraoxatridecan-13-yl)carbamoyl)-4-((17R,20R)-1-amino-17-isopropyl-15,18-dioxo-20-(3-ureidopropyl)-3,6,9,12-tetraoxa-16,19-diazahenicosan-21-amido)benzyl (2-amino-2-oxoethyl)(3-((2-amino-4-(((S)-1-hydroxyhexan-3-yl)amino)-5H-pyrimido[5,4-b]indol-5-yl)methyl)-4-methoxybenzyl)carbamate, Trifluoroacetate Salt

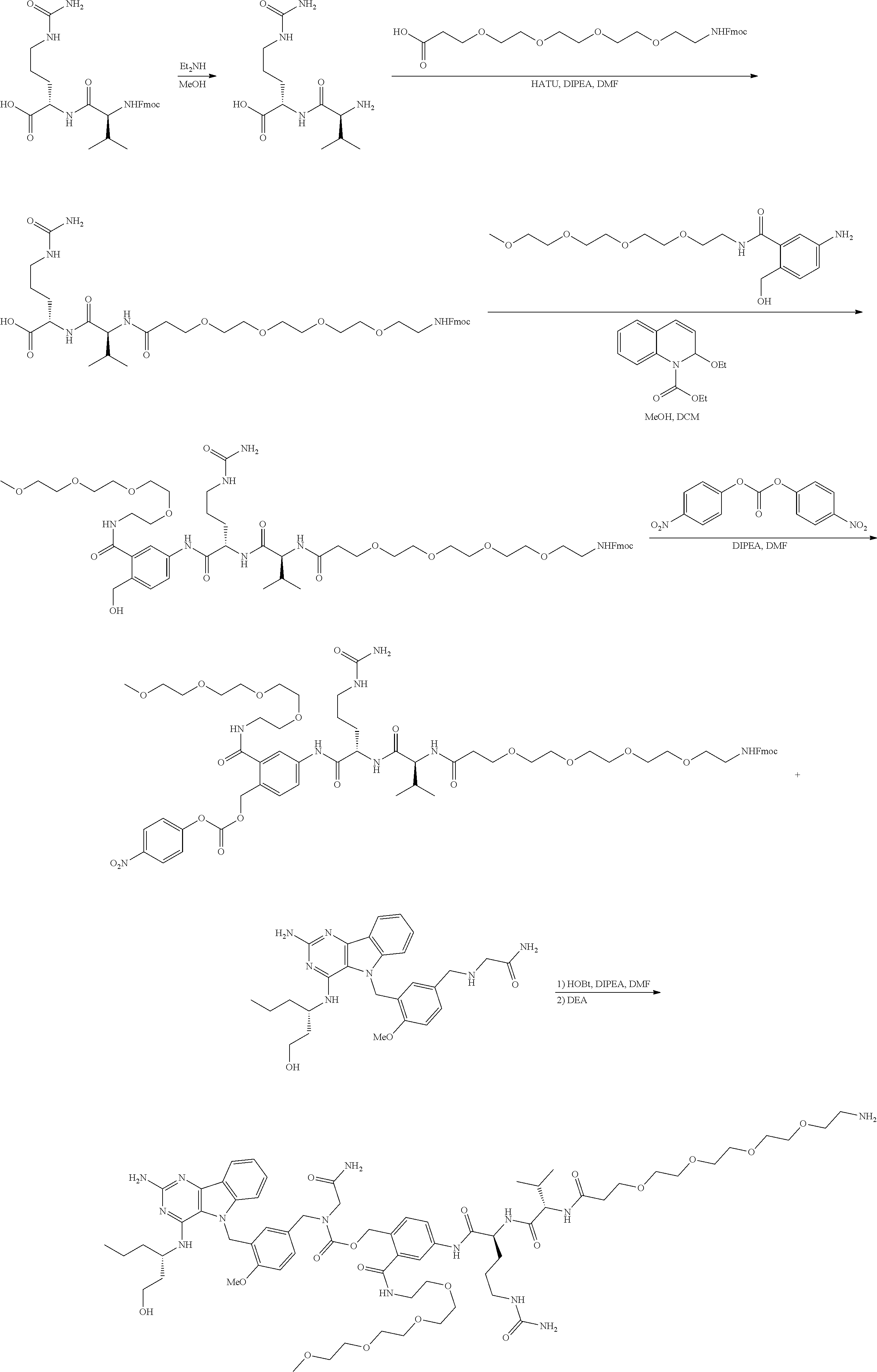

### Step A4-1. (21S,24S)-1-(9H-fluoren-9-yl)-21-isopropyl-3,19,22-trioxo-24-(3-ureidopropyl)-2,7,10,13,16-pentaoxa-4,20,23-triazapentacosan-25-oic acid

To a stirred suspension of (S)-2-((S)-2-((((9H-fluoren-9-yl)methoxy)carbonyl)amino)-3-methylbutanamido)-5-ureidopentanoic acid (2 g, 4.03 mmol) in MeOH (20 mL) was added diethylamine (1.052 mL, 10.07 mmol). The reaction was stirred at room temperature for 16 hours. The reaction mixture was evaporated to dryness, then the residue was dissolved in DMF (8 mL) and evaporated to dryness. The residue was dissolved in DMF (8 mL) and a solution of 1-(9H-fluoren-9-yl)-3-oxo-2,7,10,13,16-pentaoxa-4-azanonadecan-19-oic acid (2.062 g, 4.23 mmol), HATU (1.685 g, 4.43 mmol) and DIPEA (1.407 mL, 8.06 mmol) in DMF (4 mL) was added. The reaction was stirred for 1 hour at room temperature. The reaction mixture was diluted with water (4 mL) and MeCN (2 mL), acidified with HCl and purified using reverse-phase flash chromatography (150 g C18 column, 10 to 60% MeCN in water containing 0.05% TFA). Product fractions were combined and lyophilized to give (21S,24S)-1-(9H-fluoren-9-yl)-21-isopropyl-3,19,22-trioxo-24-(3-ureidopropyl)-2,7,10,13,16-pentaoxa-4,20,23-triazapentacosan-25-oic acid (1.06 g, 1.425 mmol, 35.4% yield). LC-MS (ES, m/z): [M+H]⁺=744.5. ¹H NMR (400 MHz, DMSO-d₆) δ 8.18 (d, J=7.3 Hz, 1H), 7.89 (d, J=7.5 Hz, 2H), 7.82 (d, J=9.1 Hz, 1H), 7.70 (d, J=7.4 Hz, 2H), 7.42 (t, J=7.5 Hz, 2H), 7.38-7.28 (m, 3H), 5.95 (br s, 1H), 5.37 (br s, 2H), 4.33-4.18 (m, 4H), 4.17-4.08 (m, 1H), 3.63-3.55 (m, 2H), 3.53-3.44 (m, 13H), 3.44-3.38 (m, 2H), 3.14 (q, J=5.8 Hz, 2H), 2.96 (br t, J=6.4 Hz, 2H), 2.50-2.31 (m, 2H), 2.00-1.90 (m, 1H), 1.74-1.52 (m, 2H), 1.46-1.33 (m, 2H), 0.87 (br d, J=6.8 Hz, 3H), 0.83 (br d, J=6.8 Hz, 3H).

### Step A4-2. (9H-fluoren-9-yl)methyl ((6S,9S)-6-((3-((2,5,8,11-tetraoxatridecan-13-yl)carbamoyl)-4-(hydroxymethyl)phenyl)carbamoyl)-1-amino-9-isopropyl-1,8,11-trioxo-14,17,20,23-tetraoxa-2,7,10-triazapentacosan-25-yl)carbamate

To a stirred solution of (21S,24S)-1-(9H-fluoren-9-yl)-21-isopropyl-3,19,22-trioxo-24-(3-ureidopropyl)-2,7,10,13,16-pentaoxa-4,20,23-triazapentacosan-25-oic acid (1.06 g, 1.425 mmol) and 5-amino-2-(hydroxymethyl)-N-(2,5,8,11-tetraoxatridecan-13-yl)benzamide (1.016 g, 2.85 mmol) in MeOH (5 mL) and DCM (5 mL) was added ethyl 2-ethoxyquinoline-1(2H)-carboxylate (0.705 g, 2.85 mmol). The reaction was stirred at room temperature for 16 hours. The reaction mixture was evaporated to dryness, then purified using flash chromatography (120 g SiO₂ column, 0 to 50% (20% MeOH in DCM) in DCM), giving (9H-fluoren-9-yl)methyl ((6S,9S)-6-((3-((2,5,8,11-tetraoxatridecan-13-yl)carbamoyl)-4-(hydroxymethyl)phenyl) carbamoyl)-1-amino-9-isopropyl-1,8,11-trioxo-14,17,20,23-tetraoxa-2,7,10-triazapentacosan-25-yl)carbamate (503 mg, 0.465 mmol, 32.6% yield) as a solid. LC-MS (ES, m/z): [M+H]⁺=1082.4.

### Step A4-3. (9H-fluoren-9-yl)methyl ((6S,9S)-6-((3-((2,5,8,11-tetraoxatridecan-13-yl)carbamoyl)-4-((((4-nitrophenoxy)carbonyl)oxy)methyl)phenyl)carbamoyl)-1-amino-9-isopropyl-1,8,11-trioxo-14,17,20,23-tetraoxa-2,7,10-triazapentacosan-25-yl)carbamate

To a stirred solution of (9H-fluoren-9-yl)methyl ((6S,9S)-6-((3-((2,5,8,11-tetraoxatridecan-13-yl)carbamoyl)-4-(hydroxymethyl)phenyl)carbamoyl)-1-amino-9-isopropyl-1,8,11-trioxo-14,17,20,23-tetraoxa-2,7,10-triazapentacosan-25-yl)carbamate (300 mg, 0.277 mmol) in DMF (3 mL) was added bis(4-nitrophenyl) carbonate (253 mg, 0.832 mmol) followed by DIPEA (0.097 mL, 0.554 mmol). The reaction was stirred at room temperature for 30 minutes. The reaction mixture was purified using flash chromatography (80 g SiO₂ column, 0 to 50% (20% MeOH in DCM) in DCM), giving (9H-fluoren-9-yl)methyl ((6S,9S)-6-((3-((2,5,8,11-tetraoxatridecan-13-yl)carbamoyl)-4-((((4-nitrophenoxy)carbonyl)oxy)methyl)phenyl) carbamoyl)-1-amino-9-isopropyl-1,8,11-trioxo-14,17,20,23-tetraoxa-2,7,10-triazapentacosan-25-yl)carbamate (204 mg, 0.164 mmol, 59.0% yield) as an oil. LC-MS (ES, m/z): [M+H]⁺=1247.6.

### Step A4-4. 2-((2,5,8,11-tetraoxatridecan-13-yl)carbamoyl)-4-((17S,20S)-1-amino-17-isopropyl-15,18-dioxo-20-(3-ureidopropyl)-3,6,9,12-tetraoxa-16,19-diazahenicosan-21-amido)benzyl (2-amino-2-oxoethyl)(3-((2-amino-4-(((S)-1-hydroxyhexan-3-yl)amino)-5H-pyrimido[5,4-b]indol-5-yl)methyl)-4-methoxybenzyl)carbamate, Trifluoroacetate Salt

To a stirred solution of (S)-2-((3-((2-amino-4-((1-hydroxyhexan-3-yl)amino)-5H-pyrimido[5,4-b]indol-5-yl)methyl)-4-methoxybenzyl)amino)acetamide, trifluoroacetate salt (40 mg, 0.065 mmol), (9H-fluoren-9-yl)methyl ((6S,9S)-6-((3-((2,5,8,11-tetraoxatridecan-13-yl)carbamoyl)-4-((((4-nitrophenoxy)carbonyl)oxy)methyl)phenyl)carbamoyl)-1-amino-9-isopropyl-1,8,11-trioxo-14,17,20,23-tetraoxa-2,7,10-triazapentacosan-25-yl)carbamate (89 mg, 0.071 mmol) and HOBt (9.89 mg, 0.065 mmol) in DMF (1 mL) was added DIPEA (0.034 mL, 0.194 mmol). The reaction was stirred at room temperature for 2 hours. Diethylamine (0.067 mL, 0.646 mmol) was added, and the reaction stirred for a further 30 minutes at room temperature. The reaction mixture was diluted with water (1 mL) and MeCN (1 mL), filtered and purified using reverse-phase flash chromatography (50 g C18 column, 0 to 50% MeCN in water containing 0.05% TFA). Product fractions were combined and lyophilized to give 2-((2,5,8,11-tetraoxatridecan-13-yl)carbamoyl)-4-((17S,20S)-1-amino-17-isopropyl-15,18-dioxo-20-(3-ureidopropyl)-3,6,9,12-tetraoxa-16,19-diazahenicosan-21-amido)benzyl (2-amino-2-oxoethyl)(3-((2-amino-4-(((S)-1-hydroxyhexan-3-yl)amino)-5H-pyrimido[5,4-b]indol-5-yl)methyl)-4-methoxybenzyl)carbamate, trifluoroacetate salt (62 mg, 0.041 mmol, 63.8% yield) as a solid. LC-MS (ES, m/z): [M+H]⁺=1392.4, r.t. 1.04 mins.

### Example A5. 2-((2,5,8,11-tetraoxatridecan-13-yl)carbamoyl)-4-((2S,5S)-28-(2,5-dioxo-2,5-dihydro-1H-pyrrol-1-yl)-5-isopropyl-4,7,23-trioxo-2-(3-ureidopropyl)-10,13,16,19-tetraoxa-3,6,22-triazaoctacosanamido)benzyl (2-amino-2-oxoethyl)(3-((2-amino-4-(((S)-1-hydroxyhexan-3-yl)amino)-5H-pyrimido[5,4-b]indol-5-yl)methyl)-4-methoxybenzyl)carbamate

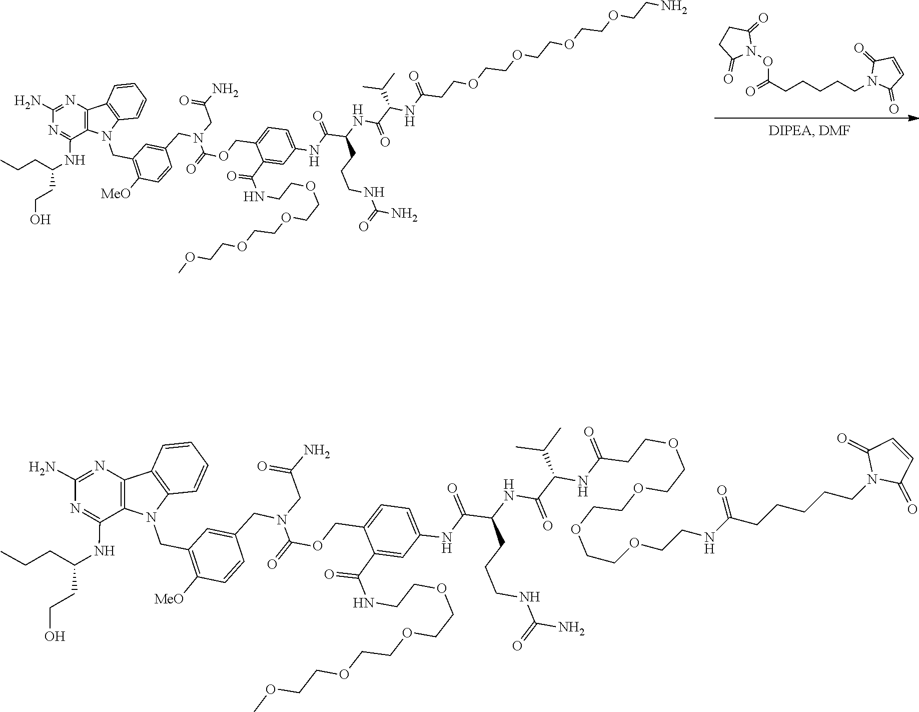 [SMILES: CCC[C@@H](CCO)Nc1nc(N)nc2c3ccccc3n(Cc3cc(CN(CC(N)=O)C(=O)OCc4ccc(NC(=O)[C@H](CCCNC(N)=O)NC(=O)[C@@H](NC(=O)CCOCCOCCOCCOCCN)C(C)C)cc4C(=O)NCCOCCOCCOCCOC)ccc3OC)c12 ; O=C(CCCCCN1C(=O)C=CC1=O)ON1C(=O)CCC1=O ; CCC[C@@H](CCO)Nc1nc(N)nc2c3ccccc3n(Cc3cc(CN(CC(N)=O)C(=O)OCc4ccc(NC(=O)[C@H](CCCNC(N)=O)NC(=O)[C@@H](NC(=O)CCOCCOCCOCCOCCNC(=O)CCCCCN5C(=O)C=CC5=O)C(C)C)cc4C(=O)NCCOCCOCCOCCOC)ccc3OC)c12]

A scintillation vial was charged with 2-((2,5,8,11-tetraoxatridecan-13-yl)carbamoyl)-4-((17S,20S)-1-amino-17-isopropyl-15,18-dioxo-20-(3-ureidopropyl)-3,6,9,12-tetraoxa-16,19-diazahenicosan-21-amido)benzyl(2-amino-2-oxoethyl)(3-((2-amino-4-(((S)-1-hydroxyhexan-3-yl)amino)-5H-pyrimido[5,4-b]indol-5-yl)methyl)-4-methoxybenzyl)carbamate (17 mg, 0.012 mmol), N-Succinimidyl 6-maleimidohexanoate (3.77 mg, 0.012 mmol) and DMF (1 mL). DIPEA (4.27 μl, 0.024 mmol) was added, and the reaction stirred at room temperature for 1 hour. The reaction mixture was quenched with 5% TFA in water (1 mL), filtered and purified using preparative HPLC (30×150 mm C18 column, 10 to 50% MeCN in water containing 0.05% TFA). Product fractions were combined and lyophilized to give 2-((2,5,8,11-tetraoxatridecan-13-yl)carbamoyl)-4-((2S,5S)-28-(2,5-dioxo-2,5-dihydro-1H-pyrrol-1-yl)-5-isopropyl-4,7,23-trioxo-2-(3-ureidopropyl)-10,13,16,19-tetraoxa-3,6,22-triazaoctacosanamido)benzyl (2-amino-2-oxoethyl)(3-((2-amino-4-(((S)-1-hydroxyhexan-3-yl)amino)-5H-pyrimido[5,4-b]indol-5-yl)methyl)-4-methoxybenzyl)carbamate (9.1 mg, 5.74 μmol, 47.0% yield) as a white solid. LC-MS (ES, m/z): [M+H]⁺=1584.9, r.t. 1.25 mins.

### Example A6. 2-((2,5,8,11-tetraoxatridecan-13-yl)carbamoyl)-4-((17S,20S)-1-amino-17-isopropyl-15,18-dioxo-20-(3-ureidopropyl)-3,6,9,12-tetraoxa-16,19-diazahenicosan-21-amido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Formic Acid Salt

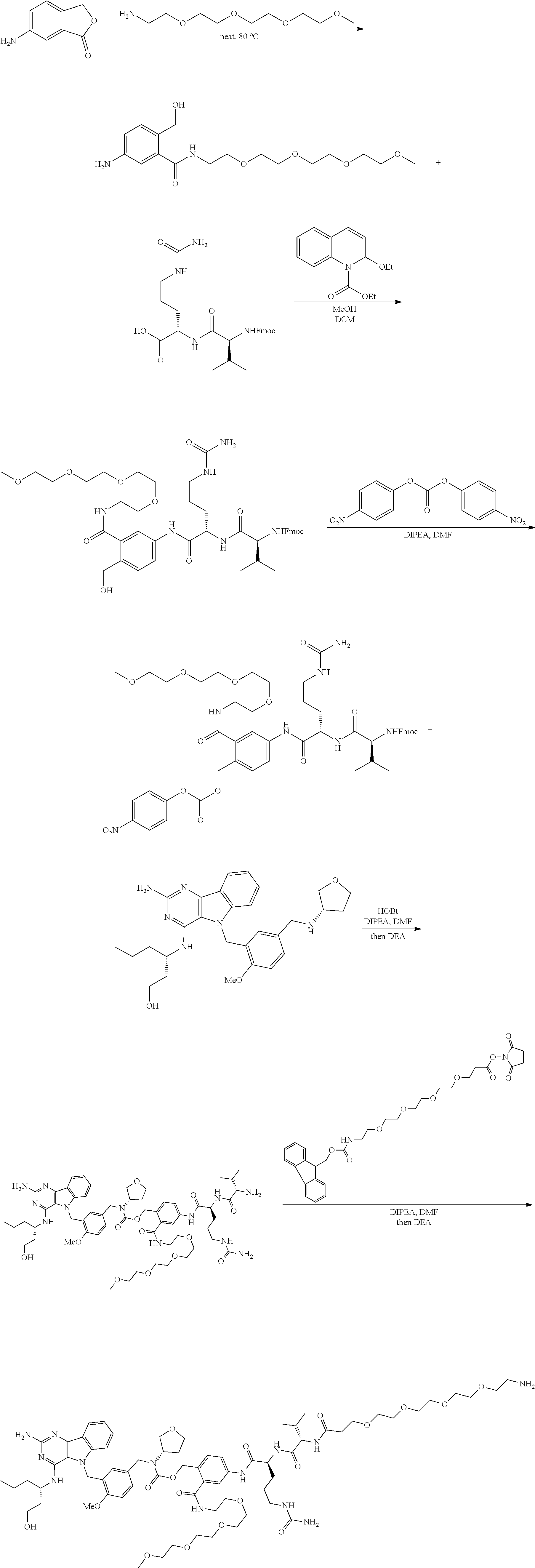

### Step A6-1. 5-amino-2-(hydroxymethyl)-N-(2,5,8,11-tetraoxatridecan-13-yl)benzamide

6-aminoisobenzofuran-1(3H)-one (2 g, 13.41 mmol) was dissolved in 2,5,8,11-tetraoxatridecan-13-amine (8.34 g, 40.2 mmol) and stirred at 80° C. for 16 hours. After cooling, the reaction mixture was diluted with DCM (10 mL) and purified using flash chromatography (220 g SiO₂ column, 0 to 10% MeOH in DCM), giving 5-amino-2-(hydroxymethyl)-N-(2,5,8,11-tetraoxatridecan-13-yl)benzamide (4.229 g, 11.87 mmol, 88% yield) as an oil. LC-MS (ES, m/z): [M+H]⁺=339.3 (product—H₂O).

### Step A6-2. (9H-fluoren-9-yl)methyl ((S)-1-(((S)-1-((3-((2,5,8,11-tetraoxatridecan-13-yl)carbamoyl)-4-(hydroxymethyl)phenyl)amino)-1-oxo-5-ureidopentan-2-yl)amino)-3-methyl-1-oxobutan-2-yl)carbamate

A solution of (S)-2-((S)-2-((((9H-fluoren-9-yl)methoxy)carbonyl)amino)-3-methylbutanamido)-5-ureidopentanoic acid (2.000 g, 4.03 mmol), 5-amino-2-(hydroxymethyl)-N-(2,5,8,11-tetraoxatridecan-13-yl)benzamide (1.507 g, 4.23 mmol) and ethyl 2-ethoxyquinoline-1(2H)-carboxylate (1.046 g, 4.23 mmol) in MeOH (15 mL) and DCM (15 mL) was stirred at room temperature for 24 hours. The reaction mixture was evaporated to dryness and the crude mixture purified using flash chromatography (120 g SiO₂ column, 0 to 20% MeOH in DCM), giving (9H-fluoren-9-yl)methyl ((S)-1-(((S)-1-((3-((2,5,8,11-tetraoxatridecan-13-yl)carbamoyl)-4-(hydroxymethyl)phenyl)amino)-1-oxo-5-ureidopentan-2-yl)amino)-3-methyl-1-oxobutan-2-yl)carbamate (2.045 g, 2.449 mmol, 60.8% yield) as a gum. LC-MS (ES, m/z): [M+H]⁺=835.5.

### Step A6-3. (9H-fluoren-9-yl)methyl ((S)-1-(((S)-1-((3-((2,5,8,11-tetraoxatridecan-13-yl)carbamoyl)-4-((((4-nitrophenoxy)carbonyl)oxy)methyl)phenyl)amino)-1-oxo-5-ureidopentan-2-yl)amino)-3-methyl-1-oxobutan-2-yl)carbamate

To a stirred solution of (9H-fluoren-9-yl)methyl ((S)-1-(((S)-1-((3-((2,5,8,11-tetraoxatridecan-13-yl)carbamoyl)-4-(hydroxymethyl)phenyl)amino)-1-oxo-5-ureidopentan-2-yl)amino)-3-methyl-1-oxobutan-2-yl)carbamate (1.6 g, 1.916 mmol) and bis(4-nitrophenyl) carbonate (1.749 g, 5.75 mmol) in DMF (8 mL) was added DIPEA (0.669 mL, 3.83 mmol). The reaction was stirred at room temperature for 2 hours. The reaction mixture was purified using flash chromatography (120 g SiO₂ column, loaded in DCM, 0 to 100% EtOAc in hexanes over 15 minutes, then 0 to 20% MeOH in DCM over 25 minutes), giving (9H-fluoren-9-yl)methyl ((S)-1-(((S)-1-((3-((2,5,8,11-tetraoxatridecan-13-yl)carbamoyl)-4-((((4-nitrophenoxy)carbonyl)oxy)methyl)phenyl)amino)-1-oxo-5-ureidopentan-2-yl)amino)-3-methyl-1-oxobutan-2-yl)carbamate (1.116 g, 1.116 mmol, 58.2% yield) as a solid. LC-MS (ES, m/z): [M+H]⁺=1000.4.

### Step A6-4. 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Trifluoroacetate Salt

A scintillation vial was charged with (S)-3-((2-amino-5-(2-methoxy-5-((((S)-tetrahydrofuran-3-yl)amino)methyl)benzyl)-5H-pyrimido[5,4-b]indol-4-yl)amino)hexan-1-ol, trifluoroacetate (170 mg, 0.269 mmol), (9H-fluoren-9-yl)methyl ((S)-1-(((S)-1-((3-((2,5,8,11-tetraoxatridecan-13-yl)carbamoyl)-4-((((4-nitrophenoxy)carbonyl)oxy)methyl)phenyl)amino)-1-oxo-5-ureidopentan-2-yl)amino)-3-methyl-1-oxobutan-2-yl)carbamate (349 mg, 0.349 mmol), HOBt (12.34 mg, 0.081 mmol) and DMF (4 mL). DIPEA (0.141 mL, 0.806 mmol) was added, and the reaction stirred at room temperature for 16 hours. Diethylamine (0.140 mL, 1.343 mmol) was added, and the reaction stirred at room temperature for 2 hours. The reaction was quenched with 2% TFA in water (4 mL), solubilized with MeCN (4 mL), filtered and purified using reverse-phase flash chromatography (100 g C18 column, 10 to 50% MeCN in water containing 0.05% TFA). Product fractions were combined and lyophilized to give 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, trifluoroacetate salt (177 mg, 0.139 mmol, 51.8% yield) as a solid. LC-MS (ES, m/z): [M+H]⁺=1157.5.

### Step A6-5. 2-((2,5,8,11-tetraoxatridecan-13-yl)carbamoyl)-4-((17S,20S)-1-amino-17-isopropyl-15,18-dioxo-20-(3-ureidopropyl)-3,6,9,12-tetraoxa-16,19-diazahenicosan-21-amido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Formic Acid Salt

A scintillation vial was charged with 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, trifluoroacetate salt (30 mg, 0.024 mmol), Alpha-(Fmoc-amino)-omega-(succinimidyl propionate)tetra(ethylene glycol) (20.69 mg, 0.035 mmol) and DMF (0.60 mL). DIPEA (0.012 mL, 0.071 mmol) was added, and the reaction stirred at room temperature for 1 hour. Diethylamine (0.025 mL, 0.236 mmol) was added, and the reaction stirred for a further hour. The reaction mixture was quenched with 5% formic acid in water (1.5 mL), solubilized with MeCN (0.5 mL), filtered and purified using HPLC (30×150 mm C₁₈ column, 10 to 40% MeCN in water containing 0.05% formic acid). Product fractions were combined and lyophilized to give 2-((2,5,8,11-tetraoxatridecan-13-yl)carbamoyl)-4-((17S,20S)-1-amino-17-isopropyl-15,18-dioxo-20-(3-ureidopropyl)-3,6,9,12-tetraoxa-16,19-diazahenicosan-21-amido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, formic acid salt (17.2 mg, 0.012 mmol, 50.2% yield) as a solid. LC-MS (ES, m/z): [M+H]⁺=1405.0, r.t. 1.10 mins.

### Example A7. 2-((2,5,8,11-tetraoxatridecan-13-yl)carbamoyl)-4-((2S,5S)-25-(2,5-dioxo-2,5-dihydro-1H-pyrrol-1-yl)-5-isopropyl-4,7,23-trioxo-2-(3-ureidopropyl)-10,13,16,19-tetraoxa-3,6,22-triazapentacosanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Formic Acid Salt

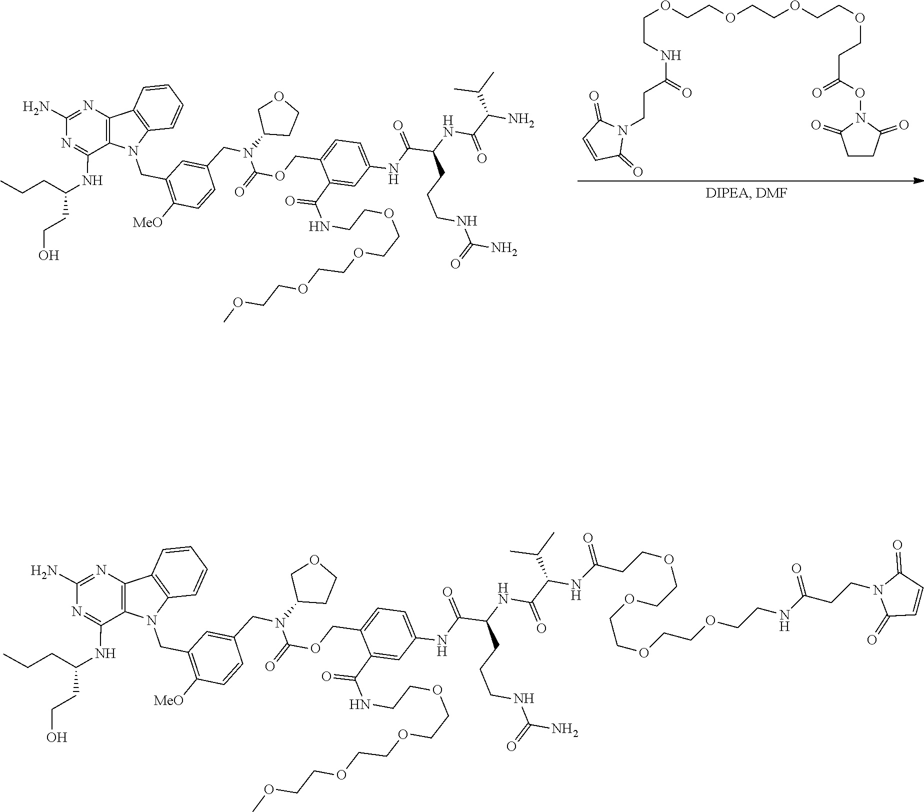 [SMILES: CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](N)C(C)C)cc4C(=O)NCCOCCOCCOCCOC)[C@H]4CCOC4)ccc3OC)c12 ; O=C(CCN1C(=O)C=CC1=O)NCCOCCOCCOCCOCCC(=O)ON1C(=O)CCC1=O ; CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](NC(=O)CCOCCOCCOCCOCCNC(=O)CCN5C(=O)C=CC5=O)C(C)C)cc4C(=O)NCCOCCOCCOCCOC)[C@H]4CCOC4)ccc3OC)c12]

A scintillation vial was charged with 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, trifluoroacetate salt (19 mg, 0.015 mmol), 3-(2-Maleimidoethylcarbamoyl)-PEG4-propionic acid N-hydroxy succinimide ester (9.21 mg, 0.018 mmol) and DMF (0.60 mL). DIPEA (7.83 μl, 0.045 mmol) was added, and the reaction stirred at room temperature for 30 minutes. The reaction mixture was quenched with 5% TFA in water (1.5 mL), filtered and purified using preparative HPLC (30×150 mm C18 column, 10 to 40% MeCN in water containing 0.05% formic acid). Product fractions were combined and lyophilized to give 2-((2,5,8,11-tetraoxatridecan-13-yl)carbamoyl)-4-((2S,5S)-25-(2,5-dioxo-2,5-dihydro-1H-pyrrol-1-yl)-5-isopropyl-4,7,23-trioxo-2-(3-ureidopropyl)-10,13,16,19-tetraoxa-3,6,22-triazapentacosanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, formic acid salt (10 mg, 6.24 μmol, 41.8% yield) as a solid. LC-MS (ES, m/z): [M+H]⁺=1556.2, r.t. 1.19 mins.

### Example A8. 2-((2,5,8,11-tetraoxatridecan-13-yl)carbamoyl)-4-((2S,5S)-15-amino-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Formic Acid Salt

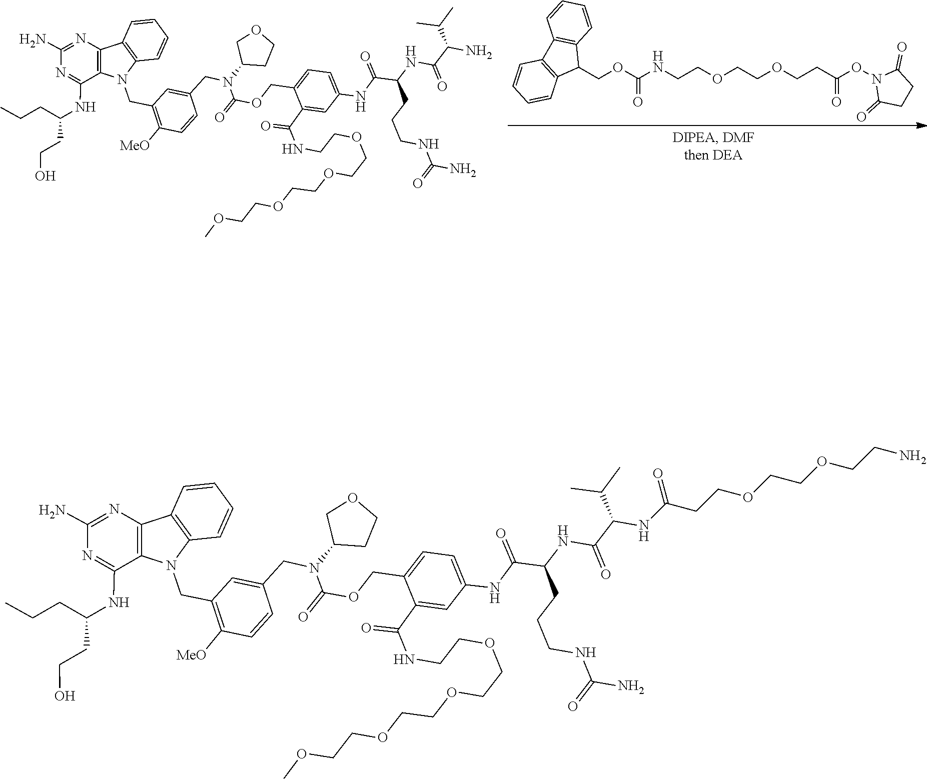 [SMILES: CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](N)C(C)C)cc4C(=O)NCCOCCOCCOCCOC)[C@H]4CCOC4)ccc3OC)c12 ; O=C(CCOCCOCCNC(=O)OCC1c2ccccc2-c2ccccc21)ON1C(=O)CCC1=O ; CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](NC(=O)CCOCCOCCN)C(C)C)cc4C(=O)NCCOCCOCCOCCOC)[C@H]4CCOC4)ccc3OC)c12]

A scintillation vial was charged with 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, trifluoroacetate (30 mg, 0.024 mmol), Fmoc-peg2-nhs ester (17.57 mg, 0.035 mmol) and DMF (0.60 mL). DIPEA (0.012 mL, 0.071 mmol) was added, and the reaction stirred at room temperature for 1 hour. Diethylamine (2.465 μl, 0.024 mmol) was added, and the reaction stirred at room temperature for a further hour. The reaction mixture was quenched with 5% formic acid in water (1.5 mL), solubilized with MeCN 0.5 mL), filtered and purified using preparative HPLC (30×150 mm C18 column, 10 to 33% MeCN in water containing 0.05% formic acid). Product fractions were combined and lyophilized to give 2-((2,5,8,11-tetraoxatridecan-13-yl)carbamoyl)-4-((2S,5S)-15-amino-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, formic acid salt (19.6 mg, 0.014 mmol, 61.0% yield) as a solid. LC-MS (ES, m/z): [M+H]⁺=1316.6, 1.09 mins.

### Example A9. 2-((2,5,8,11-tetraoxatridecan-13-yl)carbamoyl)-4-((2S,5S)-19-(2,5-dioxo-2,5-dihydro-1H-pyrrol-1-yl)-5-isopropyl-4,7,17-trioxo-2-(3-ureidopropyl)-10,13-dioxa-3,6,16-triazanonadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Formic Acid Salt

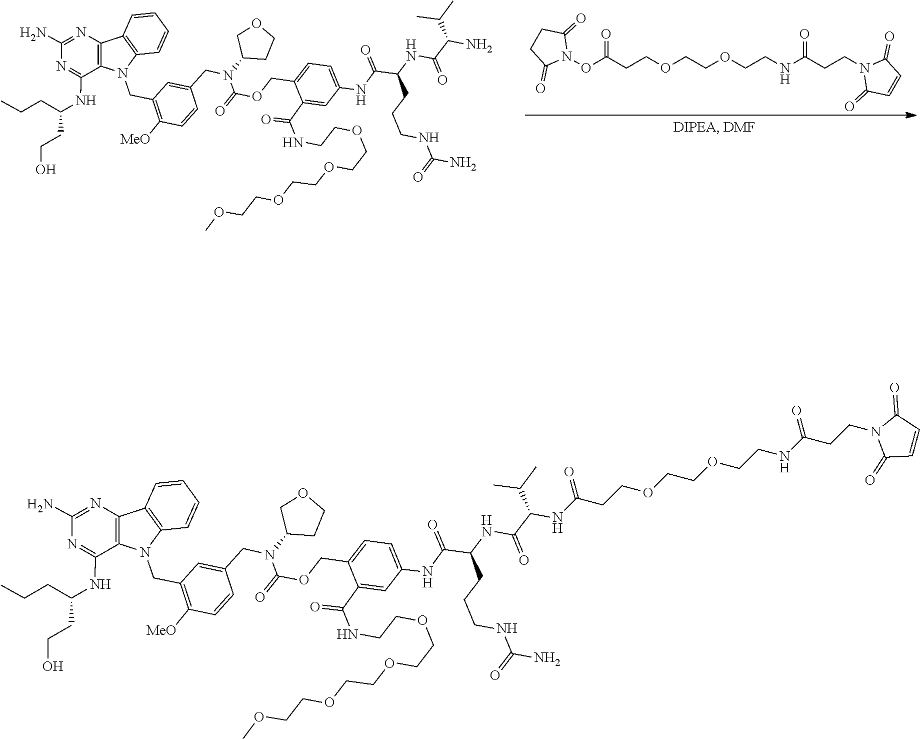 [SMILES: CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](N)C(C)C)cc4C(=O)NCCOCCOCCOCCOC)[C@H]4CCOC4)ccc3OC)c12 ; O=C(CCN1C(=O)C=CC1=O)NCCOCCOCCC(=O)ON1C(=O)CCC1=O ; CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](NC(=O)CCOCCOCCNC(=O)CCN5C(=O)C=CC5=O)C(C)C)cc4C(=O)NCCOCCOCCOCCOC)[C@H]4CCOC4)ccc3OC)c12]

A scintillation vial was charged with 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, trifluoroacetate salt (19 mg, 0.015 mmol), 2,5-dioxopyrrolidin-1-yl 3-(2-(2-(3-(2,5-dioxo-2 h-pyrrol-1(5 h)-yl)propanamido)ethoxy)ethoxy)propanoate (7.63 mg, 0.018 mmol) and DMF (0.6 mL). DIPEA (7.83 μl, 0.045 mmol) was added, and the reaction stirred at room temperature for 30 minutes. The reaction mixture was quenched with 5% TFA in water (1.5 mL), filtered and purified using preparative HPLC (30×150 mm C18 column, 10 to 40% MeCN in water containing 0.05% formic acid). Product fractions were combined and lyophilized to give 2-((2,5,8,11-tetraoxatridecan-13-yl)carbamoyl)-4-((2S,5S)-19-(2,5-dioxo-2,5-dihydro-1H-pyrrol-1-yl)-5-isopropyl-4,7,17-trioxo-2-(3-ureidopropyl)-10,13-dioxa-3,6,16-triazanonadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, formic acid salt (11 mg, 7.27 μmol, 48.6% yield). LC-MS (ES, m/z): [M+H]⁺=1468.1, r.t. 1.18 mins.

### Example A10. 2-((2,5,8,11-tetraoxatridecan-13-yl)carbamoyl)-4-((2S,5S)-16-(2,5-dioxo-2,5-dihydro-1H-pyrrol-1-yl)-5-isopropyl-4,7,14-trioxo-2-(3-ureidopropyl)-10-oxa-3,6,13-triazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Formic Acid Salt (21.4 mg, 0.015 mmol, 74.1% yield)

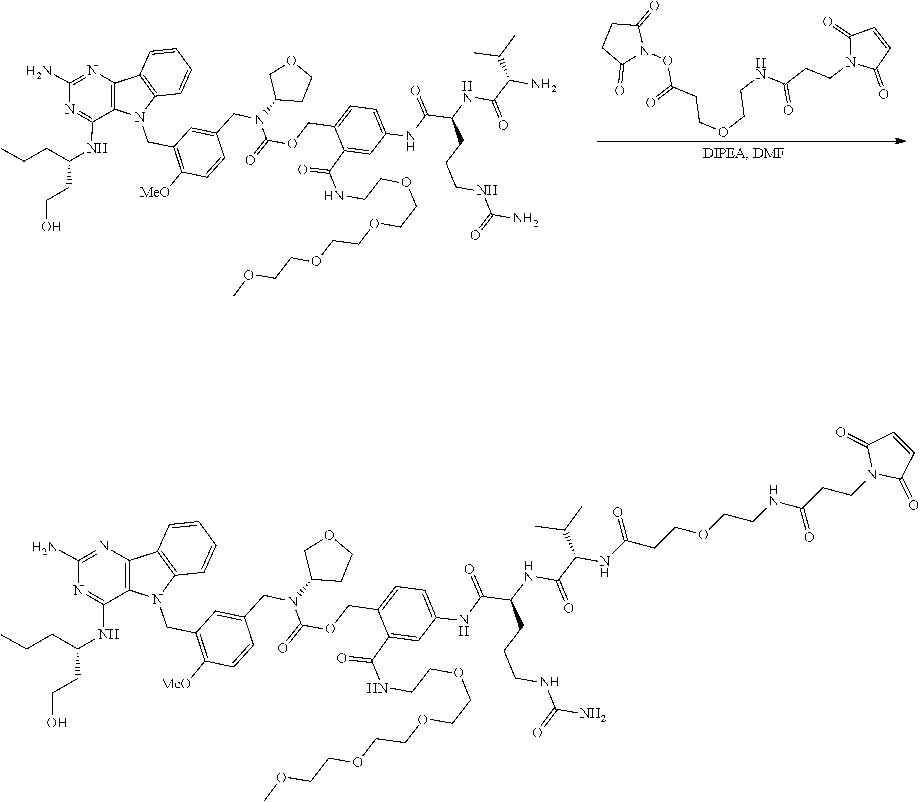 [SMILES: CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](N)C(C)C)cc4C(=O)NCCOCCOCCOCCOC)[C@H]4CCOC4)ccc3OC)c12 ; O=C(CCN1C(=O)C=CC1=O)NCCOCCC(=O)ON1C(=O)CCC1=O ; CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](NC(=O)CCOCCNC(=O)CCN5C(=O)C=CC5=O)C(C)C)cc4C(=O)NCCOCCOCCOCCOC)[C@H]4CCOC4)ccc3OC)c12]

A scintillation vial was charged with 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, trifluoroacetate (25 mg, 0.020 mmol), 2,5-Dioxopyrrolidin-1-yl 3-(2-(3-(2,5-dioxo-2,5-dihydro-1H-pyrrol-1-yl)propanamido)ethoxy)propanoate (11.25 mg, 0.029 mmol) and DMF (0.6 mL). DIPEA (10.30 μl, 0.059 mmol) was added, and the reaction stirred at room temperature for 30 minutes. The reaction mixture was quenched with 5% formic acid in water (1.5 mL), solubilized with MeCN (0.5 mL), filtered and purified using preparative HPLC (30×150 mm C₁₈ column, 10 to 45% MeCN in water containing 0.05% formic acid). Product fractions were combined and lyophilized to give 2-((2,5,8,11-tetraoxatridecan-13-yl)carbamoyl)-4-((2S,5S)-16-(2,5-dioxo-2,5-dihydro-1H-pyrrol-1-yl)-5-isopropyl-4,7,14-trioxo-2-(3-ureidopropyl)-10-oxa-3,6,13-triazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, formic acid salt (21.4 mg, 0.015 mmol, 74.1% yield). LC-MS (ES, m/z): [M+H]⁺=1424.1, r.t. 1.18 mins.

### Example A11. 2-((2,5,8,11-tetraoxatridecan-13-yl)carbamoyl)-4-((S)-2-((S)-2-(3-(2-aminoethoxy)propanamido)-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Formic Acid Salt

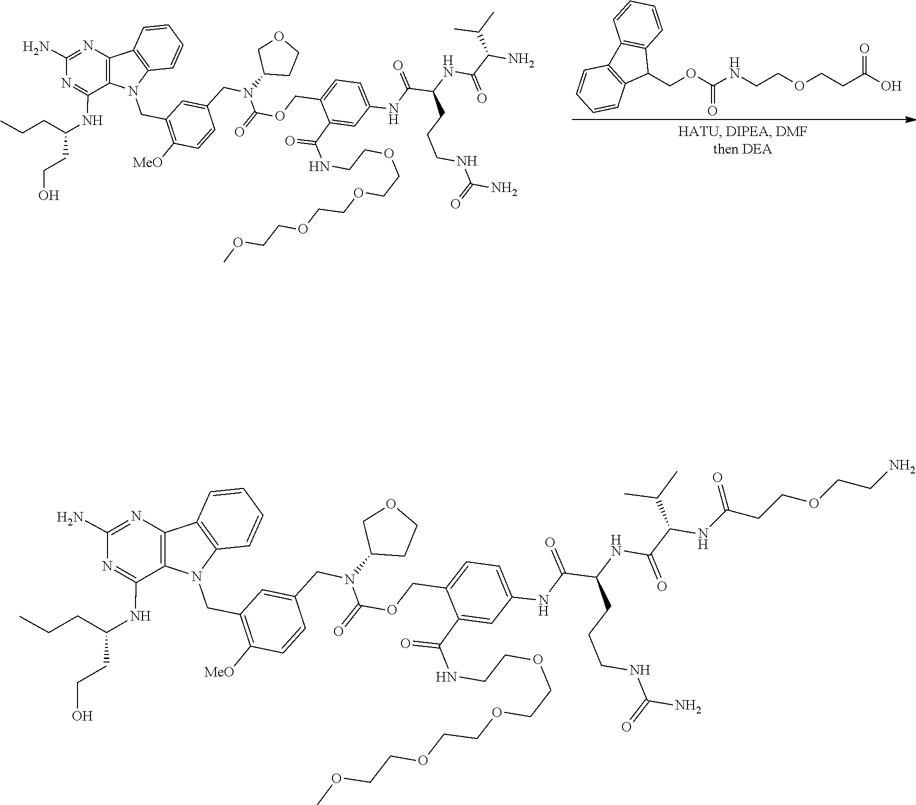 [SMILES: CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](N)C(C)C)cc4C(=O)NCCOCCOCCOCCOC)[C@H]4CCOC4)ccc3OC)c12 ; O=C(O)CCOCCNC(=O)OCC1c2ccccc2-c2ccccc21 ; CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](NC(=O)CCOCCN)C(C)C)cc4C(=O)NCCOCCOCCOCCOC)[C@H]4CCOC4)ccc3OC)c12]

A scintillation vial was charged with 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, trifluoroacetate salt (35 mg, 0.028 mmol), Fmoc-6-amino-4-oxohexanoic acid (11.74 mg, 0.033 mmol), HATU (12.56 mg, 0.033 mmol) and DMF (0.50 mL). DIPEA (0.014 mL, 0.083 mmol) was added, and the reaction stirred at room temperature for 30 minutes. Diethylamine (0.029 mL, 0.275 mmol) was added, and the reaction was stirred at room temperature for a further 30 minutes. The reaction mixture was quenched with 5% formic acid in water (1.5 mL), filtered and purified using preparative HPLC (30×150 mm C18 column, 10 to 42% MeCN in water containing 0.05% formic acid). Product fractions were combined and lyophilized to give 2-((2,5,8,11-tetraoxatridecan-13-yl)carbamoyl)-4-((S)-2-((S)-2-(3-(2-aminoethoxy)propanamido)-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, formic acid salt (16.5 mg, 0.013 mmol, 45.5% yield). LC-MS (ES, m/z): [M+H]⁺=1272.8, 1.09 mins.

### Example A12. 2-((2,5,8,11-tetraoxatridecan-13-yl)carbamoyl)-4-((S)-2-((S)-2-(3-(2,5-dioxo-2,5-dihydro-1H-pyrrol-1-yl)propanamido)-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Formic Acid Salt

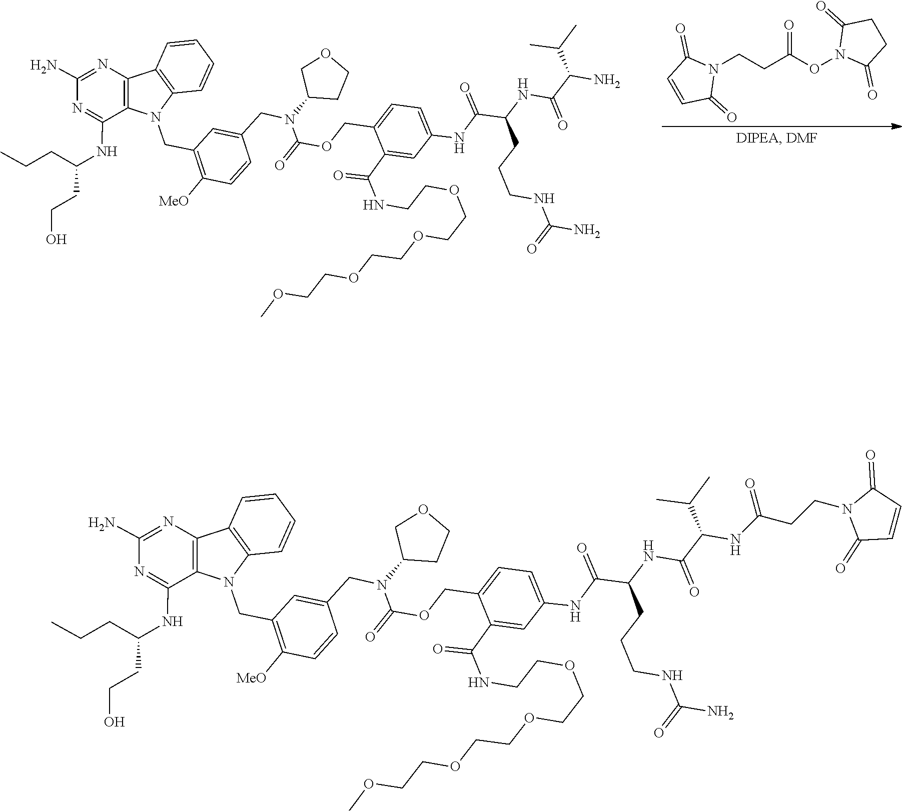 [SMILES: CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](N)C(C)C)cc4C(=O)NCCOCCOCCOCCOC)[C@H]4CCOC4)ccc3OC)c12 ; O=C(CCN1C(=O)C=CC1=O)ON1C(=O)CCC1=O ; CCC[C@@H](CCO)Nc1nc(N)nc2c3ccccc3n(Cc3cc(CN(C(=O)OCc4ccc(NC(=O)[C@H](CCCNC(N)=O)NC(=O)[C@@H](NC(=O)CCN5C(=O)C=CC5=O)C(C)C)cc4C(=O)NCCOCCOCCOCCOC)[C@H]4CCOC4)ccc3OC)c12]

A scintillation vial was charged with 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, trifluoroacetate salt (20 mg, 0.016 mmol), N-Succinimidyl 3-maleimidopropionate (6.28 mg, 0.024 mmol) and DMF (0.50 mL). DIPEA (8.24 μl, 0.047 mmol) was added, and the reaction stirred at room temperature for 30 minutes. The reaction mixture was quenched with 5% formic acid in water (1.5 mL), solubilized with MeCN (0.5 mL), filtered and purified using HPLC (30×150 mm C18 column, 10 to 42% MeCN in water containing 0.05% formic acid). Product fractions were combined and lyophilized to give 2-((2,5,8,11-tetraoxatridecan-13-yl)carbamoyl)-4-((S)-2-((S)-2-(3-(2,5-dioxo-2,5-dihydro-1H-pyrrol-1-yl)propanamido)-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, formic acid salt (15.6 mg, 0.012 mmol, 73.2% yield). LC-MS (ES, m/z): [M+H]⁺=1308.8, 1.20 mins.

### V. Synthetic Examples of Hydrolyzable Linkers

### Example 1. 2-((2,5,8,11-tetraoxatridecan-13-yl)carbamoyl)-4-((2S,5S)-15-(4-(2-(2,5-dioxo-2,5-dihydro-1H-pyrrol-1-yl)ethyl)piperazin-1-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

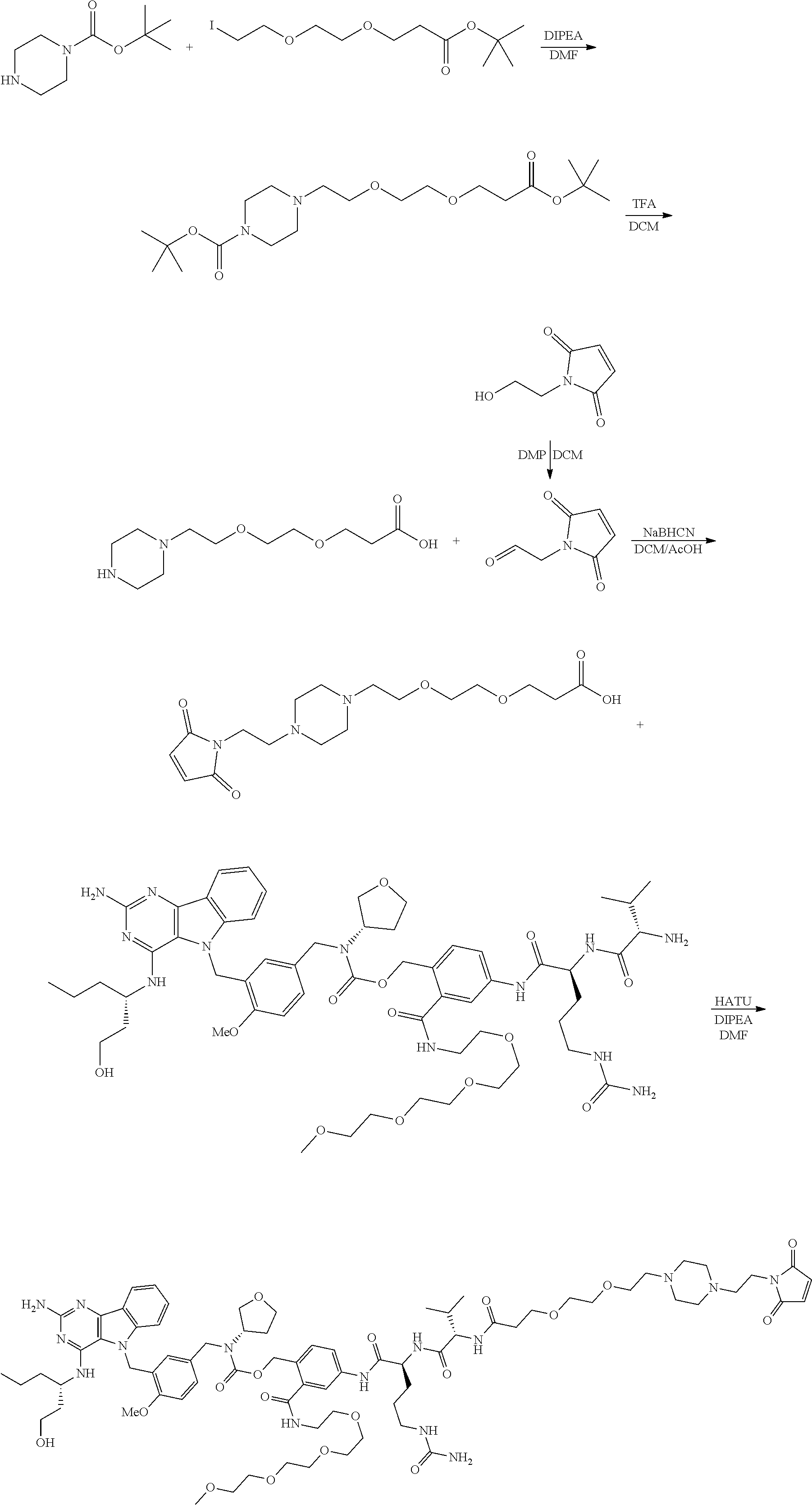

### Step 1-1. tert-Butyl 4-(2-(2-(3-(tert-butoxy)-3-oxopropoxy)ethoxy)ethyl)piperazine-1-carboxylate, Trifluoroacetate Salt

A scintillation vial was charged with tert-butyl 3-(2-(2-iodoethoxy)ethoxy)propanoate (250 mg, 0.726 mmol), 1-Boc-piperazine (271 mg, 1.453 mmol), DIPEA (0.254 mL, 1.453 mmol) and DMF (1 mL). The reaction was stirred at room temperature for 16 hours. The reaction mixture was quenched with 5% TFA in water (2 mL), diluted with MeCN (0.5 mL) a purified using reverse-phase flash chromatography (30 g C18 column, 0 to 50% MeCN in water containing 0.05% TFA). Product fractions were combined and lyophilized to give tert-butyl 4-(2-(2-(3-(tert-butoxy)-3-oxopropoxy)ethoxy)ethyl)piperazine-1-carboxylate, trifluoroacetate salt (303 mg, 0.587 mmol, 81% yield) as a solid. LC-MS (ES, m/z): [M+H]⁺=403.6. ¹H NMR (400 MHz, CHLOROFORM-d) δ 4.16 (br s, 2H), 3.91 (dd, J=5.3, 3.8 Hz, 2H), 3.71 (t, J=6.4 Hz, 3H), 3.67-3.57 (m, 5H), 3.56-3.32 (m, 2H), 3.32-3.24 (m, 2H), 2.87 (br s, 2H), 2.50 (t, J=6.4 Hz, 2H), 1.48 (d, J=7.6 Hz, 18H).

### Step 1-2. 3-(2-(2-(piperazin-1-yl)ethoxy)ethoxy)propanoic acid, bis-trifluoroacetate Salt

tert-Butyl 4-(2-(2-(3-(tert-butoxy)-3-oxopropoxy)ethoxy)ethyl)piperazine-1-carboxylate, TFA (300 mg, 0.581 mmol) was dissolved in DCM (5 mL). TFA (0.895 mL, 11.62 mmol) was added, and the reaction stirred for 16 hours at room temperature. The reaction mixture was evaporated to dryness, dissolved in MeOH (6 mL) and evaporated twice, then lyophilized, giving 3-(2-(2-(piperazin-1-yl)ethoxy)ethoxy)propanoic acid, bis-trifluoroacetate salt (250 mg, 0.527 mmol, 90% yield) as a solid. ¹H NMR (400 MHz, DMSO-d₆) δ 9.14 (s, 1H), 3.78-3.69 (m, 2H), 3.66-3.59 (m, 2H), 3.59-3.51 (m, 4H), 3.51-3.14 (m, 10H), 2.48-2.43 (m, 2H).

### Step 1-3. 2-(2,5-dioxo-2,5-dihydro-1H-pyrrol-1-yl)acetaldehyde

1-(2-Hydroxy-ethyl)-pyrrole-2,5-dione (2 g, 14.17 mmol) and Dess-Martin periodinane (8.42 g, 19.84 mmol) were stirred for 16 hours in DCM (50 mL). The reaction mixture was filtered and the filtrate evaporated down to ca. 20 mL. After standing for 2 hours, the solution was aspirated off and purified using flash chromatography (80 g SiO₂ column, 0 to 70% EtOAc in hexanes), giving 2-(2,5-dioxo-2,5-dihydro-1H-pyrrol-1-yl)acetaldehyde (170 mg, 1.222 mmol, 8.62% yield), as an oil. ¹H NMR (400 MHz, CHLOROFORM-d) δ 9.69-9.50 (m, 1H), 6.84-6.83 (m, 2H), 4.43 (s, 2H).

### Step 1-4. 3-(2-(2-(4-(2-(2,5-dioxo-2,5-dihydro-1H-pyrrol-1-yl)ethyl)piperazin-1-yl)ethoxy)ethoxy)propanoic acid, bis-trifluoroacetate Salt

To a stirred solution of 3-(2-(2-(piperazin-1-yl)ethoxy)ethoxy)propanoic acid, bis-trifluoroacetate salt (275 mg, 0.580 mmol) and 2-(2,5-dioxo-2,5-dihydro-1H-pyrrol-1-yl)acetaldehyde (161 mg, 1.159 mmol) in DCM (5 mL) was added acetic acid (0.5 mL) followed by sodium cyanoborohydride (109 mg, 1.739 mmol), and the reaction stirred for 20 minutes at room temperature. The reaction mixture was quenched with water (3 mL) and evaporated to remove the organics. The residue was diluted with water (1 mL) and MeCN (0.5 mL), filtered and purified using reverse-phase flash chromatography (50 g C18 column, 0 to 25% MeCN in water containing 0.05% TFA). Product fractions were combined and lyophilized to give 3-(2-(2-(4-(2-(2,5-dioxo-2,5-dihydro-1H-pyrrol-1-yl)ethyl)piperazin-1-yl)ethoxy)ethoxy)propanoic acid, bis-trifluoroacetate salt (62 mg, 0.104 mmol, 17.90% yield) as a solid. LC-MS (ES, m/z): [M+H]⁺=370.1. ¹H NMR (400 MHz, DMSO-d₆) δ 7.05 (s, 2H), 3.73-3.69 (m, 2H), 3.64-3.52 (m, 10H), 3.47-3.33 (m, 2H), 3.25 (br s, 2H), 3.05 (br s, 2H), 2.94 (br s, 1H), 2.71-2.57 (m, 2H), 2.47-2.44 (m, 2H).

### Step 1-5. 2-((2,5,8,11-tetraoxatridecan-13-yl)carbamoyl)-4-((2S,5S)-15-(4-(2-(2,5-dioxo-2,5-dihydro-1H-pyrrol-1-yl)ethyl)piperazin-1-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

A scintillation vial was charged with 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (27 mg, 0.023 mmol), 3-(2-(2-(4-(2-(2,5-dioxo-2,5-dihydro-1H-pyrrol-1-yl)ethyl)piperazin-1-yl)ethoxy)ethoxy)propanoic acid, bis-trifluoroacetate salt (16.73 mg, 0.028 mmol), HATU (10.64 mg, 0.028 mmol) and DMF (0.5 mL). DIPEA (4.07 μl, 0.023 mmol) was added, and the reaction stirred at room temperature for 10 minutes. The reaction mixture was quenched with 5% formic acid in water (2 mL), diluted with MeCN (0.5 mL), filtered and purified using preparatory HPLC (30×150 mm C18 column, 5 to 100% MeCN in water containing 0.05% formic acid). Product fractions were combined and lyophilized to give 2-((2,5,8,11-tetraoxatridecan-13-yl)carbamoyl)-4-((2S,5S)-15-(4-(2-(2,5-dioxo-2,5-dihydro-1H-pyrrol-1-yl)ethyl)piperazin-1-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2formic acid salt (11.2 mg, 7.00 μmol, 30.0% yield) as a solid. LC-MS (ES, m/z): [M+H]⁺=1508.7, 1.61 mins.

### Example 2. 2-((2,5,8,11-tetraoxatridecan-13-yl)carbamoyl)-4-((2S,5S)-15-((1S,4S)-5-(2-(2,5-dioxo-2,5-dihydro-1H-pyrrol-1-yl)ethyl)-2,5-diazabicyclo[2.2.1]heptan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate. 2 Formic Acid

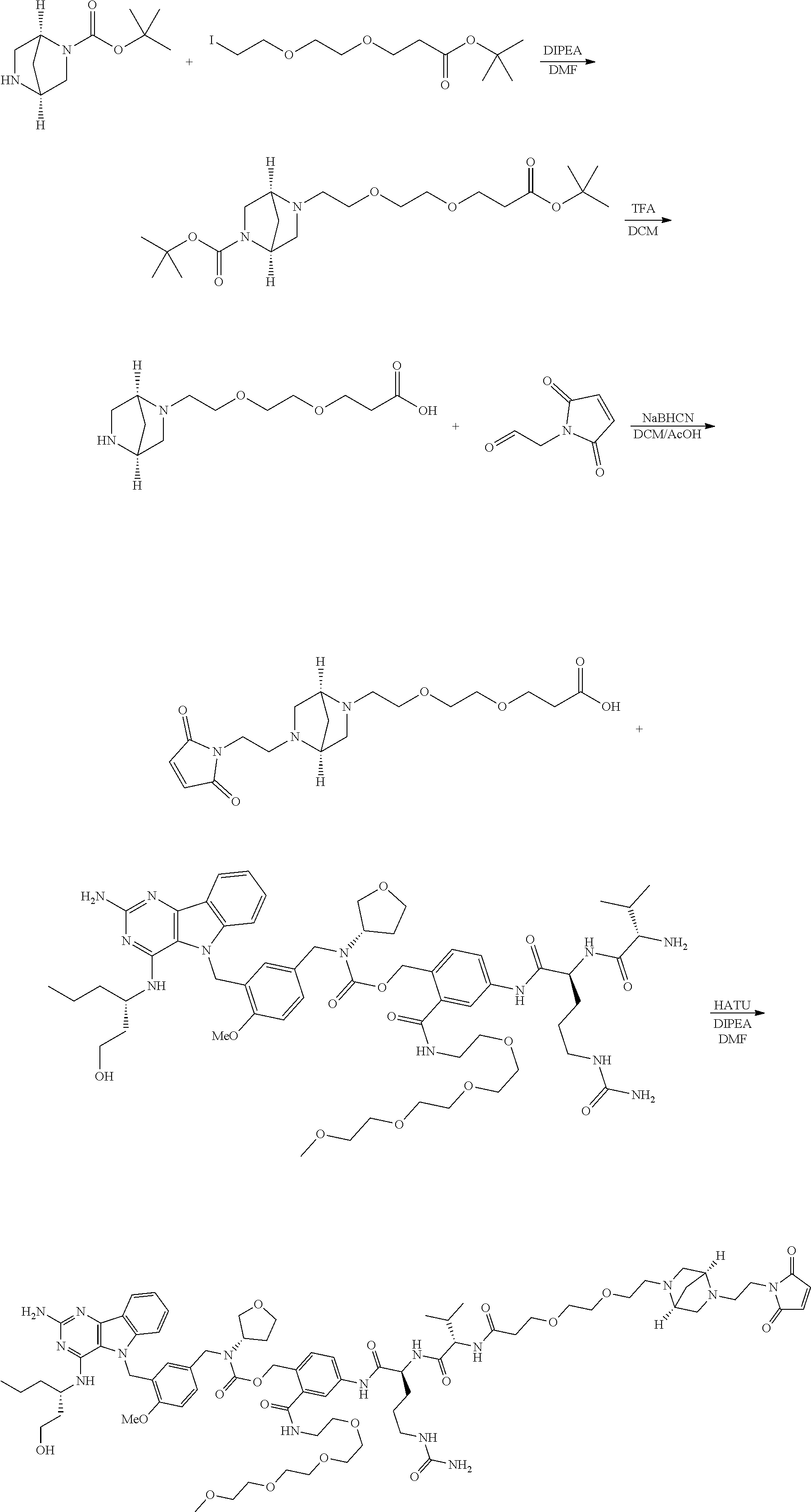

### Step 2-1. tert-butyl (1S,4S)-5-(2-(2-(3-(tert-butoxy)-3-oxopropoxy)ethoxy)ethyl)-2,5-diazabicyclo[2.2.1]heptane-2-carboxylate, Trifluoroacetate Salt

A scintillation vial was charged with tert-butyl 3-(2-(2-iodoethoxy)ethoxy)propanoate (500 mg, 1.453 mmol), tert-butyl (1S,4S)-2,5-diazabicyclo[2.2.1]heptane-2-carboxylate (288 mg, 1.453 mmol), DIPEA (0.507 mL, 2.91 mmol) and DMF (2 mL). The reaction was stirred at room temperature for 16 hours. The reaction mixture was quenched with 5% TFA in water (3 mL), diluted with MeCN (1 mL), filtered and purified using reverse-phase flash chromatography (50 g C18 column, 0 to 50% MeCN in water containing 0.05% TFA). Product fractions were combined and lyophilized to give tert-butyl (1S,4S)-5-(2-(2-(3-(tert-butoxy)-3-oxopropoxy)ethoxy)ethyl)-2,5-diazabicyclo[2.2.1]heptane-2-carboxylate, trifluoroacetate salt (425 mg, 0.804 mmol, 55.3% yield) as an oil. LC-MS (ES, m/z): [M+H]⁺=415.7. ¹H NMR (400 MHz, CHLOROFORM-d) δ 4.66-4.56 (m, 1H), 4.54-4.39 (m, 1H), 4.13-3.74 (m, 4H), 3.74-3.67 (m, 2H), 3.66-3.55 (m, 4H), 3.53-3.37 (m, 2H), 3.36-3.25 (m, 1H), 2.54-2.44 (m, 2H), 2.38-2.26 (m, 1H), 2.16-2.03 (m, 1H), 1.58-1.39 (m, 18H).

### Step 2-2. 3-(2-(2-((1S,4S)-2,5-diazabicyclo[2.2.1]heptan-2-yl)ethoxy)ethoxy)propanoic Acid, Bis-Trifluoroacetate Salt

tert-Butyl(1S,4S)-5-(2-(2-(3-(tert-butoxy)-3-oxopropoxy)ethoxy)ethyl)-2,5-diazabicyclo[2.2.1]heptane-2-carboxylate (575 mg, 1.387 mmol) was dissolved in DCM (4 mL). TFA (1.069 mL, 13.87 mmol) was added, and the reaction stirred at room temperature for 3 hours, then evaporated to dryness. The residue was dissolved in MeOH (5 mL) and evaporated to dryness twice, then lyophilized to give 3-(2-(2-((1S,4S)-2,5-diazabicyclo[2.2.1]heptan-2-yl)ethoxy)ethoxy)propanoic acid, bis-trifluoroacetate (420 mg, 0.864 mmol, 62.3% yield) as a gum. ¹H NMR (400 MHz, DMSO-d₆) δ 9.04 (br s, 1H), 3.85 (br s, 2H), 3.70 (br d, J=3.8 Hz, 2H), 3.66-3.44 (m, 6H), 3.39-2.88 (m, 7H), 2.49-2.44 (m, 2H), 2 protons obscured by the DMSO peak, exchangeable NH+ protons not visible.

### Step 2-3. 3-(2-(2-((1S,4S)-5-(2-(2,5-dioxo-2,5-dihydro-1H-pyrrol-1-yl)ethyl)-2,5-diazabicyclo[2.2.1]heptan-2-yl)ethoxy)ethoxy)propanoic Acid

To a stirred solution of 3-(2-(2-((1S,4S)-2,5-diazabicyclo[2.2.1]heptan-2-yl)ethoxy)ethoxy)propanoic acid (150 mg, 0.581 mmol) and 2-(2,5-dioxo-2,5-dihydro-1H-pyrrol-1-yl)acetaldehyde (97 mg, 0.697 mmol) in DCM (3 mL) was added acetic acid (0.300 mL) followed by sodium cyanoborohydride (73.0 mg, 1.161 mmol). The reaction was stirred at room temperature for 10 minutes. The reaction mixture was quenched with water (2 mL) and evaporated to remove the organics. The residue was diluted with water (1 mL) and MeCN (0.5 mL) and purified using reverse-phase flash chromatography (50 g C18 column, 0 to 30% MeCN in water containing 0.05% TFA). Product fractions were combined and lyophilized to give 3-(2-(2-((1S,4S)-5-(2-(2,5-dioxo-2,5-dihydro-1H-pyrrol-1-yl)ethyl)-2,5-diazabicyclo[2.2.1]heptan-2-yl)ethoxy)ethoxy)propanoic acid (60 mg, 0.157 mmol, 27.1% yield) as a gum. LC-MS (ES, m/z): [M+H]⁺=382.4, consistent with the expected product.

### Step 2-4. 2-((2,5,8,11-tetraoxatridecan-13-yl)carbamoyl)-4-((2S,5S)-15-((1S,4S)-5-(2-(2,5-dioxo-2,5-dihydro-1H-pyrrol-1-yl)ethyl)-2,5-diazabicyclo[2.2.1]heptan-2-yl)-5-isopropyl-4,7-dioxo-2-(3-ureidopropyl)-10,13-dioxa-3,6-diazapentadecanamido)benzyl(3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

A scintillation vial was charged with 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (40 mg, 0.035 mmol), 3-(2-(2-((1S,4S)-5-(2-(2,5-dioxo-2,5-dihydro-1H-pyrrol-1-yl)ethyl)-2,5-diazabicyclo[2.2.1]heptan-2-yl)ethoxy)ethoxy)propanoic acid, bis-trifluoroacetate salt (21.06 mg, 0.035 mmol), HATU (15.77 mg, 0.041 mmol) and DMF (0.5 mL). DIPEA (6.04 μl, 0.035 mmol) was added, and the reaction stirred at room temperature for 10 minutes. The reaction mixture was quenched with 5% formic acid in water (3 mL), diluted with MeCN (0.5 mL), filtered and purified using preparatory HPLC (30×150 mm C18 column, 10-35% MeCN in water containing 0.05% formic acid). Product fractions were combined and lyophilized to give 2-((2,5,8,11-tetraoxatridecan-13-yl)carbamoyl)-4-((2S,5S)-15-((1S,4S)-5-(2-(2,5-dioxo-2,5-dihydro-1H-pyrrol-1-yl)ethyl)-2,5-diazabicyclo[2.2.1]heptan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, bis-formic acid salt (22 mg, 0.014 mmol, 39.5% yield) as a white solid. LC-MS (ES, m/z):[M+H]⁺=1520.9, r.t. 1.42 mins.

### Example 3. 2-((2,5,8,11-tetraoxatridecan-13-yl)carbamoyl)-4-((2S,5S)-15-((3aR,6aS)-5-(2-(2,5-dioxo-2,5-dihydro-1H-pyrrol-1-yl)ethyl)hexahydropyrrolo[3,4-c]pyrrol-2(H)-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

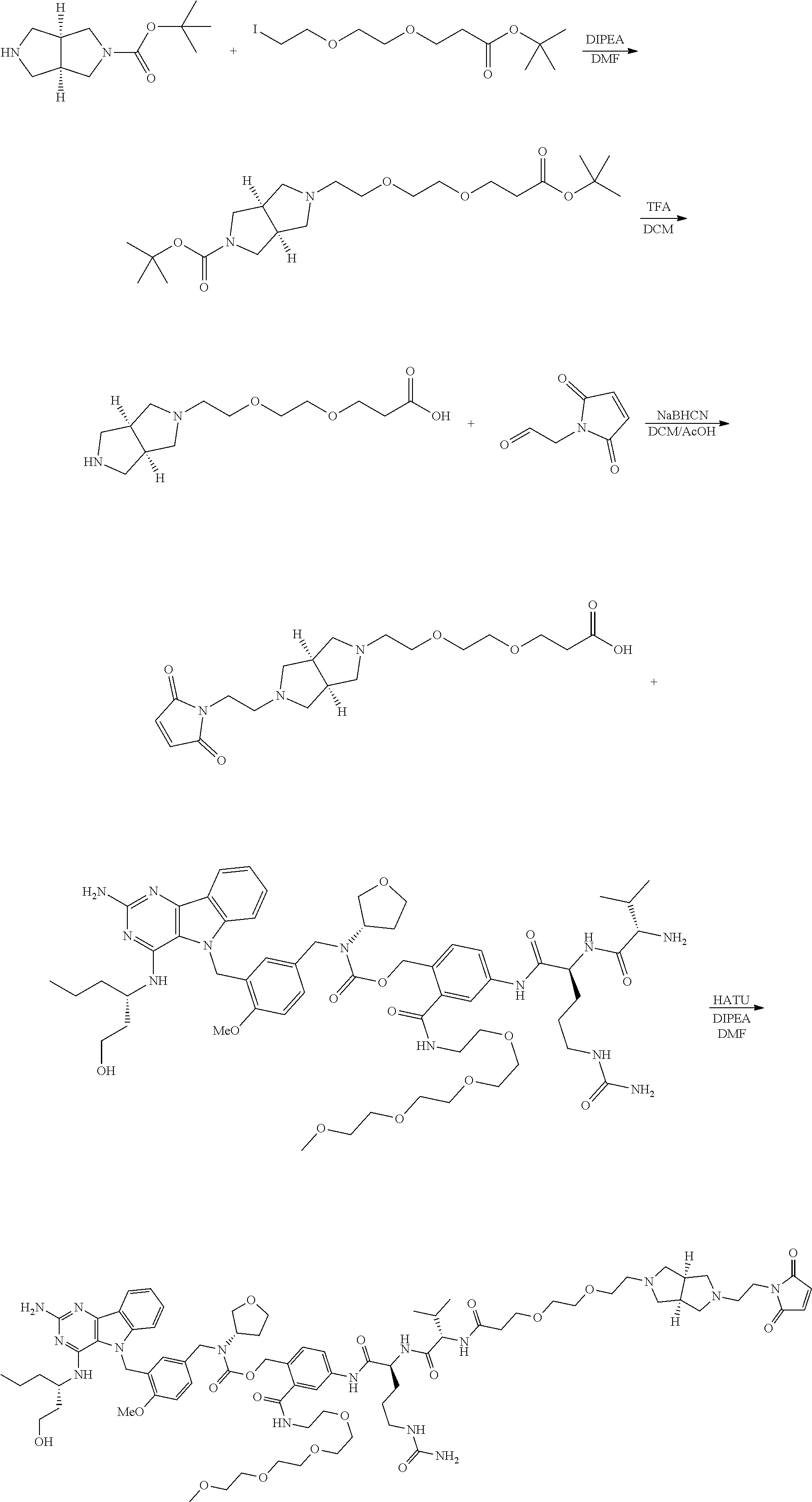

### Step 3-1. tert-butyl (3aR,6aS)-5-(2-(2-(3-(tert-butoxy)-3-oxopropoxy)ethoxy)ethyl) hexahydropyrrolo[3,4-c]pyrrole-2(1H)-carboxylate, Trifluoroacetate Salt

A scintillation vial was charged with tert-butyl 3-(2-(2-iodoethoxy)ethoxy)propanoate (500 mg, 1.453 mmol), cis-2-Boc-hexahydropyrrolo[3,4-c]pyrrole (308 mg, 1.453 mmol), DIPEA (0.507 mL, 2.91 mmol) and DMF (2 mL). The reaction was stirred at room temperature for 72 hours. The reaction mixture was quenched with 5% TFA in water (3 mL), diluted with MeCN (1 mL) a purified using reverse-phase flash chromatography (50 g C18 column, 0 to 50% MeCN in water containing 0.05% TFA). Product fractions were combined and lyophilized to give tert-butyl (3aR,6aS)-5-(2-(2-(3-(tert-butoxy)-3-oxopropoxy)ethoxy)ethyl) hexahydropyrrolo[3,4-c]pyrrole-2(1H)-carboxylate, trifluoroacetate salt (383 mg, 0.706 mmol, 48.6% yield) as an oil. LC-MS (ES, m/z): [M+H]⁺=429.7. ¹H NMR (400 MHz, CHLOROFORM-d) δ 4.31-4.03 (m, 2H), 3.90-3.80 (m, 2H), 3.75-3.68 (m, 2H), 3.67-3.58 (m, 4H), 3.55-3.36 (m, 4H), 3.36-3.29 (m, 2H), 3.22-3.09 (m, 2H), 2.87-2.60 (m, 2H), 2.55-2.45 (m, 2H), 1.57-1.39 (m, 18H).

### Step 3-2. 3-(2-(2-((3aR,6aS)-hexahydropyrrolo[3,4-c]pyrrol-2(1H)-yl)ethoxy)ethoxy)propanoic Acid, Bis-Trifluoroacetate Salt

tert-Butyl(3aR,6aS)-5-(2-(2-(3-(tert-butoxy)-3-oxopropoxy)ethoxy)ethyl) hexahydropyrrolo[3,4-c]pyrrole-2(1H)-carboxylate (617 mg, 1.440 mmol) was dissolved in DCM (4400 mL). TFA (1.109 mL, 14.40 mmol) was added, and the reaction was stirred at room temperature for 3 hours, then evaporated to dryness. The residue was dissolved in MeOH (5 mL) and evaporated to dryness twice, then lyophilized to give 3-(2-(2-((3aR,6aS)-hexahydropyrrolo[3,4-c]pyrrol-2(1H)-yl)ethoxy)ethoxy)propanoic acid, bis-trifluoroacetate (456 mg, 0.911 mmol, 63.3% yield) as a gum. ¹H NMR (400 MHz, DMSO-d₆) δ 9.52 (br s, 1H), 4.53 (br s, 1H), 4.46 (br s, 1H), 3.73 (br t, J=4.7 Hz, 2H), 3.66-3.59 (m, 3H), 3.59-3.44 (m, 6H), 3.43-3.32 (m, 2H), 2.49-2.44 (m, 2H).

### Step 3-3. 3-(2-(2-((3aR,6aS)-5-(2-(2,5-dioxo-2,5-dihydro-1H-pyrrol-1-yl)ethyl)hexahydropyrrolo[3,4-c]pyrrol-2(1H)-yl)ethoxy)ethoxy)propanoic Acid, Bis-Trifluoroacetate Salt

To a stirred solution of 3-(2-(2-((3aR,6aS)-hexahydropyrrolo[3,4-c]pyrrol-2(1H)-yl)ethoxy)ethoxy)propanoic acid, bis-trifluoroacetate salt (150 mg, 0.300 mmol) and 2-(2,5-dioxo-2,5-dihydro-1H-pyrrol-1-yl)acetaldehyde (50.0 mg, 0.360 mmol) in DCM (3 mL) was added acetic acid (0.300 mL) followed by sodium cyanoborohydride (37.7 mg, 0.600 mmol). The reaction was stirred at room temperature for 5 minutes. The reaction mixture was quenched with water (3 mL) and evaporated to remove the organics. The residue was diluted with water (1 mL) and MeCN (0.5 mL) and purified using reverse-phase flash chromatography (50 g C₁₈ column, 0 to 30% MeCN in water containing 0.05% TFA). Product fractions were combined and lyophilized to give 3-(2-(2-((3aR,6aS)-5-(2-(2,5-dioxo-2,5-dihydro-1H-pyrrol-1-yl)ethyl)hexahydropyrrolo[3,4-c]pyrrol-2(1H)-yl)ethoxy)ethoxy)propanoic acid, bis-trifluoroacetate salt (90 mg, 0.144 mmol, 48.2% yield) as a solid. LC-MS (ES, m/z): [M+H]⁺=396.1.

### Step 3-4. 2-((2,5,8,11-tetraoxatridecan-13-yl)carbamoyl)-4-((2S,5S)-15-((3aR,6aS)-5-(2-(2,5-dioxo-2,5-dihydro-1H-pyrrol-1-yl)ethyl)hexahydropyrrolo[3,4-c]pyrrol-2(H)-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

A scintillation vial was charged with 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (40 mg, 0.035 mmol), 3-(2-(2-((3aR,6aS)-5-(2-(2,5-dioxo-2,5-dihydro-1H-pyrrol-1-yl)ethyl)hexahydropyrrolo[3,4-c]pyrrol-2(1H)-yl)ethoxy)ethoxy)propanoic acid, bis-trifluoroacetate salt (25.9 mg, 0.041 mmol), HATU (15.77 mg, 0.041 mmol) and DMF (0.5 mL). DIPEA (6.04 μl, 0.035 mmol) was added, and the reaction stirred at room temperature for 10 minutes. The reaction mixture was quenched with 5% formic acid in water (3 mL), diluted with MeCN (0.5 mL), filtered and purified using preparatory HPLC (30×150 mm C₁₈ column, 10 to 35% MeCN in water containing 0.05% formic acid). Product fractions were combined and lyophilized to give 2-((2,5,8,11-tetraoxatridecan-13-yl)carbamoyl)-4-((2S,5S)-15-((3aR,6aS)-5-(2-(2,5-dioxo-2,5-dihydro-1H-pyrrol-1-yl)ethyl)hexahydropyrrolo[3,4-c]pyrrol-2(1H)-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, bis-formic acid salt (21.9 mg, 0.013 mmol, 38.9% yield) as a white solid. LC-MS (ES, m/z): [M+H]⁺=1535.3, r.t. 1.39 mins.

LCMS Method: Column: Waters ACQUITY UPLC BEH C18, 2.1 mm×50 mm, 1.7 m particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm).

### Example 4. 2-((2,5,8,11-tetraoxatridecan-13-yl)carbamoyl)-4-((2S,5S)-16-((3aR,6aS)-5-(2-(2,5-dioxo-2,5-dihydro-1H-pyrrol-1-yl)ethyl)hexahydropyrrolo[3,4-c]pyrrol-2(H)-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

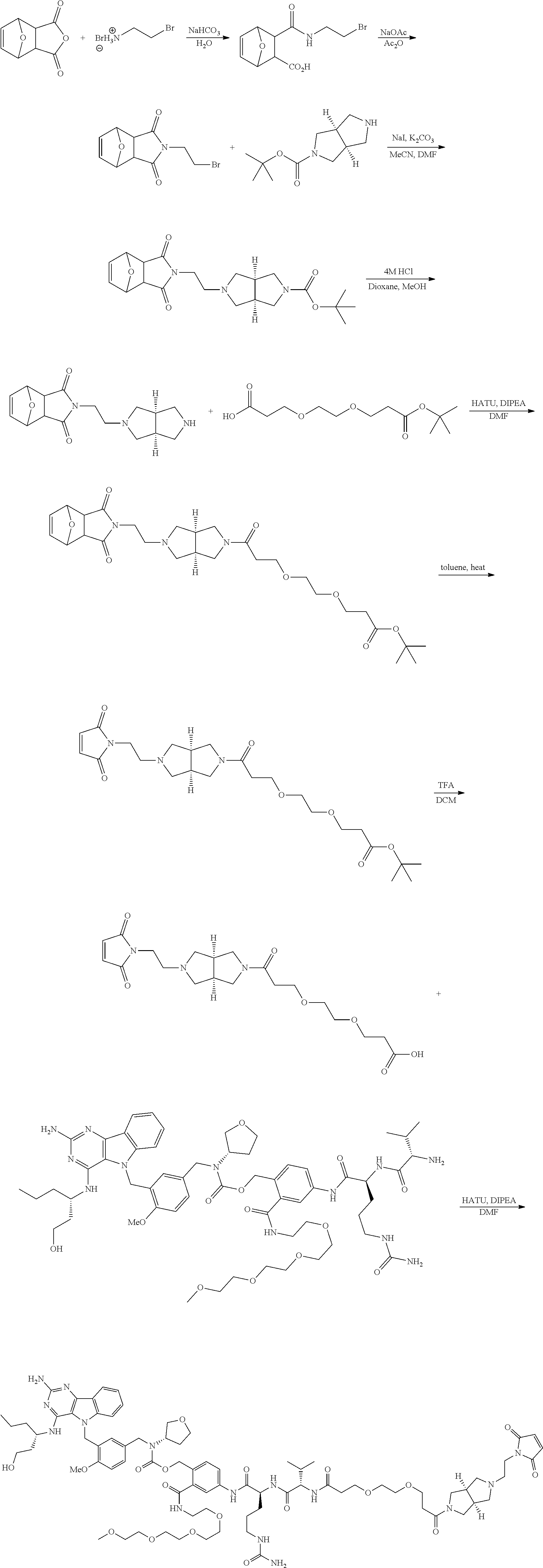

### Step 4-1. 3-((2-bromoethyl)carbamoyl)-7-oxabicyclo[2.2.1]hept-5-ene-2-carboxylic Acid

In a 250 mL round bottom flask, sodium bicarbonate (4.10 g, 48.8 mmol) and 2-bromoethylamine hydrobromide (10 g, 48.8 mmol) were dissolved in water (67 mL), followed by 3a,4,7,7a-tetrahydro-4,7-epoxyisobenzofuran-1,3-dione (8.11 g, 48.8 mmol). The mixture was stirred at room temperature for 1.5 h at which time the colorless solids were collected by filtration, washed with water twice, dried under vacuum to afford 3-((2-bromoethyl)carbamoyl)-7-oxabicyclo[2.2.1]hept-5-ene-2-carboxylic acid (8.6 g, 29.6 mmol, 61% yield). The crude was used without further purification.

### Step 4-2. 2-(2-bromoethyl)-3a,4,7,7a-tetrahydro-1H-4,7-epoxyisoindole-1,3(2H)-dione

In a 30 mL vial, acetic anhydride (6.4 mL, 67.7 mmol) was added to sodium acetate (0.22 g, 2.71 mmol) and the mixture was heated to 85° C. 3-((2-bromoethyl)carbamoyl)-7-oxabicyclo[2.2.1]hept-5-ene-2-carboxylic acid (3.93 g, 13.55 mmol) was added to the mixture. The reaction was stirred for 15 minutes, at which time the reaction changed to a brown suspension and was cooled to room temperature. The brown solution was slowly added to a vigorously stirring ice mixture and was further sonicated. The solids were filtered and dried overnight under vacuum overnight to afford 2-(2-bromoethyl)-3a,4,7,7a-tetrahydro-1H-4,7-epoxyisoindole-1,3(2H)-dione (1.38 g, 5.07 mmol, 37% yield) as a beige solid. ¹H NMR (400 MHz, CDCl₃) δ 6.52 (s, 2H), 5.29 (s, 2H), 3.90 (t, J=4 Hz, 2H), 3.49 (t, J=4 Hz, 2H), 2.89 (s, 2H).

### Step 4-3. tert-butyl (3aR,6aS)-5-(2-(1,3-dioxo-1,3,3a,4,7,7a-hexahydro-2H-4,7-epoxyisoindol-2-yl)ethyl)hexahydropyrrolo[3,4-c]pyrrole-2(1H)-carboxylate

In a 20 mL scintillation vial, potassium carbonate (406 mg, 2.94 mmol), sodium iodide (110 mg, 0.735 mmol), Cis-2-Boc-hexahydropyrrolo[3,4-c]pyrrole (203 mg, 0.956 mmol), 2-(2-bromoethyl)-3a,4,7,7a-tetrahydro-1H-4,7-epoxyisoindole-1,3(2H)-dione (200 mg, 0.735 mmol) were suspended in MeCN (2 mL) and DMF (0.1 mL). The solution was heated to 50° C. and stirred for 22 h. The reaction was cooled to room temperature and filtered through a pad of celite that was wetted with DCM. The filter cake was washed with 100 mL of DCM, and combined filtrates were concentrated under reduced pressure. The crude was purified by normal phase silica gel chromatography using the Isco system (24 g cartridge, 0-20% MeOH in DCM over 20 min) to afford the product as a beige solid. LC-MS (ES, m/z): [M+H]⁺=404.3.

### Step 4-4. 2-(2-((3aR,6aS)-hexahydropyrrolo[3,4-c]pyrrol-2(H)-yl)ethyl)-3a,4,7,7a-tetrahydro-1H-4,7-epoxyisoindole-1,3(2H)-dione, 2 HCl

In a 20 mL scintillation vial, tert-butyl (3aR,6aS)-5-(2-(1,3-dioxo-1,3,3a,4,7,7a-hexahydro-2H-4,7-epoxyisoindol-2-yl)ethyl)hexahydropyrrolo[3,4-c]pyrrole-2(1H)-carboxylate (138.8 mg, 0.344 mmol) was suspended in MeOH (1 mL) and 4 M HCl in dioxane (1.5 mL) was added. The solution was stirred at room temperature for one hour, at which time the solvent was removed under reduced pressured to afford 2-(2-((3aR,6aS)-hexahydropyrrolo[3,4-c]pyrrol-2(1H)-yl)ethyl)-3a,4,7,7a-tetrahydro-1H-4,7-epoxyisoindole-1,3(2H)-dione, 2 HCl (125 mg, 97% yield) as an orange solid. LC-MS (ES, m/z): [M+H]⁺=304.3.

### Step 4-5. tert-butyl 3-(2-(3-((3aR,6aS)-5-(2-(1,3-dioxo-1,3,3a,4,7,7a-hexahydro-2H-4,7-epoxyisoindol-2-yl)ethyl)hexahydropyrrolo[3,4-c]pyrrol-2(1H)-yl)-3-oxopropoxy)ethoxy)propanoate, TFA

In a 20 mL scintillation vial, 2-(2-((3aR,6aS)-hexahydropyrrolo[3,4-c]pyrrol-2(1H)-yl)ethyl)-3a,4,7,7a-tetrahydro-1H-4,7-epoxyisoindole-1,3(2H)-dione, 2 HCl (125.2 mg, 0.333 mmol) was dissolved in DMF (0.2 mL) and N-ethyl-N-isopropylpropan-2-amine (0.232 mL, 1.331 mmol) was added. 3-(2-(3-(tert-Butoxy)-3-oxopropoxy)ethoxy)propanoic acid (79 mg, 0.299 mmol) was added followed by HATU (133 mg, 0.349 mmol). LCMS analysis of the reaction mixture after 5 minutes suggested completion of reaction. The mixture was directly loaded onto a 30 g C18 cartridge and purified by reverse phase preparatory chromatography using the ISCO system (0-40% MeCN+0.05% TFA over 20 min). Lyophilization of relevant fractions afforded tert-butyl 3-(2-(3-((3aR,6aS)-5-(2-(1,3-dioxo-1,3,3a,4,7,7a-hexahydro-2H-4,7-epoxyisoindol-2-yl)ethyl)hexahydropyrrolo[3,4-c]pyrrol-2(1H)-yl)-3-oxopropoxy)ethoxy)propanoate, TFA (123 mg, 0.224 mmol, 56% yield) as a colorless residue. LC-MS (ES, m/z): [M+H]⁺=548.4.

### Step 4-6. 3-(2-(3-((3aR,6aS)-5-(2-(2,5-dioxo-2,5-dihydro-1H-pyrrol-1-yl)ethyl) hexahydropyrrolo[3,4-c]pyrrol-2(H)-yl)-3-oxopropoxy)ethoxy)propanoic Acid, 2 TFA

In a 20 mL vial, tert-butyl 3-(2-(3-((3aR,6aS)-5-(2-(1,3-dioxo-1,3,3a,4,7,7a-hexahydro-2H-4,7-epoxyisoindol-2-yl)ethyl)hexahydropyrrolo[3,4-c]pyrrol-2(1H)-yl)-3-oxopropoxy) ethoxy)propanoate, TFA (123 mg, 0.158 mmol) was dissolved in toluene (1.6 mL). The reaction was heated to 110° C. for six hours, at which time the reaction was cooled to room temperature, concentrated under reduced pressure and purified by reverse phase preparatory chromatography using the Isco system (0-40% MeCN+0.05% TFA over 20 min). Lyophilization of relevant fractions afforded tert-butyl 3-(2-(3-((3aR,6aS)-5-(2-(2,5-dioxo-2,5-dihydro-1H-pyrrol-1-yl)ethyl)hexahydropyrrolo[3,4-c]pyrrol-2(1H)-yl)-3-oxopropoxy)ethoxy)propanoate, TFA (92 mg, 98% yield) as a colorless residue. LC-MS (ES, m/z): [M+H]⁺=480.3.

### Step 4-7. 3-(2-(3-((3aR,6aS)-5-(2-(2,5-dioxo-2,5-dihydro-1H-pyrrol-1-yl)ethyl) hexahydropyrrolo[3,4-c]pyrrol-2(1H)-yl)-3-oxopropoxy)ethoxy)propanoic Acid, TFA

In a 20 mL vial, tert-butyl 3-(2-(3-((3aR,6aS)-5-(2-(2,5-dioxo-2,5-dihydro-1H-pyrrol-1-yl)ethyl)hexahydropyrrolo[3,4-c]pyrrol-2(1H)-yl)-3-oxopropoxy)ethoxy)propanoate, TFA (64.4 mg, 0.108 mmol) was dissolved in DCM (0.5 mL) and 0.5 mL of TFA was added. The reaction was heated to 35° C. LCMS analysis of the reaction mixture after 1.5 h suggested formation of product. The reaction mixture was concentrated under reduced pressure and dried on the lyophilizer overnight to afford 3-(2-(3-((3aR,6aS)-5-(2-(2,5-dioxo-2,5-dihydro-1H-pyrrol-1-yl)ethyl)hexahydropyrrolo[3,4-c]pyrrol-2(1H)-yl)-3-oxopropoxy)ethoxy)propanoic acid, TFA as a colorless residue (64.5 mg, 91% yield). LC-MS (ES, m/z): [M+H]⁺=424.2. Rt=0.50 min.

### Step 4-8. 2-((2,5,8,11-tetraoxatridecan-13-yl)carbamoyl)-4-((2S,5S)-16-((3aR,6aS)-5-(2-(2,5-dioxo-2,5-dihydro-1H-pyrrol-1-yl)ethyl)hexahydropyrrolo[3,4-c]pyrrol-2(H)-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 Formic Acid Salt

In a 4 mL vial, 3-(2-(3-((3aR,6aS)-5-(2-(2,5-dioxo-2,5-dihydro-1H-pyrrol-1-yl)ethyl)hexahydropyrrolo[3,4-c]pyrrol-2(1H)-yl)-3-oxopropoxy)ethoxy)propanoic acid, TFA (6.97 mg, 0.013 mmol), 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (15 mg, 0.013 mmol) and N-ethyl-N-isopropylpropan-2-amine (0.011 mL, 0.065 mmol) were dissolved in DMF (0.15 mL), and HATU (5.17 mg, 0.014 mmol) was added.

LCMS analysis of the reaction mixture immediately after the addition of HATU suggested consumption of starting material and formation of the desired product. The reaction was quenched with 400 μL of 5% formic acid in water and 200 μL of MeCN. The reaction was purified using preparatory HPLC (ACCQ Prep., 20×150 mm C18 column, 5 to 50% MeCN in water containing 0.05% FA over 24 minutes). Fractions which contained the product were combined and lyophilized to give 2-((2,5,8,11-tetraoxatridecan-13-yl)carbamoyl)-4-((2S,5S)-16-((3aR,6aS)-5-(2-(2,5-dioxo-2,5-dihydro-1H-pyrrol-1-yl)ethyl)hexahydropyrrolo[3,4-c]pyrrol-2(1H)-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 formic acid salt as a pale yellow solid (16.8 mg, 76% yield). LC-MS (ES, m/z): [M+H]⁺=1563.7. Rt=1.27 min.

### Example 5. 2-((2,5,8,11-tetraoxatridecan-13-yl)carbamoyl)-4-((2S,5S)-16-((1S,4S)-5-(2-(2,5-dioxo-2,5-dihydro-1H-pyrrol-1-yl)ethyl)-2,5-diazabicyclo[2.2.1]heptan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 Formate Salt

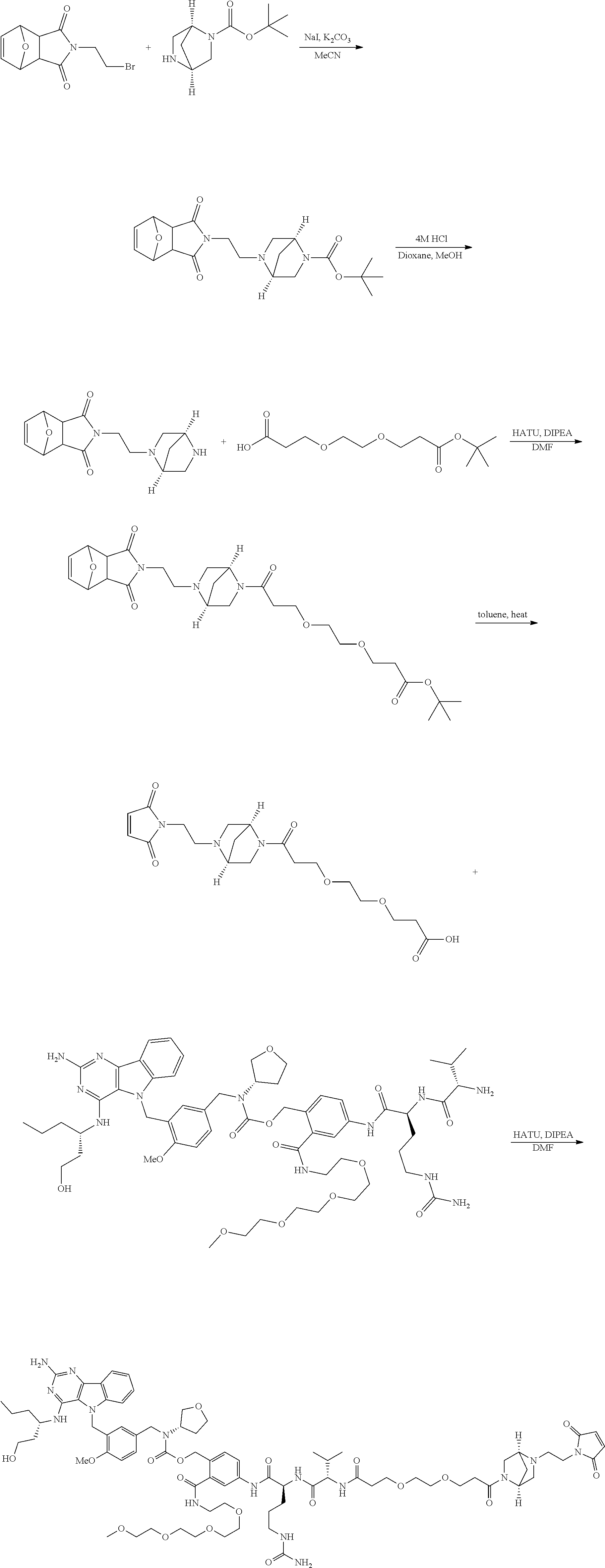

### Step 5-1. tert-butyl (1S,4S)-5-(2-(1,3-dioxo-1,3,3a,4,7,7a-hexahydro-2H-4,7-epoxyisoindol-2-yl)ethyl)-2,5-diazabicyclo[2.2.1]heptane-2-carboxylate

In a 20 mL scintillation vial, potassium carbonate (406 mg, 2.94 mmol), sodium iodide (110 mg, 0.735 mmol), (1S,4S)-2-Boc-2,5-diazabicyclo[2.2.1]heptane (189 mg, 0.956 mmol), 2-(2-bromoethyl)-3a,4,7,7a-tetrahydro-1H-4,7-epoxyisoindole-1,3(2H)-dione (200 mg, 0.735 mmol) were suspended in MeCN (1.5 mL). The solution was heated to 50° C. and stirred for 22 h. The reaction was cooled to room temperature and filtered through a pad of celite that was wetted with DCM. The filter cake was washed with 100 mL of DCM, and combined filtrates were concentrated under reduced pressure. The crude was purified by normal phase silica gel chromatography using the ISCO system (24 g cartridge, 0-20% MeOH in DCM over 20 min) to afford the product as a beige solid (222 mg, 78% yield). LC-MS (ES, m/z): [M+H]⁺=390.2. ¹H NMR (400 MHz, CDCl₃) δ 6.51 (s, 2H), 5.25 (s, 2H), 4.27 (d, J=53 Hz, 1H), 3.61-3.32 (m, 4H), 3.16-3.11 (m, 1H), 2.95-2.85 (m, 3H), 2.71-2.66 (m, 2H), 1.75-1.63 (3H), 1.45 (s, 9H).

### Step 5-2. 2-(2-((1S,4S)-2,5-diazabicyclo[2.2.1]heptan-2-yl)ethyl)-3a,4,7,7a-tetrahydro-1H-4,7-epoxyisoindole-1,3(2H)-dione, 2 HCl

In a 20 mL scintillation vial, tert-butyl (1S,4S)-5-(2-(1,3-dioxo-1,3,3a,4,7,7a-hexahydro-2H-4,7-epoxyisoindol-2-yl)ethyl)-2,5-diazabicyclo[2.2.1]heptane-2-carboxylate (221.6 mg, 0.569 mmol) was suspended in MeOH (1 mL) and 4 M HCl in dioxane (1.5 mL) was added. The solution was stirred at room temperature for 1.5 hours, at which time the solvent was removed under reduced pressured to afford 2-(2-((1S,4S)-2,5-diazabicyclo[2.2.1]heptan-2-yl)ethyl)-3a,4,7,7a-tetrahydro-1H-4,7-epoxyisoindole-1,3(2H)-dione, 2 HCl (200 mg, 97% yield) as an orange solid. LC-MS (ES, m/z): [M+H]⁺=290.2.

### Step 5-3. tert-butyl 3-(2-(3-((1S,4S)-5-(2-(1,3-dioxo-1,3,3a,4,7,7a-hexahydro-2H-4,7-epoxyisoindol-2-yl)ethyl)-2,5-diazabicyclo[2.2.1]heptan-2-yl)-3-oxopropoxy)ethoxy)propanoate, TFA

In a 20 mL scintillation vial, 2-(2-((1S,4S)-2,5-diazabicyclo[2.2.1]heptan-2-yl)ethyl)-3a,4,7,7a-tetrahydro-1H-4,7-epoxyisoindole-1,3(2H)-dione, 2 HCl (200 mg, 0.552 mmol) was dissolved in DMF (0.2 mL) and N-ethyl-N-isopropylpropan-2-amine (0.337 mL, 1.932 mmol) was added. 3-(2-(3-(tert-Butoxy)-3-oxopropoxy)ethoxy)propanoic acid (138 mg, 0.524 mmol) was added followed by HATU (220 mg, 0.580 mmol). LCMS analysis of the reaction mixture after 5 minutes suggested completion of reaction. The mixture was directly loaded onto a 30 g C18 cartridge and purified by reverse phase preparatory chromatography using the ISCO system (0-40% MeCN+0.05% TFA over 20 min). Lyophilization of relevant fractions afforded tert-butyl 3-(2-(3-((1S,4S)-5-(2-(1,3-dioxo-1,3,3a,4,7,7a-hexahydro-2H-4,7-epoxyisoindol-2-yl)ethyl)-2,5-diazabicyclo[2.2.1]heptan-2-yl)-3-oxopropoxy)ethoxy)propanoate, TFA (262 mg, 71% yield) as a colorless residue. LC-MS (ES, m/z): [M+H]⁺=534.3.

### Step 5-4. 3-(2-(3-((1S,4S)-5-(2-(2,5-dioxo-2,5-dihydro-1H-pyrrol-1-yl)ethyl)-2,5-diazabicyclo[2.2.1]heptan-2-yl)-3-oxopropoxy)ethoxy)propanoic Acid, TFA

In a 20 mL vial, tert-butyl 3-(2-(3-((1S,4S)-5-(2-(1,3-dioxo-1,3,3a,4,7,7a-hexahydro-2H-4,7-epoxyisoindol-2-yl)ethyl)-2,5-diazabicyclo[2.2.1]heptan-2-yl)-3-oxopropoxy) ethoxy)propanoate, 2 TFA (261.8 mg, 0.344 mmol) was dissolved in toluene (2 mL). The reaction was heated to 110° C. for 5.5 hours, at which time the reaction was cooled to room temperature, concentrated under reduced pressure and purified by reverse phase preparatory chromatography using the Isco system (0-40% MeCN+0.05% TFA over 20 min). Lyophilization of relevant fractions afforded 3-(2-(3-((1S,4S)-5-(2-(2,5-dioxo-2,5-dihydro-1H-pyrrol-1-yl)ethyl)-2,5-diazabicyclo[2.2.1]heptan-2-yl)-3-oxopropoxy)ethoxy)propanoic acid, TFA (96.8 mg, 44% yield) as a colorless residue. LC-MS (ES, m/z): [M+H]⁺=410.2.

### Step 5-5. 2-((2,5,8,11-tetraoxatridecan-13-yl)carbamoyl)-4-((2S,5S)-16-((3aR,6aS)-5-(2-(2,5-dioxo-2,5-dihydro-1H-pyrrol-1-yl)ethyl)hexahydropyrrolo[3,4-c]pyrrol-2(1H)-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 Formic Acid Salt

In a 4 mL vial, 3-(2-(3-((1S,4S)-5-(2-(2,5-dioxo-2,5-dihydro-1H-pyrrol-1-yl)ethyl)-2,5-diazabicyclo[2.2.1]heptan-2-yl)-3-oxopropoxy)ethoxy)propanoic acid, TFA (6.78 mg, 0.013 mmol), 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (15 mg, 0.013 mmol) and N-ethyl-N-isopropylpropan-2-amine (0.011 mL, 0.065 mmol) were dissolved in DMF (0.15 mL), and HATU (5.17 mg, 0.014 mmol) was added.

LCMS analysis of the reaction mixture immediately after the addition of HATU suggested consumption of starting material and formation of the desired product. The reaction was quenched with 400 μL of 5% formic acid in water and 200 μL of MeCN. The reaction was purified using preparatory HPLC (ACCQ Prep., 20×150 mm C18 column, 5 to 50% MeCN in water containing 0.05% FA over 24 minutes). Fractions which contained the product were combined and lyophilized to give 2-((2,5,8,11-tetraoxatridecan-13-yl)carbamoyl)-4-((2S,5S)-16-((1S,4S)-5-(2-(2,5-dioxo-2,5-dihydro-1H-pyrrol-1-yl)ethyl)-2,5-diazabicyclo[2.2.1]heptan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 formate salt as a pale yellow solid (9.7 mg, 44% yield). LC-MS (ES, m/z): [M+H]⁺=1549.6. Rt=1.26 min.

### Example 6. 2-((2,5,8,11-tetraoxatridecan-13-yl)carbamoyl)-4-((2S,5S)-16-(4-(2-(2,5-dioxo-2,5-dihydro-1H-pyrrol-1-yl)ethyl)piperazin-1-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 Formate Salt

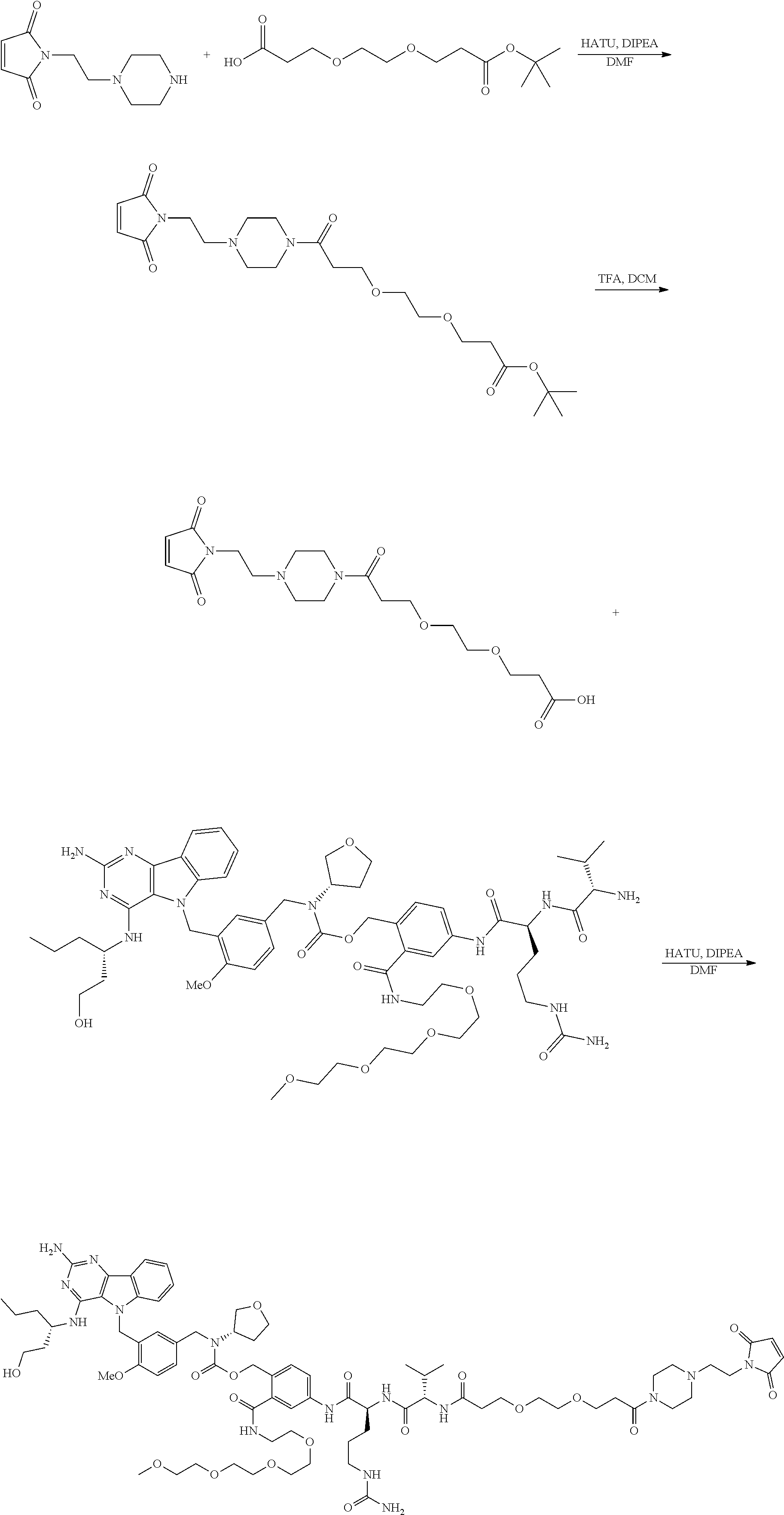

### Step 6-1. tert-butyl 3-(2-(3-(4-(2-(2,5-dioxo-2,5-dihydro-1H-pyrrol-1-yl)ethyl)piperazin-1-yl)-3-oxopropoxy)ethoxy)propanoate, TFA

In a 20 mL vial, 1-(2-(piperazin-1-yl)ethyl)-1H-pyrrole-2,5-dione, 2 HCl (150 mg, 0.532 mmol) was dissolved in DMF (0.2 mL) and N-ethyl-N-isopropylpropan-2-amine (0.296 mL, 1.701 mmol) was added. 3-(2-(3-(tert-Butoxy)-3-oxopropoxy)ethoxy)propanoic acid (132 mg, 0.505 mmol) was added followed by HATU (212 mg, 0.558 mmol). LCMS analysis of the reaction mixture after 1.5 h suggested formation of product. The reaction was quenched with 3% TFA in water, and the solution was directly loaded onto a 30 g C18 cartridge, and purified by reverse phase preparatory chromatography using the Isco system (0-40% MeCN+0.05% TFA over 20 min) Fractions containing the desired product were combined and lyophilized to afford tert-butyl 3-(2-(3-(4-(2-(2,5-dioxo-2,5-dihydro-1H-pyrrol-1-yl)ethyl)piperazin-1-yl)-3-oxopropoxy)ethoxy) propanoate, TFA as a colorless residue (46.3 mg, 12% yield). LC-MS (ES, m/z): [M+H]⁺=454.3.

### Step 6-2. 3-(2-(3-(4-(2-(2,5-dioxo-2,5-dihydro-1H-pyrrol-1-yl)ethyl)piperazin-1-yl)-3-oxopropoxy)ethoxy)propanoic Acid, 2 TFA

In a 20 mL vial, tert-butyl tert-butyl 3-(2-(3-(4-(2-(2,5-dioxo-2,5-dihydro-1H-pyrrol-1-yl)ethyl)piperazin-1-yl)-3-oxopropoxy)ethoxy)propanoate (46.3 mg, 0.102 mmol) was dissolved in DCM (0.5 mL) and TFA (0.5 mL) was added. The reaction was heated to 30° C. Three hours later, LCMS analysis of the reaction mixture suggested formation of product. The mixture was concentrated and the product 3-(2-(3-(4-(2-(2,5-dioxo-2,5-dihydro-1H-pyrrol-1-yl)ethyl)piperazin-1-yl)-3-oxopropoxy)ethoxy)propanoic acid, 2 TFA (42.8 mg, 67% yield) was used directly without further purification. LC-MS (ES, m/z): [M+H]⁺=398.2. Rt=0.43 min.

### Step 6-3. 2-((2,5,8,11-tetraoxatridecan-13-yl)carbamoyl)-4-((2S,5S)-16-(4-(2-(2,5-dioxo-2,5-dihydro-1H-pyrrol-1-yl)ethyl)piperazin-1-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 Formate Salt

In a 4 mL vial, 3-(2-(3-(4-(2-(2,5-dioxo-2,5-dihydro-1H-pyrrol-1-yl)ethyl)piperazin-1-yl)-3-oxopropoxy)ethoxy)propanoic acid, TFA (6.63 mg, 0.013 mmol), 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (15 mg, 0.013 mmol) and N-ethyl-N-isopropylpropan-2-amine (0.011 mL, 0.065 mmol) were dissolved in DMF (0.15 mL), and HATU (5.17 mg, 0.014 mmol) was added. LCMS analysis of the reaction mixture immediately after the addition of HATU suggested consumption of starting material and formation of the desired product. The reaction was quenched with 400 μL of 5% formic acid in water and 200 μL of MeCN. The reaction was purified using preparatory HPLC (ACCQ Prep., 20×150 mm C18 column, 5 to 50% MeCN in water containing 0.05% FA over 24 minutes). Fractions which contained the product were combined and lyophilized to give 2-((2,5,8,11-tetraoxatridecan-13-yl)carbamoyl)-4-((2S,5S)-16-(4-(2-(2,5-dioxo-2,5-dihydro-1H-pyrrol-1-yl)ethyl)piperazin-1-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 formate salt as a pale yellow solid (8.2 mg, 36% yield). LC-MS (ES, m/z): [M+H]⁺=1537.7. Rt=1.28 min.

### Example 7. 2-((2,5,8,11-tetraoxatridecan-13-yl)carbamoyl)-4-((2S,5S)-16-((1S,6R)-5-(2-(2,5-dioxo-2,5-dihydro-1H-pyrrol-1-yl)ethyl)-2,5-diazabicyclo[4.2.0]octan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 Formic Acid Salt

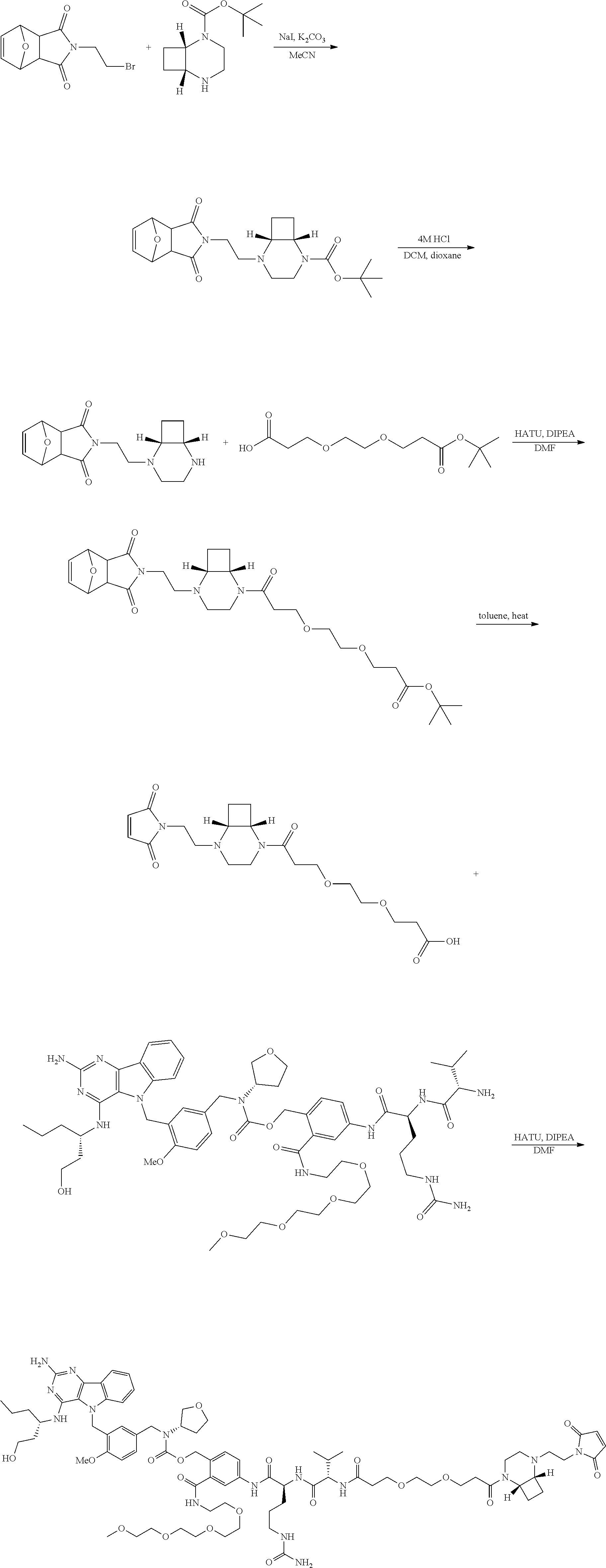

### Step 7-1. tert-butyl (1S,6R)-5-(2-(1,3-dioxo-1,3,3a,4,7,7a-hexahydro-2H-4,7-epoxyisoindol-2-yl)ethyl)-2,5-diazabicyclo[4.2.0]octane-2-carboxylate

In a 20 mL scintillation vial, potassium carbonate (406 mg, 2.94 mmol), sodium iodide (110 mg, 0.735 mmol), tert-butyl (1S,6R)-2,5-diazabicyclo[4.2.0]octane-2-carboxylate (172 mg, 0.809 mmol), 2-(2-bromoethyl)-3a,4,7,7a-tetrahydro-1H-4,7-epoxyisoindole-1,3(2H)-dione (200 mg, 0.735 mmol) were suspended in MeCN (1.5 mL). The solution was heated to 50° C. and stirred for 21 h. The reaction was cooled to room temperature and filtered through a pad of celite that was wetted with DCM. The filter cake was washed with 100 mL of DCM, and combined filtrates were concentrated under reduced pressure. The crude was purified by normal phase silica gel chromatography using the ISCO system (24 g cartridge, 0-20% MeOH in DCM over 20 min) to afford the product as a beige solid (177 mg, 60% yield). LC-MS (ES, m/z): [M+H]⁺=404.3.

### Step 7-2. 2-(2-((1R,6S)-2,5-diazabicyclo[4.2.0]octan-2-yl)ethyl)-3a,4,7,7a-tetrahydro-1H-4,7-epoxyisoindole-1,3(2H)-dione, 2 HCl

In a 20 mL scintillation vial, tert-butyl (1S,6R)-5-(2-(1,3-dioxo-1,3,3a,4,7,7a-hexahydro-2H-4,7-epoxyisoindol-2-yl)ethyl)-2,5-diazabicyclo[4.2.0]octane-2-carboxylate (177 mg, 0.439 mmol) was suspended in DCM (1 mL) and 4 M HCl in dioxane (1.5 mL) was added. The solution was stirred at room temperature for 1.5 hours, at which time the solvent was removed under reduced pressured to afford 2-(2-((1R,6S)-2,5-diazabicyclo[4.2.0]octan-2-yl)ethyl)-3a,4,7,7a-tetrahydro-1H-4,7-epoxyisoindole-1,3(2H)-dione, 2 HCl (170 mg, quant. yield) as a beige solid. LC-MS (ES, m/z): [M+H]⁺=290.2.

### Step 7-3. tert-butyl 3-(2-(3-((1S,6R)-5-(2-(1,3-dioxo-1,3,3a,4,7,7a-hexahydro-2H-4,7-epoxyisoindol-2-yl)ethyl)-2,5-diazabicyclo[4.2.0]octan-2-yl)-3-oxopropoxy)ethoxy)propanoate, TFA

In a 20 mL vial, 2-(2-((1R,6S)-2,5-diazabicyclo[4.2.0]octan-2-yl)ethyl)-3a,4,7,7a-tetrahydro-1H-4,7-epoxyisoindole-1,3(2H)-dione, 2 HCl (164 mg, 0.436 mmol) was dissolved in DMF (0.2 mL) and N-ethyl-N-isopropylpropan-2-amine (0.266 mL, 1.525 mmol) was added. 3-(2-(3-(tert-Butoxy)-3-oxopropoxy)ethoxy)propanoic acid (103 mg, 0.392 mmol) was added followed by HATU (174 mg, 0.458 mmol). LCMS analysis of the reaction mixture after 5 minutes suggested formation of product. The reaction was quenched with 3% TFA in water, and the solution was directly loaded onto a 30 g C18 cartridge, and purified by reverse phase preparatory chromatography using the Isco system (0-40% MeCN+0.05% TFA over 20 min) Fractions containing the desired product were combined and lyophilized to afford tert-butyl 3-(2-(3-((1S,6R)-5-(2-(1,3-dioxo-1,3,3a,4,7,7a-hexahydro-2H-4,7-epoxyisoindol-2-yl)ethyl)-2,5-diazabicyclo[4.2.0]octan-2-yl)-3-oxopropoxy)ethoxy)propanoate, TFA as an orange residue (186 mg, 70% yield). LC-MS (ES, m/z): [M+H]⁺=548.3.

### Step 7-4. 3-(2-(3-((1S,6R)-5-(2-(2,5-dioxo-2,5-dihydro-1H-pyrrol-1-yl)ethyl)-2,5-diazabicyclo[4.2.0]octan-2-yl)-3-oxopropoxy)ethoxy)propanoic Acid, TFA

In a 20 mL vial, tert-butyl 3-(2-(3-((1S,6R)-5-(2-(1,3-dioxo-1,3,3a,4,7,7a-hexahydro-2H-4,7-epoxyisoindol-2-yl)ethyl)-2,5-diazabicyclo[4.2.0]octan-2-yl)-3-oxopropoxy)ethoxy)propanoate (185.9 mg, 0.339 mmol) was dissolved in toluene (1.5 mL). The reaction was heated to 110° C. for 6 hours, at which time the reaction was cooled to room temperature, concentrated under reduced pressure and purified by reverse phase preparatory chromatography using the Isco system (0-40% MeCN+0.05% TFA over 20 min). Lyophilization of relevant fractions afforded 3-(2-(3-((1S,6R)-5-(2-(2,5-dioxo-2,5-dihydro-1H-pyrrol-1-yl)ethyl)-2,5-diazabicyclo[4.2.0]octan-2-yl)-3-oxopropoxy)ethoxy)propanoic acid, TFA (24 mg, 13% yield) as a colorless residue. LC-MS (ES, m/z): [M+H]⁺=424.2.

### Step 7-5. 2-((2,5,8,11-tetraoxatridecan-13-yl)carbamoyl)-4-((2S,5S)-16-((1S,6R)-5-(2-(2,5-dioxo-2,5-dihydro-1H-pyrrol-1-yl)ethyl)-2,5-diazabicyclo[4.2.0]octan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 Formic Acid Salt

In a 4 mL vial, 3-(2-(3-((1S,6R)-5-(2-(2,5-dioxo-2,5-dihydro-1H-pyrrol-1-yl)ethyl)-2,5-diazabicyclo[4.2.0]octan-2-yl)-3-oxopropoxy)ethoxy)propanoic acid, 2 TFA (7.04 mg, 10.80 μmol), 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (12.5 mg, 10.80 μmol) and N-ethyl-N-isopropylpropan-2-amine (9.41 μl, 0.054 mmol) were dissolved in DMF (0.15 mL), and HATU (4.31 mg, 0.011 mmol) was added. LCMS analysis of the reaction mixture immediately after the addition of HATU suggested consumption of starting material and formation of the desired product. The reaction was quenched with 400 μL of 5% formic acid in water and 200 μL of MeCN. The reaction was purified using preparatory HPLC (ACCQ Prep., 20×150 mm C18 column, 5 to 50% MeCN in water containing 0.05% FA over 24 minutes). Fractions which contained the product were combined and lyophilized to give 2-((2,5,8,11-tetraoxatridecan-13-yl)carbamoyl)-4-((2S,5S)-16-((1S,6R)-5-(2-(2,5-dioxo-2,5-dihydro-1H-pyrrol-1-yl)ethyl)-2,5-diazabicyclo[4.2.0]octan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 formic acid salt (10.7 mg, 58% yield). LC-MS (ES, m/z): [M+H]⁺=1563.8. r.t.=1.30 min.

### Example 8. 2-((2,5,8,11-tetraoxatridecan-13-yl)carbamoyl)-4-((2S,5S)-15-((1S,6R)-5-(2-(2,5-dioxo-2,5-dihydro-1H-pyrrol-1-yl)ethyl)-2,5-diazabicyclo[4.2.0]octan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 Formate Salt

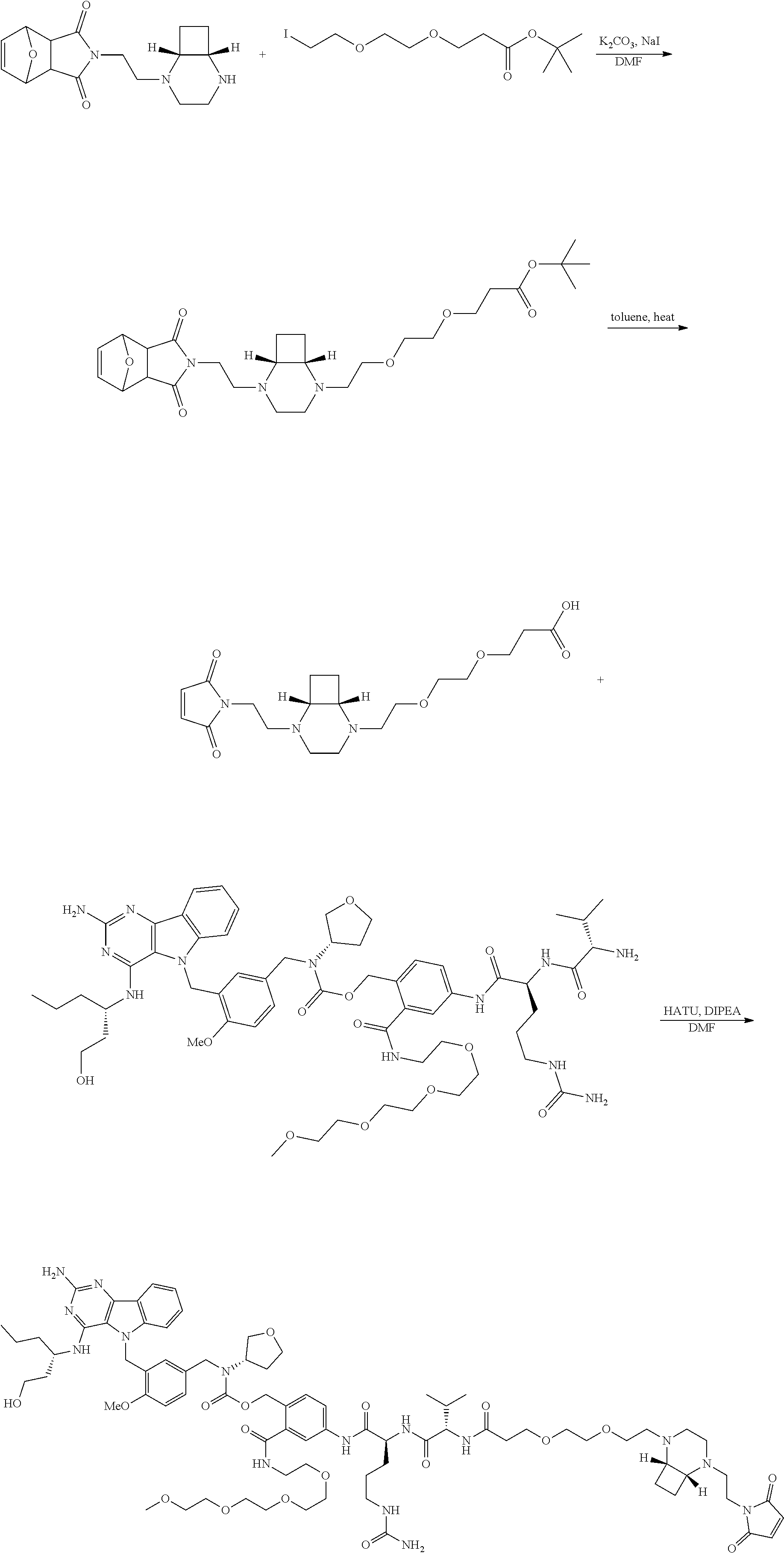

### Step 8-1. tert-butyl 3-(2-(2-((1S,6R)-5-(2-(1,3-dioxo-1,3,3a,4,7,7a-hexahydro-2H-4,7-epoxyisoindol-2-yl)ethyl)-2,5-diazabicyclo[4.2.0]octan-2-yl)ethoxy)ethoxy)propanoate, 2 TFA

In a 20 mL vial, 2-(2-((1R,6S)-2,5-diazabicyclo[4.2.0]octan-2-yl)ethyl)-3a,4,7,7a-tetrahydro-1H-4,7-epoxyisoindole-1,3(2H)-dione, 2 HCl (125 mg, 0.332 mmol) was dissolved in DMF (0.8 mL) and potassium carbonate (459 mg, 3.32 mmol) was added. tert-butyl 3-(2-(2-iodoethoxy)ethoxy)propanoate (114 mg, 0.332 mmol) was added afterwards and the mixture was stirred at 45° C. for 2.5 hours. The reaction mixture was cooled to room temperature and filtered. The crude was purified by reverse phase C18 prep chromatography using the Isco system (0-40% MeCN+0.05% TFA over 20 min). Fractions containing the product were concentrated and lyophilized to afford tert-butyl 3-(2-(2-((1S,6R)-5-(2-(1,3-dioxo-1,3,3a,4,7,7a-hexahydro-2H-4,7-epoxyisoindol-2-yl)ethyl)-2,5-diazabicyclo[4.2.0]octan-2-yl)ethoxy)ethoxy)propanoate, 2 TFA—(152 mg, 61% yield) as a brown residue. LC-MS (ES, m/z): [M+H]⁺=520.5.

### Step 8-2. 3-(2-(2-((1S,6R)-5-(2-(2,5-dioxo-2,5-dihydro-1H-pyrrol-1-yl)ethyl)-2,5-diazabicyclo[4.2.0]octan-2-yl)ethoxy)ethoxy)propanoic Acid, 2 TFA

In a 20 mL vial, tert-butyl 3-(2-(2-((1S,6R)-5-(2-(1,3-dioxo-1,3,3a,4,7,7a-hexahydro-2H-4,7-epoxyisoindol-2-yl)ethyl)-2,5-diazabicyclo[4.2.0]octan-2-yl)ethoxy)ethoxy)propanoate (152 mg, 0.293 mmol) was dissolved in toluene (1.5 mL). The reaction was heated to 110° C. for 6 hours, at which time the reaction was cooled to room temperature, concentrated under reduced pressure and purified by reverse phase preparatory chromatography using the Isco system (0-40% MeCN+0.05% TFA over 20 min). Lyophilization of relevant fractions afforded 3-(2-(2-((1S,6R)-5-(2-(2,5-dioxo-2,5-dihydro-1H-pyrrol-1-yl)ethyl)-2,5-diazabicyclo[4.2.0]octan-2-yl)ethoxy)ethoxy)propanoic acid, 2 TFA (17.2 mg, 9.4% yield) as a colorless residue. LC-MS (ES, m/z): [M+H]⁺=396.2.

### Step 8-3. 2-((2,5,8,11-tetraoxatridecan-13-yl)carbamoyl)-4-((2S,5S)-15-((1S,6R)-5-(2-(2,5-dioxo-2,5-dihydro-1H-pyrrol-1-yl)ethyl)-2,5-diazabicyclo[4.2.0]octan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 Formate Salt

In a 4 mL vial, 3-(2-(2-((1S,6R)-5-(2-(2,5-dioxo-2,5-dihydro-1H-pyrrol-1-yl)ethyl)-2,5-diazabicyclo[4.2.0]octan-2-yl)ethoxy)ethoxy)propanoic acid, 2 TFA (6.73 mg, 10.80 μmol), 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (12.5 mg, 10.80 μmol) and N-ethyl-N-isopropylpropan-2-amine (9.41 μl, 0.054 mmol) were dissolved in DMF (0.15 mL), and HATU (4.31 mg, 0.011 mmol) was added.

LCMS analysis of the reaction mixture immediately after the addition of HATU suggested consumption of starting material and formation of the desired product. The reaction was quenched with 400 μL of 5% formic acid in water and 200 μL of MeCN. The reaction was purified using preparatory HPLC (ACCQ Prep., 20×150 mm C18 column, 5 to 50% MeCN in water containing 0.05% FA over 24 minutes). Fractions which contained the product were combined and lyophilized to give 2-((2,5,8,11-tetraoxatridecan-13-yl)carbamoyl)-4-((2S,5S)-15-((1S,6R)-5-(2-(2,5-dioxo-2,5-dihydro-1H-pyrrol-1-yl)ethyl)-2,5-diazabicyclo[4.2.0]octan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2 formate salt (8 mg, 46% yield). LC-MS (ES, m/z): [M+H]⁺=1534.7. Rt=1.33 min.

### Example 9. 2-((2,5,8,11-tetraoxatridecan-13-yl)carbamoyl)-4-((2S,5S)-16-(7-(2-(2,5-dioxo-2,5-dihydro-1H-pyrrol-1-yl)ethyl)-4,7-diazaspiro[2.5]octan-4-yl)-5-isopropyl-4,7,16-trioxo-2-(3-ureidopropyl)-10,13-dioxa-3,6-diazahexadecanamido)benzyl(3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

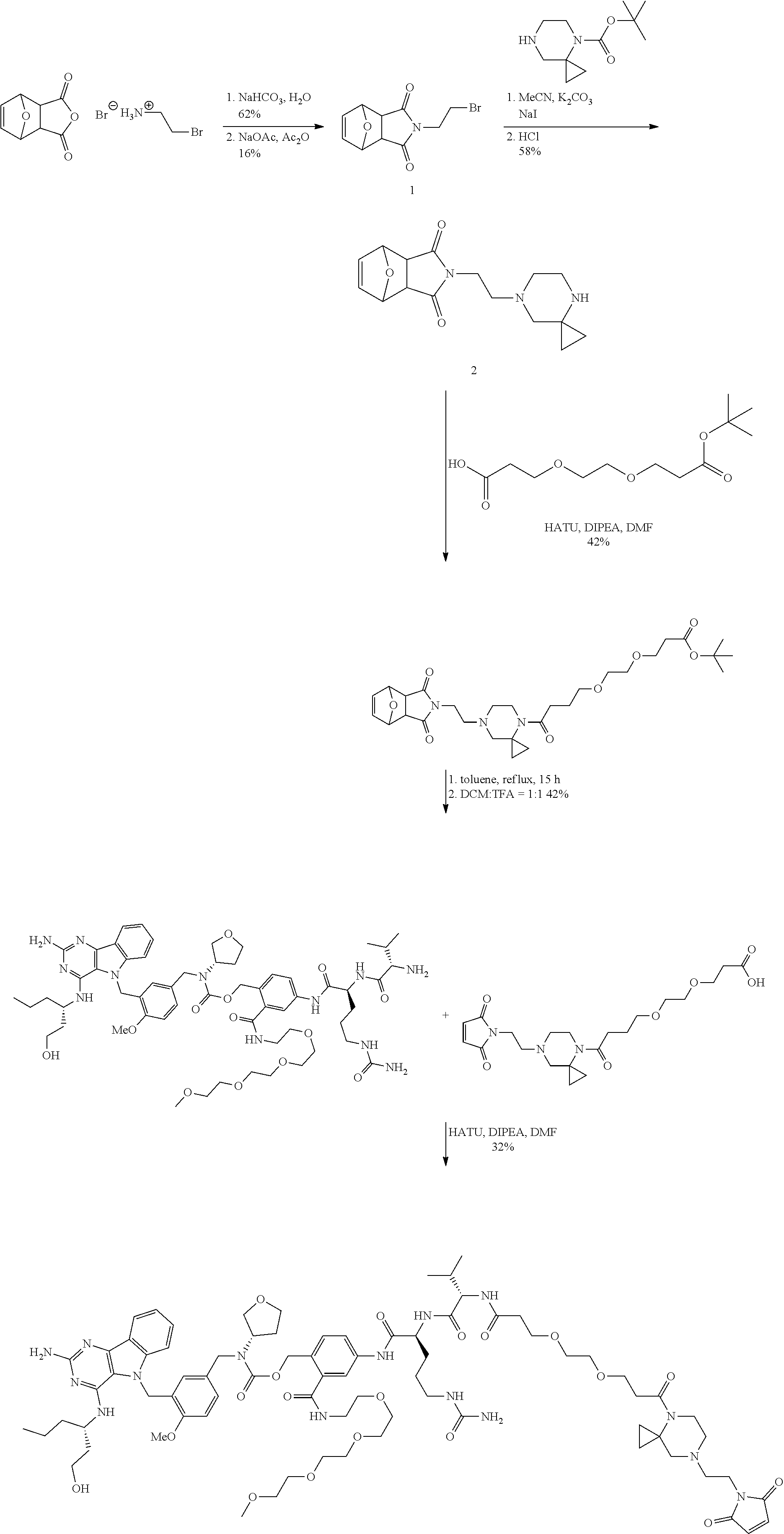

### Step 9-1: Synthesis of 2-(2-bromoethyl)-3a,4,7,7a-tetrahydro-1H-4,7-epoxyisoindole-1,3(2H)-dione

A mixture of Sodium bicarbonate (1.011 g, 12.04 mmol) and 2-Bromoethylamine hydrobromide (2.467 g, 12.04 mmol) were dissolved in water (10 ml) and stirred at RT. 3a,4,7,7a-tetrahydro-4,7-epoxyisobenzofuran-1,3-dione (2 g, 12.04 mmol) was added while stirring, a white precipitate formed. One hour later, the product 3-((2-bromoethyl)carbamoyl)-7-oxabicyclo[2.2.1]hept-5-ene-2-carboxylic acid (2.18 g, 7.51 mmol, 62% yield) was filtered and dried under vacuum and used without further purification (reference: DOI: 10.1002/macp.20170027). Reaction mixture taken to next step without further purification.

To Acetic anhydride (1.303 mL, 13.79 mmol) was added Sodium acetate (0.113 g, 1.379 mmol) and the mixture was heated to 80° C. Above product 3-((2-bromoethyl)carbamoyl)-7-oxabicyclo[2.2.1]hept-5-ene-2-carboxylic acid (2 g, 6.89 mmol) was added to the mixture. The reaction was stirred at 80° C. for 15 min, at which time the reaction changed to a brown suspension and was cooled to room temperature. The mixture was added slowly to a vigorously stirring ice water mixture and precipitate was filtered and lyophilized to give 2-(2-bromoethyl)-3a,4,7,7a-tetrahydro-1H-4,7-epoxyisoindole-1,3(2H)-dione (300 mg, 1.103 mmol, 16% yield) as a light brown solid. 1H NMR (400 MHz, CHLOROFORM-d) δ 6.55 (s, 2H), 5.33-5.27 (m, 2H), 3.92 (t, J=6.8 Hz, 2H), 3.51 (t, J=6.8 Hz, 2H), 2.95-2.88 (m, 2H).

### Step 9-2: Synthesis of 2-(2-(4,7-diazaspiro[2.5]octan-7-yl)ethyl)-3a,4,7,7a-tetrahydro-1H-4,7-epoxyisoindole-1,3(2H)-dione, Hydrochloride Salt

A mixture of 2-(2-bromoethyl)-3a,4,7,7a-tetrahydro-1H-4,7-epoxyisoindole-1,3(2H)-dione (300 mg, 1.103 mmol), tert-butyl 4,7-diazaspiro[2.5]octane-4-carboxylate (234 mg, 1.103 mmol), Potassium carbonate, 99+%, (229 mg, 1.654 mmol) and Sodium iodide (165 mg, 1.103 mmol) were added with Acetonitrile (1 mL) and stirred at 40° C. overnight. The reaction was cooled to room temperature and filtered, and the filtrate was concentrated under reduced pressure. Purification was achieved using normal phase ISCO (40 g cartridge, 0-15% MeOH in DCM 220/254 nm UV detection) to afford the product tert-butyl 7-(2-(1,3-dioxo-1,3,3a,4,7,7a-hexahydro-2H-4,7-epoxyisoindol-2-yl)ethyl)-4,7-diazaspiro[2.5]octane-4-carboxylate (299 mg, 0.741 mmol, 67.2% yield) as a white foam/semisolid. LC-MS (ES, m/z): [M+H]⁺=404.3. 4 M HCl in dioxane (2 mL) was added to the above product butyl 7-(2-(1,3-dioxo-1,3,3a,4,7,7a-hexahydro-2H-4,7-epoxyisoindol-2-yl)ethyl)-4,7-diazaspiro[2.5]octane-4-carboxylate (299 mg, 0.741 mmol) and 2 mL MeOH was added. Reaction was stirred at room temperature for 2 h. Reaction suspension was concentrated under reduced pressure using the V10 and then on lyophilizer to give 2-(2-(4,7-diazaspiro[2.5]octan-7-yl)ethyl)-3a,4,7,7a-tetrahydro-1H-4,7-epoxyisoindole-1,3(2H)-dione, HCl (220 mg, 0.647 mmol, 58% yield) as off-white solid. LC-MS (ES, m/z): [M+H]⁺=304.2.

1H NMR (400 MHz, DMSO-d₆) δ 6.56 (s, 2H), 5.16-5.10 (m, 2H), 3.82-3.60 (m, 3H), 3.59-3.53 (m, 5H), 3.40-3.23 (m, 3H), 3.17 (s, 1H), 3.03-2.91 (m, 2H), 1.29-1.16 (m, 2H), 1.08-0.95 (m, 2H).

### Step 9-3: Synthesis of tert-butyl 3-(2-(3-(7-(2-(1,3-dioxo-1,3,3a,4,7,7a-hexahydro-2H-4,7-epoxyisoindol-2-yl)ethyl)-4,7-diazaspiro[2.5]octan-4-yl)-3-oxopropoxy)ethoxy)propanoate

A mixture of 2-(2-(4,7-diazaspiro[2.5]octan-7-yl)ethyl)-3a,4,7,7a-tetrahydro-1H-4,7-epoxyisoindole-1,3(2H)-dione (145 mg, 0.478 mmol), Acid-peg2-t-butyl ester (125 mg, 0.478 mmol), HATU (200 mg, 0.526 mmol) and N,N-Diisopropylethylamine (0.208 mL, 1.195 mmol) was dissolved in DMF (1 mL) and reaction mixture was stirred at rt for 1 h. Residue was purified using reverse phase ISCO with 0-50% MeCN/H2O+0.05% TFA-20 min 220/254 UV, lyophilized overnight to give tert-butyl 3-(2-(3-(7-(2-(1,3-dioxo-1,3,3a,4,7,7a-hexahydro-2H-4,7-epoxyisoindol-2-yl)ethyl)-4,7-diazaspiro[2.5]octan-4-yl)-3-oxopropoxy)ethoxy)propanoate (110 mg, 0.201 mmol, 42.0% yield) as colorless semisolid. LC-MS (ESI) m/z=548.1, RT 1.06. 1H NMR (400 MHz, DMSO-d₆) δ 9.96 (br s, 1H), 6.60-6.54 (m, 2H), 5.19-5.11 (m, 2H), 3.83-3.54 (m, 13H), 3.18 (s, 3H), 3.04 (s, 1H), 2.98 (s, 3H), 2.74 (br s, 1H), 2.71-2.61 (m, 1H), 2.49-2.39 (m, 2H), 1.71-1.52 (m, 1H), 1.41 (s, 9H), 1.33-1.16 (m, 1H), 1.05-0.72 (m, 2H).

### Step 9-4: Synthesis of 3-(2-(3-(7-(2-(2,5-dioxo-2,5-dihydro-1H-pyrrol-1-yl)ethyl)-4,7-diazaspiro[2.5]octan-4-yl)-3-oxopropoxy)ethoxy)propanoic Acid, Bis Trifluoroacetate Salt

A mixture of tert-butyl 3-(2-(3-(7-(2-(1,3-dioxo-1,3,3a,4,7,7a-hexahydro-2H-4,7-epoxyisoindol-2-yl)ethyl)-4,7-diazaspiro[2.5]octan-4-yl)-3-oxopropoxy)ethoxy)propanoate (100 mg, 0.183 mmol) was dissolved in 1 mL of toluene and the solution was heated to 100° C. for 12 h. Solvent was removed under reduced pressure and the residue was dissolved in ACN/H2O and purified using reverse phase ISCO with 0-50% ACN/H2O with 0.05% TFA to give tert-butyl 3-(2-(3-(7-(2-(2,5-dioxo-2,5-dihydro-1H-pyrrol-1-yl)ethyl)-4,7-diazaspiro[2.5]octan-4-yl)-3-oxopropoxy)ethoxy)propanoate (41 mg, 0.085 mmol, 46.8% yield) as colorless semisolid. LC-MS (ESI) m/z=480.2 RT=1.02.

Tert-butyl 3-(2-(3-(7-(2-(2,5-dioxo-2,5-dihydro-1H-pyrrol-1-yl)ethyl)-4,7-diazaspiro[2.5]octan-4-yl)-3-oxopropoxy)ethoxy)propanoate (41 mg, 0.085 mmol) was dissolved in 1:1 DCM:TFA and stirred at 30° C. for 5 h. Solvent was removed under reduced pressure to give 3-(2-(3-(7-(2-(2,5-dioxo-2,5-dihydro-1H-pyrrol-1-yl)ethyl)-4,7-diazaspiro[2.5]octan-4-yl)-3-oxopropoxy)ethoxy)propanoic acid, bis-trifluoracetate salt (50 mg, 0.077 mmol, 42.0% yield) as a brown residue LC-MS (ESI) m/z=424.3 RT=0.61.

### Step 9-5: 2-((2,5,8,11-tetraoxatridecan-13-yl)carbamoyl)-4-((2S,5S)-16-(7-(2-(2,5-dioxo-2,5-dihydro-1H-pyrrol-1-yl)ethyl)-4,7-diazaspiro[2.5]octan-4-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

A mixture of 2-((2,5,8,11-tetraoxatridecan-13-yl)carbamoyl)-4-((S)-2-((S)-2-amino-3-methylbutanamido)-5-ureidopentanamido)benzyl(3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (15 mg, 0.013 mmol), 3-(2-(3-(7-(2-(2,5-dioxo-2,5-dihydro-1H-pyrrol-1-yl)ethyl)-4,7-diazaspiro[2.5]octan-4-yl)-3-oxopropoxy)ethoxy)propanoic acid, bis-trifluoracetate salt (10.13 mg, 0.016 mmol), HATU (5.91 mg, 0.016 mmol) and N,N-Diisopropylethylamine (2.264 μl, 0.013 mmol) in DMF (0.5 mL) was stirred at room temperature for 1 h. The reaction mixture was quenched with 5% formic acid in water (0.5 mL), diluted with 5% formic acid in MeCN (0.5 mL), filtered and purified using preparatory HPLC (ACCQ Prep., 30×150 mm C18 column, 10 to 35% MeCN in water containing 0.05% formic acid over 24 minutes). Product fractions were combined and lyophilised to give 2-((2,5,8,11-tetraoxatridecan-13-yl)carbamoyl)-4-((2S,5S)-16-(7-(2-(2,5-dioxo-2,5-dihydro-1H-pyrrol-1-yl)ethyl)-4,7-diazaspiro[2.5]octan-4-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, bis formic acid salt (7 mg, 4.23 μmol, 32% yield) as a white solid. LC-MS (ES, m/z): [M+H]⁺=1563.3, consistent with the expected product. ¹H NMR (499 MHz, DMSO-d₆) δ 10.11-10.01 (m, 1H), 8.28 (br s, 1H), 8.11-8.04 (m, 1H), 7.99-7.88 (m, 1H), 7.82-7.75 (m, 1H), 7.67-7.46 (m, 3H), 7.41-7.31 (m, 1H), 7.26-7.08 (m, 1H), 7.00-6.97 (m, 1H), 6.45 (br s, 2H), 5.94 (br s, 1H), 5.69-5.57 (m, 2H), 5.34 (br s, 2H), 4.44-4.23 (m, 2H), 4.16 (br t, J=7.6 Hz, 1H), 4.10-3.94 (m, 3H), 3.80 (s, 3H), 3.65 (br s, 1H), 3.59-3.48 (m, 7H), 3.45-3.35 (m, 33H), 3.19-3.14 (m, 5H), 3.11 (br s, 3H), 2.98-2.78 (m, 3H), 2.57 (br s, 2H), 2.41-2.35 (m, 2H), 2.35-2.22 (m, 3H), 2.19-2.10 (m, 1H), 1.95-1.83 (m, 1H), 1.65-1.52 (m, 3H), 1.52-1.42 (m, 1H), 1.40-1.20 (m, 5H), 0.93-0.84 (m, 2H), 0.78 (br dd, J=16.6, 6.7 Hz, 6H), 0.73-0.50 (m, 5H), 0.23-0.23 (m, 1H).

### Example 10. 2-((2,5,8,11-tetraoxatridecan-13-yl)carbamoyl)-4-((2S,5S)-15-(7-(2-(2,5-dioxo-2,5-dihydro-1H-pyrrol-1-yl)ethyl)-4,7-diazaspiro[2.5]octan-4-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

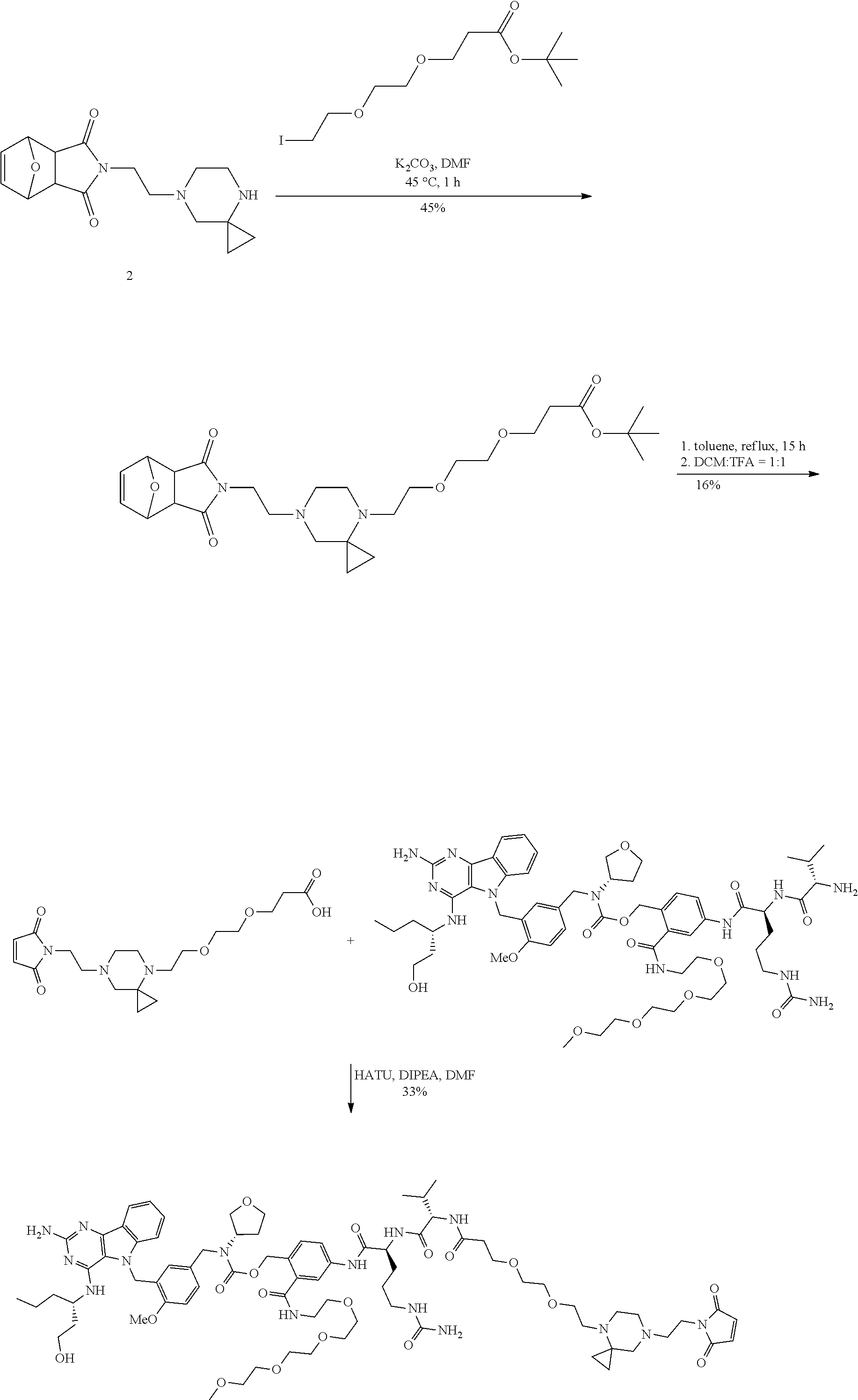

### Step 10-1: Synthesis of tert-butyl 3-(2-(2-(7-(2-(1,3-dioxo-1,3,3a,4,7,7a-hexahydro-2H-4,7-epoxyisoindol-2-yl)ethyl)-4,7-diazaspiro[2.5]octan-4-yl)ethoxy)ethoxy)propanoate

A mixture of 2-(2-(4,7-diazaspiro[2.5]octan-7-yl)ethyl)-3a,4,7,7a-tetrahydro-1H-4,7-epoxyisoindole-1,3(2H)-dione (140 mg, 0.461 mmol), tert-butyl 3-(2-(2-iodoethoxy)ethoxy)propanoate (159 mg, 0.461 mmol) and Potassium carbonate (159 mg, 1.154 mmol) was dissolved in DMF (0.5 mL) and heated at 45° C. for 1 h. solids were filtered and solvent was removed on V10 and residue was purified using reverse phase ISCO with 0-50% MeCN/H2O+0.05% TFA-20 min 220/254 UV, lyophilized overnight to give tert-butyl 3-(2-(2-(7-(2-(1,3-dioxo-1,3,3a,4,7,7a-hexahydro-2H-4,7-epoxyisoindol-2-yl)ethyl)-4,7-diazaspiro[2.5]octan-4-yl)ethoxy)ethoxy)propanoate (107 mg, 0.206 mmol, 45% yield). LC-MS (ESI) m/z=520.4. 1H NMR (400 MHz, DMSO-d₆) δ 6.57 (s, 2H), 5.14 (s, 2H), 3.71 (br s, 2H), 3.69-3.59 (m, 1H), 3.59-3.54 (m, 1H), 3.53-3.44 (m, 8H), 3.22-3.09 (m, 6H), 3.04-2.94 (m, 3H), 2.49-2.37 (m, 3H), 1.40 (s, 12H), 0.82 (br s, 2H), 0.71 (br s, 2H).

### Step 10-2: Synthesis of 3-(2-(2-(7-(2-(2,5-dioxo-2,5-dihydro-1H-pyrrol-1-yl)ethyl)-4,7-diazaspiro[2.5]octan-4-yl)ethoxy)ethoxy)propanoic Acid, Bis-Trifluoroacetate Salt

A mixture of tert-butyl 3-(2-(2-(7-(2-(1,3-dioxo-1,3,3a,4,7,7a-hexahydro-2H-4,7-epoxyisoindol-2-yl)ethyl)-4,7-diazaspiro[2.5]octan-4-yl)ethoxy)ethoxy)propanoate (105 mg, 0.202 mmol) was dissolved in 1 mL of toluene and the solution was heated to 100° C. for 12 h. Solvent was removed under reduced pressure and the residue was purified using reverse phase ISCO with 0-50% ACN/H₂O with 0.05% TFA to give tert-butyl 3-(2-(2-(7-(2-(1,3-dioxo-1,3,3a,4,7,7a-hexahydro-2H-4,7-epoxyisoindol-2-yl)ethyl)-4,7-diazaspiro[2.5]octan-4-yl)ethoxy)ethoxy)propanoate (105 mg, 0.202 mmol) as a colorless semisolid. LC-MS (ESI) m/z=452.3.

Tert-butyl 3-(2-(2-(7-(2-(2,5-dioxo-2,5-dihydro-1H-pyrrol-1-yl)ethyl)-4,7-diazaspiro[2.5]octan-4-yl)ethoxy)ethoxy)propanoate was dissolved in 1:1 DCM:TFA and stirred at 30° C. for 5 h. Solvent was removed under reduced pressure to give 3-(2-(2-(7-(2-(2,5-dioxo-2,5-dihydro-1H-pyrrol-1-yl)ethyl)-4,7-diazaspiro[2.5]octan-4-yl)ethoxy)ethoxy)propanoic acid, bis-trifluoroacetate salt (20 mg, 0.032 mmol, 16% yield) as a brown residue. LC-MS (ESI) m/z=396.3 RT=0.69.

### Step 10-3: Synthesis of 2-((2,5,8,11-tetraoxatridecan-13-yl)carbamoyl)-4-((2S,5S)-15-(7-(2-(2,5-dioxo-2,5-dihydro-1H-pyrrol-1-yl)ethyl)-4,7-diazaspiro[2.5]octan-4-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

A mixture of 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (15 mg, 0.013 mmol), 3-(2-(2-(7-(2-(2,5-dioxo-2,5-dihydro-1H-pyrrol-1-yl)ethyl)-4,7-diazaspiro[2.5]octan-4-yl)ethoxy)ethoxy)propanoic acid, bis trifluoroacetate salt (8.89 mg, 0.014 mmol), HATU (5.91 mg, 0.016 mmol) and N,N-Diisopropylethylamine (2.264 μl, 0.013 mmol) in DMF (0.5 mL) was stirred for 1 h. The reaction mixture was quenched with 5% formic acid in water (0.5 mL), diluted with MeCN (0.5 mL), filtered and purified using preparatory HPLC (ACCQ Prep., 30×150 mm C18 column, 10 to 35% MeCN in water containing 0.05% formic acid over 24 minutes). Product fractions were combined and lyophilised to give 2-((2,5,8,11-tetraoxatridecan-13-yl)carbamoyl)-4-((2S,5S)-15-(7-(2-(2,5-dioxo-2,5-dihydro-1H-pyrrol-1-yl)ethyl)-4,7-diazaspiro[2.5]octan-4-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2-formic acid salt (7 mg, 4.30 μmol, 33% yield) as a white solid. LC-MS (ES, m/z): [M+H]⁺=1534.9 consistent with the expected product. ¹H NMR (499 MHz, DMSO-d₆) δ 10.16-10.01 (m, 1H), 8.30-8.25 (m, 2H), 8.23-8.15 (m, 1H), 8.15-8.06 (m, 1H), 7.93-7.79 (m, 2H), 7.60 (br s, 2H), 7.48 (br s, 1H), 7.31 (br s, 1H), 7.03 (br t, J=7.4 Hz, 2H), 7.00-6.91 (m, 3H), 6.13 (br s, 1H), 6.06-5.95 (m, 1H), 5.93 (br s, 1H), 5.65 (br s, 2H), 5.63-5.48 (m, 2H), 5.47-5.38 (m, 1H), 5.36 (br s, 1H), 5.30 (br d, J=8.2 Hz, 1H), 4.79 (br s, 2H), 4.32 (br s, 2H), 4.26-4.10 (m, 3H), 4.09-3.89 (m, 6H), 3.81 (s, 9H), 3.67-3.54 (m, 8H), 3.37-3.21 (m, 16H), 3.15 (s, 5H), 3.05-2.84 (m, 3H), 2.82-2.74 (m, 1H), 2.74-2.65 (m, 1H), 2.61 (br s, 1H), 2.34-2.22 (m, 4H), 2.07 (br s, 1H), 1.94-1.87 (m, 1H), 1.63 (br s, 1H), 1.58-1.49 (m, 3H), 1.41-1.26 (m, 4H), 1.25-1.14 (m, 1H), 0.93-0.82 (m, 2H), 0.78 (br dd, J=16.9, 6.7 Hz, 5H), 0.64 (br t, J=7.2 Hz, 3H), 0.41-0.35 (m, 1H), 0.26 (br s, 1H).

### Example 11. 2-((2,5,8,11-tetraoxatridecan-13-yl)carbamoyl)-4-((2S,5S)-15-(7-(2-(2,5-dioxo-2,5-dihydro-1H-pyrrol-1-yl)ethyl)-2,7-diazaspiro[4.4]nonan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

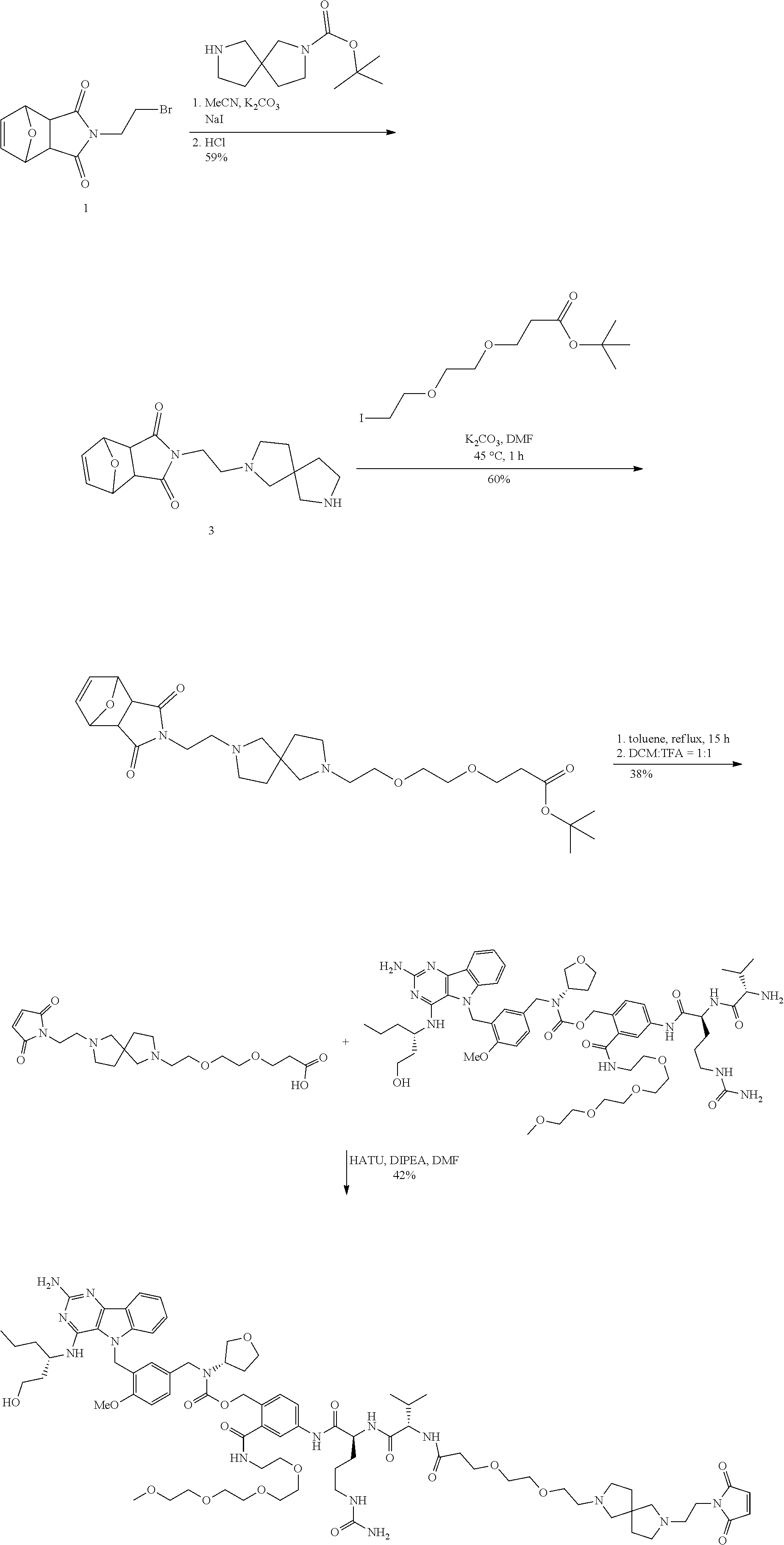

### Step 11-1: Synthesis of 2-(2-(2,7-diazaspiro[4.4]nonan-2-yl)ethyl)-3a,4,7,7a-tetrahydro-1H-4,7-epoxyisoindole-1,3(2H)-dione, Hydrochloride Salt

In a 20 mL vial, 2-(2-bromoethyl)-3a,4,7,7a-tetrahydro-1H-4,7-epoxyisoindole-1,3(2H)-dione (300 mg, 1.103 mmol), tert-Butyl 2,7-diazaspiro[4.4]nonane-2-carboxylate (250 mg, 1.103 mmol), Potassium carbonate, 99+%, (229 mg, 1.654 mmol) and Sodium iodide (165 mg, 1.103 mmol) were dissolved in Acetonitrile (1 mL) and stirred at 45° C. overnight. The reaction mixture was cooled to room temperature and filtered, and the filtrate was concentrated under reduced pressure. Purification was achieved using normal phase silica gel chromatography (25 g cartridge, 0-15% MeOH in DCM 220/254 nm UV detection) to afford tert-butyl 7-(2-(1,3-dioxo-1,3,3a,4,7,7a-hexahydro-2H-4,7-epoxyisoindol-2-yl)ethyl)-2,7-diazaspiro[4.4]nonane-2-carboxylate (240 mg, 0.575 mmol, 52% yield). LC-MS (ES, m/z): [M+H]⁺=418.3. 4 M HCl in dioxane (2 mL) was added to tert-butyl 7-(2-(1,3-dioxo-1,3,3a,4,7,7a-hexahydro-2H-4,7-epoxyisoindol-2-yl)ethyl)-2,7-diazaspiro[4.4]nonane-2-carboxylate (240 mg, 0.575 mmol) and 1 mL MeOH was added. Reaction was stirred at room temperature for 2 h. Reaction suspension was concentrated under reduced pressure using the V10 and dried on the lyophilizer to give the product 2-(2-(2,7-diazaspiro[4.4]nonan-2-yl)ethyl)-3a,4,7,7a-tetrahydro-1H-4,7-epoxyisoindole-1,3(2H)-dione, hydrochloride salt (230 mg, 0.650 mmol, 59% yield) as a yellow solid. LC-MS (ES, m/z): [M+H]⁺=318.2. 1H NMR (400 MHz, DMSO-d₆) δ 6.57 (s, 2H), 5.14 (s, 2H), 3.77-3.65 (m, 4H), 3.53-3.45 (m, 3H), 3.38-3.30 (m, 2H), 3.27-3.17 (m, 6H), 3.02-2.97 (m, 2H), 2.18-1.89 (m, 4H).

### Step 11-2: tert-butyl 3-(2-(2-(7-(2-(1,3-dioxo-1,3,3a,4,7,7a-hexahydro-2H-4,7-epoxyisoindol-2-yl)ethyl)-2,7-diazaspiro[4.4]nonan-2-yl)ethoxy)ethoxy)propanoate

A mixture of 2-(2-(2,7-diazaspiro[4.4]nonan-2-yl)ethyl)-3a,4,7,7a-tetrahydro-1H-4,7-epoxyisoindole-1,3(2H)-dione (220 mg, 0.693 mmol), tert-butyl 3-(2-(2-iodoethoxy)ethoxy)propanoate (239 mg, 0.693 mmol) and Potassium carbonate (239 mg, 1.733 mmol) in DMF (1 mL) was heated at 45° C. for 2 h. Solvent was removed on V10 and residue was purified using reverse phase ISCO with 0-50% MeCN/H2O+0.05% TFA-20 min 220/254 UV, lyophilized overnight to give tert-butyl 3-(2-(2-(7-(2-(1,3-dioxo-1,3,3a,4,7,7a-hexahydro-2H-4,7-epoxyisoindol-2-yl)ethyl)-2,7-diazaspiro[4.4]nonan-2-yl)ethoxy)ethoxy)propanoate (220 mg, 0.412 mmol, 60% yield). LC-MS (ES, m/z): [M+H]⁺=534.4. 1H NMR (400 MHz, DMSO-d₆) δ 6.58 (s, 2H), 5.16 (s, 2H), 3.83-3.71 (m, 7H), 3.63-3.53 (m, 8H), 3.37 (br s, 5H), 3.24 (br s, 3H), 3.00-2.90 (m, 2H), 2.49-2.41 (m, 2H), 2.29-2.00 (m, 3H), 1.40 (s, 9H).

### Step 11-3: Synthesis of 3-(2-(2-(7-(2-(2,5-dioxo-2,5-dihydro-1H-pyrrol-1-yl)ethyl)-2,7-diazaspiro[4.4]nonan-2-yl)ethoxy)ethoxy)propanoic Acid, Bis-Trifluoroacetate Salt

Tert-butyl 3-(2-(2-(7-(2-(1,3-dioxo-1,3,3a,4,7,7a-hexahydro-2H-4,7-epoxyisoindol-2-yl)ethyl)-2,7-diazaspiro[4.4]nonan-2-yl)ethoxy)ethoxy)propanoate (200 mg, 0.375 mmol) was dissolved in 1 mL of toluene and the solution was heated to 100° C. for 12 hours. Solvent was removed under reduced pressure and the residue was dissolved in ACN/H²O and purified using reverse phase ISCO with 0-50% ACN/H2O with 0.05% TFA to give tert-butyl 3-(2-(2-(7-(2-(2,5-dioxo-2,5-dihydro-1H-pyrrol-1-yl)ethyl)-2,7-diazaspiro[4.4]nonan-2-yl)ethoxy)ethoxy)propanoate (92 mg, 0.198 mmol, 52.7% yield) as a colorless semisolid. LC-MS (ES, m/z): [M+H]⁺=466.3.

Tert-butyl 3-(2-(2-(7-(2-(2,5-dioxo-2,5-dihydro-1H-pyrrol-1-yl)ethyl)-2,7-diazaspiro[4.4]nonan-2-yl)ethoxy)ethoxy)propanoate (92 mg, 0.198 mmol) was dissolved in 1:1 DCM:TFA and stirred at 30° C. for 5 h. Solvent was removed under reduced pressure to give 3-(2-(2-(7-(2-(2,5-dioxo-2,5-dihydro-1H-pyrrol-1-yl)ethyl)-2,7-diazaspiro[4.4]nonan-2-yl)ethoxy)ethoxy)propanoic acid, bis trifluoracetate salt (90 mg, 0.141 mmol, 38% yield) as a brown residue. LC-MS (ES, m/z): [M+H]⁺=410.3.

### Step 11-4: 2-((2,5,8,11-tetraoxatridecan-13-yl)carbamoyl)-4-((2S,5S)-15-(7-(2-(2,5-dioxo-2,5-dihydro-1H-pyrrol-1-yl)ethyl)-2,7-diazaspiro[4.4]nonan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

A mixture of 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (15 mg, 0.013 mmol), 3-(2-(2-(7-(2-(2,5-dioxo-2,5-dihydro-1H-pyrrol-1-yl)ethyl)-2,7-diazaspiro[4.4]nonan-2-yl)ethoxy)ethoxy)propanoic acid, 2 TFA (9.09 mg, 0.014 mmol), HATU (5.91 mg, 0.016 mmol) and N N-Diisopropylethylamine (2.264 μl, 0.013 mmol) in DMF (0.5 mL) was stirred for 1 h. The reaction mixture was quenched with 5% formic acid in water (0.5 mL), diluted with MeCN (0.5 mL), filtered and purified using preparatory HPLC (ACCQ Prep., 30×150 mm C18 column, 10 to 35% MeCN in water containing 0.05% formic acid over 24 minutes). Product fractions were combined and lyophilized to give 2-((2,5,8,11-tetraoxatridecan-13-yl)carbamoyl)-4-((2S,5S)-15-(7-(2-(2,5-dioxo-2,5-dihydro-1H-pyrrol-1-yl)ethyl)-2,7-diazaspiro[4.4]nonan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, 2formic acid salt (9 mg, 5.48 μmol, 42% yield) as a white solid. LC-MS (ES, m/z): [M+H]⁺=1549.8. 1H NMR (499 MHz, DMSO-d₆) δ 10.12-10.02 (m, 1H), 8.18 (br s, 2H), 8.12-8.02 (m, 1H), 7.89-7.77 (m, 2H), 7.59 (br s, 1H), 7.48 (br s, 1H), 7.37-7.28 (m, 1H), 7.04 (br t, J=7.3 Hz, 1H), 7.00-6.92 (m, 3H), 6.08-6.00 (m, 1H), 5.98-5.88 (m, 2H), 5.73-5.63 (m, 2H), 5.63-5.49 (m, 2H), 5.40-5.29 (m, 3H), 4.78 (br s, 2H), 4.36-4.28 (m, 1H), 4.25-4.11 (m, 2H), 4.08-3.89 (m, 4H), 3.81 (s, 5H), 3.68-3.51 (m, 11H), 3.45-3.42 (m, 19H), 3.15 (s, 8H), 3.08-2.85 (m, 4H), 2.53-2.45 (m, 4H), 2.40-2.24 (m, 5H), 1.66-1.49 (m, 7H), 1.42-1.26 (m, 5H), 1.26-1.08 (m, 2H), 0.89 (sxt, J=7.5 Hz, 2H), 0.78 (dd, J=16.7, 6.7 Hz, 6H), 0.64 (br t, J=7.3 Hz, 3H).

### Example 12. 2-((2,5,8,11-tetraoxatridecan-13-yl)carbamoyl)-4-((2S,5S)-16-(7-(2-(2,5-dioxo-2,5-dihydro-1H-pyrrol-1-yl)ethyl)-2,7-diazaspiro[4.4]nonan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

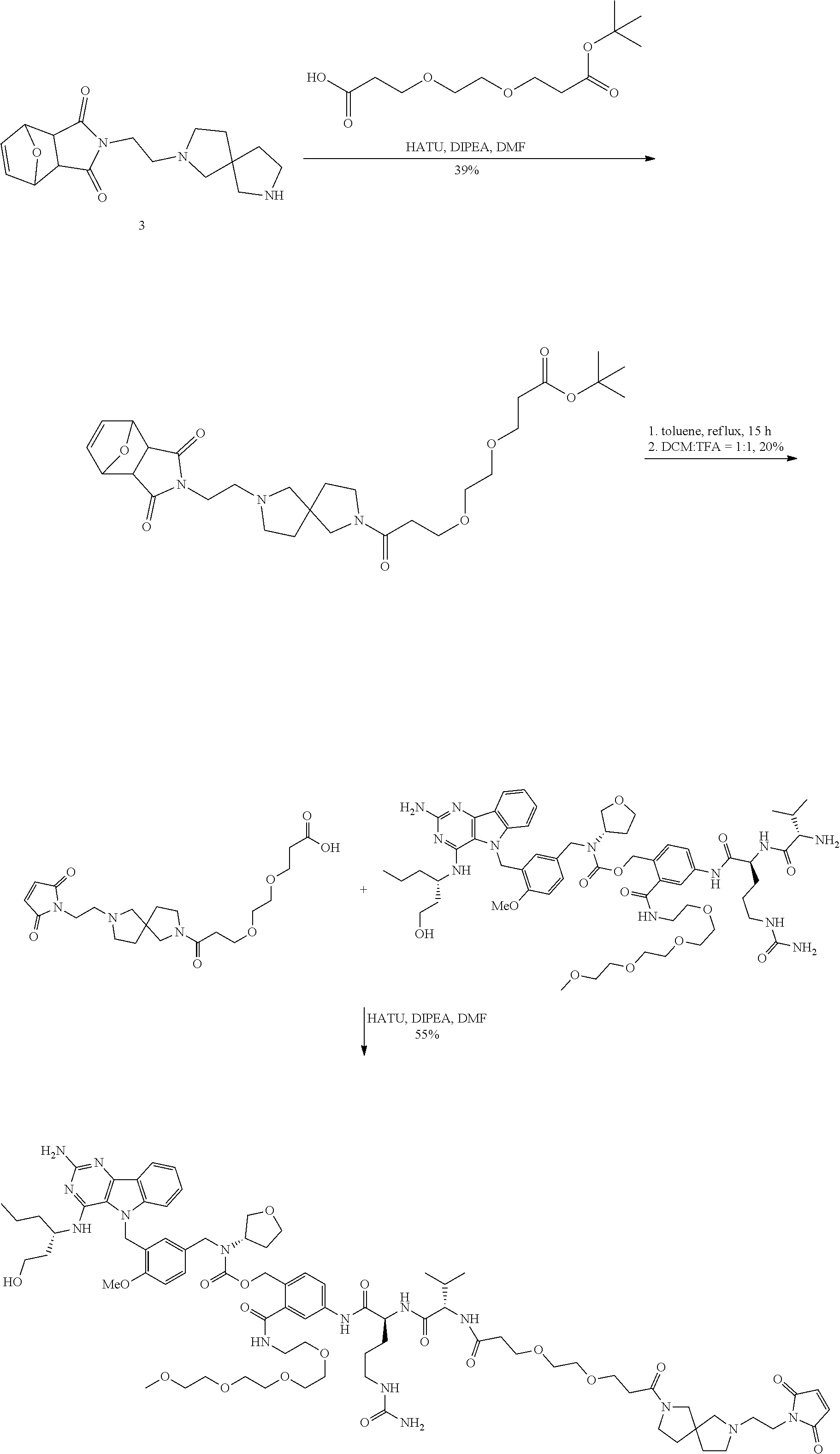

### Step 12-1: Synthesis of tert-butyl 3-(2-(3-(7-(2-(1,3-dioxo-1,3,3a,4,7,7a-hexahydro-2H-4,7-epoxyisoindol-2-yl)ethyl)-2,7-diazaspiro[4.4]nonan-2-yl)-3-oxopropoxy)ethoxy)propanoate

A mixture of 2-(2-(2,7-diazaspiro[4.4]nonan-2-yl)ethyl)-3a,4,7,7a-tetrahydro-1H-4,7-epoxyisoindole-1,3(2H)-dione (180 mg, 0.567 mmol), Acid-peg2-t-butyl ester (149 mg, 0.567 mmol), HATU (237 mg, 0.624 mmol) and N,N-Diisopropylethylamine (0.247 mL, 1.418 mmol) was dissolved in DMF (1 mL) and reaction mixture was stirred at rt for 1 h. Residue was purified using reverse phase ISCO with 0-50% MeCN/H2O+0.05% TFA-20 min 220/254 UV, lyophilized overnight to give tert-butyl 3-(2-(3-(7-(2-(1,3-dioxo-1,3,3a,4,7,7a-hexahydro-2H-4,7-epoxyisoindol-2-yl)ethyl)-2,7-diazaspiro[4.4]nonan-2-yl)-3-oxopropoxy)ethoxy)propanoate (125 mg, 0.223 mmol, 39% yield) as colorless semisolid. LC-MS (ES, m/z): [M+H]⁺=562.3. ¹H NMR (400 MHz, DMSO-d₆) δ 6.58 (s, 2H), 5.18-5.13 (m, 2H), 3.83-3.67 (m, 4H), 3.65-3.56 (m, 15H), 3.40-3.30 (m, 4H), 2.49-2.39 (m, 4H), 2.10-1.93 (m, 2H), 1.93-1.80 (m, 2H), 1.40 (s, 9H).

### Step 12-2: Synthesis of 3-(2-(3-(7-(2-(2,5-dioxo-2,5-dihydro-1H-pyrrol-1-yl)ethyl)-2,7-diazaspiro[4.4]nonan-2-yl)-3-oxopropoxy)ethoxy)propanoic Acid, Bis-Trifluoroacetate Salt

A mixture of tert-butyl 3-(2-(3-(7-(2-(1,3-dioxo-1,3,3a,4,7,7a-hexahydro-2H-4,7-epoxyisoindol-2-yl)ethyl)-2,7-diazaspiro[4.4]nonan-2-yl)-3-oxopropoxy)ethoxy)propanoate (125 mg, 0.223 mmol) was dissolved in 1 mL of toluene and the solution was heated to 100° C. for 12 hours. Solvent was removed under reduced pressure and the residue was dissolved in ACN/H2O and purified using reverse phase ISCO with 0-50% ACN/H2O with 0.05% TFA to give tert-butyl 3-(2-(3-(7-(2-(2,5-dioxo-2,5-dihydro-1H-pyrrol-1-yl)ethyl)-2,7-diazaspiro[4.4]nonan-2-yl)-3-oxopropoxy)ethoxy)propanoate (33 mg, 0.067 mmol, 30.0% yield) as colorless semisolid. LCMS (ESI) m/z=494.3 RT=0.96.

Tert-butyl 3-(2-(3-(7-(2-(2,5-dioxo-2,5-dihydro-1H-pyrrol-1-yl)ethyl)-2,7-diazaspiro[4.4]nonan-2-yl)-3-oxopropoxy)ethoxy)propanoate (33 mg, 0.067 mmol) was dissolved in 1:1 DCM:TFA and stirred at 30° C. for 5 h. Solvent was removed under reduced pressure to give 3-(2-(3-(7-(2-(2,5-dioxo-2,5-dihydro-1H-pyrrol-1-yl)ethyl)-2,7-diazaspiro[4.4]nonan-2-yl)-3-oxopropoxy)ethoxy)propanoic acid, 2 TFA (30 mg, 0.045 mmol, 20% yield) as a brown residue. MS(ESI) m/z=438.3 RT=0.53.

### Step 12-3: 2-((2,5,8,11-tetraoxatridecan-13-yl)carbamoyl)-4-((2S,5S)-16-(7-(2-(2,5-dioxo-2,5-dihydro-1H-pyrrol-1-yl)ethyl)-2,7-diazaspiro[4.4]nonan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

A mixture of 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (15 mg, 0.013 mmol), 3-(2-(3-(7-(2-(2,5-dioxo-2,5-dihydro-1H-pyrrol-1-yl)ethyl)-2,7-diazaspiro[4.4]nonan-2-yl)-3-oxopropoxy)ethoxy)propanoic acid, 2 TFA (9.49 mg, 0.014 mmol), HATU (5.91 mg, 0.016 mmol) and N,N-Diisopropylethylamine (2.264 μl, 0.013 mmol) in DMF (0.5 mL) and stirred at rt for 1 h. The reaction mixture was quenched with 5% formic acid in water (0.5 mL), diluted with MeCN (0.5 mL), filtered and purified using preparatory HPLC (ACCQ Prep., 30×150 mm C18 column, 10 to 35% MeCN in water containing 0.05% formic acid over 24 minutes). Product fractions were combined and lyophilised to give 2-((2,5,8,11-tetraoxatridecan-13-yl)carbamoyl)-4-((2S,5S)-16-(7-(2-(2,5-dioxo-2,5-dihydro-1H-pyrrol-1-yl)ethyl)-2,7-diazaspiro[4.4]nonan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, bis-formic acid salt (12 mg, 7.19 μmol, 55% yield) as a white solid. LC-MS (ES, m/z): [M+H]⁺=1577.6. ¹H NMR (499 MHz, DMSO-d₆) δ 10.11 (br s, 1H), 8.35 (br s, 1H), 8.20-8.11 (m, 2H), 7.95 (t, J=8.3 Hz, 1H), 7.91-7.84 (m, 1H), 7.65 (br s, 1H), 7.59 (br s, 1H), 7.41 (br s, 1H), 7.21-7.09 (m, 1H), 7.08-7.00 (m, 3H), 6.29-6.09 (m, 1H), 6.08-5.94 (m, 3H), 5.80-5.58 (m, 3H), 5.41 (br s, 2H), 4.95 (br s, 1H), 4.81 (br s, 1H), 4.39 (br d, J=5.6 Hz, 2H), 4.31 (br d, J=7.6 Hz, 1H), 4.24 (br t, J=7.5 Hz, 1H), 4.16-3.97 (m, 4H), 3.88 (s, 5H), 3.74-3.56 (m, 12H), 3.43-3.38 (m, 14H), 3.34 (br d, J=4.7 Hz, 6H), 3.24-3.21 (m, 5H), 3.03-3.03 (m, 1H), 3.06-2.94 (m, 2H), 2.68-2.54 (m, 3H), 2.48-2.34 (m, 6H), 2.02-1.90 (m, 1H), 1.79-1.58 (m, 7H), 1.47 (br dd, J=13.8, 5.5 Hz, 3H), 1.43-1.34 (m, 3H), 1.34-1.22 (m, 2H), 1.00-0.91 (m, 2H), 0.87 (br d, J=6.5 Hz, 3H), 0.84 (br d, J=6.7 Hz, 3H), 0.72 (br t, J=7.2 Hz, 3H).

### Example 13. 2-((2,5,8,11-tetraoxatridecan-13-yl)carbamoyl)-4-((2S,5S)-15-(7-(2-(2,5-dioxo-2,5-dihydro-1H-pyrrol-1-yl)ethyl)-2,7-diazaspiro[3.5]nonan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

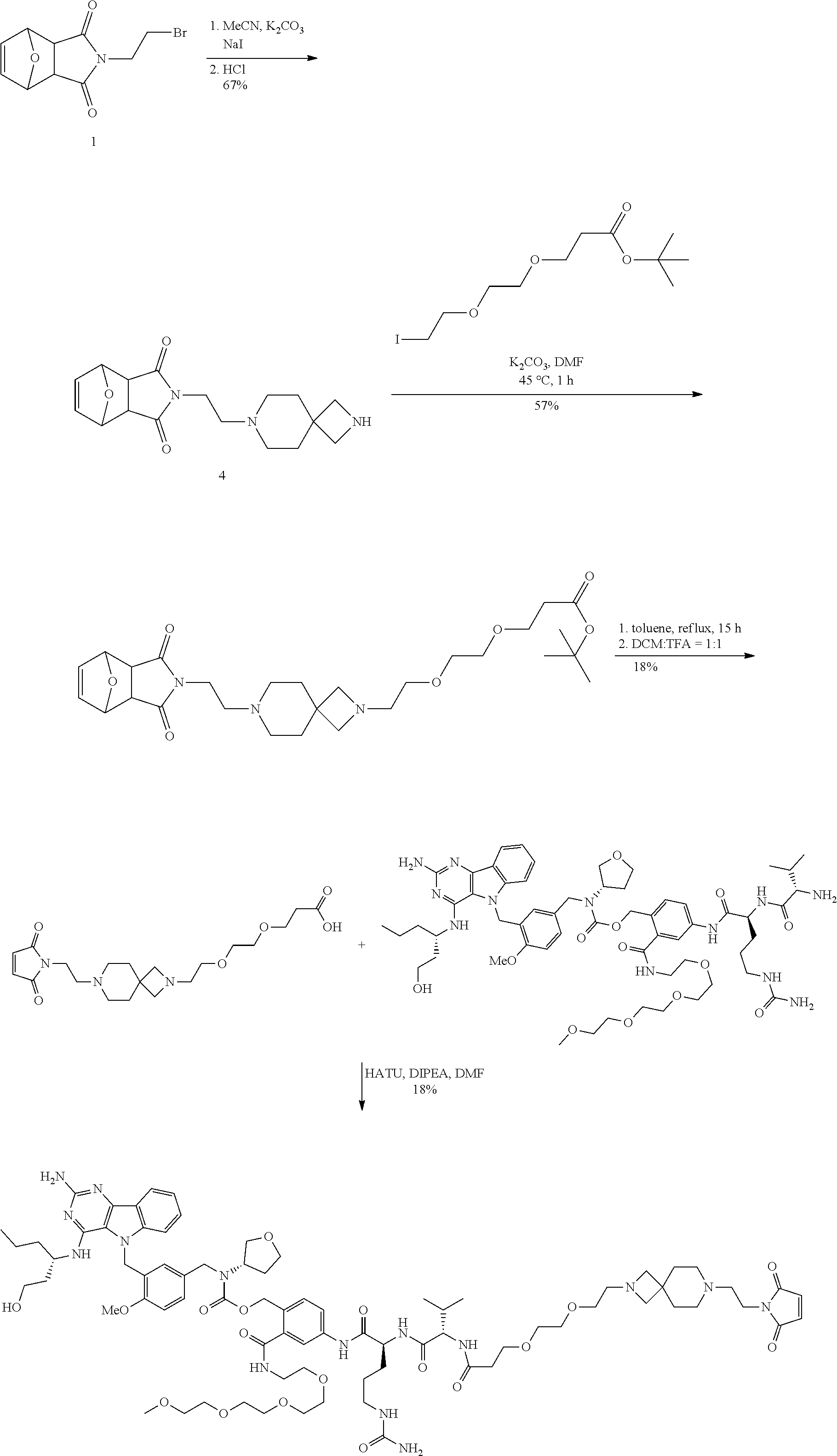

### Step 13-1: Synthesis of 2-(2-(2,7-diazaspiro[3.5]nonan-7-yl)ethyl)-3a,4,7,7a-tetrahydro-1H-4,7-epoxyisoindole-1,3(2H)-dione, Hydrochloride Salt

In a 20 mL vial, 2-(2-bromoethyl)-3a,4,7,7a-tetrahydro-1H-4,7-epoxyisoindole-1,3(2H)-dione (250 mg, 0.919 mmol), tert-butyl 2,7-diazaspiro[3.5]nonane-2-carboxylate (208 mg, 0.919 mmol), Potassium carbonate, 99+%, (190 mg, 1.378 mmol) and Sodium iodide (138 mg, 0.919 mmol) were dissolved in Acetonitrile (1 mL) and stirred at 40° C. overnight. The reaction was cooled to room temperature and filtered, and the filtrate was concentrated under reduced pressure. Purification was achieved using normal phase silica gel chromatography (25 g cartridge, 0-15% MeOH in DCM 220/254 nm UV detection) to afford the product tert-butyl 7-(2-(1,3-dioxo-1,3,3a,4,7,7a-hexahydro-2H-4,7-epoxyisoindol-2-yl)ethyl)-2,7-diazaspiro[3.5]nonane-2-carboxylate (334 mg, 0.800 mmol, 87% yield) as colorless solid. LC-MS (ES, m/z): [M+H]⁺=418.3. 4 M HCl in dioxane (2 mL) was added to tert-butyl 7-(2-(1,3-dioxo-1,3,3a,4,7,7a-hexahydro-2H-4,7-epoxyisoindol-2-yl)ethyl)-2,7-diazaspiro[3.5]nonane-2-carboxylate (334 mg, 0.800 mmol), and 1 mL MeOH was added. Reaction was stirred at room temperature for 2 h. Reaction suspension was concentrated under reduced pressure using the V10 and dried on the lyophilizer to give the product 2-(2-(2,7-diazaspiro[3.5]nonan-7-yl)ethyl)-3a,4,7,7a-tetrahydro-1H-4,7-epoxyisoindole-1,3(2H)-dione, HCl (220 mg, 0.622 mmol, 67% yield) as a yellow solid. LC-MS (ES, m/z): [M+H]⁺=318.3. ¹H NMR (400 MHz, DMSO-d₆) δ 6.56 (s, 2H), 5.19-5.10 (m, 2H), 3.82-3.64 (m, 6H), 3.52-3.44 (m, 2H), 3.21-3.10 (m, 2H), 3.00-2.90 (m, 4H), 2.20 (br d, J=14.0 Hz, 2H), 2.09-1.95 (m, 2H).

### Step 13-2: tert-butyl 3-(2-(2-(7-(2-(1,3-dioxo-1,3,3a,4,7,7a-hexahydro-2H-4,7-epoxyisoindol-2-yl)ethyl)-2,7-diazaspiro[3.5]nonan-2-yl)ethoxy)ethoxy)propanoate

A mixture of 2-(2-(2,7-diazaspiro[3.5]nonan-7-yl)ethyl)-3a,4,7,7a-tetrahydro-1H-4,7-epoxyisoindole-1,3(2H)-dione (200 mg, 0.630 mmol) was added Potassium carbonate (218 mg, 1.575 mmol) and then tert-butyl 3-(2-(2-iodoethoxy)ethoxy)propanoate (217 mg, 0.630 mmol), in DMF (1 mL) and heated at 45° C. for 2 h. Solids were filtered and solvent was removed on V10 and residue was purified using reverse phase ISCO with 0-50% MeCN/H2O+0.05% TFA-20 min 220/254 UV, lyophilized overnight to give tert-butyl 3-(2-(2-(7-(2-(1,3-dioxo-1,3,3a,4,7,7a-hexahydro-2H-4,7-epoxyisoindol-2-yl)ethyl)-2,7-diazaspiro[3.5]nonan-2-yl)ethoxy) ethoxy)propanoate (190 mg, 0.356 mmol, 57% yield) as colorless solid. LC-MS (ES, m/z): [M+H]⁺=534.4. ¹H NMR (400 MHz, DMSO-d₆) δ 6.57 (s, 2H), 5.15 (s, 2H), 4.11-3.86 (m, 7H), 3.86-3.68 (m, 4H), 3.65-3.54 (m, 5H), 3.40 (br s, 2H), 3.21-3.08 (m, 3H), 3.01-2.88 (m, 3H), 2.86-2.73 (m, 1H), 2.43 (t, J=6.2 Hz, 2H), 2.36-2.09 (m, 2H), 2.08-1.82 (m, 2H), 1.40 (s, 9H).

### Step 13-3: Synthesis of 3-(2-(2-(7-(2-(2,5-dioxo-2,5-dihydro-1H-pyrrol-1-yl)ethyl)-2,7-diazaspiro[3.5]nonan-2-yl)ethoxy)ethoxy)propanoic Acid, Bis-Trifluoroacetate Salt

Tert-butyl 3-(2-(2-(7-(2-(1,3-dioxo-1,3,3a,4,7,7a-hexahydro-2H-4,7-epoxyisoindol-2-yl)ethyl)-2,7-diazaspiro[3.5]nonan-2-yl)ethoxy)ethoxy)propanoate (190 mg, 0.356 mmol) was dissolved in 1 mL of toluene and the solution was heated to 100° C. for 12 hours. Solvent was removed under reduced pressure and the residue was dissolved in ACN/H2O and purified using reverse phase ISCO with 0-50% ACN/H2O with 0.05% TFA to give tert-butyl 3-(2-(2-(7-(2-(2,5-dioxo-2,5-dihydro-1H-pyrrol-1-yl)ethyl)-2,7-diazaspiro[3.5]nonan-2-yl)ethoxy)ethoxy)propanoate (20 mg, 0.043 mmol, 12.07% yield) as a colorless semisolid. LC-MS (ES, m/z): [M+H]⁺=466.3.

Tert-butyl 3-(2-(2-(7-(2-(2,5-dioxo-2,5-dihydro-1H-pyrrol-1-yl)ethyl)-2,7-diazaspiro[3.5]nonan-2-yl)ethoxy)ethoxy)propanoate (20 mg, 0.043 mmol) was dissolved in 1:1 DCM:TFA and stirred at 30° C. for 5 h. Solvent was removed under reduced pressure to give 3-(2-(2-(7-(2-(2,5-dioxo-2,5-dihydro-1H-pyrrol-1-yl)ethyl)-2,7-diazaspiro[3.5]nonan-2-yl)ethoxy)ethoxy)propanoic acid, 2 TFA (30 mg, 0.063 mmol, 18% yield) as a brown residue. LC-MS (ES, m/z): [M+H]⁺=410.3.

### Step 13-4: 2-((2,5,8,11-tetraoxatridecan-13-yl)carbamoyl)-4-((2S,5S)-15-(7-(2-(2,5-dioxo-2,5-dihydro-1H-pyrrol-1-yl)ethyl)-2,7-diazaspiro[3.5]nonan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

A mixture of 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (15 mg, 0.013 mmol), 3-(2-(2-(7-(2-(2,5-dioxo-2,5-dihydro-1H-pyrrol-1-yl)ethyl)-2,7-diazaspiro[3.5]nonan-2-yl)ethoxy)ethoxy)propanoic acid, 2 TFA (8.26 mg, 0.013 mmol), HATU (5.91 mg, 0.016 mmol) and N,N-Diisopropylethylamine (2.264 μl, 0.013 mmol) in DMF (0.5 mL) and stirred at rt for 1 h. The reaction mixture was quenched with 5% formic acid in water (0.5 mL), diluted with MeCN (0.5 mL), filtered and purified using preparatory HPLC (ACCQ Prep., 30×150 mm C18 column, 10 to 35% MeCN in water containing 0.05% formic acid over 24 minutes). Product fractions were combined and lyophilised to give 2-((2,5,8,11-tetraoxatridecan-13-yl)carbamoyl)-4-((2S,5S)-15-(7-(2-(2,5-dioxo-2,5-dihydro-1H-pyrrol-1-yl)ethyl)-2,7-diazaspiro[3.5]nonan-2-yl)-5-isopropyl-4,7-dioxo-2-(3-ureidopropyl)-10,13-dioxa-3,6-diazapentadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, bis formic acid salt (4 mg, 2.389 μmol, 18% yield) as a white solid. LC-MS (ES, m/z): [M+H]⁺=1549.4. 1H NMR (499 MHz, DMSO-d₆) δ 10.09 (br s, 1H), 8.27 (br s, 1H), 8.19 (s, 2H), 8.13-8.04 (m, 1H), 7.90-7.78 (m, 2H), 7.59 (br s, 1H), 7.48 (br s, 1H), 7.31 (br s, 1H), 7.09-7.00 (m, 2H), 7.00-6.92 (m, 2H), 5.98 (br s, 1H), 5.92 (br s, 1H), 5.73 (br s, 2H), 5.65-5.43 (m, 2H), 5.42-5.31 (m, 3H), 4.77 (br s, 2H), 4.32 (br d, J=5.5 Hz, 2H), 4.26-4.10 (m, 3H), 4.09-3.89 (m, 5H), 3.81 (s, 5H), 3.78-3.56 (m, 11H), 3.55-3.48 (m, 5H), 3.44-3.42 (m, 5H), 3.39-3.29 (m, 8H), 3.30-3.22 (m, 6H), 3.21-3.08 (m, 5H), 3.05-2.83 (m, 3H), 2.67 (s, 1H), 2.59 (br t, J=5.7 Hz, 2H), 2.40-2.36 (m, 1H), 2.36-2.18 (m, 2H), 1.99-1.85 (m, 2H), 1.66 (br s, 2H), 1.60-1.48 (m, 3H), 1.38 (br d, J=5.5 Hz, 2H), 1.34-1.26 (m, 2H), 1.26-1.13 (m, 1H), 1.06-0.99 (m, 1H), 0.93-0.84 (m, 2H), 0.78 (br dd, J=17.5, 6.7 Hz, 5H), 0.64 (br t, J=7.2 Hz, 3H).

### Example 14. 2-((2,5,8,11-tetraoxatridecan-13-yl)carbamoyl)-4-((2S,5S)-16-(7-(2-(2,5-dioxo-2,5-dihydro-1H-pyrrol-1-yl)ethyl)-2,7-diazaspiro[3.5]nonan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

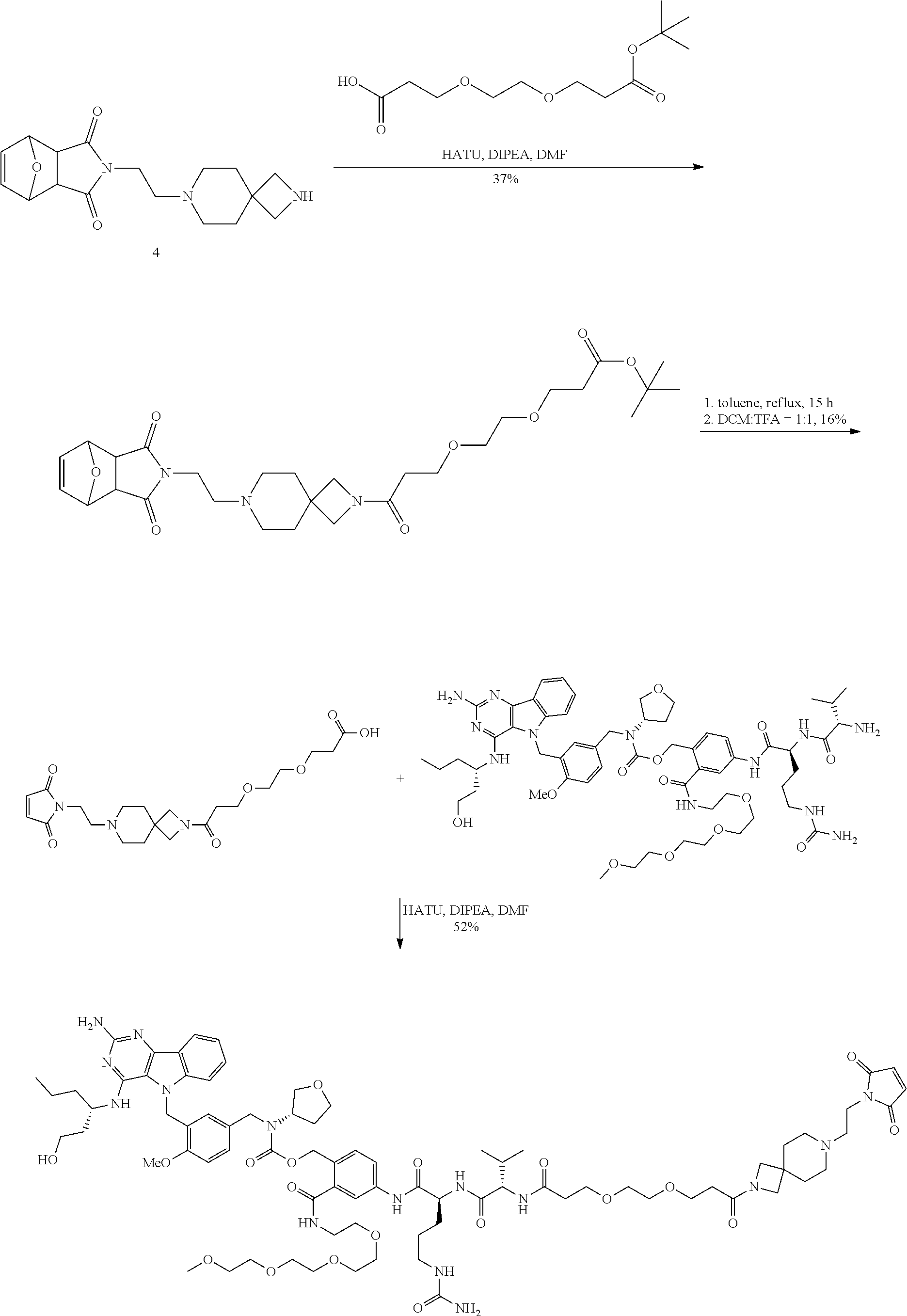

### Step 14-1: Synthesis of tert-butyl 3-(2-(3-(7-(2-(1,3-dioxo-1,3,3a,4,7,7a-hexahydro-2H-4,7-epoxyisoindol-2-yl)ethyl)-2,7-diazaspiro[3.5]nonan-2-yl)-3-oxopropoxy)ethoxy)propanoate

A mixture of 2-(2-(2,7-diazaspiro[3.5]nonan-7-yl)ethyl)-3a,4,7,7a-tetrahydro-1H-4,7-epoxyisoindole-1,3(2H)-dione (160 mg, 0.504 mmol), Acid-peg2-t-butyl ester (132 mg, 0.504 mmol), HATU (211 mg, 0.555 mmol) and N,N-Diisopropylethylamine (0.220 mL, 1.260 mmol) was dissolved in DMF (1 mL) and reaction mixture was stirred at rt for 1 h. solvent was removed under reduced pressure and the residue was purified using reverse phase ISCO with 0-50% MeCN/H2O+0.05% TFA-20 min 220/254 UV, lyophilized overnight to give tert-butyl 3-(2-(3-(7-(2-(1,3-dioxo-1,3,3a,4,7,7a-hexahydro-2H-4,7-epoxyisoindol-2-yl)ethyl)-2,7-diazaspiro[3.5]nonan-2-yl)-3-oxopropoxy)ethoxy)propanoate (105 mg, 0.187 mmol, 37% yield) as colorless semisolid. LC-MS (ES, m/z): [M+H]⁺=562.3. ¹H NMR (400 MHz, DMSO-d₆) δ 9.51 (br s, 1H), 6.58 (s, 2H), 5.16 (s, 2H), 3.91 (br s, 1H), 3.85 (br s, 1H), 3.78-3.69 (m, 2H), 3.65-3.54 (m, 8H), 3.25-3.14 (m, 4H), 3.05-2.93 (m, 4H), 2.49-2.39 (m, 2H), 2.27 (t, J=6.4 Hz, 2H), 2.03 (br s, 2H), 1.91-1.73 (m, 2H), 1.41 (s, 9H).

### Step 14-2: Synthesis of 3-(2-(3-(7-(2-(2,5-dioxo-2,5-dihydro-1H-pyrrol-1-yl)ethyl)-2,7-diazaspiro[3.5]nonan-2-yl)-3-oxopropoxy)ethoxy)propanoic Acid, Bis-Trifluoroacetate Salt

Tert-butyl 3-(2-(3-(7-(2-(1,3-dioxo-1,3,3a,4,7,7a-hexahydro-2H-4,7-epoxyisoindol-2-yl)ethyl)-2,7-diazaspiro[3.5]nonan-2-yl)-3-oxopropoxy)ethoxy)propanoate (105 mg, 0.187 mmol) was dissolved in 1 mL of toluene and the solution was heated to 100° C. for 12 hours. Solvent was removed under reduced pressure and the residue was dissolved in ACN/H₂O and purified using reverse phase ISCO with 0-50% ACN/H2O with 0.05% TFA to give tert-butyl 3-(2-(3-(7-(2-(2,5-dioxo-2,5-dihydro-1H-pyrrol-1-yl)ethyl)-2,7-diazaspiro[3.5]nonan-2-yl)-3-oxopropoxy)ethoxy) propanoate (35 mg, 0.071 mmol, 37.9% yield) as colorless semisolid. LC-MS (ES, m/z): [M+H]⁺=494.3.

Tert-butyl 3-(2-(3-(7-(2-(2,5-dioxo-2,5-dihydro-1H-pyrrol-1-yl)ethyl)-2,7-diazaspiro[3.5]nonan-2-yl)-3-oxopropoxy)ethoxy)propanoate (35 mg, 0.071 mmol) was dissolved in 1:1 DCM:TFA and stirred at 30° C. for 5 h. Solvent was removed under reduced pressure to give 3-(2-(3-(7-(2-(2,5-dioxo-2,5-dihydro-1H-pyrrol-1-yl)ethyl)-2,7-diazaspiro[3.5]nonan-2-yl)-3-oxopropoxy)ethoxy)propanoic acid, 2 TFA (20 mg, 0.030 mmol, 16% yield) as a brown residue. LC-MS (ES, m/z): [M+H]⁺=438.2.

### Step 14-3: Synthesis of 2-((2,5,8,11-tetraoxatridecan-13-yl)carbamoyl)-4-((2S,5S)-16-(7-(2-(2,5-dioxo-2,5-dihydro-1H-pyrrol-1-yl)ethyl)-2,7-diazaspiro[3.5]nonan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

A mixture of 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (16 mg, 0.014 mmol), 3-(2-(3-(7-(2-(2,5-dioxo-2,5-dihydro-1H-pyrrol-1-yl)ethyl)-2,7-diazaspiro[3.5]nonan-2-yl)-3-oxopropoxy)ethoxy)propanoic acid, 2 TFA (10.12 mg, 0.015 mmol), HATU (6.31 mg, 0.017 mmol) and N,N-Diisopropylethylamine (2.415 μl, 0.014 mmol) in DMF (0.5 mL) was stirred at rt for 1 h, The reaction mixture was quenched with 5% formic acid in water (0.5 mL), diluted with MeCN (0.5 mL), filtered and purified using preparatory HPLC (ACCQ Prep., 30×150 mm C18 column, 10 to 35% MeCN in water containing 0.05% formic acid over 24 minutes). Product fractions were combined and lyophilized to give 2-((2,5,8,11-tetraoxatridecan-13-yl)carbamoyl)-4-((2S,5S)-16-(7-(2-(2,5-dioxo-2,5-dihydro-1H-pyrrol-1-yl)ethyl)-2,7-diazaspiro[3.5]nonan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, bis-formic acid salt (12 mg, 7.19 μmol, 52% yield) as a white solid. LC-MS (ES, m/z): [M+H]⁺=1576.8. 1H NMR (499 MHz, DMSO-d₆) δ 10.03 (br s, 1H), 8.27 (br s, 1H), 8.12-8.03 (m, 2H), 7.88 (br d, J=7.6 Hz, 1H), 7.78 (br d, J=8.7 Hz, 1H), 7.57 (br s, 1H), 7.52 (br s, 1H), 7.34 (br s, 1H), 7.13-7.01 (m, 1H), 6.98 (br s, 1H), 6.96-6.92 (m, 2H), 6.26-6.05 (m, 1H), 6.00 (br s, 1H), 5.89 (br d, J=15.4 Hz, 2H), 5.73-5.52 (m, 3H), 5.34 (br s, 2H), 4.31 (br d, J=6.4 Hz, 1H), 4.25 (br s, 1H), 4.16 (br t, J=7.5 Hz, 1H), 4.09-3.89 (m, 4H), 3.81 (s, 4H), 3.75-3.59 (m, 4H), 3.52-3.46 (m, 6H), 3.36-3.29 (m, 10H), 3.26 (br s, 12H), 3.15 (s, 8H), 3.00 (br s, 1H), 2.98-2.76 (m, 3H), 2.40-2.35 (m, 1H), 2.34-2.21 (m, 5H), 2.18 (br t, J=6.3 Hz, 3H), 1.94-1.86 (m, 1H), 1.64 (br s, 2H), 1.52 (br s, 6H), 1.46-1.33 (m, 3H), 1.30 (br s, 2H), 1.27-1.15 (m, 2H), 0.93-0.84 (m, 2H), 0.82-0.72 (m, 6H), 0.64 (br t, J=7.0 Hz, 3H).

### Example 15. 2-((2,5,8,11-tetraoxatridecan-13-yl)carbamoyl)-4-((2S,5S)-16-(6-(2-(2,5-dioxo-2,5-dihydro-1H-pyrrol-1-yl)ethyl)-2,6-diazaspiro[3.4]octan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

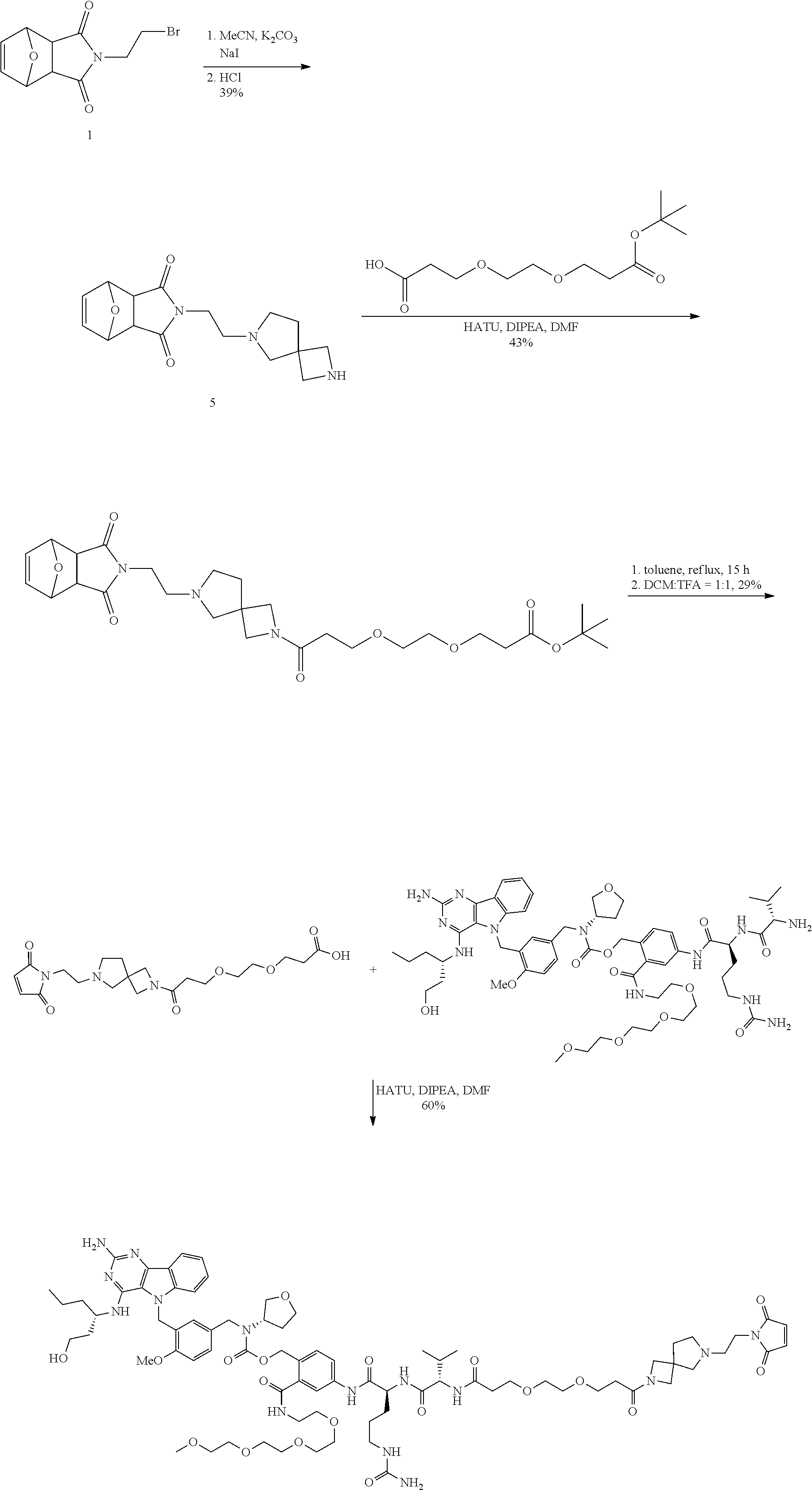

### Step 15-1: Synthesis of 2-(2-(2,6-diazaspiro[3.4]octan-6-yl)ethyl)-3a,4,7,7a-tetrahydro-1H-4,7-epoxyisoindole-1,3(2H)-dione, Hydrochloride Salt

In a 20 mL vial, 2-(2-bromoethyl)-3a,4,7,7a-tetrahydro-1H-4,7-epoxyisoindole-1,3(2H)-dione (300 mg, 1.103 mmol), tert-butyl 2,6-diazaspiro[3.4]octane-2-carboxylate (234 mg, 1.103 mmol), Potassium carbonate, 99+%, (229 mg, 1.654 mmol) and Sodium iodide (165 mg, 1.103 mmol) were dissolved in Acetonitrile (1 mL) and the reaction mixture was stirred at 40° C. overnight. The reaction was cooled to room temperature and filtered, and the filtrate was concentrated under reduced pressure. Purification was achieved using normal phase silica gel chromatography (25 g cartridge, 0-15% MeOH in DCM 220/254 nm UV detection) to afford the product tert-butyl 6-(2-(1,3-dioxo-1,3,3a,4,7,7a-hexahydro-2H-4,7-epoxyisoindol-2-yl)ethyl)-2,6-diazaspiro[3.4]octane-2-carboxylate (136 mg, 0.337 mmol, 30.6% yield) as colorless solid. LC-MS (ES, m/z): [M+H]⁺=404.2. 4 M HCl in dioxane (2 mL) was added to tert-butyl 6-(2-(1,3-dioxo-1,3,3a,4,7,7a-hexahydro-2H-4,7-epoxyisoindol-2-yl)ethyl)-2,6-diazaspiro[3.4]octane-2-carboxylate (136 mg, 0.337 mmol) and 1 mL MeOH was added. Reaction was stirred at room temperature for 2 h. Reaction suspension was concentrated under reduced pressure using the V10 and dried on the lyophilizer to give the product 2-(2-(2,6-diazaspiro[3.4]octan-6-yl)ethyl)-3a,4,7,7a-tetrahydro-1H-4,7-epoxyisoindole-1,3(2H)-dione (130 mg, 0.429 mmol, 39% yield) as a yellow solid. LC-MS (ES, m/z): [M+H]⁺=304.2. 1H NMR (400 MHz, DMSO-d₆) δ 6.57 (s, 2H), 5.17-5.10 (m, 2H), 4.12-3.91 (m, 3H), 3.87-3.79 (m, 2H), 3.75-3.70 (m, 3H), 3.53-3.44 (m, 3H), 3.38-3.27 (m, 3H), 3.24-3.06 (m, 2H), 3.05-2.92 (m, 2H), 2.49-2.23 (m, 2H).

### Step 15-2: Synthesis of tert-butyl 3-(2-(3-(6-(2-(1,3-dioxo-1,3,3a,4,7,7a-hexahydro-2H-4,7-epoxyisoindol-2-yl)ethyl)-2,6-diazaspiro[3.4]octan-2-yl)-3-oxopropoxy)ethoxy)propanoate

A mixture of 2-(2-(2,6-diazaspiro[3.4]octan-6-yl)ethyl)-3a,4,7,7a-tetrahydro-1H-4,7-epoxyisoindole-1,3(2H)-dione (130 mg, 0.429 mmol), Acid-peg2-t-butyl ester (112 mg, 0.429 mmol), HATU (179 mg, 0.471 mmol) and N,N-Diisopropylethylamine (0.187 mL, 1.071 mmol) was dissolved in DMF (1 mL) and reaction mixture was stirred at rt for 1 h. Residue was purified using reverse phase ISCO with 0-50% MeCN/H2O+0.05% TFA-20 min 220/254 UV, lyophilized overnight to give tert-butyl 3-(2-(3-(6-(2-(1,3-dioxo-1,3,3a,4,7,7a-hexahydro-2H-4,7-epoxyisoindol-2-yl)ethyl)-2,6-diazaspiro[3.4]octan-2-yl)-3-oxopropoxy)ethoxy)propanoate (100 mg, 0.183 mmol, 43% yield) as colorless semisolid. LC-MS (ES, m/z): [M+H]⁺=548.3. 1H NMR (400 MHz, DMSO-d₆) δ 10.15 (br s, 1H), 6.58 (s, 2H), 5.16 (s, 2H), 4.16 (br s, 1H), 4.07 (br s, 1H), 3.94-3.81 (m, 2H), 3.81-3.65 (m, 3H), 3.58 (t, J=6.6 Hz, 3H), 3.59 (t, J=6.2 Hz, 3H), 3.37-3.26 (m, 4H), 3.18 (br s, 1H), 2.98 (s, 2H), 2.49-2.38 (m, 3H), 2.37-2.13 (m, 4H), 1.41 (s, 9H).

### Step 15-3: Synthesis of 3-(2-(3-(6-(2-(2,5-dioxo-2,5-dihydro-1H-pyrrol-1-yl)ethyl)-2,6-diazaspiro[3.4]octan-2-yl)-3-oxopropoxy)ethoxy)propanoic Acid, Bis-Trifluoroacetate Salt

Tert-butyl 3-(2-(3-(6-(2-(1,3-dioxo-1,3,3a,4,7,7a-hexahydro-2H-4,7-epoxyisoindol-2-yl)ethyl)-2,6-diazaspiro[3.4]octan-2-yl)-3-oxopropoxy)ethoxy)propanoate (100 mg, 0.183 mmol) was dissolved in 1 mL of toluene and the solution was heated to 100° C. for 12 hours. Solvent was removed under reduced pressure and the residue was dissolved in ACN/H2O and purified using reverse phase ISCO with 0-50% ACN/H2O with 0.05% TFA to give tert-butyl 3-(2-(3-(6-(2-(2,5-dioxo-2,5-dihydro-1H-pyrrol-1-yl)ethyl)-2,6-diazaspiro[3.4]octan-2-yl)-3-oxopropoxy)ethoxy)propanoate (17 mg, 0.035 mmol, 19.41% yield) as colorless semisolid. LC-MS (ES, m/z): [M+H]⁺=480.2.

Tert-butyl 3-(2-(3-(6-(2-(2,5-dioxo-2,5-dihydro-1H-pyrrol-1-yl)ethyl)-2,6-diazaspiro[3.4]octan-2-yl)-3-oxopropoxy)ethoxy)propanoate (17 mg, 0.035 mmol, 19.41% yield) was dissolved in 1:1 DCM:TFA and stirred at 30° C. for 5 h. Solvent was removed under reduced pressure to give 3-(2-(3-(6-(2-(2,5-dioxo-2,5-dihydro-1H-pyrrol-1-yl)ethyl)-2,6-diazaspiro[3.4]octan-2-yl)-3-oxopropoxy)ethoxy)propanoic acid, 2 TFA (35 mg, 0.054 mmol, 29% yield) as a brown residue LC-MS (ES, m/z): [M+H]⁺=424.2 and RT=0.51.

### Step 15-4: Synthesis of 2-((2,5,8,11-tetraoxatridecan-13-yl)carbamoyl)-4-((2S,5S)-16-(6-(2-(2,5-dioxo-2,5-dihydro-1H-pyrrol-1-yl)ethyl)-2,6-diazaspiro[3.4]octan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, Bis-Formic Acid Salt

A mixture of 2-((2,5,8,11-tetraoxatridecan-13-yl)carbamoyl)-4-((S)-2-((S)-2-amino-3-methylbutanamido)-5-ureidopentanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate (16 mg, 0.014 mmol), 3-(2-(3-(6-(2-(2,5-dioxo-2,5-dihydro-1H-pyrrol-1-yl)ethyl)-2,6-diazaspiro[3.4]octan-2-yl)-3-oxopropoxy)ethoxy)propanoic acid, 2 TFA (9.91 mg, 0.015 mmol), HATU (6.31 mg, 0.017 mmol) and N N-Diisopropylethylamine (2.415 μl, 0.014 mmol) in DMF (0.5 mL) was stirred at rt for 1 h, The reaction mixture was quenched with 5% formic acid in water (0.5 mL), diluted with MeCN (0.5 mL), filtered and purified using preparatory HPLC (ACCQ Prep., 30×150 mm C18 column, 10 to 35% MeCN in water containing 0.05% formic acid over 24 minutes). Product fractions were combined and lyophilized to give 2-((2,5,8,11-tetraoxatridecan-13-yl)carbamoyl)-4-((2S,5S)-16-(6-(2-(2,5-dioxo-2,5-dihydro-1H-pyrrol-1-yl)ethyl)-2,6-diazaspiro[3.4]octan-2-yl)-5-isopropyl-4,7,16-trioxo-2-(3-ureidopropyl)-10,13-dioxa-3,6-diazahexadecanamido)benzyl (3-((2-amino-4-(((S)-1-hydroxyhexan-3-yl)amino)-5H-pyrimido[5,4-b]indol-5-yl)methyl)-4-methoxybenzyl)((S)-tetrahydrofuran-3-yl)carbamate, bis-formic acid salt (14 mg, 7.86 μmol, 60% yield) as a white solid. LC-MS (ES, m/z): [M+H]⁺=1563.7. 1H NMR (499 MHz, DMSO-d₆) δ 10.11 (br s, 1H), 8.36 (br s, 1H), 8.21-8.11 (m, 2H), 7.92 (br d, J=7.9 Hz, 1H), 7.86 (br d, J=8.4 Hz, 1H), 7.66 (br s, 1H), 7.56 (br s, 1H), 7.39 (br s, 1H), 7.12 (br t, J=7.6 Hz, 1H), 7.08-7.00 (m, 4H), 5.98 (br s, 2H), 5.82 (br s, 2H), 5.69-5.57 (m, 2H), 5.46 (br s, 1H), 5.41 (br s, 2H), 4.93 (br s, 1H), 4.85 (br s, 1H), 4.39 (br d, J=4.7 Hz, 1H), 4.34-4.19 (m, 2H), 4.16-4.01 (m, 3H), 3.98 (s, 2H), 3.89 (s, 3H), 3.79-3.64 (m, 4H), 3.63-3.54 (m, 7H), 3.50-3.44 (m, 17H), 3.22 (s, 10H), 3.08 (br s, 1H), 3.05-2.86 (m, 3H), 2.70-2.54 (m, 3H), 2.39 (br dd, J=13.5, 6.9 Hz, 2H), 2.25 (br t, J=6.2 Hz, 2H), 2.01-1.89 (m, 3H), 1.70 (br s, 1H), 1.65-1.56 (m, 3H), 1.49-1.41 (m, 3H), 1.38 (br d, J=7.8 Hz, 2H), 1.26 (br dd, J=14.0, 6.4 Hz, 2H), 1.00-0.92 (m, 2H), 0.90-0.79 (m, 6H), 0.72 (br t, J=7.0 Hz, 3H).

### VI. BIOLOGICAL EXAMPLES

### Antibodies to LIV1 and ADC Constructs Thereof

Antibodies to human LIV1 were raised, selected and optimized (See Table 3). Antibody 5A11 was selected for use in the ADCs of the present invention.

Anti-LIV1 antibody 5A11 was subjected to cell-based and biochemical characterization. The anti-human LIV1 antibody was shown to cross-react with cynomolgus monkey LIV1 by FACS analysis using LIV1 endogenous expressor Expi293, and also with MCF7, Baf3 and CHO—S cells stably transfected with human and cynomolgus monkey LIV1 antigen. Cells were exposed to the antibody and then stained with Alexa Fluor 647 anti-human antibody to detect binding. The LIV1 antibody showed no binding to un-transfected Baf3 and CHO—S cells, validating binding specificity to the LIV1 protein.

### Generation and Selection of Anti-LIV1 Antibodies

Anti-huLIV1 antibodies for use in the ADCs of the present invention were generated as follows. Briefly, eight cohorts of human B-cell receptor (BCR) transgenic mice were immunized with immunogens including the extracellular domain (ECD) of human LIV1 protein, a mixture of the ECDs of human and cynomolgus monkey (cyno) LIV1 protein, huLIV1 encoding DNA or mRNA, or with a mixture of three peptides (SEQ ID NOs: 13-15) comprising 15 amino acid segments of the ECD of huLIV1 protein, with or without complete Freund's adjuvant (CFA), incomplete Freund's adjuvant (IFA), or Ribi's adjuvant. Immunization was performed by routes including gene gun delivery, injections into footpad or hock, or by intraperitoneal (IP), subcutaneous (SC) or intramuscular (IM) injection. 849 binders were obtained by either hybridoma creation, single B-cell cloning, or microfluidic light-directed hybridoma sorting methods. A panel of 129 binders was selected based on desired affinity, cyno cross-reactivity, lack of cross-reactivity with ZIP10 (SLC39A10, another member of the LIV1 subfamily of zinc transporters) as measured by ELISA. Fluorescence Activated cell sorting (FACS) was used to select 74 of these clones, which were then subjected to heavy chain/light chain shuffling with the 11 best binders, and other selected heavy chain variable regions (VH) and light chain variable regions (VL). 33 of these mAbs were selected for analytical binding and epitope binning by surface plasmon resonance (SPR) and FACS. Nine antibodies were selected for full functional profiling as ADC candidates.

Antibody 5A11 comprises a shuffled heavy chain/light chain pairing that combines the heavy and light chains of separate antibodies obtained from immunization with the mixture of three huLIV1 ECD peptides described above.

### Optimization of Anti-LIV1 Antibodies

Anti-LIV1 mAbs 5A11 and 5B11, which share a light chain, were selected for further optimization with respect to immunogenicity and developability. Antibody immunogenicity was predicted by a heat map produced by EpiVax® immunogenicity assessment technology (EpiVax Inc., Providence, Rhode Island, USA). Hydrophobicity was also compared to 48 other phase 3 and approved therapeutic antibodies. Ten sequence variants of each of 5A11 and 5B11 were generated to minimize immunogenicity, and then evaluated for binding affinity and kinetics, cyno cross-reactivity, in vitro activity in a DC-T cell proliferation assay, and in vitro PD-L1 induction as an ADC in an MCF7 co-culture assay.

Ultimately, the original heavy/light chain shuffled mAb 5A11 was selected based on favorable characteristics, including cyno cross-reactivity (<5-fold difference), binding affinity (86 μM K_{D} by KinExA®, Sapidyne Instruments, Inc., Boise, Idaho, USA), internalization, good early developability assessment, thermal stability (T_{agg}=70.3° C.), low self-interaction (by clone self-interaction bio-layer-interferometry, CSI-BLI), low immunogenicity risk, and favorable long term stability and solubility.

Comparative in vitro PD experiments in a human tumor explant model using Fc active (hIgG1) and Fc inert (hIgG1.3) constant regions indicate that FcγR interaction potentiates ADC activity, so the Fc active hIgG1 heavy chain constant region was selected for the ADCs of the present invention intended for clinical use.

### ADC Preparation

Antibody was subjected to mild reduction in 20 mM L-histidine and 250 mM sucrose, pH5.5, 5 mM EDTA at 25° C. by the addition of 12-fold molar (per antibody) excess TCEP for 2 hours until separation of light chain, heavy chain is observed on RP-UPLC (Agilent Advanced Bio Diphenyl 2.1×150 mm). The mobile phase consisted of 0.1% TFA in water (A) and 0.1% TFA in acetonitrile (B). The system was operated at a flow rate of 1 ml/min. The gradient condition was as follows: 0-1 min., 20-30% B; 1-6 min, 30-42%, 6.05-6.5 min, 42-95%. Wavelength 280 nM was used to monitor the reduction. TCEP was removed by Zeba column. Buffer was exchanged to 50 mM Sodium Acetate, pH5.0. The maleimide-linked reagent (12 eq.) was incubated with the activated antibody for 2 hr at 25° C. The reaction mixture was monitored by LCMS till full conversion of light chain and heavy chain signals were observed. Examples ADC #1, 2, 3, 4, 5, 6, 7, 9 and showed full conversion after 2-hrs incubation. Examples ADC #8, 11 and 14 required addition of additional 6 eq. and 9 eq. of linker drug (L-D) for full conversion to furnish DAR 8. Examples ADC #10, and 13 required additional 12 eq. of L-P to provide DAR of 7.84 and 7.68, respectively. The antibody conjugate was purified on Zeba Spin column to remove excess reagents.

To evaluate binding and internalization in LIV1-expressing cell lines, we used MCF7 cells, which are reported to express the LIV1 antigen on their surface. The antibody was labeled with the pH-sensitive dye pHrodo to enable visualization of late endosome and lysosomal trafficking. This internalization profile was comparable to that observed with the naked, unconjugated antibody.

ADC LCMS and SEC characterization. The samples were analyzed by LC-MS using an Agilent 1290 Infinity UPLC coupled to a 6530 Accurate-Mass Q-TOF mass spectrometer (Agilent, Santa Clara, CA). The samples were diluted to 1 mg/mL in 100 mM Tris, pH 7.5. Twenty microliters of sample were reduced by adding 2 μL of 0.5 M TCEP. The samples were analyzed by LC-MS using an Agilent 1290 Infinity UPLC coupled to a 6530 Accurate-Mass Q-TOF mass spectrometer (Agilent, Santa Clara, CA). The analytical column used was a Waters BEH C4 column, 1.7 μm, 2.1 mm×50 mm, held at 60° C. The mobile phase consisted of 0.1% formic acid in water (A) and 0.1% formic acid in acetonitrile (B). The system was operated at a flow rate of 200 μL/min. The gradient condition was as follows: 0-2 min, held at 27% B; 2-9 min, slow ramp from 27% to 37% B; 9-9.5 min, linear ramp from 37% to 90% B; 9.5-12.3 min, held at 90% B. The MS settings were as follows: Polarity=Positive, Capillary Voltage=4.2 kV, Sample Cone=40 V, Source Offset=15 V, Source Temperature=140° C., Desolvation Temperature=325° C. The data acquisition range was 900-3200 m/z. Deconvolution was performed using Agilent MassHunter Walkup. SEC analysis was performed with Zorbax GF250 4.6 um ID×250 mm, SEC column (P/N 884973-901). Mobile phase (isocratic: 0.1 Na Carbonate, 0.2 M NaCl, pH7.2, 5 mM glycine, 15% Acetonitrile) was used for elution at a flow rate of 1 mL/min.

As stated previously, depending on the structure of the ADC construct, thiosuccinimide ADCs display slow hydrolysis, typically requiring heating the ADCs in a basic buffer, which results in an increased risk of deconjugation and denaturation of the final product.

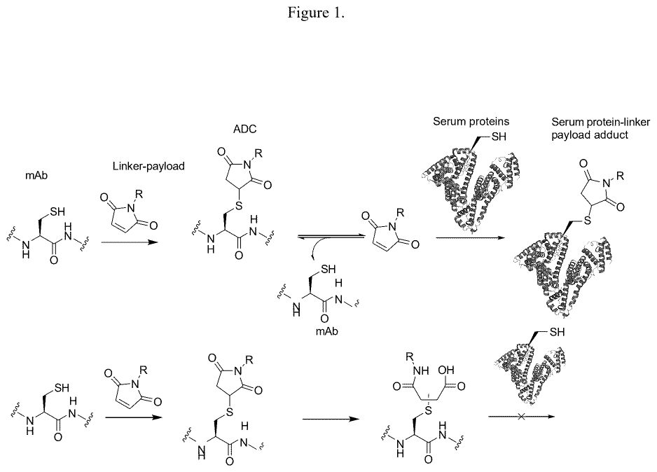

The present invention has invented a novel linker that hydrolyzes upon reaction with the antibody without requiring heat or basic conditions, facilitating the generation of ADCs that are resistant to thiol-exchange reactions occurring in serum. FIG. 1 illustrates the enhanced stability of the hydrolyzable linker due to ring opening hydrolysis.

Table 2 illustrates Examples ADC 1-15 of the present invention.

Table 3 lists the Sequence Listings.

Table 4 lists DAR ratios of the ADCs of Examples ADC 1-15.

Table 5 lists the hydrolysis percentages of the exemplified Examples ADC 1-15 over 2 hr, 1 day, 4 days and 5 days.

Table 6. SEC Data: Aggregation Percentages

### ADC Examples

Ab represents antibody Anti-LIV1 mAb 5A11.

“q” ranges from 1 to 10, preferably 1 to 8, and refers to Drug Antibody Ratio (DAR).

TABLE 2

| ADC | |
|---|---|
| Examples | ADC structure |
| 1 | 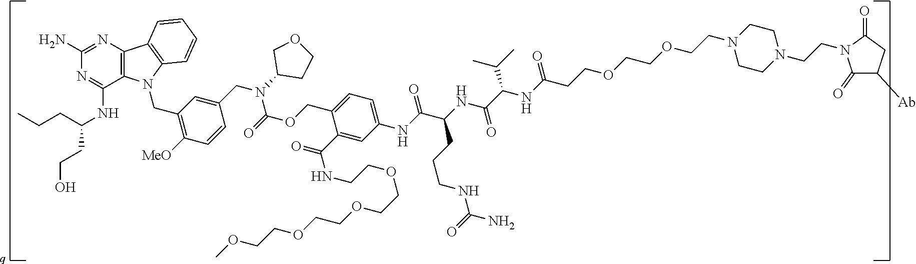 |
| 2 | 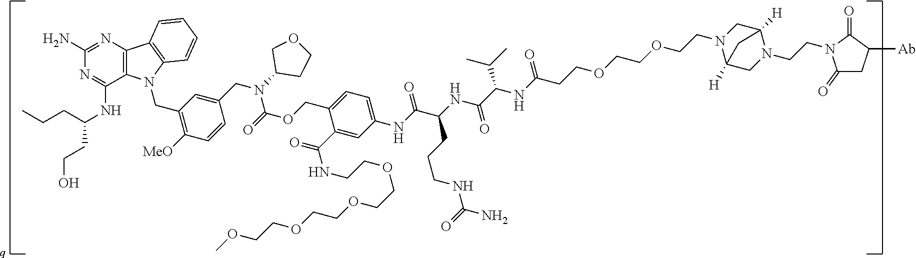 |
| 3 | 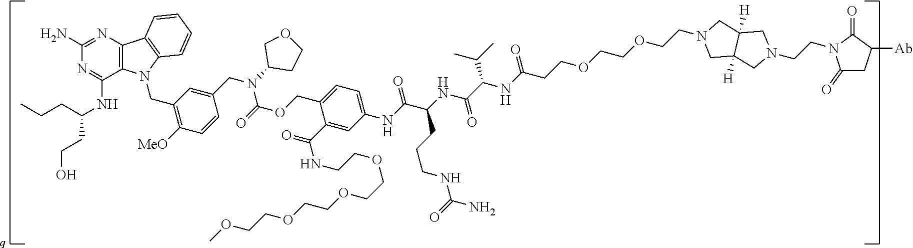 |
| 4 | 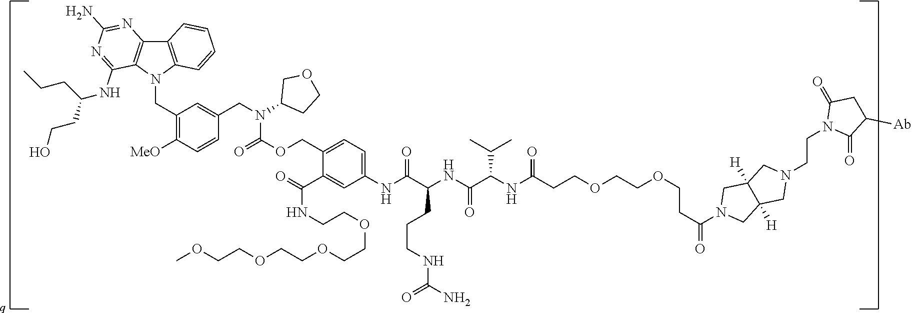 |
| 5 | 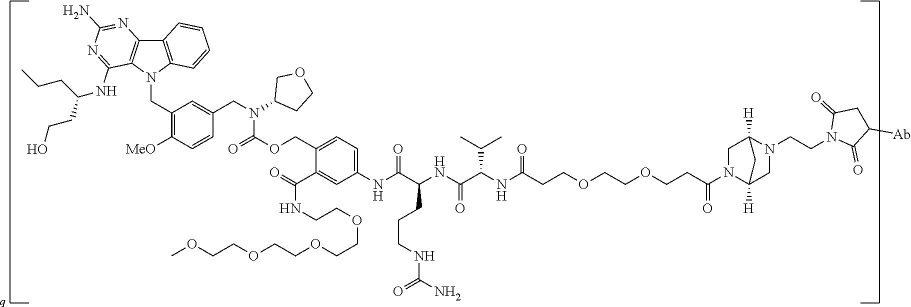 |
| 6 | 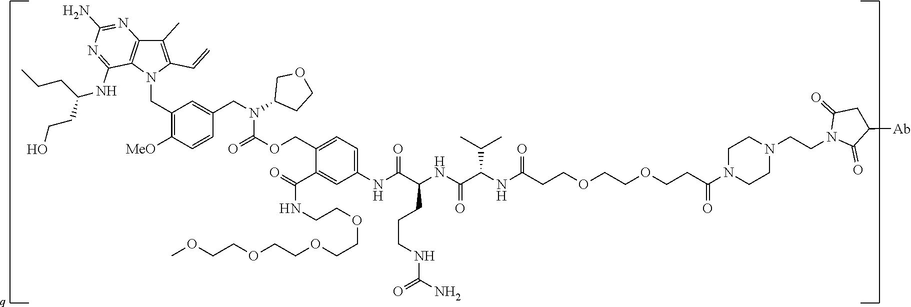 |
| 7 | 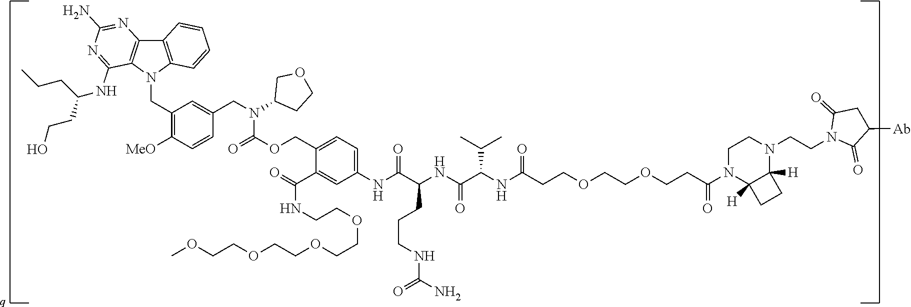 |
| 8 | 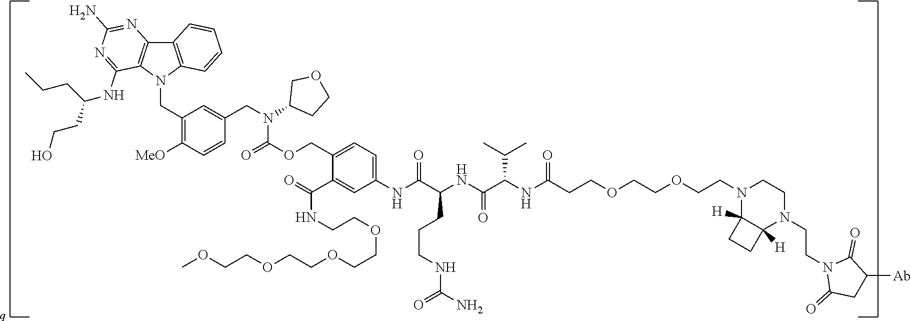 |
| 9 | 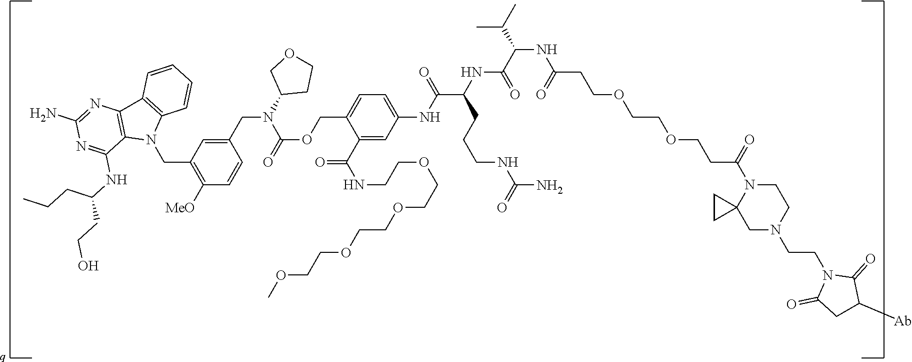 |
| 10 | 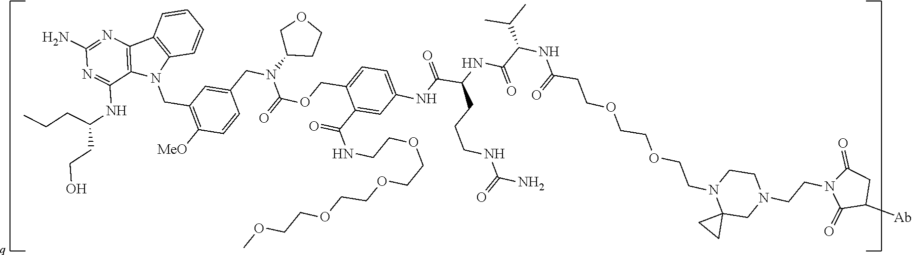 |
| 11 | 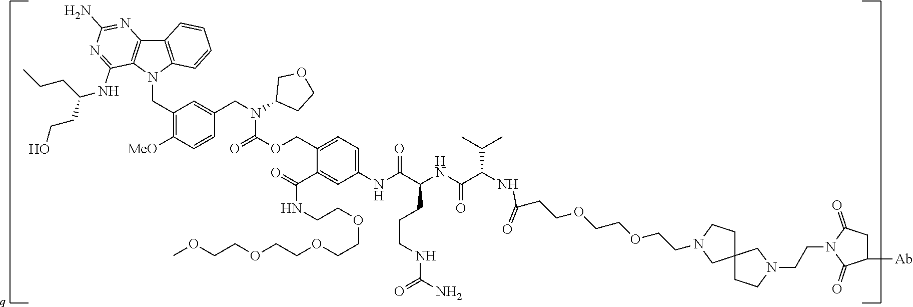 |
| 12 | 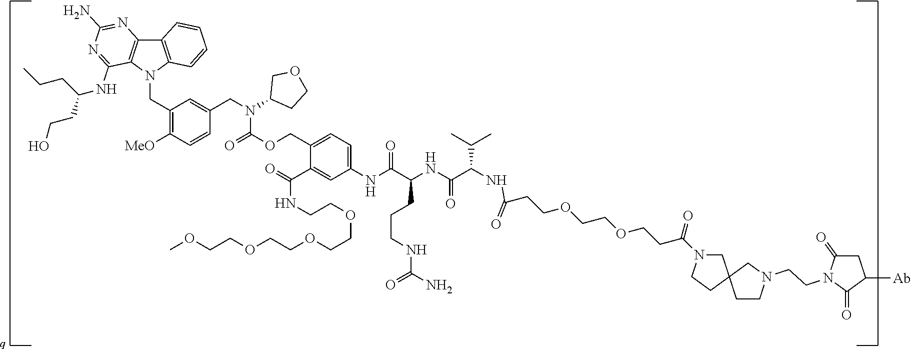 |
| 13 | 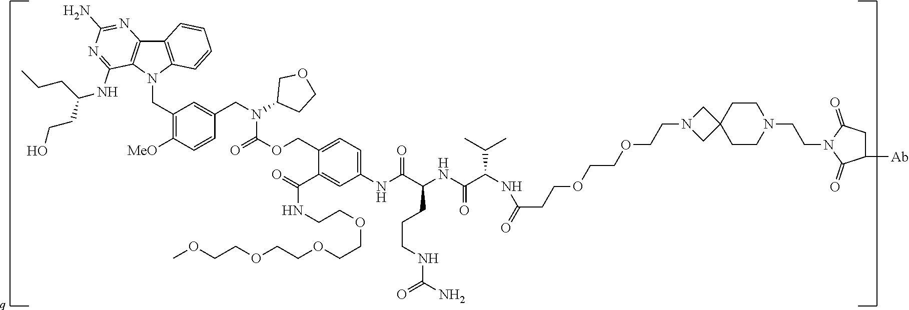 |
| 14 | 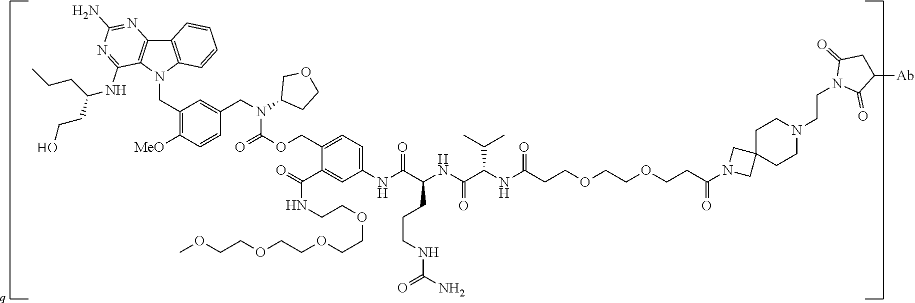 |
| 15 | 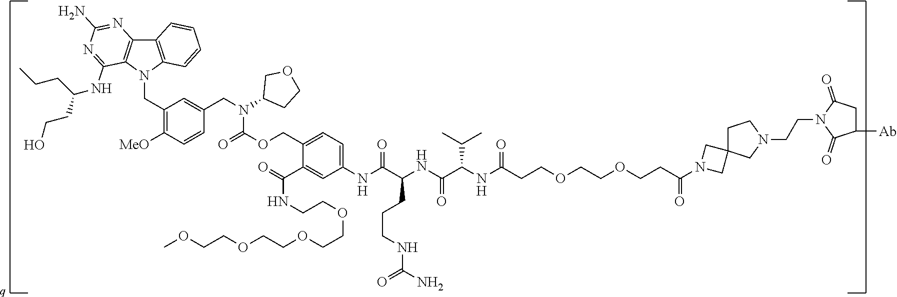 |

TABLE 3
Summary of the Sequence Listing

| SEQ ID NO. | Description |
|---|---|
| 1 | CDRH1 5A11 |
| 2 | CDRH2 5A11 |
| 3 | CDRH3 5A11 |
| 4 | VH 5A11 |
| 5 | HC 5A11 |
| 6 | CDRL1 5A11 |
| 7 | CDRL2 5A11 |
| 8 | CDRL3 5A11 |
| 9 | VL 5A11 |
| 10 | LC 5A11 |
| 11 | huLIV1, Isoform 1 - NP_036451.4 |
| 12 | huLIV1, Isoform 2 - NP_001092876.1 |
| 13 | huLIV1 immunogen peptide 1 |
| 14 | huLIV1 immunogen peptide 2 |
| 15 | huLIV1 immunogen peptide 3 |

With regard to antibody sequences, the Sequence Listing provides the sequences of the mature variable regions of the heavy and light chains, i.e. the sequences do not include signal peptides. Any signal sequence suitable for use in the production cell line being used may be used in production of the antibodies of the present invention. Heavy chain amino acid sequences are provided without a C-terminal lysine residue, but in some embodiments such residue is encoded in the nucleic acid construct for the antibody. Numbering in the specification and FIGURES is often according to the Kabat numbering system and thus may not correspond to the numbering in the sequence listing. CDR boundaries are by the Kabat numbering system.

The ADC Example numbers (#) are the ADCs of the present invention, with the LIV1 antibody conjugated to the corresponding synthetic example numbers listed above. Thus, Example ADC #1 is the conjugated ADC of Synthetic Linker-Payload Example #1 above.

TABLE 4
DAR Ratios of the ADCs

| | | Conc | Endotoxin |
|---|---|---|---|
| ADC | DAR | (mg/mL) | (EU/mg) |
| Example ADC #1 | 8 | 2.71 | 0.324 |
| Example ADC #2 | 8 | 3.68 | 0.123 |
| Example ADC #3 | 8 | 3.59 | 0.227 |
| Example ADC #4 | 8 | 7.26 | 0.029 |
| Example ADC #5 | 8 | 6.87 | 0.034 |
| Example ADC #6 | 8 | 7.29 | 0.043 |
| Example ADC #7 | 8 | 7.05 | 0.352 |
| Example ADC #8 | 8 | 7.34 | 0.028 |
| Example ADC #9 | 8 | 7.13 | 0.037 |
| Example ADC #10 | 7.84 | 6.74 | 0.142 |
| Example ADC #11 | 6.91 | 6.9 | 0.072 |
| Example ADC #12 | 8 | 6.14 | 0.08 |
| Example ADC #13 | 7.68 | 6.77 | 0.066 |
| Example ADC #14 | 8 | 6.02 | 0.037 |
| Example ADC #15 | 8 | 6.95 | 0.037 |

TABLE 5
Hydrolysis of ADCs over 2 hr, 1 day, 4 days and 5 days

| | % hydrolysis | % hydrolysis | % hydrolysis | % hydrolysis |
|---|---|---|---|---|
| | (based on LC | (based on LC | (based on LC | (based on LC |
| | MS signal) | MS signal) | MS signal) | MS signal) |
| | Reaction mixture | Purified | Purified | Purified |
| ADC | 2 hr at RT | ADC (day 1) | ADC (day 4) | ADC (day 5) |
| Example ADC #1 | 19.67 | 22.32 | NA | 80.30 |
| Example ADC #2 | 31.1 | 29.98 | NA | 93.16 |
| Example ADC #3 | 32.19 | 32.8 | NA | 40.96 |
| Example ADC #4 | 27.89 | 71.33 | 100 | 100 |
| Example ADC #5 | 8.3 | 53.22 | 91.28 | 95.84 |
| Example ADC #6 | 15.54 | 25.6 | 50.05 | 55.44 |
| Example ADC #7 | 11.29 | 17.11 | 28.17 | 35.97 |
| Example ADC #8 | 11.55 | 17.58 | 34.91 | 38.66 |
| Example ADC #9 | 16.35 | 42.96 | 79.75 | 81.7 |
| Example ADC #10 | 27.07 | 47.7 | 100 | 100 |
| Example ADC #11 | 28.20 | 81.00 | 100 | 100 |
| Example ADC #12 | 28.22 | 63.96 | 100 | 100 |
| Example ADC #13 | NA | 22.37 | 44.6 | 46.6 |
| Example ADC #14 | 37.55 | 84.55 | 100 | 100 |
| Example ADC #15 | 27.34 | 70.74 | 100 | 100 |

TABLE 6
SEC Data: Aggregation Percentages

| | | Aggregation % after |
|---|---|---|
| | Aggregation % after | purification and 3 freeze- |
| ADC | purification | thaw cycles |
| Example ADC #1 | <1% | <1% |
| Example ADC #2 | <1% | <1% |
| Example ADC #3 | <3% | <3% |
| Example ADC #4 | <1% | <1% |
| Example ADC #5 | <1% | <1% |
| Example ADC #6 | <1% | <1% |
| Example ADC #7 | <1% | <1% |
| Example ADC #8 | <1% | <1% |
| Example ADC #9 | <1% | <1% |
| Example ADC #10 | <1% | <1% |
| Example ADC #12 | <1% | <1% |
| Example ADC #12 | <1% | <1% |
| Example ADC #13 | <1% | <1% |
| Example ADC #14 | <1% | <1% |
| Example ADC #15 | <1% | <1% |

Those skilled in the art will recognize or be able to ascertain using no more than routine experimentation, many equivalents of the specific aspects disclosed herein. Such equivalents are intended to be encompassed by the following claims.

What is claimed is:

1. A compound of Formula (I): 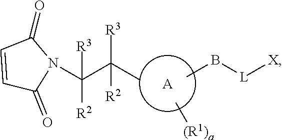
or a pharmaceutically acceptable salt thereof,
wherein:
A is selected from an unsubstituted or substituted 4 to 12 membered heterocyclic or heterospirocyclic ring;
B is independently selected from a bond, —CH₂—, —C═O—, —O—, —(—(CR¹¹R¹²)ₙ—)—O)ₘ—, —C═O—(CR¹¹R¹²)ₙ—O—)ₘ—, —C═O—(NR¹¹)—, —S—, —SO—, —SO₂—, or —SO₂—(NR¹¹)—;
R¹ is independently selected from hydrogen, halogen, —OR¹¹, —N(R¹¹R¹²)—NHR¹¹, —(—(CH₂)₂—)ₙ-O—)ₘ, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —N(R¹¹R¹²)—NHR¹¹ or —OR¹¹;
R² is independently selected from hydrogen, halogen, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —NHR¹¹ or —OR¹¹;
R³ is independently selected from hydrogen, halogen, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —NHR¹¹ or —OR¹¹;
wherein two of any R¹, R² or R³ substituents together with the carbon atoms they are attached to, may join to form a 5 or 6 membered ring that may be unsaturated, saturated, partially saturated;
and may further optionally be substituted with 1 or 2 R¹¹ substituents;
R¹¹ is independently selected from hydrogen, halogen, —C₁-C₆ alkyl, —C₂-C₆ alkenyl, —C₁-C₆ haloalkyl, —(—(CH₂)ₙ—)—O—)ₘ, a 3 to 12 membered cycloalkyl, a 4 to 12 membered heterocyclic, a 5 to 12 membered aryl or a 5 to 12 membered heteroaryl ring;
R¹² is independently selected from hydrogen, halogen, —C₁-C₆ alkyl, —C₂-C₆ alkenyl, —C₁-C₆ haloalkyl, —(—(CH₂)ₙ—)—O—)ₘ, a 3 to 12 membered cycloalkyl, a 4 to 12 membered heterocyclic, a 5 to 12 membered aryl or a 5 to 12 membered heteroaryl ring;
wherein the alkyl, alkenyl, haloalkyl, cycloalkyl, heterocyclic, aryl or heteroaryl rings in R¹¹ and R¹² are each independently unsubstituted or substituted with 1, 2, or 3 R¹³ substituents;
R¹³ is independently hydrogen, halo, —C₁-C₆ alkyl, —C₁-C₆ haloalkyl, —C₁-C₆ alkoxyalkyl, oxo, —CN, —NR¹⁴R¹⁴, —CH₃—NH—CH₂—C(═O)—OH, hydroxyl or —C₁-C₆ alkoxy;
R¹⁴ is independently hydrogen, —C₁-C₆ alkyl, —C₁-C₆ haloalkyl, or —C₁-C₆ alkoxyalkyl;
L is a linker;
X is a payload;
a is 0, 1, 2, 3, 4, 5, 6, 7, 8, 9, 10;
m is an integer selected from 0 to 36;
n is 1, 2, 3, 4, 5, 6, 7, 8, 9, 10, 11 or 12; and further
wherein the heterocyclic and heteroaryl cyclic ring in each A, R¹, R², R³, R¹¹, and R¹² may include 1, 2 or 3 heteroatoms independently selected from O, N or S.

2. The compound of claim 1, wherein 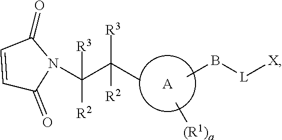
wherein
A is selected from an unsubstituted or substituted 6 to 9 membered heterocyclic or heterospirocyclic ring;
B is independently selected from a bond, —CH₂—, —C═O—, —(—(CR¹¹R¹²)ₙ—)—O)ₘ—, —C═O—(CR¹¹R¹²)ₙ—O—)ₘ—;
R¹ is independently selected from hydrogen, halogen, —OR¹¹, —(—(CH₂)₂—)ₙ-O—)ₘ, —C₁-C₆ alkyl, a 3 to 6-membered cycloalkyl, a 4 to 12-membered heterocyclic, a 5 to 12-membered aryl or a 5 to 12-membered heteroaryl ring, wherein the alkyl, cycloalkyl, heterocyclic and heteroaryl may be optionally substituted with —R¹¹, —N(R¹¹R¹²)—NHR¹¹ or —OR¹¹;
R² is independently selected from hydrogen, halogen, —C₁-C₆ alkyl, a 3 to 6 membered cycloalkyl ring, wherein the alkyl, cycloalkyl ring may be optionally substituted with —R¹¹, —NHR¹¹ or —OR¹¹; and
R³ is independently selected from hydrogen, halogen, —C₁-C₆ alkyl, a 3 to 6 membered cycloalkyl ring wherein the alkyl, cycloalkyl ring may be optionally substituted with —R¹¹, —NHR¹¹ or —OR¹¹.

3. The compound of claim 2, wherein A is a 6 to 9 membered heterocyclic ring.

4. The compound of claim 1, of Formula (II): 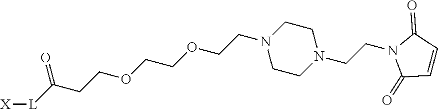
or a pharmaceutically acceptable salt thereof, wherein:
L is a linker, and
X is a payload.

5. The compound of claim 1, having Formula (III): 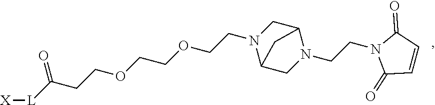
or a pharmaceutically acceptable salt thereof, wherein:
L is a linker, and
X is a payload.

6. The compound of claim 1, having Formula (IV): 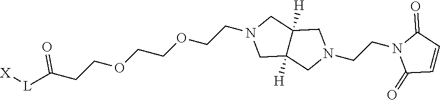
or a pharmaceutically acceptable salt thereof, wherein:
L is a linker, and
X is a payload.

7. The compound of claim 1, having Formula (V): 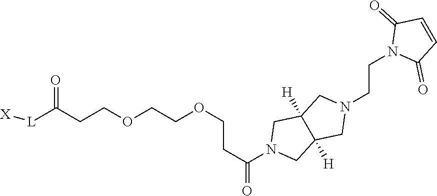
or a pharmaceutically acceptable salt thereof, wherein:
L is a linker, and
X is a payload.

8. The compound of claim 1, having Formula (VI): 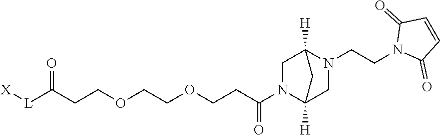
or a pharmaceutically acceptable salt thereof, wherein:
L is a linker, and
X is a payload.

9. The compound of claim 1, having Formula (VII): 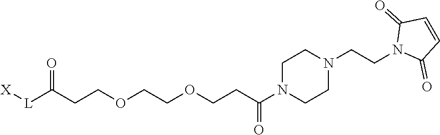
or a pharmaceutically acceptable salt thereof, wherein:
L is a linker, and
X is a payload.

10. The compound of claim 1, having Formula (VIII): 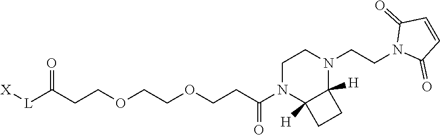
or a pharmaceutically acceptable salt thereof, wherein:
L is a linker, and
X is a payload.

11. The compound of claim 1, having Formula (IX): 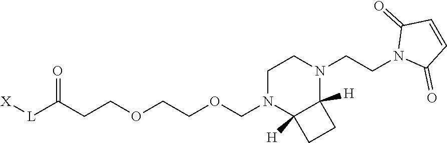
or a pharmaceutically acceptable salt thereof, wherein:
L is a linker, and
X is a payload.

12. The compound of claim 1, having Formula (X): 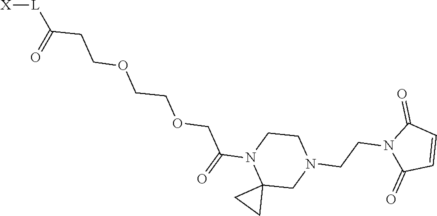
or a pharmaceutically acceptable salt thereof, wherein:
L is a linker, and
X is a payload.

13. The compound of claim 1, having Formula (XI): 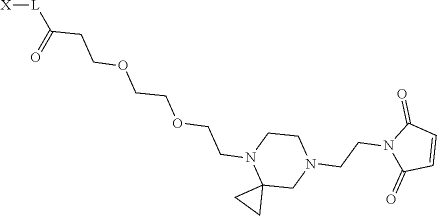
or a pharmaceutically acceptable salt thereof, wherein:
L is a linker, and
X is a payload.

14. The compound of claim 1, having Formula (XII): 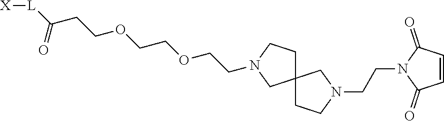
or a pharmaceutically acceptable salt thereof, wherein:
L is a linker, and
X is a payload.

15. The compound of claim 1, having Formula (XIII 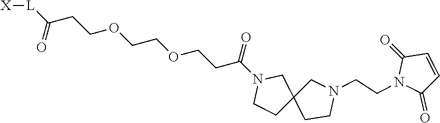
or a pharmaceutically acceptable salt thereof, wherein:
L is a linker, and
X is a payload.

16. The compound of claim 1, having Formula (IVX): 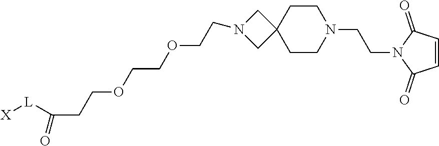
or a pharmaceutically acceptable salt thereof, wherein:
L is a linker, and
X is a payload.

17. The compound of claim 1, having Formula (XV): 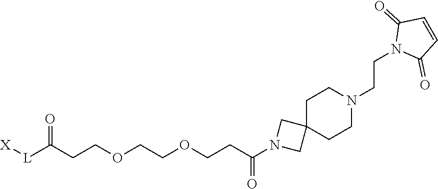
or a pharmaceutically acceptable salt thereof, wherein:
L is a linker, and
X is a payload.

18. The compound of claim 1, having Formula (XVI): 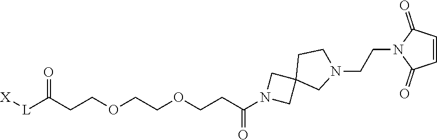
or a pharmaceutically acceptable salt thereof, wherein:
L is a linker, and
X is a payload.

19. The compound of claim 1, wherein L is selected from:

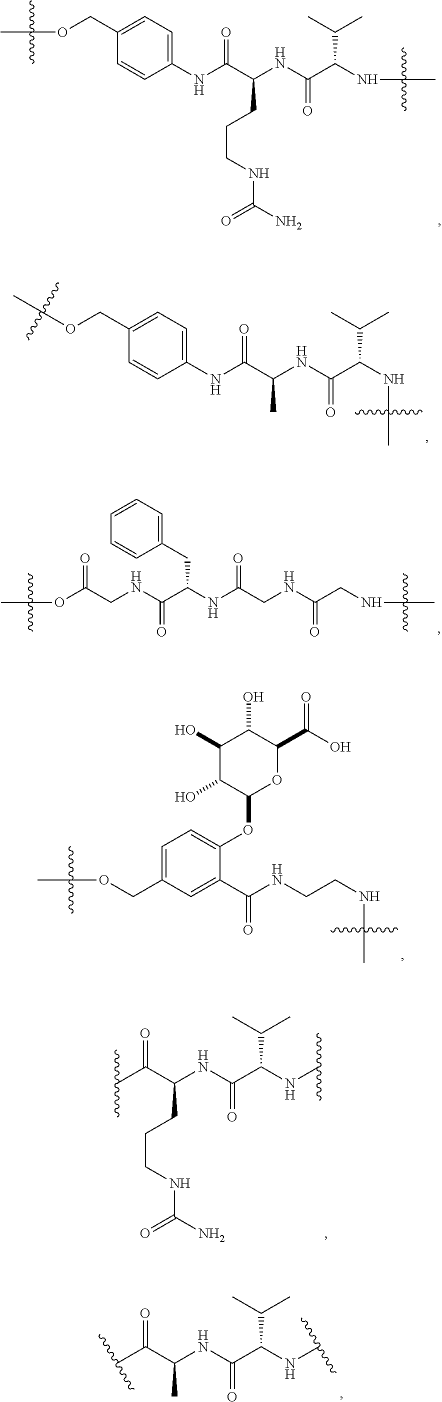

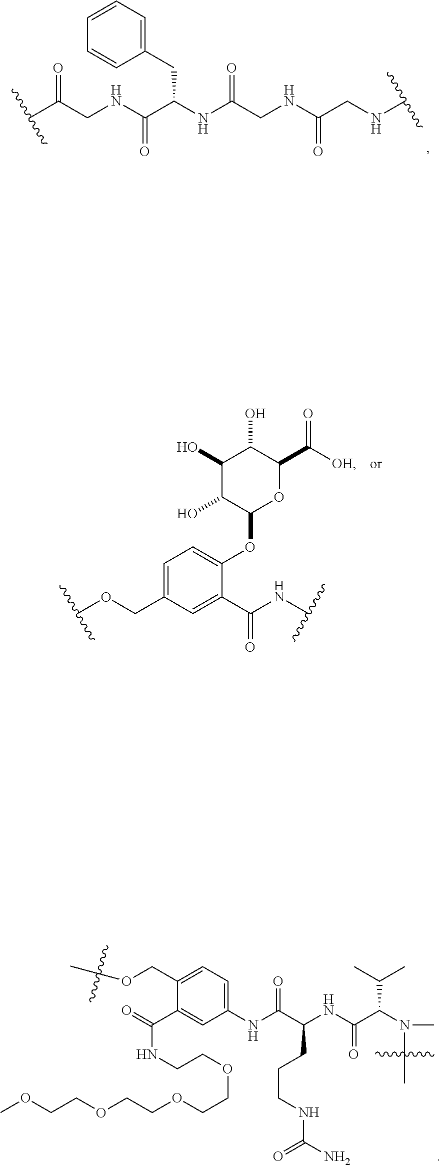

20. The compound of claim 1, wherein the small molecule is selected from exatecan, DXd, MMAE, MMAF, 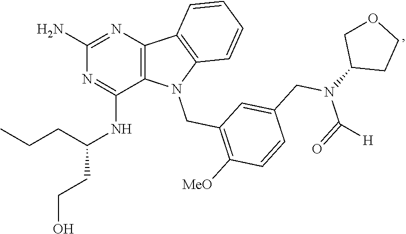 [SMILES: CCC[C@@H](CCO)Nc1nc(N)nc2c3ccccc3n(Cc3cc(CN(C=O)[C@H]4CCOC4)ccc3OC)c12]
and eribulin.

21. A compound selected from: 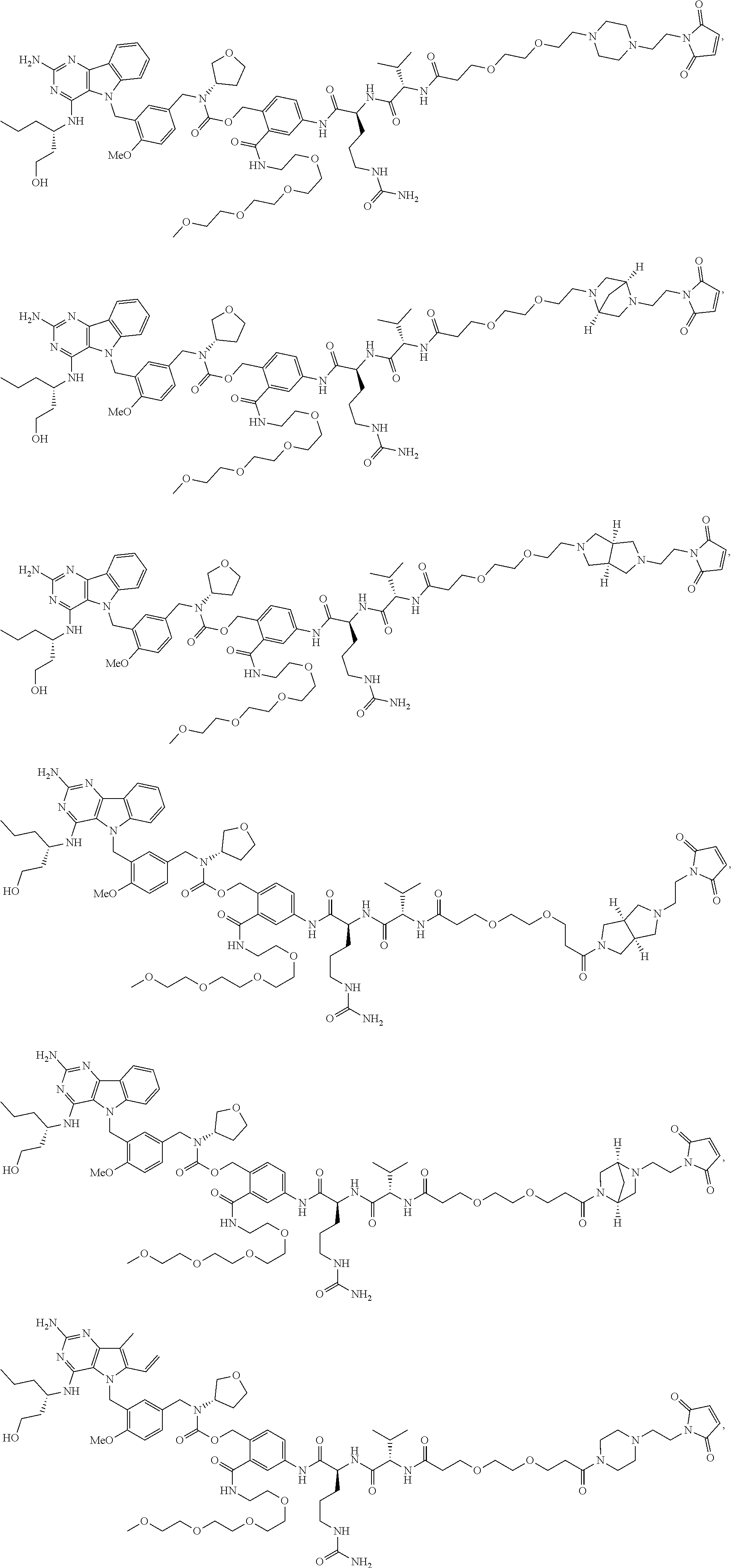 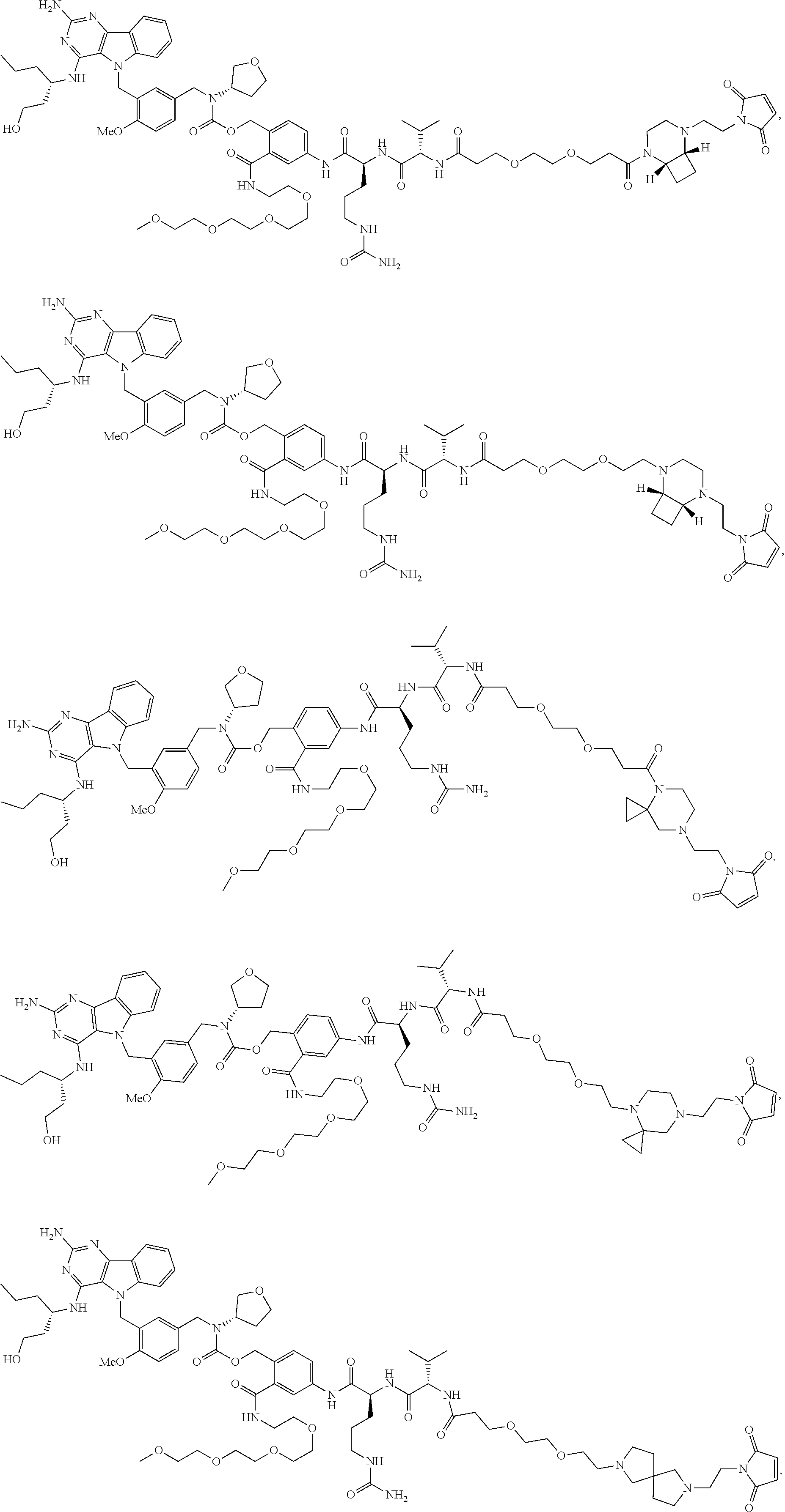 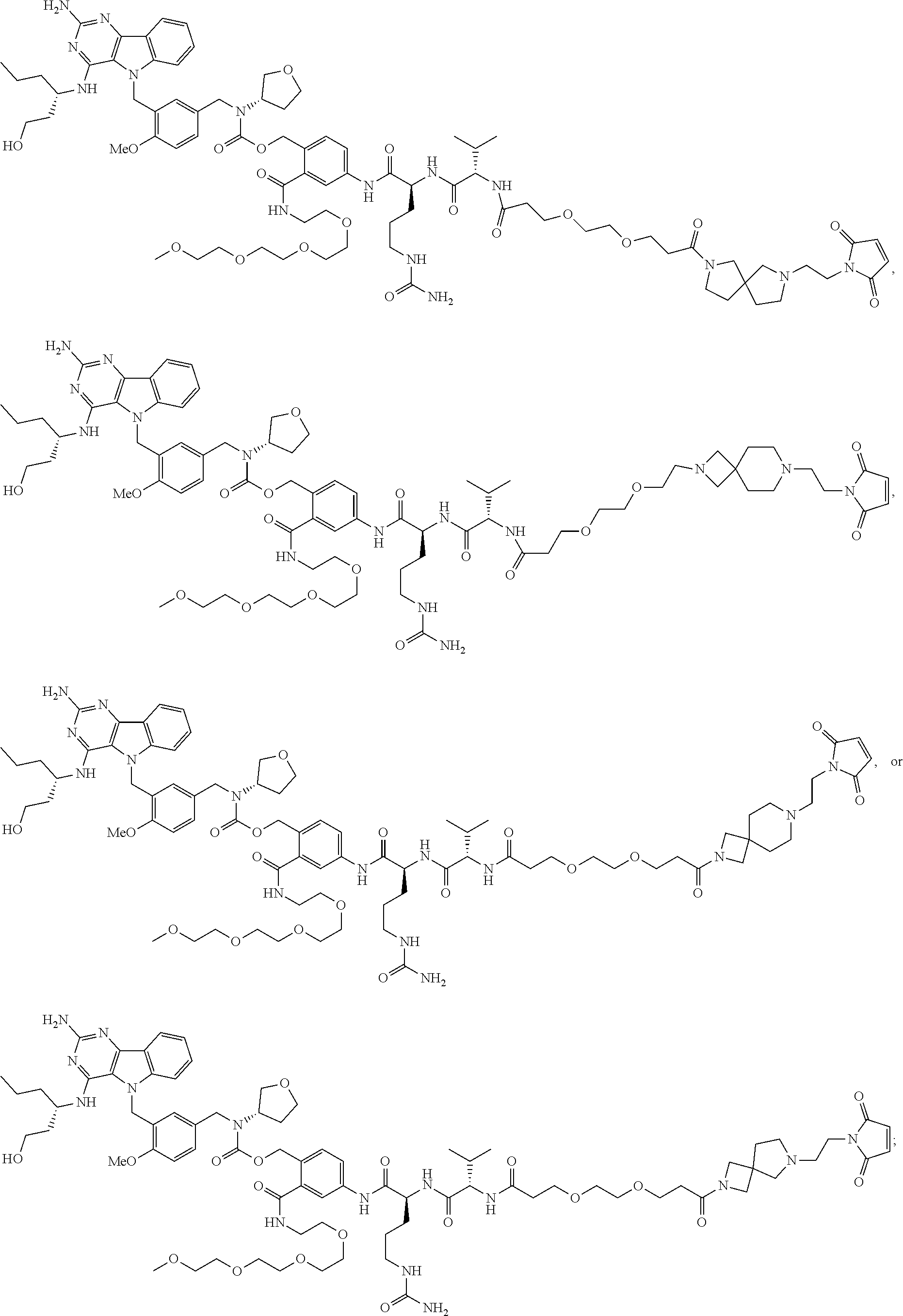
or a pharmaceutically acceptable salt thereof.

22. An antibody-drug conjugate, of Formula XVII: 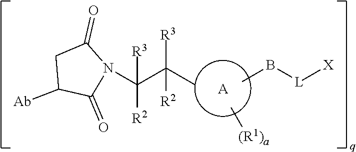
or a pharmaceutically acceptable salt thereof, wherein:
the maleimide ring may be a mixture of a hydrolyzed and a non-hydrolyzed ring;
A is selected from an unsubstituted or substituted 4 to 12 membered heterocyclic or heterospirocyclic ring;
B is independently selected from a bond, —CH₂—, —C═O—, —O—, —(—(CR¹¹R¹²)ₙ—)—O)ₘ—, —C═O—(CR¹¹R¹²)ₙ—O—)ₘ—, —C═O—(NR¹¹)—, —S—, —SO—, —SO₂—, or —SO₂—(NR¹¹)—;
R¹ is independently selected from hydrogen, halogen, —OR¹¹, —N(R¹¹R¹²)—NHR¹¹, —(—(CH₂)₂—)ₙ-O—)ₘ, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —N(R¹¹R¹²)—NHR¹¹ or —OR¹¹;
R² is independently selected from hydrogen, halogen, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —NHR¹¹ or —OR¹¹;
R³ is independently selected from hydrogen, halogen, —C₁-C₆ alkyl, a 3 to 6 membered cycloalkyl, a 4 to 12 membered heterocyclic, a 5 to 12 membered aryl or a 5 to 12 membered heteroaryl ring, wherein the alkyl, cycloalkyl, heterocyclic and heteroaryl may be optionally substituted with —R¹¹, —NHR¹¹ or —OR¹¹;
wherein two of any R¹, R² or R³ substituents together with the carbon atoms they are attached to, may join to form a 5 or 6 membered ring that may be unsaturated, saturated, partially saturated;
and may further optionally be substituted with 1 or 2 R¹¹ substituents;
R¹¹ is independently selected from hydrogen, halogen, —C₁-C₆ alkyl, —C₂-C₆ alkenyl, —C₁-C₆ haloalkyl, —(—(CH₂)ₙ—)—O—)ₘ, a 3 to 12 membered cycloalkyl, a 4 to 12 membered heterocyclic, a 5 to 12 membered aryl or a 5 to 12 membered heteroaryl ring;
R¹² is independently selected from hydrogen, halogen, —C₁-C₆ alkyl, —C₂-C₆ alkenyl, —C₁-C₆ haloalkyl, —(—(CH₂)ₙ—)—O—)ₘ, a 3 to 12 membered cycloalkyl, a 4 to 12 membered heterocyclic, a 5 to 12 membered aryl or a 5 to 12 membered heteroaryl ring;
wherein the alkyl, alkenyl, haloalkyl, cycloalkyl, heterocyclic, aryl or heteroaryl rings in R¹¹ and R¹² are each independently unsubstituted or substituted with 1, 2, or 3 R¹³ substituents;
R¹³ is independently hydrogen, halo, —C₁-C₆ alkyl, —C₁-C₆ haloalkyl, —C₁-C₆ alkoxyalkyl, oxo, —CN, —NR¹⁴R¹⁴, —CH₃—NH—CH₂—C(═O)—OH, hydroxyl or —C₁-C₆ alkoxy;
R¹⁴ is independently hydrogen, —C₁-C₆ alkyl, —C₁-C₆ haloalkyl, or —C₁-C₆ alkoxyalkyl;
L is a linker;
X is a payload;
a is 0, 1, 2, 3, or 4;
m is an integer selected from 0 to 36;
n is 1, 2, 3, 4, 5, 6, 7, 8, 9, 10, 11 or 12;
q is 1, 2, 3, 4, 5, 6, 7 or 8; and further
wherein the heterocyclic and heteroaryl cyclic ring in each A, R¹, R², R³, R¹¹, and R¹² may include 1, 2 or 3 heteroatoms independently selected from O, N or S; and
Ab is an antibody or antigen binding fragment thereof.

23. The antibody-drug conjugate of claim 22, wherein the antibody is an anti-LIV1 antibody, or antigen binding fragment thereof, comprising heavy and light chain variable regions of mAb 5A11 comprising:
a. CDRH1 consisting of SEQ ID NO: 1;
b. CDRH2 consisting of SEQ ID NO: 2;
c. CDRH3 consisting of SEQ ID NO: 3;
d. CDRL1 consisting of SEQ ID NO: 6;
e. CDRL2 consisting of SEQ ID NO: 7; and
f. CDRL3 consisting of SEQ ID NO: 8.

24. The antibody-drug conjugate of claim 22, wherein L is selected from:

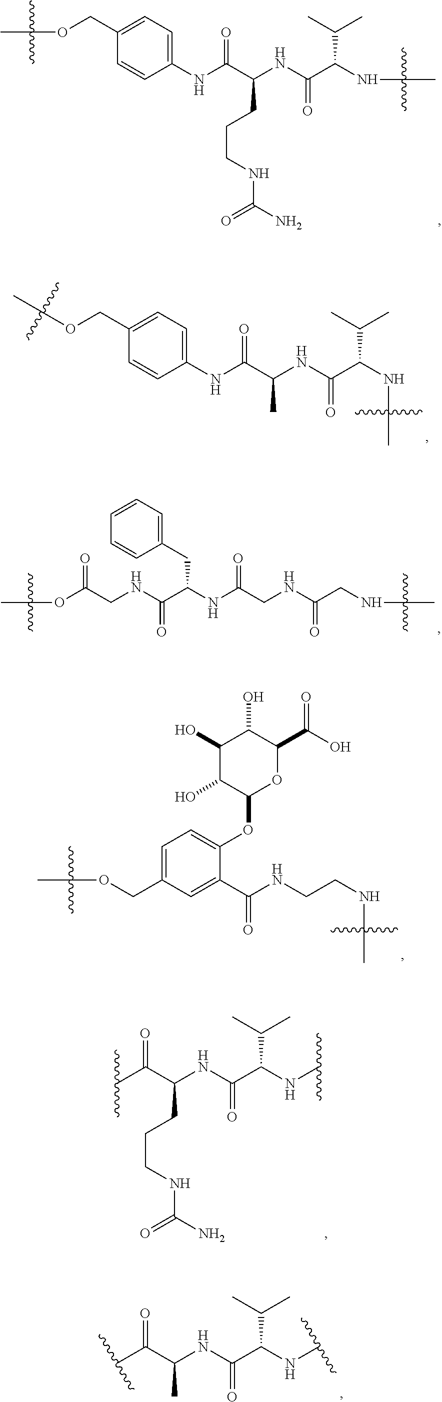

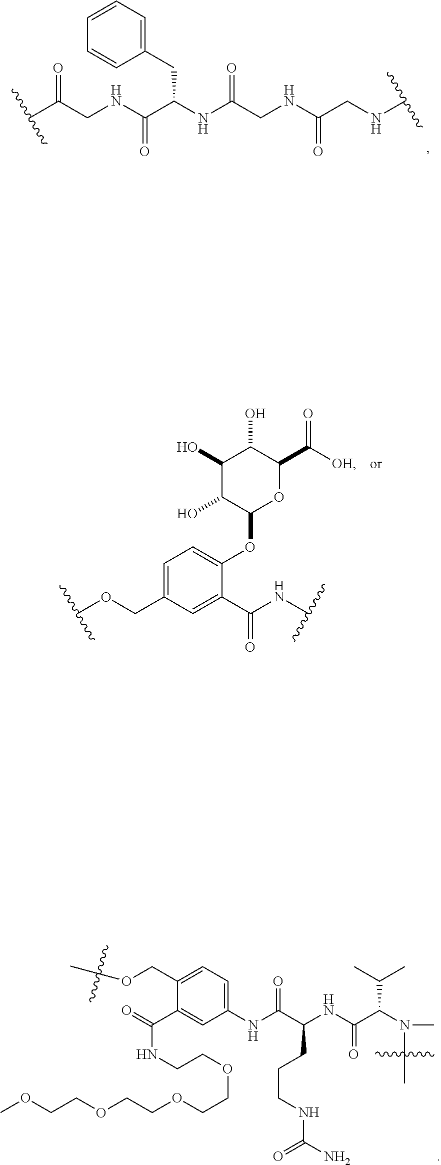

25. The antibody-drug conjugate of claim 22, having Formula XVIII, selected from: 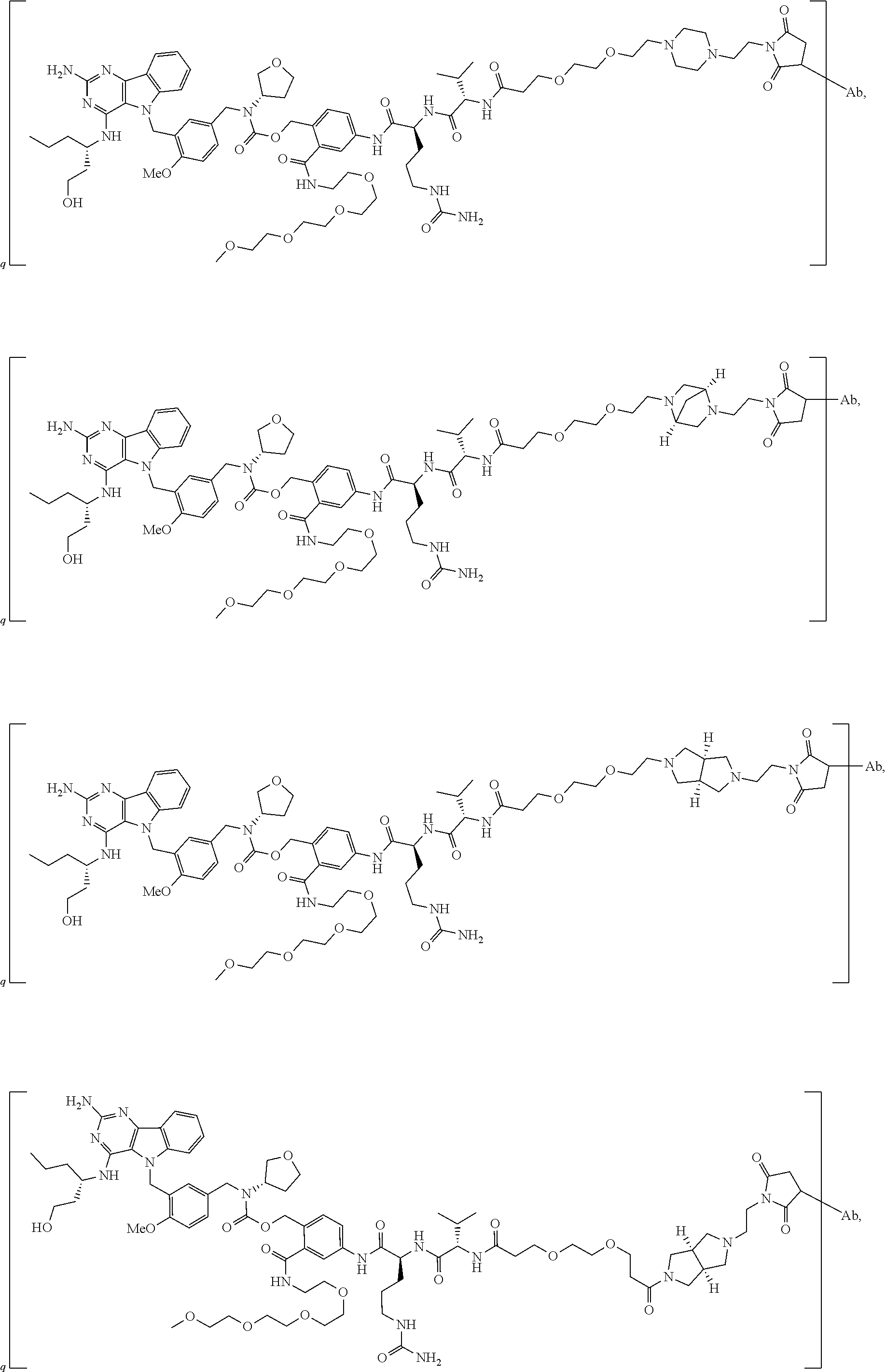 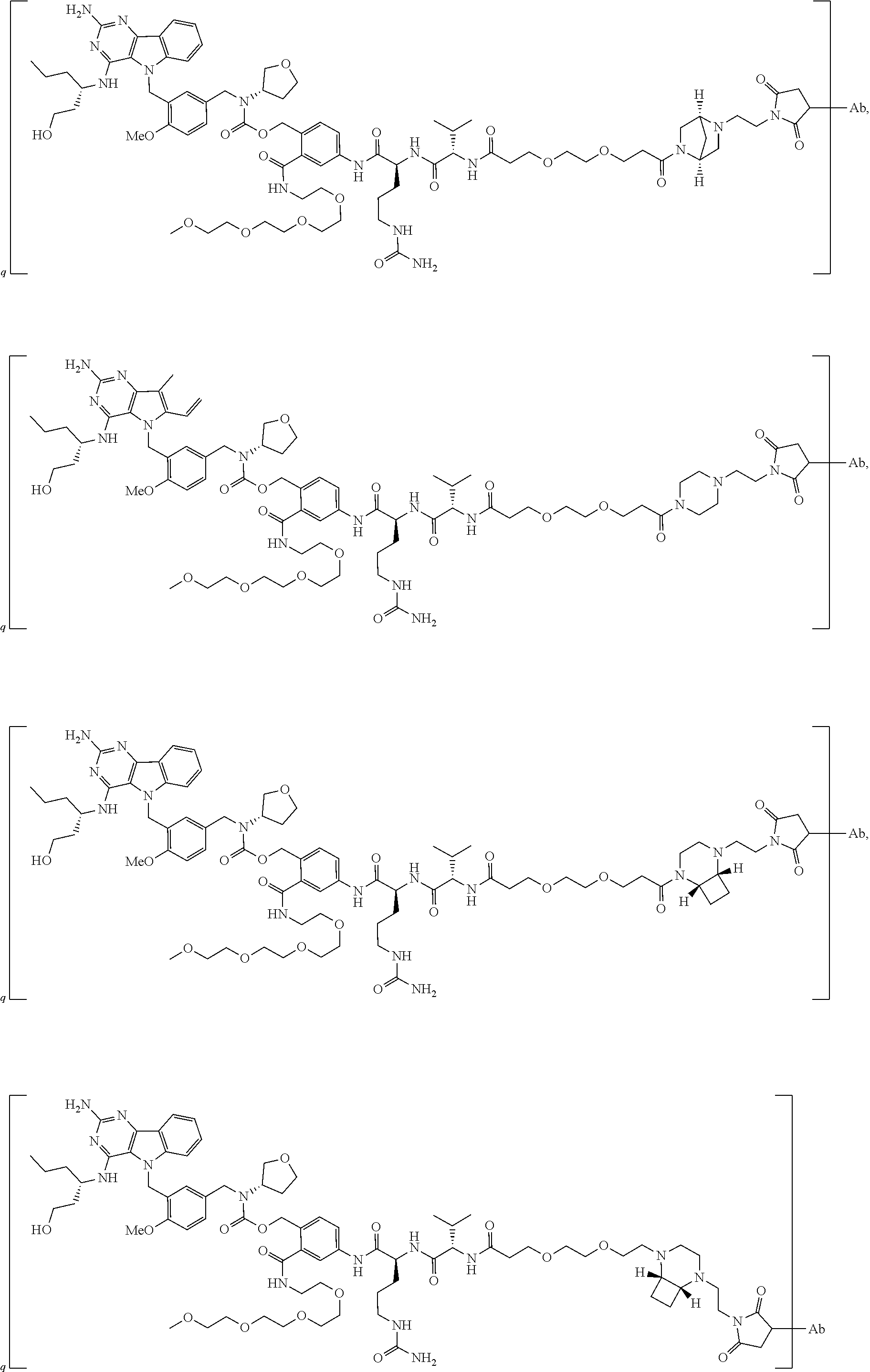 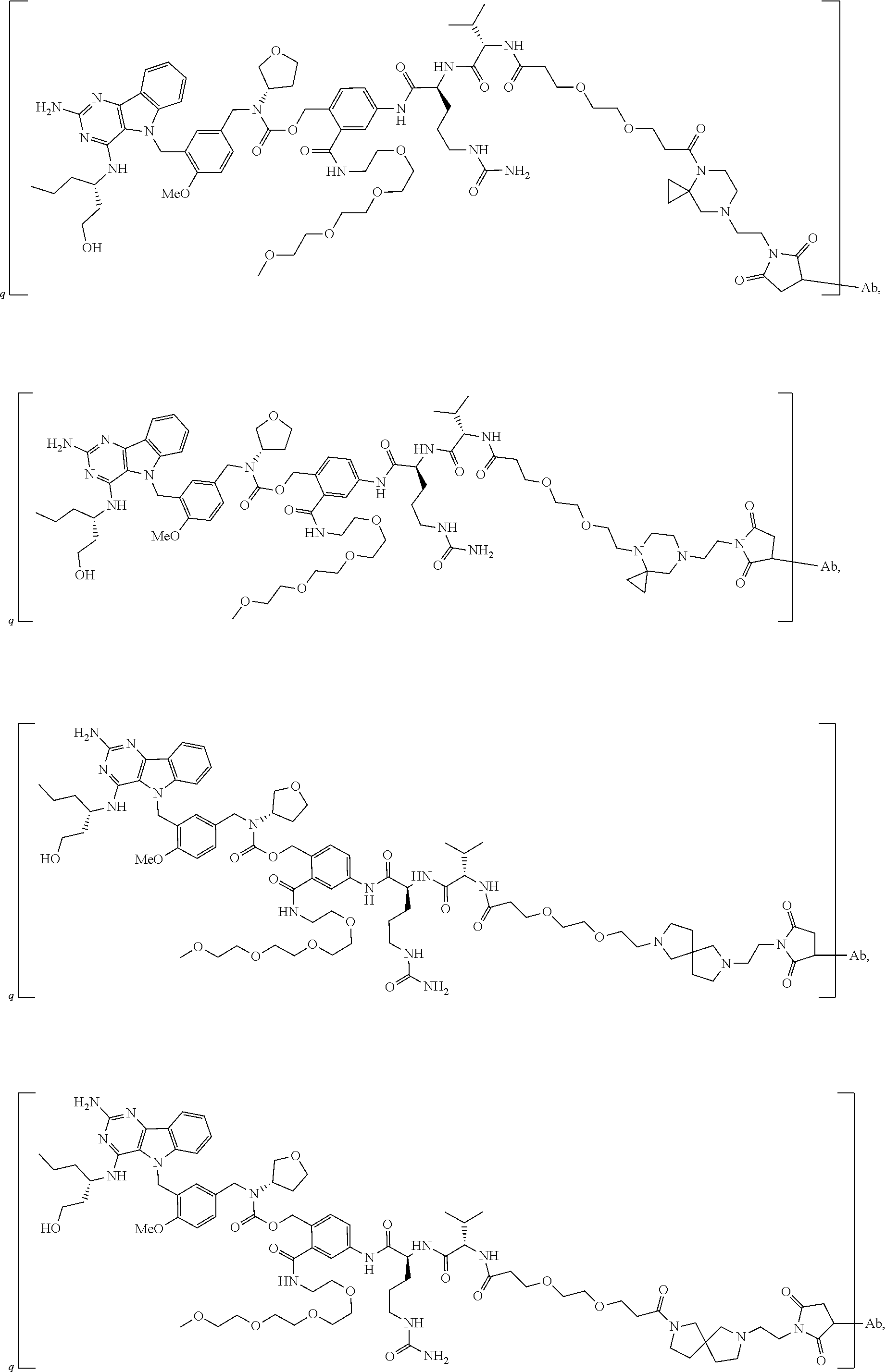 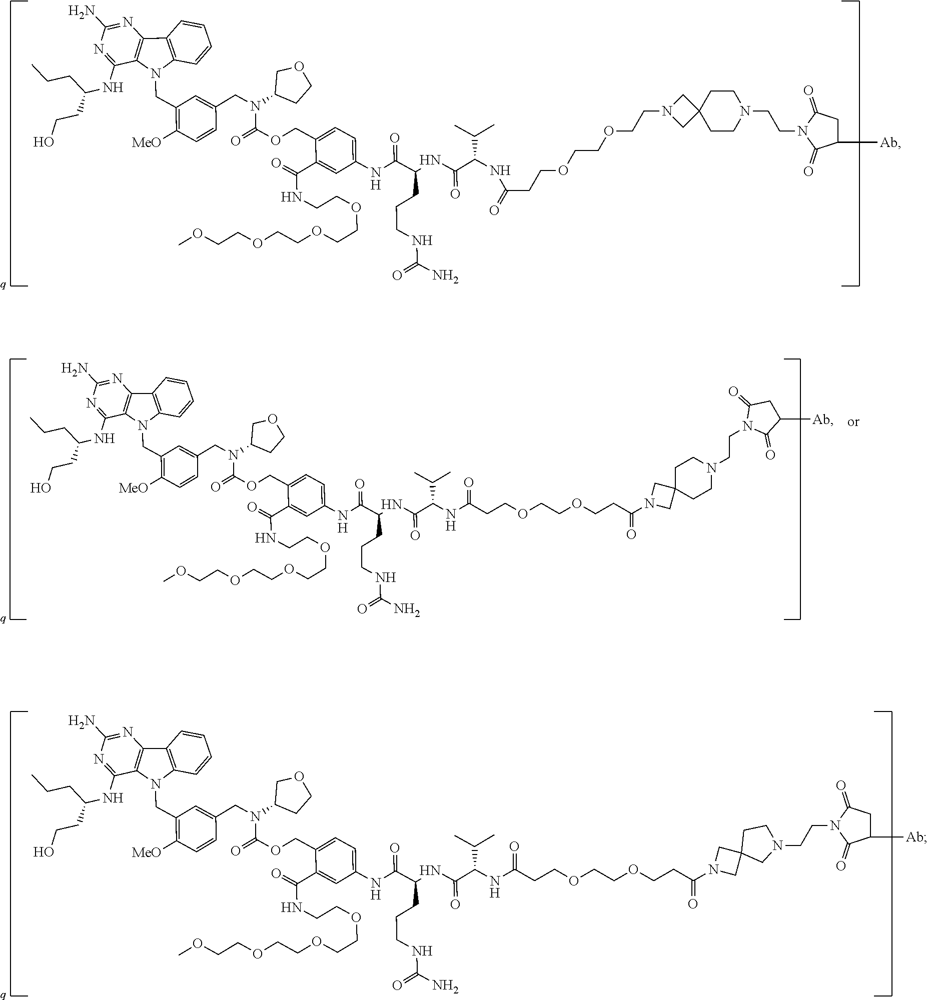
or a pharmaceutically acceptable salt thereof;
wherein q is 1, 2, 3, 4, 5, 6, 7 or 8; and
Ab is an antibody or antigen binding fragment thereof.

26. A pharmaceutical composition comprising the antibody-drug conjugate of claim 1 and a pharmaceutically acceptable carrier.

27. A method of treating cancer in a subject in need thereof, comprising administering to the subject a therapeutically effective amount of the antibody-drug conjugate of claim 1.

28. The method of claim 27, wherein the cancer is leukemia.

29. The method of claim 27, wherein the cancer is solid tumor.

30. The method of claim 27, wherein the subject is a human.


SEQUENCE LISTING (15 sequences)

SEQ ID NO: 1 - protein, 5 aa, Homo sapiens
AYYWS

SEQ ID NO: 2 - protein, 16 aa, Homo sapiens
EINPSGRIKYNPSLKS

SEQ ID NO: 3 - protein, 6 aa, Homo sapiens
TGDLDY

SEQ ID NO: 4 - protein, 114 aa, Homo sapiens
QVQLQQWGAGLLKPSETLSLTCAVYGGSFSAYYWSWIRQSPGKGLEYIGEINPSGRIKYNPSLKSRVTISLDTSTNQFSLNLNSVTAADTAVYYCAETGDLDYWGQGTLVTVSS

SEQ ID NO: 5 - protein, 443 aa, Homo sapiens
QVQLQQWGAGLLKPSETLSLTCAVYGGSFSAYYWSWIRQSPGKGLEYIGEINPSGRIKYNPSLKSRVTISLDTSTNQFSLNLNSVTAADTAVYYCAETGDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 16 aa, Homo sapiens
RSSQSLVYSDGKIYLN

SEQ ID NO: 7 - protein, 7 aa, Homo sapiens
RVSNRES

SEQ ID NO: 8 - protein, 9 aa, Homo sapiens
MQGTHWPFT

SEQ ID NO: 9 - protein, 112 aa, Homo sapiens
EIVLTQSPLSLPVTLGQPASISCRSSQSLVYSDGKIYLNWFQQRPGQSPRRLFYRVSNRESGVPDRFSGSGSGTDFTLKISRVEAEDVGIYYCMQGTHWPFTFGPGTRVEIK

SEQ ID NO: 10 - protein, 219 aa, Homo sapiens
EIVLTQSPLSLPVTLGQPASISCRSSQSLVYSDGKIYLNWFQQRPGQSPRRLFYRVSNRESGVPDRFSGSGSGTDFTLKISRVEAEDVGIYYCMQGTHWPFTFGPGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 755 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSEHEHHSDHDHHSHHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 12 - protein, 433 aa, Homo sapiens
MGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVSF

SEQ ID NO: 13 - protein, 15 aa, Homo sapiens
DPRNSQGKGAHRPEH

SEQ ID NO: 14 - protein, 15 aa, Homo sapiens
RNSQGKGAHRPEHAS

SEQ ID NO: 15 - protein, 15 aa, Homo sapiens
SQGKGAHRPEHASGR